SEQUENCE LISTING (155 sequences)

SEQ ID NO: 1 - protein, 348 aa, Homo sapiens
MAATLGPLGSWQQWRRCLSARDGSRMLLLLLLLGSGQGPQQVGAGQTFEYLKREHSLSKPYQGVGTGSSSLWNLMGNAMVMTQYIRLTPDMQSKQGALWNRVPCFLRDWELQVHFKIHGQGKKNLHGDGLAIWYTKDRMQPGPVFGNMDKFVGLGVFVDTYPNEEKQQERVFPYISAMVNNGSLSYDHERDGRPTELGGCTAIVRNLHYDTFLVIRYVKRHLTIMMDIDGKHEWRDCIEVPGVRLPRGYYFGTSSITGDLSDNHDVISLKLFELTVERTPEEEKLHRDVFLPSVDNMKLPEMTAPLPPLSGLALFLIVFFSLVFSVFAIVIGIILYNKWQEQSRKRFY

SEQ ID NO: 2 - protein, 194 aa, Homo sapiens
MGMSSLKLLKYVLFFFNLLFWICGCCILGFGIYLLIHNNFGVLFHNLPSLTLGNVFVIVGSIIMVVAFLGCMGSIKENKCLLMSFFILLLIILLAEVTLAILLFVYEQKLNEYVAKGLTDSIHRYHSDNSTKAAWDSIQSFLQCCGINGTSDLDSGSPASCPSDRKVEGCYAKEDFGFIQFPVYRNHHHLCMCD

SEQ ID NO: 3 - protein, 342 aa, Homo sapiens
MSLHGKRKEIYKYEAPWTVYAMNWSVRPDKRFRLALGSFVEEYNNKVQLVGLDEESSEFICRNTFDHPYPTTKLMWIPDTKGVYPDLLATSGDYLRVWRVGETETRLECLLNNNKNSDFCAPLTSFDWNEVDPYLLGTSSIDTTCTIWGLETGQVLGRVNLVSGHVKTQLIAHDKEVYDIAFSRAGGGRDMFASVGADGSVRMFDLRHLEHSTIIYEDPQHHPLLRLCWNKQDPNYLATMAMDGMEVVILDVRVPCTPVARLNNHRACVNGIAWAPHSSCHICTAADDHQALIWDIQQMPRAIEDPILAYTAEGEINNVQWASTQPDWIAICYNNCLEILRV

SEQ ID NO: 4 - protein, 656 aa, Homo sapiens
MEELDGEPTVTLIPGVNSKKNQMYFDWGPGEMLVCETSFNKKEKSEMVPSCPFIYIIRKDVDVYSQILRKLFNESHGIFLGLQRIDEELTGKSRKSQLVRVSKNYRSVIRACMEEMHQVAIAAKDPANGRQFSSQVSILSAMELIWNLCEILFIEVAPAGPLLLHLLDWVRLHVCEVDSLSADVLGSENPSKHDSFWNLVTILVLQGRLDEARQMLSKEADASPASAGICRIMGDLMRTMPILSPGNTQTLTELELKWQHWHEECERYLQDSTFATSPHLESLLKIMLGDEAALLEQKELLSNWYHFLVTRLLYSNPTVKPIDLHYYAQSSLDLFLGGESSPEPLDNILLAAFEFDIHQVIKECSIALSNWWFVAHLTDLLDHCKLLQSHNLYFGSNMREFLLLEYASGLFAHPSLWQLGVDYFDYCPELGRVSLELHIERIPLNTEQKALKVLRICEQRQMTEQVRSICKILAMKAVRNNRLGSALSWSIRAKDAAFATLVSDRFLRDYCERGCFSDLDLIDNLGPAMMLSDRLTFLGKYREFHRMYGEKRFADAASLLLSLMTSRIAPRSFWMTLLTDALPLLEQKQVIFSAEQTYELMRCLEDLTSRRPVHGESDTEQLQDDDIETTKVEMLRLSLARNLARAIIREGSLEGS

SEQ ID NO: 5 - protein, 236 aa, Homo sapiens
MAPDPWFSTYDSTCQIAQEIAEKIQQRNQYERKGEKAPKLTVTIRALLQNLKEKIALLKDLLLRAVSTHQITQLEGDRRQNLLDDLVTRERLLLASFKNEGAEPDLIRSSLMSEEAKRGAPNPWLFEEPEETRGLGFDEIRQQQQKIIQEQDAGLDALSSIISRQKQMGQEIGNELDEQNEIIDDLANLVENTDEKLRNETRRVNMVDRKSASCGMIMVILLLLVAIVVVAVWPTN

SEQ ID NO: 6 - protein, 195 aa, Homo sapiens
MLLDTVQKVFQKMLECIARSFRKQPEEGLRLLYSVQRPLHEFITAVQSRHTDTPVHRGVLSTLIAGPVVEISHQLRKVSDVEELTPPEHLSDLPPFSRCLIGIIIKSSNVVRSFLDELKACVASNDIEGIVCLTAAVHIILVINAGKHKSSKVREVAATVHRKLKTFMEITLEEDSIERFLYESSSRTLGELLNS

SEQ ID NO: 7 - protein, 608 aa, Homo sapiens
MTKTDETTLVASWETREKTAKTTLFLPLEFWSYKAEVPHLPELAYSARSKMAELNTHVNVKEKIYAVRSVVPNKSNNEIVLVLQQFDFNVDKAVQAFVDGSAIQVLKEWNMTGKKKNNKRKRSKSKQHQGNKDAKDKVERPEAGPLQPQPPQIQNGPMNGCEKDSSSTDSANEKPALIPREKKISILEEPSKALRGVTEGNRLLQQKLSLDGNPKPIHGTTERSDGLQWSAEQPCNPSKPKAKTSPVKSNTPAAHLEIKPDELAKKRGPNIEKSVKDLQRCTVSLTRYRVMIKEEVDSSVKKIKAAFAELHNCIIDKEVSLMAEMDKVKEEAMEILTARQKKAEELKRLTDLASQMAEMQLAELRAEIKHFVSERKYDEELGKAARFSCDIEQLKAQIMLCGEITHPKNNYSSRTPCSSLLPLLNAHAATSGKQSNFSRKSSTHNKPSEGKAANPKMVSSLPSTADPSHQTMPANKQNGSSNQRRRFNPQYHNNRLNGPAKSQGSGNEAEPLGKGNSRHEHRRQPHNGFRPKNKGGAKNQEASLGMKTPEAPAHSEKPRRRQHAADTSEARPFRGSVGRVSQCNLCPTRIEVSTDAAVLSVPAVTLVA

SEQ ID NO: 8 - protein, 267 aa, Homo sapiens
MVISWHLASDMDCVVTLTTDAARRIYDETQGRQQVLPLDSIYKKTLPDWKRSLPHFRNGKLYFKPIGDPVFARDLLTFPDNVEHCETVFGMLLGDTIILDNLDAANHYRKEVVKITHCPTLLTRDGDRIRSNGKFGGLQNKAPPMDKLRGMVFGAPVPKQCLILGEQIDLLQQYRSAVCKLDSVNKDLNSQLEYLRTPDMRKKKQELDEHEKNLKLIEEKLGMTPIRKCNDSLRHSPKVETTDCPVPPKRMRREATRQNRIITKTDV

SEQ ID NO: 9 - protein, 285 aa, Homo sapiens
MVMRPLWSLLLWEALLPITVTGAQVLSKVGGSVLLVAARPPGFQVREAIWRSLWPSEELLATFFRGSLETLYHSRFLGRAQLHSNLSLELGPLESGDSGNFSVLMVDTRGQPWTQTLQLKVYDAVPRPVVQVFIAVERDAQPSKTCQVFLSCWAPNISEITYSWRRETTMDFGMEPHSLFTDGQVLSISLGPGDRDVAYSCIVSNPVSWDLATVTPWDSCHHEAAPGKASYKDVLLVVVPVSLLLMLVTLFSAWHWCPCSGKKKKDVHADRVGPETENPLVQDLP

SEQ ID NO: 10 - protein, 76 aa, Homo sapiens [sequence omitted]

SEQ ID NO: 11 - protein, 147 aa, Homo sapiens [sequence omitted]

SEQ ID NO: 12 - protein, 261 aa, Homo sapiens
MELLQVTILFLLPSICSSNSTGVLEAANNSLVVTTTKPSITTPNTESLQKNVVTPTTGTTPKGTITNELLKMSLMSTATFLTSKDEGLKATTTDVRKNDSIISNVTVTSVTLPNAVSTLQSSKPKTETQSSIKTTEIPGSVLQPDASPSKTGTLTSIPVTIPENTSQSQVIGTEGGKNASTSATSRSYSSIILPVVIALIVITLSVFVLVGLYRMCWKADPGTPENGNDQPQSDKESVKLLTVKTISHESGEHSAQGKTKN

SEQ ID NO: 13 - protein, 213 aa, Homo sapiens
MAGCPADRSILAPLAWDLGLLLLFVGQHSLMAAERVKAWTSRYFGVLQRSLYVACTALALQLVMRYWEPIPKGPVLWEARAEPWATWVPLLCFVLHVISWLLIFSILLVFDYAELMGLKQVYYHVLGLGEPLALKSPRALRLFSHLRHPVCVELLTVLWVVPTLGTDRLLLAFLLTLYLGLAHGLDQQDLRYLRAQLQRKLHLLSRPQDGEAE

SEQ ID NO: 14 - protein, 67 aa, Homo sapiens
MQPRPRGRPPRTRGDEAPQWHLPDAAALLPVRLPLAVLVRGTQRPERRRCGRLPAGVPGAARSVARS

SEQ ID NO: 15 - protein, 161 aa, Homo sapiens
MLAPQRTRAPSPRAAPRPTRSMLPAAMKGLGLALLAVLLCSAPAHGLWCQDCTLTTNSSHCTPKQCQPSDTVCASVRITDPSSSRKDHSVNKMCASSCDFVKRHFFSDYLMGFINSGILKVDVDCCEKDLCNGAAGAGHSPWALAGGLLLSLGPALLWAGP

SEQ ID NO: 16 - protein, 141 aa, Homo sapiens [sequence omitted]

SEQ ID NO: 17 - protein, 152 aa, Homo sapiens
MEDEEVAESWEEAADSGEIDRRLEKKLKITQKESRKSKSPPKVPIVIQDDSLPAGPPPQIRILKRPTSNGVVSSPNSTSRPTLPVKSLAQREAEYAEARKRILGSASPEEEQEKPILDRPTRISQPEDSRQPNNVIRQPLGPDGSQGFKQRR

SEQ ID NO: 18 - protein, 742 aa, Homo sapiens
MASVHESLYFNPMMTNGVVHANVFGIKDWVTPYKIAVLVLLNEMSRTGEGAVSLMERRRLNQLLLPLLQGPDITLSKLYKLIEESCPQLANSVQIRIKLMAEGELKDMEQFFDDLSDSFSGTEPEVHKTSVVGLFLRHMILAYSKLSFSQVFKLYTALQQYFQNGEKKTVEDADMELTSRDEGERKMEKEELDVSVREEEVSCSGPLSQKQAEFFLSQQASLLKNDETKALTPASLQKELNNLLKFNPDFAEAHYLSYLNNLRVQDVFSSTHSLLHYFDRLILTGAESKSNGEEGYGRSLRYAALNLAALHCRFGHYQQAELALQEAIRIAQESNDHVCLQHCLSWLYVLGQKRSDSYVLLEHSVKKAVHFGLPRAFAGKTANKLMDALKDSDLLHWKHSLSELIDISIAQKTAIWRLYGRSTMALQQAQMLLSMNSLEAVNAGVQQNNTESFAVALCHLAELHAEQGCFAAASEVLKHLKERFPPNSQHAQLWMLCDQKIQFDRAMNDGKYHLADSLVTGITALNSIEGVYRKAVVLQAQNQMSEAHKLLQKLLVHCQKLKNTEMVISVLLSVAELYWRSSSPTIALPMLLQALALSKEYRLQYLASETVLNLAFAQLILGIPEQALSLLHMAIEPILADGAILDKGRAMFLVAKCQVASAASYDQPKKAEALEAAIENLNEAKNYFAKVDCKERIRDVVYFQARLYHTLGKTQERNRCAMLFRQLHQELPSHGVPLINHL

SEQ ID NO: 19 - protein, 805 aa, Homo sapiens
MDGILDESLLETCPIQSPLQVFAGMGGLALIAERLPMLYPEVIQQVSAPVVTSTTQEKPYDSDQFEWVTIEQSGELVYEAPETVAAEPPPIKSAVQTMSPIPAHSLAAFGLFLRLPGYAEVLLKERKHAQCLLRLVLGVTDDGEGSHILQSPSANVLPTLPFHVLRSLFSTTPLTTDDGVLLRRMALEIGALHLILVCLSALSHHSPRVPNSSVNQTEPQVSSSHNPTSTEEQQLYWAKGTGFGTGSTASGWDVEQALTKQRLEEEHVTCLLQVLASYINPVSSAVNGEAQSSHETRGQNSNALPSVLLELLSQSCLIPAMSSYLRNDSVLDMARHVPLYRALLELLRAIASCAAMVPLLLPLSTENGEEEEEQSECQTSVGTLLAKMKTCVDTYTNRLRSKRENVKTGVKPDASDQEPEGLTLLVPDIQKTAEIVYAATTSLRQANQEKNWVNTPRRRLMNPKPLSVLKSLEEKYVAVMKKLQFDTFEMVSEDEDGKLGFKVNYHYMSQVKNANDANSAARARRLAQEAVTLSTSLPLSSSSSVFVRCDEERLDIMKVLITGPADTPYANGCFEFDVYFPQDYPSSPPLVNLETTGGHSVRFNPNLYNDGKVCLSILNTWHGRPEEKWNPQTSSFLQVLVSVQSLILVAEPYFNEPGYERSRGTPSGTQSSREYDGNIRQATVKWAMLEQIRNPSPCFKEVIHKHFYLKRVEIMAQCEEWIADIQQYSSDKRVGRTMSHHAAALKRHTAQLREELLKLPCPEGLDPDTDDAPEVCRATTGAEETLMHDQVKPSSSKELPSDFQL

SEQ ID NO: 20 - protein, 195 aa, Homo sapiens
MKASQCCCCLSHLLASVLLLLLLPELSGPLAVLLQAAEAAPGLGPPDPRPRTLPPLPPGPTPAQQPGRGLAEAAGPRGSEGGNGSNPVAGLETDDHGGKAGEGSVGGGLAVSPNPGDKPMTQRALTVLMVVSGAVLVYFVVRTVRMRRRNRKTRRYGVLDTNIENMELTPLEQDDEDDDNTLFDANHPRRRECAF

SEQ ID NO: 21 - protein, 161 aa, Homo sapiens [sequence omitted]

SEQ ID NO: 22 - protein, 160 aa, Homo sapiens
MDKLKKVLSGQDTEDRSGLSEVVEASSLSWSTRIKGFIACFAIGILCSLLGTVLLWVPRKGLHLFAVFYTFGNIASIGSTIFLMGPVKQLKRMFEPTRLIATIMVLLCFALTLCSAFWWHNKGLALIFCILQSLALTWYSLSFIPFARDAVKKCFAVCLA

SEQ ID NO: 23 - protein, 76 aa, Homo sapiens
MQAKYSSTRDMLDDDGDTTMSLHSQASATTRHPEPRRTEHRAPSSTWRPVALTLLTLCLVLLIGLAALGLLCKSAL

SEQ ID NO: 24 - protein, 336 aa, Homo sapiens [sequence omitted]

SEQ ID NO: 25 - protein, 150 aa, Homo sapiens
MNLWLLACLVAGFLGAWAPAVHAQGVFEDCCLAYHYPIGWAVLRRAWTYRIQEVSGSCNLPAAIFYLPKRHRKVCGNPKSREVQRAMKLLDARNKVFAKLRHNTQTFQAGPHAVKKLSSGNSKLSSSKFSNPISSSKRNVSLLISANSGL

SEQ ID NO: 26 - protein, 217 aa, Homo sapiens
MAPPALQRGQRVAAVAVGSQAVLQILSRVSGRQAPPQPSGSGGVGAGPVVVPDGGGEGPQPHPSSSQSPPDLPLKAGDTVMGKQAQRDIRLRVRAEYCEHGPALEQGVASRRPQALARQLDVFGQATAVLRSRDLGSVVCDIKFSELSYLDAFWGDYLSGALLQALRGVFLTEALREAVGREAVRLLVSVDEADYEAGRRRLLLMAEEGGRRPTEAS

SEQ ID NO: 27 - protein, 504 aa, Homo sapiens
MSQPRTPEQALDTPGDCPPGRRDEDAGEGIQCSQRMLSFSDALLSIIATVMILPVTHTEISPEQQFDRSVQRLLATRIAVYLMTFLIVTVAWAAHTRLFQVVGKTDDTLALLNLACMMTITFLPYTFSLMVTFPDVPLGIFLFCVCVIAIGVVQALIVGYAFHFPHLLSPQIQRSAHRALYRRHVLGIVLQGPALCFAAAIFSLFFVPLSYLLMVTVILLPYVSKVTGWCRDRLLGHREPSAHPVEVFSFDLHEPLSKERVEAFSDGVYAIVATLLILDICEDNVPDPKDVKERFSGSLVAALSATGPRFLAYFGSFATVGLLWFAHHSLFLHVRKATRAMGLLNTLSLAFVGGLPLAYQQTSAFARQPRDELERVRVSCTIIFLASIFQLAMWTTALLHQAETLQPSVWFGGREHVLMFAKLALYPCASLLAFASTCLLSRFSVGIFHLMQIAVPCAFLLLRLLVGLALATLRVLRGLARPEHPPPAPTGQDDPQSQLLPAPC

SEQ ID NO: 28 - protein, 320 aa, Homo sapiens
MAARLDGGFAAVSRAFHEIRARNPAFQPQTLMDFGSGTGSVTWAAHSIWGQSLREYMCVDRSAAMLVLAEKLLTGGSESGEPYIPGVFFRQFLPVSPKVQFDVVVSAFSLSDQLLTFILSCNSSLLHIFPFCEQVLVENGTKAGHSLLMDARDLVLKGKEKSPLDPRPGFVFAPCPHELPCPQLTNLACSFSQAYHPIPFSWNKKPKEEKFSMVILARGSPEEAHRWPRITQPVLKRPRHVHCHLCCPDGHMQHAVLTARRHGRYGGCDQNQWDVAGSCSPRQHLFPQGFVSLCPCQLLGRSFTCAYSVCVSSIYGSGSL

SEQ ID NO: 29 - protein, 117 aa, Homo sapiens
MDNKGIYPGAVFYHDSFTESRVVLLRIRTLVPYSPPDCPTTTTAYSPFPNHGQQIELLTEVSFRWISQPFPHRPHRETVTDCYSPNTQVKSNAGRNNSKSFNFLILLLKILTEASRF

SEQ ID NO: 30 - protein, 298 aa, Homo sapiens
MARRSRHRLLLLLLRYLVVALGYHKAYGFSAPKDQQVVTAVEYQEAILACKTPKKTVSSRLEWKKLGRSVSFVYYQQTLQGDFKNRAEMIDFNIRIKNVTRSDAGKYRCEVSAPSEQGQNLEEDTVTLEVLVAPAVPSCEVPSSALSGTVVELRCQDKEGNPAPEYTWFKDGIRLLENPRLGSQSTNSSYTMNTKTGTLQFNTVSKLDTGEYSCEARNSVGYRRCPGKRMQVDDLNISGIIAAVVVVALVISVCGLGVCYAQRKGYFSKETSFQKSNSSSKATTMSENDFKHTKSFII

SEQ ID NO: 31 - protein, 118 aa, Homo sapiens
MQHRGFLLLTLLALLALTSAVAKKQDKVKKGGPGSECAEWAWGPCTPSSKGFAAVGFPRGPPWGGPRTQPAVLVERVAPGKLERKEFWAPGLWKVGQIFWKKTWRVCRSVKWGRGQKN

SEQ ID NO: 32 - protein, 248 aa, Homo sapiens
MQTCPLAFPGHVSQALGTLLFLAASLSAQNEGWDSPICTEGVVSVSWGENTVMSCNISNAFSHVNIKLRAHGQESAIFNEVAPGYFSRDGWQLQVQGGVAQLVIKGARDSHAGLYMWHLVGHQRNNRQVTLEVSGAEPQSAPDTGFWPVPAVVTAVFILLVALVMFAWYRCRCSQQRREKKFFLLEPQMKVAALRAGAQQGLSRASAELWTPDSEPTPRPLALVFKPSPLGALELLSPQPLFPYAADP

SEQ ID NO: 33 - protein, 150 aa, Homo sapiens
MLEEGSFRGRTADFVFMFLFGGVLMTVSFPQALEPRARAPRRPACVGPGANTAMPERDTVAVSSLAPFLPWALMGFSLLLGNSILVDLLGIAVGHIYYFLEDVFPNQPGGKRLLQTPGFLKLLLDAPAEDPNYLPLPEEQPGPHLPPPQQ

SEQ ID NO: 34 - protein, 431 aa, Homo sapiens
MWALGQAGFANLTEGLKVWLGIMLPVLGIKSLSPFAITYLDRLLLMHPNLTKGFGMIGPKDFFPLLDFAYMPNNSLTPSLQEQLCQLYPRLKMLAFGAKPDSTLHTYFPSFLSRATPSCPPEMKKELLSSLTECLTVDPLSASVWRQLYPKHLSQSSLLLEHLLSSWEQIPKKVQKSLQETIQSLKLTNQELLRKGSSNNQDVVTCDMACKGLLQQVQGPRLPWTRLLLLLLVFAVGFLCHDLRSHSSFQASLTGRLLRSSGFLPASQQACAKLYSYSLQGYSWLGETLPLWGSHLLTVVRPSLQLAWAHTNATVSFLSAHCASHLAWFGDSLTSLSQRLQIQLPDSVNQLLRYLRELPLLFHQNVLLPLWHLLLEALAWAQEHCHEACRGEVTWDCMKTQLSEAVHWTWLCLQDITVAFLDWALALISQQ

SEQ ID NO: 35 - protein, 278 aa, Homo sapiens
MQWLRVRESPGEATGHRVTMGTAALGPVWAALLLFLLMCEIPMVELTFDRAVASGCQRCCDSEDPLDPAHVSSASSSGRPHALPEIRPYINITILKGDKGDPGPMGLPGYMGREGPQGEPGPQGSKGDKGEMGSPGAPCQKRFFAFSVGRKTALHSGEDFQTLLFERVFVNLDGCFDMATGQFAAPLRGIYFFSLNVHSWNYKETYVHIMHNQKEAVILYAQPSERSIMQSQSVMLDLAYGDRVWVRLFKRQRENAIYSNDFDTYITFSGHLIKAEDD

SEQ ID NO: 36 - protein, 286 aa, Homo sapiens
MEEKRRRARVQGAWAAPVKSQAIAQPATTAKSHLHQKPGQTWKNKEHHLSDREFVFKEPQQVVRRAPEPRVIDREGVYEISLSPTGVSRVCLYPGFVDVKEADWILEQLCQDVPWKQRTGIREDITYQQPRLTAWYGELPYTYSRITMEPNPHWHPVLRTLKNRIEENTGHTFNSLLCNLYRNEKDSVDWHSDDEPSLGRCPIIASLSFGATRTFEMRKKPPPEENGDYTYVERVKIPLDHGTLLIMEGATQADWQHRVPKEYHSREPRVNLTFRTVYPDPRGAPW

SEQ ID NO: 37 - protein, 404 aa, Homo sapiens
MKMEEAVGKVEELIESEAPPKASEQETAKEEDGSVELESQVQKDGVADSTVISSMPCLLMELRRDSSESQLASTESDKPTTGRVYESDPSNHCMLSPSSSGHLADSDTLSSAEENEPSQAETAVEGDPSGVSGATVGRKSRRSRSESETSTMAAKKNRQSSDKQNGRVAKVKGHRSQKHKERIRLLRQKREAAARKKYNLLQDSSTSDSDLTCDSSTSSSDDDEEVSGSSKTITAEIPDGPPVVAHYDMSDTNSDPEVVNVDNLLAAAVVQEHSNSVGGQDTGATWRTSGLLEELNAEAGHLDPGFLASDKTSAGNAPLNEEINIASSDSEVEIVGVQEHARCVHPRGGVIQSVSSWKHGSGTQYVSTRQTQSWTAVTPQQTWASPAEVVDLTLDEDSRRKYLL

SEQ ID NO: 38 - protein, 405 aa, Homo sapiens
MFVQEEKIFAGKVLRLHICASDGAEWLEEATEDTSVEKLKERCLKHCAHGSLEDPKSITHHKLIHAASERVLSDARTILEENIQDQDVLLLKKKRAPSPLPKMADVSAEEKKKQDQKAPDKEAILRATANLPSYNMDRAAVQTNMRDFQTELRKILVSLIEVAQKLLALNPDAVELFKKANAMLDEDEDERVDEAALRQLTEMGFPENRATKALQLNHMSVPQAMEWLIEHAEDPTIDTPLPGQAPPEAEGATAAASEAAAGASATDEEARDELTEIFKKIRRKREFRADARAVISLMEMGFDEKEVIDALRVNNNQQNAACEWLLGDRKPSPEELDKGIDPDSPLFQAILDNPVVQLGLTNPKTLLAFEDMLENPLNSTQWMNDPETGPVMLQISRIFQTLNRT

SEQ ID NO: 39 - protein, 177 aa, Homo sapiens
  MOD_RES (170)..(171): variable amino acid
MVMHNSDPNLHLLAEGAPIDWGEEYSNSGGGGSPAPAPRSQPPSRKSDGAPSRWSLWSRMRRWGCPLRLALSHHHLRPRTVSLRSEACWPKVCGLRAPHQPAPCSTGPPLGRVPSLRPPPRPPRRLPHPSSISCLERLWTLGPPSPATRRLESRCPAPAATPPSTPPPRXXFKGCKN

SEQ ID NO: 40 - protein, 197 aa, Homo sapiens
MITCRVCQSLINVEGKMHQHVVKCGVCNEATPIKNAPPGKKYVRCPCNCLLICKVTSQRIACPRPYCKRIINLGPVHPGPLSPEPQPMGVRVICGHCKNTFLWTEFTDRTLARCPHCRKVSSIGRRYPRKRCICCFLLGLLLAVTATGLAFGTWKHARRYGGIYAAWAFVILLAVLCLGRALYWACMKVSHPVQNFS

SEQ ID NO: 41 - protein, 302 aa, Homo sapiens [sequence omitted]

SEQ ID NO: 42 - protein, 164 aa, Homo sapiens
MRTLENQGFKILPFLGVKEVWQKQNKLISRFITCQFFLYNFLDSGSIWVQADFPPILQCGCFLFHPWTLQEIAPCFCLCITEKGSMKVAQVRPFHCPPGAGFALPILGLLQGLVILHSPLHISQVSAQKSPFGGVSTCHCVCKSSFSFFLAHLTLVMSLITTTI

SEQ ID NO: 43 - protein, 235 aa, Homo sapiens
MSPTLSSITQGVPLDTSKLSTDQRLPPYPYSSPSLVLPTQPHTPKSLQQPGLPSQSCSVQSSGGQPPGRQSHYGTPYPPGPSGHGQQSYHRPMSDFNLGNLEQFSMESPSASLVLDPPGFSEGPGFLGGEGPMGGPQDPHTFNHQNLTHCSRHGSGPNIILTGDSSPGFSKEIAAALAGVPGFEVSAAGLELGLGLEDELRMEPLGLEGLNMLSDPCALLPDPAVEESFRSDRLQ

SEQ ID NO: 44 - protein, 203 aa, Homo sapiens [sequence omitted]

SEQ ID NO: 45 - protein, 359 aa, Homo sapiens
MERGNVLSRAPSRAHGTHFGDDRFEDLEEANPFSFREFLKTKNLGLSKEDPASRIYAKEASRHSLGLDHNSPPSQTGGYGLEYQQPFFEDPTGAGDLLDEEEDEDTGWSGAYLPSAIEQTHPERVPAGTSPCSTYLSFFSTPSELAGPESLPSWALSDTDSRVSPASPAGSPSADFAVHGESLGDRHLRTLQISYDALKDENSKLRRKLNEVQSFSEAQTEMVRTLERKLEAKMIKEESDYHDLESVVQQVEQNLELMTKRAVKAENHVVKLKQEISLLQAQVSNFQRENEALRCGQGASLTVVKQNADVALQNLRVVMNSAQASIKQLVSGAETLNLVAEILKSIDRISEVKDEEEDS

SEQ ID NO: 46 - protein, 150 aa, Homo sapiens
  MOD_RES (33): variable amino acid
MGGKPHKEPRAKGPLSIFYPGSTAPVITQRTPXAALKPPPIKGAGPTIAPIKGXXNFGKRPTVTXPXWXISPNWGKRGXCXXXGIKWVXPRVSQARTFKTTANELXFXDTFEEXXRXXHAXVSXEPQPRCPLGESRSLGAAVCRWDSFDF

SEQ ID NO: 47 - protein, 402 aa, Homo sapiens
MPPVSRSSYSEDIVGSRRRRRSSSGSPPSPQSRCSSWDGCSRSHSRGREGLRPPWSELDVGALYPFSRSGSRGRLPRFRNYAFASSWSTSYSGYRYHRHCYAEERQSAEDYEKEESHRQRRLKERERIGELGAPEVWGPSPKFPQLDSDEHTPVEDEEEVTHQKSSSSDSNSEEHRKKKTSRSRNKKKRKNKSSKRKHRKYSDSDSNSESDTNSDSDDDKKRVKAKKKKKKKKHKTKKKKNKKTKKESSDSSCKDSEEDLSEATWMEQPNVADTMDLIGPEAPIIHTSQDEKPLKYGHALLPGEGAAMAEYVKAGKRIPRRGEIGLTSEEIGSFECSGYVMSGSRHRRMEAVRLRKENQIYSADEKRALASFNQEERRKRESKILASFREMVHKKTKEKDDK

SEQ ID NO: 48 - protein, 311 aa, Homo sapiens
MHPAGLAAAAAGTPRLPSKRRIPVSQPGMADPHQLFDDTSSAQSRGYGAQRAPGGLSYPAASPTPHAAFLADPVSNMAMAYGSSLAAQGKELVDKNIDRFIPITKLKYYFAVDTMYVGRKLGLLFFPYLHQDWEVQYQQDTPVAPRFDVNAPDLYIPAMAFITYVLVAGLALGTQDRFSPDLLGLQASSALAWLTLEVLAILLSLYLVTVNTDLTTIDLVAFLGYKYVGMIGGVLMGLLFGKIGYYLVLGWCCVAIFVFMIRTLRLKILADAAAEGVPVRGARNQLRMYLTMAVAAAQPMLMYWLTFHLVR

SEQ ID NO: 49 - protein, 316 aa, Homo sapiens
MASADELTFHEFEEATNLLADTPDAATTSRSDQLTPQGHVAVAVGSGGSYGAEDEVEEESDKAALLQEQQQQQQPGFWTFSYYQSFFDVDTSQVLDRIKGSLLPRPGHNFVRHHLRNRPDLYGPFWICATLAFVLAVTGNLTLVLAQRRDPSIHYSPQFHKVTVAGISIYCYAWLVPLALWGFLRWRKGVQERMGPYTFLETVCIYGYSLFVFIPMVVLWLIPVPWLQWLFGALALGLSAAGLVFTLWPVVREDTRLVATVLLSVVVLLHALLAMGCKLYFFQSLPPENVAPPPQITSLPSNIALSPTLPQSLAPS

SEQ ID NO: 50 - protein, 346 aa, Homo sapiens [sequence omitted]

SEQ ID NO: 51 - protein, 299 aa, Homo sapiens
MGTKAQVERKLLCLFILAILLCSLALGSVTVHSSEPEVRIPENNPVKLSCAYSGFSSPRVEWKFDQGDTTRLVCYNNKITASYEDRVTFLPTGITFKSVTREDTGTYTCMVSEEGGNSYGEVKVKLIVLVPPSKPTVNIPSSATIGNRAVLTCSEQDGSPPSEYTWFKDGIVMPTNPKSTRAFSNSSYVLNPTTGELVFDPLSASDTGEYSCEARNGYGTPMTSNAVRMEAVERNVGVIVAAVLVTLILLGILVFGIWFAYSRGHFDRTKKGTSSKKVIYSQPSARSEGEFKQTSSFLV

SEQ ID NO: 52 - protein, 351 aa, Homo sapiens
MASTGSQASDIDEIFGFFNDGEPPTKKPRKLLPSLKTKKPRELVLVIGTGISAAVAPQVPALKSWKGLIQALLDAAIDFDLLEDEESKKFQKCLHEDKNLVHVAHDLIQKLSPRTSNVRSTFFKDCLYEVFDDLESKMEDSGKQLLQSVLHLMENGALVLTTNFDNLLELYAADQGKQLESLDLTDEKKVLEWAQEKRKLSVLHIHGVYTNPSGIVLHPAGYQNVLRNTEVMREIQKLYENKSFLFLGCGWTVDDTTFQALFLEAVKHKSDLEHFMLVRRGDVDEFKKLRENMLDKGIKVISYGDDYADLPEYFKRLTCEISTRGTSAGMVREGQLNGSSAAHSEIRGCST

SEQ ID NO: 53 - protein, 662 aa, Homo sapiens
MTAKKQCLLRLGVLRQDWPDTNRLLGSANVVPEALQRFTRAAADFATHGKLGKLEFAQDAHGQPDVSAFDFTSMMRAESSARVQEKHGARLLLGLVGDCLVEPFWPLGTGVARGFLAAFDAAWMVKRWAEGAESLEVLAERESLYQLLSQTSPENMHRNVAQYGLDPATRYPNLNLRAVTPNQVRDLYDVLAKEPVQRDNDKTDTGMPATGSAGTQEELLRWCQEQTAGYPGVHVSDLSSSWADGLALCALVYRLQPGLLEPSELQGLGALEATAWALKVAENELGITPVVSAQAVVAGSDPLGLIAYLSHFHSAFKSMAHSPGPVSQASPGTSSAVLFLSKLQRTLQRSRAKENAEDAGGKKLRLEMEAETPSTEVPPDPEPGVPLTPPSQHQEAGAGDLCALCGEHLYVLERLCVNGHFFHRSCFRCHTCEATLWPGGYEQHPGSRTSQFFFSALVAMEKEEKESPFSSEEEEEDVPLDSDVEQALQTFAKTSGTMNNYPTWRRTLLRRAKEEEMKRFCKAQTIQRRLNEIEAALRELEAEGVKLELALRRQSSSPEQQKKLWVGQLLQLVDKKNSLVAEEAELMITVQELNLEEKQWQLDQELRGYMNREENLKTAADRQAEDQVLRKLVDLVNQRDALIRFQEERRLSELALGTGAQG

SEQ ID NO: 54 - protein, 115 aa, Homo sapiens
  MOD_RES (83): variable amino acid
MASWPASPLQWGPPLASCPSCCCCCFHCWQPRVGVACRQRCWPLRWGWWVWGPPTCSFVQPCTCPPVFSYSWPRVPHWGPSWXMSWRRRLMGVPLGLWNCLVLKLXQGLAPTSGG

SEQ ID NO: 55 - protein, 157 aa, Homo sapiens
MEALRRAHEVALRLLLCRPWASRAAARPKPSASEVLTRHLLQRRLPHWTSFCVPYSAVRNDQFGLSHFNWPVQGANYHVLRTGCFPFIKYHCSKAPWQDLARQNRFFTALKVVNLGIPTLLYGLGSWLFARVTETVHTSYGPITVYFLNKEDEGAMY

SEQ ID NO: 56 - protein, 197 aa, Homo sapiens
MPPAGLRRAAPLTAIALLVLGAPLVLAGEDCLWYLDRNGSWHPGFNCEFFTFCCGTCYHRYCCRDLTLLITERQQKHCLAFSPKTIAGIASAVILFVAVVATTICCFLCSCCYLYRRRQQLQSPFEGQEIPMTGIPVQPVYPYPQDPKAGPAPPQPGFMYPPSGPAPQYPLYPAGPPVYNPAAPPPYMPPQPSYPGA

SEQ ID NO: 57 - protein, 245 aa, Homo sapiens [sequence omitted]

SEQ ID NO: 58 - protein, 310 aa, Homo sapiens
MLLPQLCWLPLLAGLLPPVPAQKFSALTFLRVDQDKDKDCSLDCAGSPQKPLCASDGRTFLSRCEFQRAKCKDPQLEIAYRGNCKDVSRCVAERKYTQEQARKEFQQVFIPECNDDGTYSQVQCHSYTGYCWCVTPNGRPISGTAVAHKTPRCPGSVNEKLPQREGTGKTDDAAAPALETQPQGDEEDIASRYPTLWTEQVKSRQNKTNKNSVSSCDQEHQSALEEAKQPKNDNVVIPECAHGGLYKPVQCHPSTGYCWCVLVDTGRPIPGTSTRYEQPKCDNTGQGPPSQSPGPVQGPPATRLSGCQKA

SEQ ID NO: 59 - protein, 256 aa, Homo sapiens
MRPAALRGALLGCLCLALLCLGGADKRLRDNHEWKKLIMVQHWPETVCEKIQNDCRDPPDYWTIHGLWPDKSEGCNRSWPFNLEEIKDLLPEMRAYWPDVIHSFPNRSRFWKHEWEKHGTCAAQVDALNSQKKYFGRSLELYRELDLNSVLLKLGIKPSINYYQVADFKDALARVYGVIPKIQCLPPSQDEEVQTIGQIELCLTKQDQQLQNCTEPGEQPSPKQEVWLANGAAESRGLRVCEDGPVFYPPPKKTKH

SEQ ID NO: 60 - protein, 160 aa, Homo sapiens
MQFMLLFSRQGKLRLQKWYVPLSDKEKRKITRELVQTVLARKPKMCSFLEWRDLKIVYKRYASLYFCCAIEDQDNELITLEIIHRYVELLDKYFGSVCELDIIFNFEKAYFILDEFLLGGEVQETSKKNVLKAIEQADLLQEDAKEAETPRSVLEEIGLT

SEQ ID NO: 61 - protein, 341 aa, Homo sapiens [sequence omitted]

SEQ ID NO: 62 - protein, 430 aa, Homo sapiens
MAEGEDVGWWRSWLQQSYQAVKEKSSEALEFMKRDLTEFTQVVQHDTACTIAATASVVKEKLATEGSSGATEKMKKGLSDFLGVISDTFAPSPDKTIDCDVITLMGTPSGTAEPYDGTKARLYSLQSDPATYCNEPDGPPELFDAWLSQFCLEEKKGEISELLVGSPSIRALYTKMVPAAVSHSEFWHRYFYKVHQLEQEQARRDALKQRAEQSISEEPGWEEEEEELMGISPISPKEAKVPVAKISTFPEGEPGPQSPCEENLVTSVEPPAEVTPSESSESISLVTQIANPATAPEARVLPKDLSQKLLEASLEEQGLAVDVGETGPSPPIHSKPLTPAGHTGGPEPRPPARVETLREEAPTDLRVFELNSDSGKSTPSNNGKKGSSTDISEDWEKDFDLDMTEEEVQMALSKVDASGELEDVEWEDWE

SEQ ID NO: 63 - protein, 143 aa, Homo sapiens
MGPVRLGILLFLFLAVHEAWAGMLKEEDDDTERLPSKCEVCKLLSTELQAELSRTGRSREVLELGQVLDTGKRKRHVPYSVSETRLEEALENLCERILDYSVHAERKGSLRYAKGQSQTMATLKGLVQKGVKVDLGIPLELLG

SEQ ID NO: 64 - protein, 301 aa, Homo sapiens
MEDMNEYSNIEEFAEGSKINASKNQQDDGKMFIGGLSWDTSKKDLTEYLSRFGEVVDCTIKTDPVTGRSRGFGFVLFKDAASVDKVLELKEHKLDGKLIDPKRAKALKGKEPPKKVFVGGLSPDTSEEQIKEYFGAFGEIENIELPMDTKTNERRGFCFITYTDEEPVKKLLESRYHQIGSGKCEIKVAQPKEVYRQQQQQQKGGRGAAAGGRGGTRGRGRGQGQNWNQGFNNYYDQGYGNYNSAYGGDQNYSGYGGYDYTGYNYGNYGYGQGYADYSGQQSTYGKASRGGGNHQNNYQPY

SEQ ID NO: 65 - protein, 233 aa, Homo sapiens
MGEPQQVSALPPPPMQYIKEYTDENIQEGLAPKPPPPIKDSYMMFGNQFQCDDLIIRPLESQGIERLHPMQFDHKKELRKLNMSILINFLDLLDILIRSPGSIKREEKLEDLKLLFVHVHHLINEYRPHQARETLRVMMEVQKRQRLETAERFQKHLERVIEMIQNCLASLPDDLPHSEAGMRVKTEPMDADDSNNCTGQNEHQRENSGHRRDQIIEKDAALCVLIDEMNERP

SEQ ID NO: 66 - protein, 354 aa, Homo sapiens
MAGAGAGAGARGGAAAGVEARARDPPPAHRAHPRHPRPAAQPSARRMDGGSGGLGSGDNAPTTEALFVALGAGVTALSHPLLYVKLLIQVGHEPMPPTLGTNVLGRKVLYLPSFFTYAKYIVQVDGKIGLFRGLSPRLMSNALSTVTRGSMKKVFPPDEIEQVSNKDDMKTSLKKVVKETSYEMMMQCVSRMLAHPLHVISMRCMVQFVGREAKYSGVLSSIGKIFKEEGLLGFFVGLIPHLLGDVVFLWGCNLLAHFINAYLVDDSFSQALAIRSYTKFVMGIAVSMLTYPFLLVGDLMAVNNCGLQAGLPPYSPVFKSWIHCWKYLSVQGQLFRGSSLLFRRVSSGSCFALE

SEQ ID NO: 67 - protein, 235 aa, Homo sapiens [sequence omitted]

SEQ ID NO: 68 - protein, 221 aa, Homo sapiens
MWSAGRGGAAWPVLLGLLLALLVPGGGAAKTGAELVTCGSVLKLLNTHHRVRLHSHDIKYGSGSGQQSVTGVEASDDANSYWRIRGGSEGGCPRGSPVRCGQAVRLTHVLTGKNLHTHHFPSPLSNNQEVSAFGEDGEGDDLDLWTVRCSGQHWEREAAVRFQHVGTSVFLSVTGEQYGSPIRGQHEVHGMPSANTHNTWKAMEGIFIKPSVEPSAGHDEL

SEQ ID NO: 69 - protein, 483 aa, Homo sapiens
MKAFHTFCVVLLVFGSVSEAKFDDFEDEEDIVEYDDNDFAEFEDVMEDSVTESPQRVIITEDDEDETTVELEGQDENQEGDFEDADTQEGDTESEPYDDEEFEGYEDKPDTSSSKNKDPITIVDVPAHLQNSWESYYLEILMVTGLLAYIMNYIIGKNKNSRLAQAWFNTHRELLESNFTLVGDDGTNKEATSTGKLNQENEHIYNLWCSGRVCCEGMLIQLRFLKRQDLLNVLARMMRPVSDQVQIKVTMNDEDMDTYVFAVGTRKALVRLQKEMQDLSEFCSDKPKSGAKYGLPDSLAILSEMGEVTDGMMDTKMVHFLTHYADKIESVHFSDQFSGPKIMQEEGQPLKLPDTKRTLLFTFNVPGSGNTYPKDMEALLPLMNMVIYSIDKAKKFRLNREGKQKADKNRARVEENFLKLTHVQRQEAAQSRREEKKRAEKERIMNEEDPEKQRRLEEAALRREQKKLEKKQMKMKQIKVKAM

SEQ ID NO: 70 - protein, 371 aa, Homo sapiens [sequence omitted]

SEQ ID NO: 71 - protein, 402 aa, Homo sapiens
MMNNRFRKDMMKNASESKLSKDNLKKRLKEEFQHAMGGVPAWAETTKRKTSSDDESEEDEDDLLQRTGNFISTSTSLPRGILKMKNCQHANAERPTVARISSVQFHPGAQIVMVAGLDNAVSLFQVDGKTNPKIQSIYLERFPIFKACFSANGEEVLATSTHSKVLYVYDMLAGKLIPVHQVRGLKEKIVRSFEVSPDGSFLLINGIAGYLHLLAMKTKELIGSMKINGRVAASTFSSDSKKVYASSGDGEVYVWDVNSRKCLNRFVDEGSLYGLSIATSRNGQYVACGSNCGVVNIYNQDSCLQETNPKPIKAIMNLVTGVTSLTFNPTTEILAIASEKMKEAVRLVHLPSCTVFSNFPVIKNKNISHVHTMDFSPRSGYFALGNEKGKALMYRLHHYSDF

SEQ ID NO: 72 - protein, 640 aa, Homo sapiens
MALSRGLPRELAEAVAGGRVLVVGAGGIGCELLKNLVLTGFSHIDLIDLDTIDVSNLNRQFLFQKKHVGRSKAQVAKESVLQFYPKANIVAYHDSIMNPDYNVEFFRQFILVMNALDNRAARNHVNRMCLAADVPLIESGTAGYLGQVTTIKKGVTECYECHPKPTQRTFPGCTIRNTPSEPIHCIVWAKYLFNQLFGEEDADQEVSPDRADPEAAWEPTEAEARARASNEDGDIKRISTKEWAKSTGYDPVKLFTKLFKDDIRYLLTMDKLWRKRKPPVPLDWAEVQSQGEETNASDQQNEPQLGLKDQQVLDVKSYARLFSKSIETLRVHLAEKGDGAELIWDKDDPSAMDFVTSAANLRMHIFSMNMKSRFDIKSMAGNIIPAIATTNAVIAGLIVLEGLKILSGKIDQCRTIFLNKQPNPRKKLLVPCALDPPNPNCYVCASKPEVTVRLNVHKVTVLTLQDKIVKEKFAMVAPDVQIEDGKGTILISSEEGETEANNHKKLSEFGIRNGSRLQADDFLQDYTLLINILHSEDLGKDVEFEVVGDAPEKVGPKQAEDAAKSITNGSDDGAQPSTSTAQEQDDVLIVDSDEEDSSNNADVSEEERSRKRKLDEKENLSAKRSRIEQKEELDDVIALD

SEQ ID NO: 73 - protein, 237 aa, Homo sapiens [sequence omitted]

SEQ ID NO: 74 - protein, 432 aa, Homo sapiens
MDARWWAVVVLAAFPSLGAGGETPEAPPESWTQLWFFRFVVNAAGYASFMVPGYLLVQYFRRKNYLETGRGLCFPLVKACVFGNEPKASDEVPLAPRTEAAETTPMWQALKLLFCATGLQVSYLTWGVLQERVMTRSYGATATSPGERFTDSQFLVLMNRVLALIVAGLSCVLCKQPRHGAPMYRYSFASLSNVLSSWCQYEALKFVSFPTQVLAKASKVIPVMLMGKLVSRRSYEHWEYLTATLISIGVSMFLLSSGPEPRSSPATTLSGLILLAGYIAFDSFTSNWQDALFAYKMSSVQMMFGVNFFSCLFTVGSLLEQGALLEGTRFMGRHSEFAAHALLLSICSACGQLFIFYTIGQFGAAVFTIIMTLRQAFAILLSCLLYGHTVTVVGGLGVAVVFAALLLRVYARGRLKQRGKKAVPVESPVQKV

SEQ ID NO: 75 - protein, 252 aa, Homo sapiens
MSFPPHLNRPPMGIPALPPGTPPPQFPGFPPPVPPGTPMIPVPMSIMAPAPTVLVPTVSMVGKHLGARKDHPGLKAKENDENCGPTTTVFVGNISEKASDMLIRQLLAKCGLVLSWKRVQGASGKLQAFGFCEYKEPESTLRALRLLHDLQIGEKKLLVKVDAKTKAQLDEWKAKKKASNGNARPETVTNDDEEALDEETKRRDQMIKGAIEVLIREYSSELNAPSQESDSHPRKKKKEKKEDIFGRFQWAH

SEQ ID NO: 76 - protein, 523 aa, Homo sapiens
MGPQAAPLTIRGPSSAGQSTPSPHLVPSPAPSPGPGPVPPRPPAAEPPPCLRRGAAAADLLSSSPESQHGGTQSPGGGQPLLQPTKVDAAEGRRPQALRLIERDPYEHPERLRQLQQELEAFRGQLGDVGALDTVWRELQDAQEHDARGRSIAIARCYSLKNRHQDVMPYDSNRVVLRSGKDDYINASCVEGLSPYCPPLVATQAPLPGTAADFWLMVHEQKVSVIVMLVSEAEMEKQKVARYFPTERGQPMVHGALSLALSSVRSTETHVERVLSLQFRDQSLKRSLVHLHFPTWPELGLPDSPSNLLRFIQEVHAHYLHQRPLHTPIIVHCSSGVGRTGAFALLYAAVQEVEAGNGIPELPQLVRRMRQQRKHMLQEKLHLRFCYEAVVRHVEQVLQRHGVPPPCKPLASASISQKNHLPQDSQDLVLGGDVPISSIQATIAKLSIRPPGGLESPVASLPGPAEPPGLPPASLPESTPIPSSSQTPFPPHYLRLPSLRRSRQCLKPPARGPPPPPWNCWPP

SEQ ID NO: 77 - protein, 621 aa, Homo sapiens
MGLLSDPVRRRALARLVLRLNAPLCVLSYVAGIAWFLALVFPPLTQRTYMSENAMGSTMVEEQFAGGDRARAFARDFAAHRKKSGALPVAWLERTMRSVGLEVYTQSFSRKLPFPDETHERYMVSGTNVYGILRAPRAASTESLVLTVPCGSDSTNSQAVGLLLALAAHFRGQIYWAKDIVFLVTEHDLLGTEAWLEAYHDVNVTGMQSSPLQGRAGAIQAAVALELSSDVVTSLDVAVEGLNGQLPNLDLLNLFQTFCQKGGLLCTLQGKLQPEDWTSLDGPLQGLQTLLLMVLRQASGRPHGSHGLFLRYRVEALTLRGINSFRQYKYDLVAVGKALEGMFRKLNHLLERLHQSFFLYLLPGLSRFVSIGLYMPAVGFLLLVLGLKALELWMQLHEAGMGLEEPGGAPGPSVPLPPSQGVGLASLVAPLLISQAMGLALYVLPVLGQHVATQHFPVAEAEAVVLTLLAIYAAGLALPHNTHRVVSTQAPDRGWMALKLVALIYLALQLGCIALTNFSLGFLLATTMVPTAALAKPHGPRTLYAALLVLTSPAATLLGSLFLWRELQEAPLSLAEGWQLFLAALAQGVLEHHTYGALLFPLLSLGLYPCWLLFWNVLFWK

SEQ ID NO: 78 - DNA, 2347 bp, Homo sapiens
ccctcgagaagatggcggcgactctgggaccccttgggtcgtggcagcagtggcggcgatgtttgtcggctcgggatgggtccaggatgttactccttcttcttttgttggggtctgggcaggggccacagcaagtcggggcgggtcaaacgttcgagtacttgaaacgggagcactcgctgtcgaagccctaccagggtgtgggcacaggcagttcctcactgtggaatctgatgggcaatgccatggtgatgacccagtatatccgccttaccccagatatgcaaagtaaacagggtgccttgtggaaccgggtgccatgtttcctgagagactgggagttgcaggtgcacttcaaaatccatggacaaggaaagaagaatctgcatggggatggcttggcaatctggtacacaaaggatcggatgcagccagggcctgtgtttggaaacatggacaaatttgtggggctgggagtatttgtagacacctaccccaatgaggagaagcagcaagagcgggtattcccctacatctcagccatggtgaacaacggctccctcagctatgatcatgagcgggatgggcggcctacagagctgggaggctgcacagccattgtccgcaatcttcattacgacaccttcctggtgattcgctacgtcaagaggcatttgacgataatgatggatattgatggcaagcatgagtggagggactgcattgaagtgcccggagtccgcctgccccgcggctactacttcggcacctcctccatcactggggatctctcagataatcatgatgtcatttccttgaagttgtttgaactgacagtggagagaaccccagaagaggaaaagctccatcgagatgtgttcttgccctcagtggacaatatgaagctgcctgagatgacagctccactgccgcccctgagtggcctggccctcttcctcatcgtctttttctccctggtgttttctgtatttgccatagtcattggtatcatactctacaacaaatggcaggaacagagccgaaagcgcttctactgagccctcctgctgccaccacttttgtgactgtcacccatgaggtatggaaggagcaggcactggcctgagcatgcagcctggagagtgttcttgtctctagcagctggttggggactatattctgtcactggagttttgaatgcagggaccccgcattcccatggttgtgcatggggacatctaactctggtctgggaagccacccaccccagggcaatgctgctgtgatgtgcctttccctgcagtccttccatgtgggagcagaggtgtgaagagaatttacgtggttgtgatgccaaaatcacagaacagaatttcatagcccaggctgccgtgttgtttgactcagaaggcccttctacttcagttttgaatccacaaagaattaaaaactggtaacaccacaggctttctgaccatccattcgttgggttttgcatttgacccaaccctctgcctacctgaggagctttctttggaaaccaggatggaaacttcttccctgccttaccttcctttcactccattcattgtcctctctgtgtgcaacctgagctgggaaaggcatttggatgcctctctgttggggcctggggctgcagaacacacctgcgtttcactggccttcattaggtggccctagggagatggctttctgctttggatcactgttccctagcatgggtcttgggtctattggcatgtccatggccttcccaatcaagtctcttcaggccctcagtgaagtttggctaaaggttggtgtaaaaatcaagagaagcctggaagacatcatggatgccatggattagctgtgcaactgaccagctccaggtttgatcaaaccaaaagcaacatttgtcatgtggtctgaccatgtggagatgtttctggacttgctagagcctgcttagctgcatgttttgtagttacgatttttggaatcccactttgagtgctgaaagtgtaaggaagctttcttcttacaccttgggcttggatattgcccagagaagaaatttggctttttttttcttaatggacaagagacagttgctgttctcatgttccaagtctgagagcaacagaccctcatcatctgtgcctggaagagttcactgtcattgagcagcacagcctgagtgctggcctctgtcaacccttattccactgccttatttgacaaggggttacatgctgctcaccttactgccctgggattaaatcagttacaggccagagtctccttggagggcctggaactctgagtcctcctatgaacctctgta

SEQ ID NO: 79 - DNA, 1529 bp, Homo sapiens
cccacgcgtccgccagccttgtctcggccacctcaaggataatcactaaattctgccgaaaggactgaggaacggtgcctggaaaagggcaagaatatcacggcatgggcatgagtagcttgaaactgctgaagtatgtcctgtttttcttcaacttgctcttttggatctgtggctgctgcattttgggctttgggatctacctgctgatccacaacaacttcggagtgctcttccataacctgccctccctcacgctgggcaatgtgtttgtcatcgtgggctctattatcatggtagttgccttcctgggctgcatgggctctatcaaggaaaacaagtgtctgcttatgtcgttcttcatcctgctgctgattatcctccttgctgaggtgaccttggccatcctgctctttgtatatgaacagaagctgaatgagtatgtggctaagggtctgaccgacagcatccaccgttaccactcagacaatagcaccaaggcagcgtgggactccatccagtcatttctgcagtgttgtggtataaatggcacgagtgatttggacagtggctcaccagcatcttgcccctcagatcgaaaagtggaggggtgctatgcgaaagaagactttggtttcattcaatttcctgtatatcggaatcatcaccatctgtgtatgtgtgattgaggtgttgggggatgtcctttgcactgaccctgaactgccagattgacaaaaccagccagaccatagggctatgatctgcagtagttctgtggtgaagagacttgtttcatctccggaaatgcaaaaccatttatagcatgaagccctacatgatcactgcaggatgatcctcctcccatcctttccctttttaggtccctgtcttatacaaccagagaagtgggtgttggccaggcacatcccatctcaggcagcaagacaatctttcactcactgacggcagcagccatgtctctcaaagtggtgaaactaatatctgagcatcttttagacaagagaggcaaagacaaactggatttaatggcccaacatcaaagggtgaacccaggatatgaatttttgcatcttcccattgtcgaattagtctccagcctctaaataatgcccagtcttctccccaaagtcaagcaagagactagttgaagggagttctggggccaggctcactggaccattgtcacaaccctctgtttctctttgactaagtgccctggctacaggaattacacagttctctttctccaaagggcaagatctcatttcaatttctttattagagggccttattgatgtgttctaagtctttccagaaaaaaactatccagtgatttatatcctgatttcaaccagtcacttagctgataatcacagtaagaagacttctggtattatctctctatcagataagattttgttaatgtactattttactcttcaataaataaaacagtttattatctcaaaaaaaaaaaaaaaaaaaaaaaaaaaaaaaaaaaaaaa

SEQ ID NO: 80 - DNA, 4387 bp, Homo sapiens
gcatccccgcttccgggttaggccgttcctgcccgccccctcctctcctcccttcggacccatagatctcaggctcggctccccgcccgccgcagcccactgttgacccggcccgtactgcggccccgtggccaccatgtccctgcacggcaaacggaaggagatctacaagtatgaagcgccctggacagtctacgcgatgaactggagtgtgcggcccgataagcgctttcgcttggcgctgggcagcttcgtggaggagtacaacaacaaggttcagcttgttggtttagatgaggagagttcagagtttatttgcagaaacacctttgaccacccataccccaccacaaagctcatgtggatccctgacacaaaaggcgtctatccagacctactggcaacaagcggtgactatctccgtgtgtggagggttggtgaaacagagaccaggctggagtgtttgctaaacaataataagaactctgatttctgtgctcccctgacctcctttgactggaatgaggtggatccttatcttttaggtacctcaagcattgatacgacatgcaccatctgggggctggagacagggcaggtgttagggcgagtgaatctcgtgtctggccacgtgaagacccagctgatcgcccatgacaaagaggtctatgatattgcatttagccgggccgggggtggcagggacatgtttgcctctgtgggtgctgatggctcggtgcggatgtttgacctccgccatctagaacacagcaccatcatttacgaagacccacagcatcacccactgcttcgcctctgctggaacaagcaggaccctaactacctggccaccatggccatggatggaatggaggtggtgattctagatgtccgggttccctgcacacctgtcgccaggttaaacaaccatcgagcatgtgtcaatggcattgcttgggccccacattcatcctgccacatctgcactgcagcggatgaccaccaggctctcatctgggacatccagcaaatgccccgagccattgaggaccctatcctggcctacacagctgaaggagagatcaacaatgtgcagtgggcatcaactcagcccgactggatcgccatctgctacaacaactgcctggagatactcagagtgtagtgttggtggcgctgtgcccacgaggcaggggcttttgtatttcctgcctctgccccacccccaaagtaagaagaaacatgtttccagtggccagtatgtctttcattgctttgcacccactgttaccagaagctgctctaggagttcctggccagtcaccccatcgccctctgtggcagactcagtgctgtgtggcgcctcctcagcccagggctgagttttaagattttctctcctttcctcttctcctttggttcctcaattaaaaaatgtgtgtatatttgtttgtcaggcgttgtgttgaggagcagttcacgcactggctgtgtctattcctctgcccaggtgtctctgtttgctgcccaaggcagcagttcatgtctcgtccatgtccatgttcgtgttagcacttacgtgggaacaaataccaatttgtcttttctcctagtatcagtgtgtttaacaaattttaactttgtatatttgttatctatcaggctaatttttttatgaaaagaattttactctcctgcttcatttctttgtcttatagtcctccctctttgcaccttcttctcttccctcagtgcctggagctggtactgggcccctgggccccatgagcagtttgccttcttgagtcactgcctgtgtagtacatacctgaccgggagtccaaaccaccttggtgctctgaagtccactgactcatcacacctttcttagcctggctcctctcaagggcattctgggcttgtaaacagacataggaagcctctgtttaccctgaagcaccactgtccagcccattggttcccactggcagcatggtagagctgagagaaacaggctctcagggtacctgacttgaggggaatcgtttcatgaagctgaacttcaagcatatttccagtacattctttcagagtctgtttttccatccaaatataagccccaggccattccacttagtgtcttttcaatgataggcaagaatgatatctgagttgaacttcggtgcttctgttgtttgagtttactgtgcctggtggtatattgggcattctttggattgagtgttctgaggtgagagagtcttcccgaggcatcctgtctgtgcttccaaccctgaacaagaccttacatgagagatggactgatggactgcggcaatcctgggctgtcaagtggatagatagttaaaaagcattatactgtgggtaatgaaaagggaggaaaaaaaaagaaggaaaaggaattatagacccccagggtcagccagttaagagctctacccacacctgtcaacccctctctcccccagtttaggttctgagcagtattggacttgtagcctgcagttgtcttttgacttgcaggccgcaggtgtctttctgttatgtgaatgagttccatggaggggcatatgtgtgattccaccgttagatgagcccttggggcaggcagtttgggatgtgctcttgggggaaagttggctgtttccttgcgctctgctcctacccgaaggtttttaagtccctctgaattgctcatctgagattagtagagtagcaggcctgaaggatgatggttttgtcctctttggttctcacctgcttgagaagtaaaacagtaactttgttcttctgggcccttaagcttttttggttaagtcttccttttcagaagtagatgtcattatatgccaaaagtctagctctttgctttaccatacagggacctgtcccaaagaaaaaggctctttttttagccagcatatttccccttctacccttttactttgttgttctgattttaggactctggctggccatgtgcttgtggttgcctctcctgcatttgccactggatttgcactgcatcgtttggagatacaaagcgagcagttcttggtcagaaccctcctctgcttttcattgtgtttgataatggttactgggtccttctctcaagggtagcaaggccaagctgatggctgcttgtttaggaggccatcagttccttcctgtggagaagggtctgaaatggaagtcagtggtagaaggggctggtctgctgggcagggcttacatccactgagttctaagattcctttcctgatctgcacctacgcctggtctgtatggtggaatttgtcagctggaactcagaaacaacaacttgaaaaaaaaataataattagaacatatttgcataagatagctatttactctggaaaccaacaacttttgagatttcccttgccctgtggacgcccagctcctgtcatccttccttaggtcctgcagtacagtcttcccctgaatgccaccggggacccagggggactccacccccctaagcaagcacacacatactcacagttgatgagttgctggtctttgagtcccagctctcttaccctccctttactccaccagcccgacgacccatgactgaggaggggatttctacagtctcaggatttagaaagtctgtaagccatccatgctccagaaagcaccgatctgttgtagttgcaaaaacaactctgtaatttgttgaggttctcaaactgacagccagcgagactgggtgggaggccctggatctgttctccctgactgcgggaggagcagccactaggactttagcaggaagcccacatggaggctccgccaggctgtggcccagctggtgatggcccttttgctcctggcagcctgaggcacagctgcctgtattgtcctcatctgttctgactgaaggatggaggtgctgaataaattaggcctcaggcctctaccaccagagagctggagaatgggtccacgtcattcaaggacctgaattttttatgctcaggagcattggaatcctcttcttccagggaggaattagcctgcaaggttaggacttgaagagggaaggtatttaataactgggcgaggatgggtgtggtggctcacacctgtaatcccagcattttgggaggctgaggtggccagatcccaaggtcagaagatcgagaccatcctggctaacatggtgaaaccccatctctactaaaaatacaaaaaaaaattagccgggggtggtggcgggtacctgtagtcctagctacttgggaggctgaggcaggagaatggcgtgaacctgggaggtggagcttgcagtgagccaagatcgtccactcactgcagcctggcgacagagcaagcg

SEQ ID NO: 81 - DNA, 2117 bp, Homo sapiens
gcctgagcgggaagcattggcgtccgagcgacttctaggagcctggggttcggcgctatggaggagctcgatggcgagccaacagtcactttgattccaggcgtgaattccaagaagaaccaaatgtattttgactggggtccaggggagatgctggtatgtgaaacctccttcaacaaaaaagaaaaatcagagatggtgccaagttgcccctttatctatatcatccgtaaggatgtagatgtttactctcaaatcttgagaaaactcttcaatgaatcccatggaatctttctgggcctccagagaattgacgaagagttgactggaaaatccagaaaatctcaattggttcgagtgagtaaaaactaccgatcagtcatcagagcatgtatggaggaaatgcaccaggttgcaattgctgctaaagatccagccaatggccgccagttcagcagccaggtctccattttgtcagcaatggagctcatctggaacctgtgtgagattctttttattgaagtggccccagctggccctctcctcctccatctccttgactgggtccggctccatgtgtgcgaggtggacagtttgtcggcagatgttctgggcagtgagaatccaagcaaacatgacagcttctggaacttggtgaccatcttggtgctgcagggccggctggatgaggcccgacagatgctctccaaggaagccgatgccagccccgcctctgcaggcatatgccgaatcatgggggacctgatgaggacaatgcccattcttagtcctgggaacacccagacactgacagagctggagctgaagtggcagcactggcacgaggaatgtgagcggtacctccaggacagcacattcgccaccagccctcacctggagtctctcttgaagattatgctgggagacgaagctgccttgttagagcagaaggaacttctgagtaattggtatcatttcctagtgactcggctcttgtactccaatcccacagtaaaacccattgatctgcactactatgcccagtccagcctggacctgtttctgggaggtgagagcagcccagaacccctggacaacatcttgttggcagcctttgagtttgacatccatcaagtaatcaaagagtgcagcatcgccctgagcaactggtggtttgtggcccacctgacagacctgctggaccactgcaagctcctccagtcacacaacctctatttcggttccaacatgagagagttcctcctgctggagtacgcctcgggactgtttgctcatcccagcctgtggcagctgggggtcgattactttgattactgccccgagctgggccgagtctccctggagctgcacattgagcggatacctctgaacaccgagcagaaagccctgaaggtgctgcggatctgtgagcagcggcagatgactgaacaagttcgcagcatttgtaagatcttagccatgaaagccgtccgcaacaatcgcctgggttctgccctctcttggagcatccgtgctaaggatgccgcctttgccacgctcgtgtcagacaggttcctcagggattactgtgagcgaggctgcttttctgatttggatctcattgacaacctggggccagccatgatgctcagtgaccgactgacattcctgggaaagtatcgcgagttccaccgtatgtacggggagaagcgttttgccgacgcagcttctctccttctgtccttgatgacgtctcggattgcccctcggtctttctggatgactctgctgacagatgccttgccccttttggaacagaaacaggtgattttctcagcagaacagacttatgagttgatgcggtgtctggaggacttgacgtcaagaagacctgtgcatggagaatctgataccgagcagctccaggatgatgacatagagaccaccaaggtggaaatgctgagactttctctggcacgaaatcttgctcgggcaattataagagaaggctcactggaaggttcctgagaactgcttcaatgtggtatctttgtatggcaatgtatatagattttttaaaagaataaatgttgtttgcaaaaaaaaaaaaaaaaaaa

SEQ ID NO: 82 - DNA, 846 bp, Homo sapiens
ggcgggcggagtctgcaggatggcaccggacccctggttctccacatacgattctacttgtcaaattgcccaagaaattgctgagaaaattcaacaacgaaatcaatatgaaagaaaaggtgaaaaggcaccaaagcttaccgtgacaatcagagctttgttgcagaacctgaaggaaaagatcgcccttttgaaggacttattgctaagagctgtgtcaacacatcagataacacagcttgaaggggaccgaagacagaacctcttggatgatcttgtaactcgagagagactacttctggcatcctttaagaatgagggtgccgaaccagatctaatcaggtccagcctgatgagtgaagaggctaagcgaggagcacccaacccttggctctttgaggagccagaggagaccagaggcttgggttttgatgaaatccggcaacagcagcagaaaattatccaagaacaggatgcaggccttgatgccctttcctctatcataagtcgccaaaaacaaatggggcaggaaattgggaatgaattggatgaacaaaatgagataattgacgaccttgccaacctagtggagaacacagatgaaaaacttcgcaatgaaaccaggcgggtaaacatggtggacagaaagtcagcctcttgtgggatgatcatggtgattttactgctgcttgtggctatcgtggttgttgcagtctggccgaccaactgatggcagtaaagagaccaccagcagtgacacctggcaatgacagatgcaagcccaacacccttttggtacgcaaaacctgctctcaataaattcccccaaagctctgaaaaaaaaaa

SEQ ID NO: 83 - DNA, 1011 bp, Homo sapiens
gaaagagataactggaagttccttgattcagaaaacagattcagatgaagaagttgcaatgctgttggacacagtccagaaagtatttcagaaaatgttggaatgtattgcacggagcttcaggaagcagccggaagaaggcctgcggctgctttattctgttcagaggcctcttcatgagttcattactgctgttcagtctcggcacacagacacccctgtgcaccggggtgtactttctactctgatcgctgggcctgtggttgagataagtcaccagctacggaaggtttctgacgtagaagagcttacccctccagagcatctttctgatcttccaccattttcaaggtgtttaataggaataataataaagtcttcgaatgtggtcaggtcatttttggatgaattaaaggcatgtgtggcttctaatgatattgaaggcattgtgtgcctcacggctgctgtgcatattatcctggttattaatgcaggtaaacataaaagctcaaaagtgagggaggttgcagccactgttcacagaaaactaaagacattcatggaaattactttggaagaggatagcattgaaagatttctctatgaatcatcatcaagaactctgggagaacttttgaattcataaccaagccaacatctccagacatgtaaaaatagggaaaagtgattcaaattgaaatgcctgtgtattttcctattgtttttaatgttaataacccatataatagggaaagggtgggatttttttgtgggaatgtgggaaggtgggggttatggaggagataactcaaaacttcttcaattttgcctagtgcctgcgtaaataatatatttaatataaaggactccaggtatgaatggtgtagaaatccatgattccaagaaaaaacacttttctagcaaacctggttgtttttaaaatgacttttatatatgtaatattgcttggaaactatgagtaataaagcaatgacaacatcaaaaaaaaaa

SEQ ID NO: 84 - DNA, 2478 bp, Homo sapiens
cccacgcgtccgcccacgcgtccgcagcgctgtgtttgcgagcgggagcgaggggcgccggctggggtgtgtgctcctgagctcttcagaaaccaggctgctttcaggaacattgctgtggattcccagctttcagacaacacatgactaagacagatgagaccactctagttgcctcatgggaaactcgggaaaagactgcaaaaacaacattgtttctccctttggaattctggagttataaggcagaggtcccccatcttcccgaactggcctattccgctagaagcaagatggctgaactcaatactcatgtgaatgtcaaggaaaagatctatgcagttagatcagttgttcccaacaaaagcaataatgaaatagtcctggtgctccaacagtttgattttaatgtggataaagccgtgcaagcctttgtggatggcagtgcaattcaagttctaaaagaatggaatatgacaggcaaaaagaagaacaataaaagaaaaagaagcaagtccaagcagcatcaaggcaacaaagatgctaaagacaaggtggagaggcctgaggcagggcccctgcagccgcagccaccacagattcaaaacggccccatgaatggctgcgagaaggacagctcgtccacagattctgctaacgaaaaaccagcccttatccctcgtgagaaaaagatctcgatacttgaggaaccttcaaaggcacttcgtggggtcacagaaggcaacagactactgcaacagaaactatccttagatgggaaccccaaacctatacatggaacaacagagaggtcagatggcctacagtggtcagctgagcagccttgtaacccaagcaagcctaaggcaaaaacatctcctgttaagtccaatacccctgcagctcatcttgaaataaagccagatgagttggcaaagaaaagaggcccaaatattgagaaatcagtgaaggatttgcaacgctgcaccgtttctctaactagatatcgcgtcatgattaaggaagaagtggatagttccgtgaagaagatcaaagctgcctttgctgaattacacaactgcatcattgacaaagaagtttcattaatggcagaaatggataaagttaaagaagaagccatggaaatcctgactgctcgtcagaagaaagcagaagaactaaagagactcactgaccttgccagtcagatggcagagatgcagctggccgaactcagggcagaaattaagcactttgtcagcgagcgtaaatatgacgaggagctcgggaaagctgcccggttttcctgtgacatcgaacagctgaaggcccaaatcatgctctgcggagaaattacacatccaaagaacaactattcctcaagaactccctgcagctccctgctgcctctgctgaatgcgcacgcagcaacctctgggaaacagagtaacttttcccgaaaatcatccactcacaataagccctctgaaggcaaagcggcaaaccccaaaatggtgagcagtctccccagcaccgccgacccctctcaccagaccatgccggccaacaagcagaatggatcttctaaccaaagacggagatttaatccacagtatcataacaacaggctaaatgggcctgccaagtcgcagggcagtgggaatgaagccgagccactgggaaagggcaacagccgccacgaacacagaagacagccgcacaacggcttccggcccaaaaacaaaggcggtgccaaaaatcaagaggcttccttggggatgaagacccccgaggccccggcccattctgaaaagccccggcgaaggcagcacgctgcagacacctcggaggccaggcccttccggggtagtgtcggtagggtttcacagtgcaatctctgccccacgagaatagaagtttccacagatgcagcagttctctcagtcccggctgtgacgttggtggcctgagctaggaggaaaaagagcagttttcactcagttttggttccctgcccgaggtgctgacccaattcgctgccaaaagagtgtcaatcagaatatacaaatcccgtatggttgtgtcatcctctcttaatcatttttactaattctaataatcagctctagcttgcttcataattttcatggctttgcttgatctgttgatgctttctctcatcaagactttgcagcattttagccaggcagtatttactcattattaggaaaatcaagatgtggctgaagatcagaggctcagttagcaacctgtgttgtagcagtgatgtcagtccattgattgtctttagagagttaatgttacaaaaaagaattcttaataatcagacaaacatgatctgctgaggacacatgcgcttttgtagaatttaacatctggtgtttttctgaaaaaatatatatacatatattgctttatttgaaacaaattaaaatatgctgcatttgaaaaaaaaaa

SEQ ID NO: 85 - DNA, 1897 bp, Homo sapiens
tgcacatctagcacaaattgaagatgatagagctgcgatggttatttcttggcatctggcaagtgacatggactgtgtagtcaccctaaccactgacgctgcacgtcgtatctatgatgaaacccaaggtcgtcagcaggtgttgccccttgattctatttacaagaagactcttccagattggaaaagatctctacctcatttccgaaatggaaaattgtattttaaacccattggagatccagtctttgctcgagacttgttaacatttccagataatgtagaacattgtgaaacagtatttggtatgctgttaggagacaccattattttggataatctggatgcggccaatcattatagaaaagaggttgttaaaattacacactgtcctacactgctgaccagagatggagatcgaattcgaagtaatggaaagtttgggggccttcagaataaagctcctccaatggataaacttcggggaatggtatttggagctccagttccaaaacagtgtctgatcttaggggaacaaatagatcttcttcagcagtatcgttctgctgtgtgcaaactagacagtgtgaataaggatcttaacagtcaattagagtaccttcgcactccggatatgaggaagaaaaagcaagaacttgatgaacatgagaaaaatctcaaactaatagaggaaaaactaggtatgactcccatacgtaagtgtaatgactcattgcgtcattcaccaaaggttgagacgacagattgtccagttcctcctaaaagaatgagacgagaagctacaagacaaaataggattataaccaaaacagatgtatgagaggtgacagagagaagaggccattggtctcagtaagaatgccctgctttctgcatctctgtttcagaagaccaagagggtgacttaccagactgagtatttctggggacaatacaagtacctgggcatgaatttccatttcgattcagatgggactggaaacaaccattcaattttatgaatcttactggacattatggatttactggaattattccagacattatgccctttggttgtcactaccttgcaaatgtgtaagaggaaaatgtgctaatgtggcagtgactgtaaaactggcacatggcatttattaatcctgaagaaaagtacatgtactatttttcagtataaatataatgaacatgtcagaactatttcttgaaaacctttttattacttttgcgtgaatttatttaacaaagatgttttgtcttttgtgtaagggaggttctagaggctagatgtttaattgtaaatatgtgaggaaactcaatgcagaattcaggataaaaattttaaaagcacaggtatttgggaattgaaatgttaagatacccagaacaacattaaatcaatgagtgaacttgtgacagtggtagcatttcaaatttcaaaagacttatcctgtgtgtgtgtgtgtgtgtgtatatatatatatatatatataaatatatatatataaaatattcagcagcaccaagttttataactattgtttgtttgactttattaatactagaatatgtagtctcagccttaattttacatttacattattttgtaattttttattactatttttaaggggttaaagagaacatacattctcacattagtgtactttctggtagaaagttgctgcaaaaacatttgaaatgtatattaacctaatgtatgtcatatatatgtctttgtgtaagttcaagactattgatctgtgaagttattttgtaaggacatacatttggtaagtaagtttgtgtcccaggaaatgtatgtgtttttaaaccctttctaaatatgcaggccattaataaataagattgtgtctca

SEQ ID NO: 86 - DNA, 1488 bp, Homo sapiens
cccacgcgtccggggacatcctgttctgagtcaagattcctccttctgaacatgggactttccagaaggaccacagctcctcccgtgcatccactcggcctgggaggttctggattttggctgtcgagggagtttgcctgcctctccagagaaagatggtcatgaggcccctgtggagtctgcttctctgggaagccctacttcccattacagttactggtgcccaagtgctgagcaaagtcgggggctcggtgctgctggtggcagcgcgtccccctggcttccaagtccgtgaggctatctggcgatctctctggccttcagaagagctcctggccacgtttttccgaggctccctggagactctgtaccattcccgcttcctgggccgagcccagctacacagcaacctcagcctggagctcgggccgctggagtctggagacagcggcaacttctccgtgttgatggtggacacaaggggccagccctggacccagaccctccagctcaaggtgtacgatgcagtgcccaggcccgtggtacaagtgttcattgctgtagaaagggatgctcagccctccaagacctgccaggttttcttgtcctgttgggcccccaacatcagcgaaataacctatagctggcgacgggagacaaccatggactttggtatggaaccacacagcctcttcacagacggacaggtgctgagcatttccctgggaccaggagacagagatgtggcctattcctgcattgtctccaaccctgtcagctgggacttggccacagtcacgccctgggatagctgtcatcatgaggcagcaccagggaaggcctcctacaaagatgtgctgctggtggtggtgcctgtctcgctgctcctgatgctggttactctcttctctgcctggcactggtgcccctgctcagggaaaaagaaaaaggatgtccatgctgacagagtgggtccagagacagagaacccccttgtgcaggatctgccataaaggacaatatgaactgatgcctggactatcagtaaccccactgcacaggcacacgatgctctgggacataactggtgcctggaaatcaccatggtcctcatatctcccatgggaatcctgtcctgcctcgaaggagcagcctgggcagccatcacaccacgaggacaggaagcaccagcacgtttcacacctcccccttccctctcccatcttctcatatcctggctcttctctgggcaagatgagccaagcagaacattccatccaggacactggaagttctccaggatccagatccatggggacattaatagtccaaggcattccctcccccaccactattcataaagtactaaccaactggcaccaagaaaaaatcctcactaaccgcatcatccgacaactaataattcacactacatccaaacatcacttaggcggcggggccgccgactggttccgggcttagggtggg

SEQ ID NO: 87 - DNA, 1357 bp, Homo sapiens
ccgactttgtagcatttttatttaagctaaaacagagcacatgtatatgtacataagacacattaaatctataaatactatttattcattttatataaactaatgtaatggaaaacaaattcttatgactttgtggttttatagatgttctagaaactttgtatgtaggtatctacaaaattagttcattcccctgaatatttttgcattcatatttttgaggtcttgatgttttcagcctctggcgaatctttttcattgaatttgaaccatttgtaaaatctgtgatgctgaagcagagtgtgtcacaaagtgatgagaacattactaaaatccacggacgcactgcgacctaagggctcaacggctgactcggcagcgggcagccaccccacgctcccctgcggtcactcgcacaccacagcctgaagctcccccagcgcctgcacctcgcacacagctaaggtcaaagttcaaacgcactccacacggaagctcattctatacccgaagagcagtctcagaaagcaagattacttttgtgttttttaaaaaatgattctttaatgtatttttctaaacattctgattggaagtagtggattcctaaatgattccaaagtcatctgtaattcttctgtttttgttttgttctgtcttttcttcattttggctttgggtggggggaggggcaggtgacacaaaggatttttttttttttttttttttaatttttggaatcttttccaataaccagctaaagatttgcactgaaatacaacttgtatgccttttgcatttttaaagcctgcttcctggatttaagcagagtgatagtgttcaaagagccagttcagcctgtaacatatttgaaaaagatatgtctgcactttgaggtcccttttgaatgccattcactagacctctcaagcattttgtttcattgctacatccaagcgcctcacaagtccacaatgcgggacagcatcaaaagctcaagactttggaaaaagcttgtgggcttgcactgggggagggaagggaacaaaatttgtgtacttctttgtttaatttagaaataaggcatccaagagatgccattattttctgtgtttcaattgttgtgcctttgagttaaactgcatttttgtcttttggttgaaatctgaaatgtactgtcccaatataaaacagtaattatttgacctttgcactgtttgtctggtccttttcagtttgattgcatataaatgtggaacttgatagatctctatatttttaatgcacttgtgataaactggcagcagggttagacattactttcaaagcttgaggtagaccgagtcagcatgctaga

SEQ ID NO: 88 - DNA, 2330 bp, Homo sapiens
cctacttgttcccaccttgggagaggacgatgacttgggagggacgcgtgaagggagaaggggtcctcccatgaggctgaggatggcctgaacctggagcagcggaccaggcagacgggctgaagtggggtcccaaattccatgtccagaggtgtggggagcctgcctccctagctcctggcccctgccaggggcttacatcaaaacacctcagagggctgccctccagaggctgcacccagaacagtgggacatgagcaggggtgtgggcttggagggtgaagaggatgtggtcctatcagatgctgggcctcctcagccatagccccctgctcctaccccctgactggctcttgtgtcctcacctctcaccctctccttcctgggaggccctgggaggtgatcattgacacccagccaagcagacagctgcgggtgcccaagcccttgctgggcctgcgcgtgaggagtcccactgcttctaaaggaagtcctgggcaggaggtggctttggtggttggttccaaagttgaaaatgcttgcagtttgaccttagaagaagtgggaagaagaaggagctctacagggtcagctttgtttgatttgtccagtctaagaagtcccattgccaaagctttctgcaggagggtgaatgccgcagcttggcagcccctgggtttctcttggaaatggtcagtttcccctcaaagtacccaaagtagccttggcttgagtttttgtccttgcctcctttttagagaagagggcatttagactgcattttcctggttaaagaaggttaaagcaaatgtttattgccttttctagtgaactaactcgtagagatgttctcagcaggaagacagtcttagcactgtcacttagcagattgcacttaagtcccttgtgctggccagatggcgtggctggttgccttaatatgtcccaggacccctgacagggctgcctggcctctccctcgtgctcctcaagagcccagtccatacactgtggatgtcattgctgtcgggttaggaagtcttgtcctagaacgccctggctggtatgaccacagttcatggcggctcttctcgcttgggtcatggtcatcttccagcacctgctgtgctgggaaggccgaggatgggggcccagcactgtccaggcctgctggggcctggctgggagtcctgtgggcagcatggaacatgcagctgggcttcctgtgaccaggcaccctctggcactgttgcttgccctgtgccctggaccttttcctgcccttctccttcctctgctcccttggggctaccccttggcccctcctggtctgtgcaaactccctcagggagcccccctgccctgtagctctcacttaacttcctaggggctgctgagcccacccagaggttgttggagttcagcggggcagcttgtctcccttgtcagcaggggcgtaagggctgggtttggccatacaaggttggctacgccctcaatccctgaccgttccaggcactgagctgggcacccacggaaggacatgctgtccagactgtgatgactgccagcacagggcatctcgggcttggctggtctgcgaggccttgcccctgtggaactctgggttcctgttttctcagtctttttgcggctttgctgtggttggcagctgccgtactccaggcttgtgtcggccactcagatgagggctgtggtgcgagccagtgcaggagagctgcgcttgggattgtgccctctcctgtgtctgtcctccggacctacccaggtctccaccatcaggaccctgtctttgggtttagaagaccaagtatggggaaaaccagacaccagcctctgcagcaatgggtccctctagcctgtggacaccagctgggggatccagggtcaggccccctcctctccccagtttccctctgctgtgggttctgggctgtcatgtctccaccacttaaggatgtctttacactgacttcaggatagatgctgggatgcctgggcatggccacatgttacatgtacagaactttgtctacagcacaaattaagttatataaacacagtgactggtatttaatgctgatctactataaggtattctatatttatatgacttcagagacgcgtatgtaataaaggacgccctccctccagtgtccacatccagttcaccccagagggtcgggcaggttgacatatttatttttgtctattctgtaggcttccatgtccagaatcctgcttaaggttttagggtaccttcagtactttttgcaataaaagtatttcctatccaaaaaaaaaaaaaa

SEQ ID NO: 89 - DNA, 2729 bp, Homo sapiens
ctacaccttttccatttgctaataaggccctgccaggctgggagggaattgtccctgcctgcttctggagaaagaagatattgacaccatctacgggcaccatggaactgcttcaagtgaccattctttttcttctgcccagtatttgcagcagtaacagcacaggtgttttagaggcagctaataattcacttgttgttactacaacaaaaccatctataacaacaccaaacacagaatcattacagaaaaatgttgtcacaccaacaactggaacaactcctaaaggaacaatcaccaatgaattacttaaaatgtctctgatgtcaacagctacttttttaacaagtaaagatgaaggattgaaagccacaaccactgatgtcaggaagaatgactccatcatttcaaacgtaacagtaacaagtgttacacttccaaatgctgtttcaacattacaaagttccaaacccaagactgaaactcagagttcaattaaaacaacagaaataccaggtagtgttctacaaccagatgcatcaccttctaaaactggtacattaacctcaataccagttacaattccagaaaacacctcacagtctcaagtaataggcactgagggtggaaaaaatgcaagcacttcagcaaccagccggtcttattccagtattattttgccggtggttattgctttgattgtaataacactttcagtatttgttctggtgggtttgtaccgaatgtgctggaaggcagatccgggcacaccagaaaatggaaatgatcaacctcagtctgataaagagagcgtgaagcttcttaccgttaagacaatttctcatgagtctggtgagcactctgcacaaggaaaaaccaagaactgacagcttgaggaattctctccacacctaggcaataattacgcttaatcttcagcttctatgcaccaagcgtggaaaaggagaaagtcctgcagaatcaatcccgacttccatacctgctgctggactgtaccagacgtctgtcccagtaaagtgatgtccagctgacatgcaataatttgatggaatcaaaaagaaccccggggctctcctgttctctcacatttaaaaattccattactccatttacaggagcgttcctaggaaaaggaattttaggaggagaatttgtgagcagtgaatctgacagcccaggaggtgggctcgctgataggcatgactttccttaatgtttaaagttttccgggccaagaatttttatccatgaagactttcctacttttctcggtgttcttatattacctactgttagtatttattgtttaccactatgttaatgcagggaaaagttgcacgtgtattattaaatattaggtagaaatcataccatgctactttgtacatataagtattttattcctgctttcgtgttacttttaataaataactactgtactcaatactctaaaaatactataacatgactgtgaaaatggcaatgttattgtcttcctataattatgaatatttttggatggattattagaatacatgaactcactaatgaaaggcatttgtaataagtcagaaagggacataggattcacatatcagactgttagggggagagtaatttatcagttctttggtctttctatttgtcattcatactatgtgatgaagatgtaagtgcaagggcatttataacactatactgcattcattaagataataggatcatgatttttcattaactcatttgattgatattatctccatgcattttttatttcttttagaaatgtaattatttgttctagcaatcattgctaacctctagtttgtagaaaatcaacactttataaatacataattatgatattatttttcattgtatcactgttctaaaaataccatatgattatagctgccactccatcaggagcaaattcttctgttaaaagctaactgatcaaccttgaccacttttttgacatgtgagatcaaagtgtcaagttggctgaggttttttggaaagctttagaactaataagctgctggtggcagctttgtaacgtatgattatctaagctgattttgatgctaaattatcttagtgatctaaggggcagtttagtgaagatggaatcttgtatttaaaatagccttttaaaatttgttttgtggtgatgtattttgacaacttccatctttaggagttatataatcaccttgattttagtttcctgatgtttggactatttataatcaaggacaccaagcaagcataagcatatctatatttctgactggtgtctctttgagaaggatgggaagtagaaaaaaaaaaaagaaagaaaggaaaggaagagaggagagaagaaggcagggatctccactatgtatgttttcactttagaactgttgagcccatgcttaattttaatctagaagtctttaaatggtgagacagtgactggagcatgccaatcagagagcatttgtcttcagaaaaaaaaaaaatctgagtttgagactagcctggccaacatgttgaaaccccatatctactaaaaatacaaaaattagcctggtgtggtggcgcacgcctgtagtcccagctactctggagcctgaggaacgtgaatcgcttgaacccagaagacagaggttgcagtgagctgagatggcactattgcactccagactggtgacacacgcaga

SEQ ID NO: 90 - DNA, 1386 bp, Homo sapiens
ggcccctgcactgctcctgatccctgctgccctcgcctctttcatcctggcctttggcaccggagtggagttcgtgcgctttacctcccttcggccacttcttggagggatcccggagtctggtggtccggatgcccgccagggatggctggctgccctgcagaccgcagcatccttgcccccctggcatgggatctggggctcctgcttctatttgttgggcagcacagcctcatggcagctgaaagagtgaaggcatggacatcccggtactttggggtccttcagaggtcactgtatgtggcctgcactgccctggccttgcagctggtgatgcggtactgggagcccatacccaaaggccctgtgttgtgggaggctcgggctgagccatgggccacctgggtgccgctcctctgctttgtgctccatgtcatctcctggctcctcatctttagcatccttctcgtctttgactatgctgagctcatgggcctcaaacaggtatactaccatgtgctggggctgggcgagcctctggccctgaagtctccccgggctctcagactcttctcccacctgcgccacccagtgtgtgtggagctgctgacagtgctgtgggtggtgcctaccctgggcacggaccgtctcctccttgctttcctccttaccctctacctgggcctggctcacgggcttgatcagcaagacctccgctacctccgggcccagctacaaagaaaactccacctgctctctcggccccaggatggggaggcagagtgaggagctcactctggttacaagccctgttcttcctctcccactgaattctaaatccttaacatccaggccctggctgcttcatgccagaggcccaaatccatggactgaaggagatgccccttctactacttgagactttattctctgggtccagctccataccctaaattctgagtttcagccactgaactccaaggtccacttctcaccagcaaggaagagtggggtatggaagtcatctgtcccttcactgtttagagcatgacactctccccctcaacagcctcctgagaaggaaaggatctgccctgaccactcccctggcactgttacttgcctctgcgcctcaggggtccccttctgcaccgctggcttccactccaagaaggtggaccagggtctgcaagttcaacggtcatagctgtccctccaggccccaaccttgcctcaccactcccggccctagtctctgcacctccttaggccctgcctctgggctcagaccccaacctagtcaaggggattctcctgctcttaactcgatgacttggggctccctgctctcccgaggaagatgctctgcaggaaaataaaagtcag

SEQ ID NO: 91 - DNA, 542 bp, Homo sapiens
cccgggccatgcagcctcggccccgcgggcgcccgccgcgcacccgaggagatgaggctccgcaatggcaccttcctgacgctgctgctcttctgcctgtgcgccttcctctcgctgtcctggtacgcggcactcagcggccagaaaggcgacgttgtggacgtttaccagcgggagttcctggcgctgcgcgatcggttgcacgcagctgagcaggagagcctcaagcgctccaaggagctcaacctggtgctggacgagatcaagagggccgtgtcagaaaggcaggcgctgcgagacggagacggcaatcgcacctggggccgcctaacagaggacccccgattgacgccgtggaacggctcacaccggcacgtgctgcacctgcccaccgtcttccatcacctgccacacctgctggccaaggagagcagtctgcagcccgcggtgcgcgtgggccagggccgcaccggagtgtcggtggtgatgggcatcccgagcgtgcggcgcgaggtgcactcgtacctgactgacactctgca

SEQ ID NO: 92 - DNA, 772 bp, Homo sapiens
  modified_base (665): a, c, g, t, unknown or other
cgagcccggagtgcggacacccccgggatgcttgcgccccagaggacccgcgccccaagcccccgcgccgcccccaggcccacccggagcatgctgcctgcagccatgaagggcctcggcctggcgctgctggccgtcctgctgtgctcggcgcccgctcatggcctgtggtgccaggactgcaccctgaccaccaactccagccattgcaccccaaagcagtgccagccgtccgacacggtgtgtgccagtgtccgaatcaccgatcccagcagcagcaggaaggatcactcggtgaacaagatgtgtgcctcctcctgtgacttcgttaagcgacactttttctcagactatctgatggggtttattaactctgggatcttaaaggtcgacgtggactgctgcgagaaggatttgtgcaatggggcggcaggggcagggcacagcccctgggccctggccggggggctcctgctcagcctggggcctgccctcctctgggctgggccctgatgtctcctgcttcccacggggcttctgagcttgctcccctgagcctgtggctgccctctccccagcctggcgtggctggggctgggggcagccttgggccagctccgtggctgtggcctgtgggtctgaattcttccccgacgtgaagcctncctgtctctccggcagctctgagtcccaggcagctggacattccaggggaacaagccattnggcaggagggctgggatgaggttgggggggaccggaggtcccggag

SEQ ID NO: 93 - DNA, 1738 bp, Homo sapiens
tgtccatccaaaaaccataaaatcactgggttccacatcagcctccatgaggccaagccttgtacctgcaagctcttggcctaaccattcctctgtcctcttctctggcctgcctggggagcccgtgaaggccgcacgggtgcctccagcctgagacatcaggggagagcctgcagctgagttcagcagaaaggaggaatcctggccctcaggaagaagatagtcacatgtttttcttccttgtccccacagcccccagaacaacattctccctgctggcagcccttccatgtctccaaacctgggtcagagtgaaaggacctttgggggtgggtgggagcaaagggcccacctgctggttggtgaaagcagtggtgccggagtgctaggtaccgcacgagtagtggtgcgggggcttgggaagcagaccagggttggacaaaaccccatgagggcggggagctggaagaaaagtctcttggggacctctggggcaaggagctgagaagtcctgcagcaccaggtgagacttgcttacagtggatgccacttctaggcctctggaccgcagatgccctcctccctcctgcacacctggcctcctgggcctccaggtaaagagagagagccagcccagccctgtttcccctcagtcctcctttgctcctgctgcttctcccaacagcccactgttaggaggtagtagaccccagcctcaaggctctgaccttcttcatgtgggcacagagggtcctgacactctggcagggcctgagctggggcaggcctccctcagggccaggggcgatggcaccccggggacaggcagacctccttcctgccgtcagcacccccttccttatcactgtctggtctccgagcttcggctgcagcctgaggtgtgtcctgggctcctcagagcctgaagcaagcttttggaagcctgcagtcctcccagctccagtgcagaagcctctctctccagcctttccccaggcaggagttggggttgggggcctctgtccctcatcgcttaccttggaaaggtgggaagctggcaatctgcaccttggggcctgggctccccctctctgtgccagcggcttcccagcacctgggaggggctgcagccccagctggactccagcctgtccctcttagcactctagctgcccactccagggcagggactcgaaaccccctccgtcctgagcagccacctccagggccctgtttgggaccactctctcagtccccaggtcctcagggccccagagcgggagggtctcctacctggaagtccccctgagctccagggcccagccctacctgccagtgctggtgtcagggcactcaacaccgagtgtgggggccacgccccttgccatgcccacggcctcctcctgtagcccctgcctgcacccacgatgctgcacgggcccgccctggtggggctcggcgagtaatgtgttttgtccccagttaaccaccattctgcggcctggttctgcaaggaaccagggctgccccaccgcccgccgtctgccgccctaggcttcctgactccattagttccgacacttgtgaaactccgagaagtgctgtggtctcagcaatgcacctgttttgtacatgattgtgtaatttaaaggtatataaatacaaatatatatatatatcagttgtgattgtatgactgtggataaaatccagaactgtgtcaacctgaaaaaaaaaa

SEQ ID NO: 94 - DNA, 2100 bp, Homo sapiens
  modified_base (2087): a, c, g, t, unknown or other
gggaaagcggcgagtaagatggaagatgaggaggtcgctgagagctgggaagaggcggcagacagcggggaaatagacagacggttggaaaaaaaactgaagatcacacaaaaagagagcaggaaatccaaatctcctcccaaagtgcccattgtgattcaggacgatagccttcccgcggggccccctccacagatccgcatcctcaagaggcccaccagcaacggtgtggtcagcagccccaactccaccagcaggcccacccttccagtcaagtccctagcacagcgagaggccgagtacgccgaggcccggaagcggatcctgggcagcgccagccccgaggaggagcaggagaaacccatcctcgacaggccaaccaggatctcccaacccgaagacagcaggcagcccaataatgtgatcagacagcctttgggtcctgatgggtctcaaggcttcaaacagcgcagataaatgcaggcaagaaaagatgccgccgttgctgccgtcaccgcctcctgggtcgtccgccacgggttgcactgccgtggcagacagctggacttgagcagagggaacgacctgacttacttgcactgtgatcccccttgctccgcccactgtgaccttgaaccccatgcactgtgacctccccccttctcccccttcccactgtgattggcacatcgacaagggctgtcccaagtcaatggaaagggaaagggtgggggttaggggaaggttggggggacccagcaaggactcagagagtcagacagtgccacttggccacttggggtaaagccagtgccagcaataacagtttatcatgctcattaatttgggatttcaaaacacaaatgaaaactcacacccacccacccccaagtgcatgtctccatcacttaaaaagtaagttccatttgaaaatatcctttcttttttttttcttcctatttttgtttgtttatacaaatatctgatttgcaagaaaaagtgcatgggaggggttttagtggtttaatgaatttttaattaagaaagggtagtttggtagtctacttaaaaatgtttctgggaaattcactagaaacattaaccaataggattttggtgagcttagcttctgtattcctactgccgcccagaaaaggggcagggctctgcagccgccaggacagacgagcaccccatgcctatacctccctccccgagctaagtcccagggcatctgggccttgcctggagactgggctagctctgtaggctcggagagcctggggagggtgccaaccccacctctagtattttgggagatagggaaagtgaaccgacttccccttcccatacccctcagggtggttccctaccagccaggcttactacttctagaagaaagcagagtgccagggagtgagattgcatccctgggcttagaagtgacggagagaagacttgtttagtattttgccatcagcacaaggaaaaccaggagagagtctgcctccaggactctgagccttctgcctcgtatgttcagaaggtggataggtcttcccactccagcatggcttgaactcttaggggtctgcagtgctccatctccattggtggccccagctcagtaactatacctggtacatttcctgtgtgcaatcagtaccttgaaggcagaacattctgaataaagttggaaaaagaacagctttgctttgcaaagattgatgacagactggttcctcagaggcctaggctacccgtcacccctttttccagagcgagggcctggaatgaaggcagtttatcctctgtccctggagcctggggtttgctttggctccttgaggtggaagagactaagagggcagctgcccagagcagctgtgtgtacctggctcctctcaggcttcctgatcccttccattgcactgcgccttatccctcagccagccagacagcctccctgctcctgaccagcagatacgtttcggagtggttggtgtggtttttgtgatgagggcagcacatggtggccaaggtgggcaaagctgagtctcacaaggctcaaatcccttcggttgggntccccttgtgggg

SEQ ID NO: 95 - DNA, 2458 bp, Homo sapiens
gcgggcggagatgtagacccggtagtgttgtgccttgtggtgacaactggcggcagcgcgccgcgggcccgagacttagtctcgggccgccatggccagcgtccacgagagcctctacttcaatcccatgatgaccaatggggttgtgcacgccaatgtgttcggcatcaaggactgggtgacgccgtacaagatcgcggtgctggtgctgctgaacgagatgagccgcacaggcgagggcgccgtcagcctcatggagcggcggaggctcaaccagctgctcctgcccctgctgcagggcccagatattacactgtcaaaactttacaagttaattgaagagtcttgtccacagctggcaaattcagtgcagatcagaatcaaactgatggctgaaggcgagttgaaggatatggaacagttttttgatgacctttcagattctttctctggaactgaaccagaggttcacaaaacaagtgtagtaggtttgtttctgcgtcacatgatcttggcctacagtaagctttctttcagccaagtgtttaaactgtacactgcccttcagcagtacttccagaatggtgagaaaaagacagtggaggatgctgatatggaactgaccagtagagatgagggtgaaagaaaaatggaaaaagaagaacttgatgtatctgtaagagaagaggaggtatcttgcagtgggcctctgtcccaaaaacaagcagaattttttctttctcaacaggcttctttgctaaagaatgatgagactaaggccctcactccagcttccttgcagaaggaattaaacaatttgttgaaatttaatcctgattttgctgaagcgcattatctcagctacttaaacaacctccgtgtccaagatgttttcagttcaacacacagtctcctccattattttgatcgtctgattcttaccggagccgaaagcaaaagtaatggggaagagggctatggccggagcttgagatacgccgctctgaatcttgccgccctgcactgccgcttcggtcactatcaacaggcagagctcgccctgcaggaggcaattaggattgcccaggagtccaacgatcacgtgtgtctccagcactgtttgagctggctttatgtgctggggcagaagagatccgatagctatgttctgctggagcattctgtgaagaaggcagtacattttgggttaccgagagcttttgctgggaagacggcaaacaagctgatggatgccctaaaggactccgacctcctgcactggaaacacagcctgtcagagctcatcgatatcagcatcgcacagaaaacggccatctggaggctgtatggccgcagcaccatggcactgcaacaggcccagatgttgctgagcatgaacagcctggaggcggtgaatgcgggcgtgcagcagaacaacacagagtcctttgctgtcgcactctgccacctcgcagagctacacgcggagcagggctgttttgctgcagcttctgaagtgttaaagcacttgaaggaacgatttccgcctaatagtcagcacgcccagttatggatgctatgtgatcaaaaaatacagtttgacagagcaatgaatgatggcaaatatcatttggctgattcacttgttacaggaatcacagctctcaatagcatagagggtgtttataggaaagcggttgtattacaagctcagaaccaaatgtcagaggcacataagcttttacaaaaattgttggttcattgtcagaaactgaagaacacagaaatggtgatcagtgtcctactgtccgtggcagagctgtactggcgatcttcctcccctaccatcgcgctgcccatgctcctgcaggctctggccctctccaaggagtaccggttacagtacttggcctctgaaacagtgctgaacttggcttttgcgcagctcattcttggaatcccagaacaggccttaagtcttctccacatggccatcgagcccatcttggctgacggggctatcctggacaaaggtcgtgccatgttcttagtggccaagtgccaggtggcttcagcagcttcctacgatcagccgaagaaagcagaagctctggaggctgccatcgagaacctcaatgaagccaagaactattttgcaaaggttgactgcaaagagcgcatcagggacgtcgtttacttccaggccagactctaccataccctggggaagacccaggagaggaaccggtgtgcgatgctcttccggcagctgcatcaggagctgccctctcatggggtacccttgataaaccatctctagagaggacatccctgctgggctgctgtgcagagtataagattttggacttgttcatgtcccctctctccctataaatgatgtatttgtgacaccctatcttgtcaataaacagcattctgattaaaaaaaaaaaaaaaa

SEQ ID NO: 96 - DNA, 2900 bp, Homo sapiens
  modified_base (2890): a, c, g, t, unknown or other
tgcatggatgggatactggatgaatctttgcttgaaacctgtccaattcagtcaccattacaagtttttgcaggaatgggtggactggctcttattgctgaaagactacccatgctatatccagaagtaattcaacaggtgagtgctccagttgtaacatctaccactcaggaaaagccgtatgatagcgatcagtttgaatgggtgaccattgaacagtcaggggagttagtttatgaagcaccagaaactgttgcggctgaacctccacctatcaagtcagcagtacagaccatgtctcccatacctgcccattctttggctgcttttggattatttcttcgtcttccgggctatgcggaagtgctactgaaagagagaaaacatgcccagtgccttcttcgattggtattgggagtgacagatgatggagaaggaagtcatattcttcaatctccatcagccaatgtgcttccaacccttcctttccacgtccttcgtagcttgtttagcactacacctttgacaactgatgatggtgtacttctaaggcggatggcattggaaattggagccttacacctcattcttgtctgtctctctgctttgagccaccattccccacgagttccaaactctagcgtgaatcaaactgagccacaggtgtcaagctctcataaccctacatcaacagaagaacaacagttatattgggccaaagggactggctttggaacaggctctacagcttctgggtgggatgtggaacaagccttaactaagcaaaggctggaagaggaacatgttacctgccttctgcaggttcttgccagttacataaatcccgtcagtagtgcggtaaatggagaagctcagtcatctcatgagactagagggcagaacagtaatgcccttccttctgtacttctcgagcttctcagtcagtcctgcctcatcccagccatgtcatcttatctacgaaatgattcagttctggacatggcaagacatgtgccactctatcgggcactgctggaattgcttcgggccattgcttcttgtgctgccatggtgcccctattgttgcccctttctacagagaacggtgaagaggaagaagaacagtcagaatgtcaaacttctgttggtacattgttagccaaaatgaagacctgtgttgatacctataccaaccgtttaagatctaaaagggaaaatgttaaaacaggagtaaaaccagatgcgtctgatcaagaaccagaaggacttactcttttggtaccagacatccaaaagactgctgagatagtttatgcagccaccaccagtttgcggcaagcaaatcaggaaaaaaactgggtgaatactccaagaaggcggctaatgaaccccaaacctttgtcagtattaaagtcacttgaagaaaaatatgtggctgttatgaagaaattacagtttgatacgtttgaaatggtttctgaagatgaagatgggaaattgggatttaaagtaaattaccactacatgtctcaggtgaaaaatgctaatgatgcgaacagtgctgccagagctcgccgccttgcccaggaagctgtgacgctttcaacctcactgcctctgtcttcatcctctagtgtgtttgtacgctgtgatgaggagcgacttgatatcatgaaggttctaataactggtccagcggacaccccttatgcaaatggctgctttgagtttgatgtgtattttcctcaagattatcccagttcaccccctcttgtgaatctagagacaactggtggtcatagcgtgcgattcaatccaaacctttataatgatggcaaggtttgtttaagcatcttaaacacgtggcatggaagaccagaagagaagtggaatcctcagacctcaagctttttgcaagtgttggtgtctgtccagtcccttatattagtagctgagccttattttaatgaaccgggatatgaacggtctagaggcactcccagtggcacacagagttctcgagaatatgatggaaacattcgacaagcaacagttaagtgggcaatgctagaacaaatcagaaacccttcaccatgttttaaagaggtaatacacaaacatttttacttgaaaagagttgagataatggcccaatgtgaggagtggattgcggatatccagcagtacagcagtgataagcgggtaggcaggactatgtctcaccatgcagcagctctcaagcgtcacactgctcagctccgcgaagagttgctgaaacttccctgccctgaaggcttggatcctgacactgacgatgccccagaggtgtgcagagccacaacaggtgctgaggagactctaatgcatgatcaggttaaacccagcagcagcaaagaactccccagtgacttccagttatgagctgcattgatgtggacttcatagacacaaaggcttcgaagcacaagccaaatatgtcaatatttgtatgtaagaaactaattatgtaataggtaatgaaactgaaactatactatgcccttaaggagatccagtttaattcaaggtgatcttttatttacctgtacaggagtgtaaacttttttgtgcttttatttttcaattgtgagaaccactgattggtatgttcaacaaatttgtgtatacaaagaaatggataaatcactgctatataagggaaactaccttaggaaagaatgtttactgaatgtttattttattttattttttttttactatagagtgaggggttgttaacaaagaatatatattggtcgttcttacaactactatttaaagtcagcaacttttcactgaatttgatagattttatgtttgggggtacgagcttgtaaagctcgggtgcctnatgagtgacc

SEQ ID NO: 97 - DNA, 1310 bp, Homo sapiens
ccgctgagatgtacgaacttccggttctccgggcagctgccactgctgtagcttctgccacctgccacgaccgggcctctccctggcgtttggtcacctctgcttcattctccaccgcgcctatggtccctcttggagccagcgtggcgggcctggcggctcccgggtggtgagagagcggtccgggaacgatgaaggcctcgcagtgctgctgctgtctcagccacctcttggcttccgtcctcctcctgctgttgctgcctgaactaagcgggcccctggcagtcctgctgcaggcagccgaggccgcgccaggtcttgggcctcctgaccctagaccacggacattaccgccgctgccaccgggccctacccctgcccagcagccgggccgtggtctggctgaagctgcggggccgcggggctccgagggaggcaatggcagcaaccctgtggccgggcttgagacggacgatcacggagggaaggccggggaaggctcggtgggtggcggccttgctgtgagccccaaccctggcgacaagcccatgacccagcgggccctgaccgtgttgatggtggtgagcggcgcggtgctggtgtacttcgtggtcaggacggtcaggatgagaagaagaaaccgaaagactaggagatatggagttttggacactaacatagaaaatatggaattgacacctttagaacaggatgatgaggatgatgacaacacgttgtttgatgccaatcatcctcgaagaagagaatgtgccttttgatgaaagaactttatctttctacaatgaagagtggaatttctatgtttaaggaataagaagccactatatcaatgttgggggggtatttaagttacatatattttaacaacctttaatttgctgttgcaataaataccgtatccttttattatatctttatatgtatagaagtactctattaatgggctcagagatgttggggataaagtatactgtaataatttatctgtttgaaaattactataaaacggtgttttctgatcggtttttgtttcctgcttaccatatgattgtaaattgttttatgtattaatcagttaatgctaattatttttgctgatgtcatatgttaaagagctataaattccaacaaccaactggtgtgtaaaaataatttaaaatttcctttactgaaaggtatttcccatttttgtggggaaaagaagccaaatttattactttgtgttggggtttttaaaatattaagaaatgtctaagttattgtttgcaaaacaataaatatgattttag

SEQ ID NO: 98 - DNA, 2272 bp, Homo sapiens
ccatgctccaggcatacagatgtggtttctcggctgcaccgggccaggctgcgggtgtgcaggcgtctgcaaagttgtgccatgtatcagcacaggctttgagacgtctggaccctgtccttcctcccgtgaggggttcttgttctttctgactcaggtgacttttcagcccttccaattcccctctttttctgccctcccctccaactcagccaacccaggtgtgggcagtcagggagggagggagtgtcccaccacgttctcagggcagcccttgactcctaagccccttcctccttccattctgcatcccctccccatccaacctaaatgcccacagctggggctgagctgtattcctgtggagggacctctgccgtgcctctctgaggtcaggctgtgctgtgtgatgggcaggctttgccccagcccacccctggcaaggtgcacttgttttctggtttgtacaaggtgtcctgggggcccgtcgcttccctgccagtgaggagtgacttctccctctcttccagtcctgtaggggagacaaaaccagattggggggcccaaggggagcatggaaaaggccggctcccctgtctttccttggctgtcagagtcagggtaacacacaccaagagtggagtgcggccagcaagtttgagacctgcccgccctcctcgcagctctgctctgtgtcctcaggaagtcacagagtctactgaggcaaggagagggtgattctttccccaaatcccttcttccctggttcccaaaccaaagacagcctgcagccctttctgcatggggtgctctgttgacaggcttcccagatccctgagtctctctttccttcctcctcgatctttagttgtccacggtcaattcagtgcttccattgggggacagtcccctccgggatgacctgattcacctccagcccagggaatggaatctagaggaatacgtggggtgggtctggacaaggagcggcaggaatcaccacccatctccagctgtggagccctgtggaggggaaggggaagcttggggttcagagggaactcttccaggagaggggtgcccagcggaggtaaagatgatagagggttgtggggggtctctagttgaatgttttggcccatgactttggaacatggctggcagcttccagcagaagtcacgctccccatcccccaggggacataggacctttttcctgcttcctggtcactttcaaagaactatttgcgcaatctgtgggtctgtggattcacggggctttctgtgtgggtgctgcagttgcttttgtctgcagcagcaggacacatctttcctcttactcagccctttatggcccatggggaactccgtggctcagggagagctgaactccaggggtgtgacctgggacaggtgggcctgaggtgcccagctcagggcagccaggtggctcatgggctgtagtgagccagctccctgggggaaaaggctgtgggccgttaggaccatcctccaggacaggtgacctctatgaggtcacctacggctgtggccgtgcaggcctccttccagcccagagtggcccagtagagcaaggcagacagtgacctccacccccgcagccctcttaaaaggccagtactcttgggggtggggggagggtttagaaagcatttgcccatctgcctttctttcccccagcccccacccgctttgaatgtagagacccgtgggcacttttccttttgtggtggggggtgcggaggaggtacccccacccctggcacagccgcctggaatgcaggactgtcactgctgttcgggtgatgacctcgttgccaagctcctcctgtccccttgttctgggggcaggcgctgtgcttctgtgaggtggtttagcttttgctttcgaagtggccagctgcggccaccaggtctcagcacaagagcgcttcctttgcacagaatgagcttcgagctttgttcagactaaatgaatgtatctgggaggggtcgggggcacgagttgattccaagcacatgcctttgctgagtgtgtgtgtgctgggagagtcagagtggatgtagagcgcggttttatttttgtactgacattggtaagagactgtatagcatctatttatttagatgatttatctggtaaatgaggcaaaaaaattattaaaaatacattaaagatgatttaaaaaaaagaccaaaaaaccaagaaacccaaagcccaagaatgcgcgtagcatccaaaaaaaaaagg

SEQ ID NO: 99 - DNA, 1060 bp, Homo sapiens
gtcaacttagcgagcgcaacaggctgccgctgaggagctggagctggtggggactgggccgcaatggacaagctgaagaaggtgctgagcgggcaggacacggaggaccggagcggcctgtccgaggttgttgaggcatcttcattaagctggagtaccaggataaaaggcttcattgcgtgttttgctataggaattctctgctcactgctgggtactgttctgctgtgggtgcccaggaagggactacacctcttcgcagtgttttatacctttggtaatatcgcatcaattgggagtaccatcttcctcatgggaccagtgaaacagctgaagcgaatgtttgagcctactcgtttgattgcaactatcatggtgctgttgtgttttgcacttaccctgtgttctgccttttggtggcataacaagggacttgcacttatcttctgcattttgcagtctttggcattgacgtggtacagcctttccttcataccatttgcaagggatgctgtgaagaagtgttttgccgtgtgtcttgcataattcatggccagttttatgaagctttggaaggcactatggacagaagctggtggacagttttgtaactatcttcgaaacctctgtcttacagacatgtgccttttatcttgcagcaatgtgttgcttgtgattcgaacatttgagggttacttttggaagcaacaatacattctcgaacctgaatgtcagtagcacaggatgagaagtgggttctgtatcttgtggagtggaatcttcctcatgtacctgtttcctctctggatgttgtcccactgaattcccatgaatacaaacctattcagcaacagcacataagccttgggtgcaagtgattcccaggtggcaaaaggcagccccatcagagatcacgggagcaacagtaagggacagagttttggggtccacttgtccctcagcatggaagccatcaccgtggtcctgcatagagtgagtctgcttctactctggcatctgagaacaagtgactctgctttagacaagcccctggagaggg

SEQ ID NO: 100 - DNA, 543 bp, Homo sapiens
gctcacagtagcccggcggccagggcaatccgaccacatttcactctcaccgctgtaggaatccagatgcaggccaagtacagcagcacaagggacatgctggatgatgatggggacaccaccatgagcctgcattctcaagcctctgccacaactcggcatccagagccccggcgcacagagcacagggctccctcttcaacgtggcgaccagtggccctgaccctgctgactttgtgcttggtgctgctgatagggctggcagccctggggcttttgtgtaagtctgcgctctgacctgggggaggatcctggttccaagtttttcagtactaccagctctccaatactggtcaagacaccatttctcaaatggaagaaagattaggaaatacgtcccaagagttgcaatctcttcaagtccagaatataaagcttgcaggaagtctgcagcatgtggctgaaaaactctgtcgtgagctgtataacaaagctggagcacacaggtgcagcccttgtacagaacaatggaaatggcatgga

SEQ ID NO: 101 - DNA, 2281 bp, Homo sapiens
agctggctcaccttccagattcacctgcaggagctgctgcagtacaagaggcagaatccagctcagttctgcgttcgagtctgctctggctgtgctgtgttggctgtgttgggacactatgttccagggattatgatttcctacattgtcttgttgagtatcctgctgtggcccctggtggtttatcatgagctgatccagaggatgtacactcgcctggagcccctgctcatgcagctggactacagcatgaaggcagaagccaatgccctgcatcacaaacacgacaagaggaagcgtcaggggaagaatgcacccccaggaggtgatgagccactggcagagacagagagtgaaagcgaggcagagctggctggcttctccccagtggtggatgtgaagaaaacagcattggccttggccattacagactcagagctgtcagatgaggaggcttctatcttggagagtggtggcttctccgtatcccgggccacaactccgcagctgactgatgtctccgaggatttggaccagcagagcctgccaagtgaaccagaggagaccctaagccgggacctaggggagggagaggagggagagctggcccctcccgaagacctactaggccgtcctcaagctctgtcaaggcaagccctggactcggaggaagaggaagaggatgtggcagctaaggaaaccttgttgcggctctcatcccccctccactttgtgaacacgcacttcaatggggcagggtccccccaagatggagtgaaatgctcccctggaggaccagtggagacactgagccccgagacagtgagtggtggcctcactgctctgcccggcaccctgtcacctccactttgccttgttggaagtgacccagccccctccccttccattctcccacctgttccccaggactcaccccagcccctgcctgcccctgaggaagaagaggcactcaccactgaggactttgagttgctggatcagggggagctggagcagctgaatgcagagctgggcttggagccagagacaccgccaaaaccccctgatgctccacccctggggcccgacatccattctctggtacagtcagaccaagaagctcaggccgtggcagagccatgagccagccgttgaggaaggagctgcaggcacagtagggcttcttggctaggagtgttgctgtttcctcctttgcctaccactctggggtggggcagtgtgtggggaagctggctgtcggatggtagctattccaccctctgcctgcctgcctgcctgctgtcctgggcatggtgcagtacctgtgcctaggattggttttaaatttgtaaataattttccatttgggttagtggatgtgaacagggctagggaagtccttcccacagcctgcgcttgcctccctgcctcatctctattctcattccactatgccccaagccctggtggtctggccctttctttttcctcctatcctcagggacctgtgctgctctgccctcatgtcccacttggttgtttagttgaggcactttataatttttctcttgtcttgtgttcctttctgctttatttccctgctgtgtcctgtccttagcagctcaaccccatcctttgccagctcctcctatcccgtgggcactggccaagctttagggaggctcctggtctgggaagtaaagagtaaacctggggcagtgggtcaggccagtagttacactcttaggtcactgtagtctgtgtaaccttcactgcatccttgccccattcagcccggcctttcatgatgcaggagagcagggatcccgcagtacatggcgccagcactggagttggtgagcatgtgctctctcttgagattaggagcttccttactgctcctctgggtgatccaagtgtagtgggaccccctactagggtcaggaagtggacactaacatctgtgcaggtgttgacttgaaaaataaagtgttgattggctagaactgctgcctccctgactgtgagctgccttccacaccctgcactgcactgtgttctctcctcacccttaacctgcttcactccagtctgttctggctgtttattaccttgttgcaaaacagggccgaagcaaggattaccttgacaaccctagcttctccttagccatcttccttgacagtgtgatctgtttagtgagatttagcatgtgtgaataaagtatatgcaggaggaaattgctttgtcttcccaatcggtagaaattcgagacctagcc

SEQ ID NO: 102 - DNA, 992 bp, Homo sapiens
gacagcttggcctacagcccggcgggcatcagctcccttgacccagtggatatcggtggccccgttattcgtccaggtgcccagggaggaggacccgcctgcagcatgaacctgtggctcctggcctgcctggtggccggcttcctgggagcctgggcccccgctgtccacgcccaaggtgtctttgaggactgctgcctggcctaccactaccccattgggtgggctgtgctccggcgcgcctggacttaccggatccaggaggtgagcgggagctgcaatctgcctgctgcgatattctacctccccaagagacacaggaaggtgtgtgggaaccccaaaagcagggaggtgcagagagccatgaagctcctggatgctcgaaataaggtttttgcaaagctccgccacaacacgcagaccttccaagcaggccctcatgctgtaaagaagttgagttctggaaactccaagttatcatcatccaagtttagcaatcccatcagcagcagcaagaggaatgtctccctcctgatatcagctaattcaggactgtgagccggctcatttctgggctccatcggcacaggaggggccggatctttctccgataaaaccgtcgccctacagacccagctgtccccacgcctctgtcttttgggtcaagtcttaatccctgcacctgagttggtcctccctctgcacccccaccacctcctgcccgtctggcaactggaaagagggagttggcctgattttaagccttttgccgctccggggaccagcagcaatcctgggcagccagtggctcttgtagagaagacttaggatacctctctcactttctgtttcttgccgtccaccccgggccatgccagtgtgtccctctgggtccctccaaaactctggtcagttcaaggatgcccctcccaggctatgcttttctataacttttaaataaaccttgggggttgatggagtcaaaaaaaaaa

SEQ ID NO: 103 - DNA, 1554 bp, Homo sapiens
tcgcccaggagtcatcggacgccagaatctgtgtctccagaacgctatagctatggcacctccagctcttcaaagaggacagagggtagctgccgtcgccgtcggcagtcaagcagttctgcaaattctcagcagggtcagtgggagacaggctcccccccaaccaagcggcagcggcggagtcggggccggcccagtggtggtgccagacggcggcggagaggggccccagccgcaccccagcagcagtcagagcccgccagaccttcctctgaaggcaggtgacactgtgatggggaaacaggctcagagagacatccggctccgggttcgagcagagtactgcgagcatgggccagccttggagcagggcgtggcatcccggcggccccaggcgctggcgcggcagctggacgtgtttgggcaggccaccgcagtgctgcgctcaagggacctgggctctgtggtttgtgacatcaagttctcagagctctcctatctggacgccttctggggcgactacctgagtggcgccctgctgcaggccctgcggggcgtgttcctgactgaggccctgcgagaggctgtgggccgggaggctgttcgcctgctggtcagtgtggatgaggctgactatgaggctggccggcgccgcctgttgctgatggcggaggaaggggggcggcgcccgacagaggcctcctgatccaggactggcaggattgatcccacctccaagtctccgggccaccttctcctgggaggacgaccatctctacccctagaggactgtcactctagcatctttgaggactgcgacaggaccgggacagcaggccccttgacagcccctcccacaggatgtgggctctgaggcctaaaccatttccagctgagtttccttcccagactcctcctacccccaggtgtgcccccttagcctccggaggcgggggctgggcctgtatctcagaagggaggggcacagctacacactcaccaaaggcccccctgcacattgtatctctgatcttgggctgtctgcactgtcacaggtgcacacactcgctcatgctcacactgcccctgctgagatcttccctgggcctctgccctggcctgcttcccagcacacacttctttggcctaagggcttctctctcaggacctctaatttgaccacaaccaacctgggcttcagccacatcagtgggcactggagctggggtgcacatggggcctgctcaccttgcccacacatctccagccagccagggccctgcccagcttcaatttacagacctgactctcctcaccttcccccctgctgtccagagctgaacatagacttgcacttggatgtcacctggagtgtcacatgggagtgttatggcagcatcataccaaggcctactgttgcacatggggccaaaaccagtaaacagccaccttcttggaaagggaatgcaaaggctttgggggtgatggaaaagaccttttacaaatgataccaattaaactgccctggaaagggcataggtgggaaaaaaaaaa

SEQ ID NO: 104 - DNA, 1802 bp, Homo sapiens
gtcgccgggcttgcgatgaacttccggctgtcaagctcccggccgggctgactcaagcggaggcgcgcggaacagtcgccgaggcgattcccgcccaggctcctgtaaccgccaggcagcggccccgccatgtcccagccccggaccccagagcaggcactggatacaccgggggactgccccccaggcaggagagacgaggacgctggggaggggatccagtgctcccaacgcatgctcagcttcagtgacgccctgctgtccatcatcgccaccgtcatgatcctgcctgtgacccacacggagatctccccagaacagcagttcgacagaagtgtacagaggcttctggcaacacggattgccgtctacctgatgacctttctcatcgtgacagtggcctgggcagcacacacaaggttgttccaagttgttgggaaaacagacgacacacttgccctgctcaacctggcctgcatgatgaccatcaccttcctgccttacacgttttcgttaatggtgaccttccctgatgtgcctctgggcatcttcttgttctgtgtgtgtgtgatcgccatcggggtcgtgcaggcactgattgtggggtacgcattccacttcccgcacctgctgagcccgcagatccagcgctctgcccacagggctctgtaccgacgacacgtcctgggcatcgtcctccaaggcccggccctgtgctttgcagcggccatcttctctctcttctttgtccccttgtcttacctgctgatggtgactgtcatcctcctcccctatgtcagcaaggtcaccggctggtgcagagacaggctcctgggccacagggagccctcggctcacccagtggaagtcttctcgtttgacctccacgagccactcagcaaggagcgcgtggaagccttcagcgacggagtctacgccatcgtggccacgcttctcatcctggacatctgcgaagacaacgtcccggaccccaaggatgtgaaggagaggttcagcggcagcctcgtggccgccctgagtgcgaccgggccgcgcttcctggcgtacttcggctccttcgccacagtgggactgctgtggttcgcccaccactcactcttcctgcatgtgcgcaaggccacgcgggccatggggctgctgaacacgctctcgctggccttcgtgggtggcctcccactagcctaccagcagacctcggccttcgcccggcagccccgcgatgagctggagcgcgtgcgtgtcagctgcaccatcatcttcctggccagcatcttccagctggccatgtggaccacggcgctgctgcaccaggcggagacgctgcagccctcggtgtggtttggcggccgggagcatgtgctcatgttcgccaagctggcgctgtacccctgtgccagcctgctggccttcgcctccacctgcctgctgagcaggttcagtgtgggcatcttccacctcatgcagatcgccgtgccctgcgccttcctgttgctgcgcctgctcgtgggcctggccctggccaccctgcgggtcctgcggggcctcgcccggcccgaacaccccccgccagcccccacgggccaggacgacccacagtcccagctcctccctgccccctgctagcagccacagagcccactcccagccgtcctcaccagagatggaccagggaggacaggatgctgggcaggggaagccaagtcacgggcaggccgcagtggttcttgcgtggcctggttttattttcattgtgaaatatcatgctcttatttcagtcctca

SEQ ID NO: 105 - DNA, 1395 bp, Homo sapiens
gtacctcggcttatttcataaacaggtactgaaggaagcagaggcatgtggaggacttccccacctcgtgcagctatttgggccgtggcatctgaaatttcttatttcagagtcacccctttgatgaccttggcagtgaactgcagtcatctgtttaggcctttccatggcccacgtcaatgccggtatttctgtttgttgcacatttgatttccttgttgttggcatttagaaggccctcgagccgcactgagggactgagcctggtgtatatggcagcaagactggatggtggctttgcagcagtctccagagcattccatgagatccgggctcgaaatccagcatttcagccacaaactttgatggactttggctcaggtactggttctgtcacctgggctgctcacagtatttggggccagagcctacgtgaatatatgtgtgtggacagatcagctgccatgttggttttggcagaaaaactactgacaggtggttcagaatctggggagccttatattccaggtgtctttttcagacagtttctacctgtatcacccaaggtgcagtttgatgtagtagtgtcagctttttccttaagtgaccagctactgacatttatactttcgtgtaattcaagtcttctgcatattttccccttttgtgaacaggtactggtggagaatggaacaaaagctgggcacagccttctcatggatgccagggatctggtccttaagggaaaagagaagtcacctttggaccctcgacctggttttgtctttgccccgtgtccccatgaactcccttgtccccagttgaccaacctggcctgtagcttctcacaggcgtaccatcccatccccttcagctggaacaagaaaccaaaggaagaaaagttctctatggtgatccttgctcgggggtctccagaggaggctcatcgctggccccgtatcactcagcctgtccttaaacggcctcgccatgtgcattgtcacttgtgctgtccagatgggcacatgcagcatgctgtgctcacagcccgccggcacggcaggtatggggggtgtgaccaaaatcagtgggatgtggcaggaagctgcagcccacgccagcatctgtttccacagggatttgtatcgttgtgcccgtgtcagctcctggggagatcttttacctgtgcttactccgtctgcgtttcctccatctacggctcaggatccctctgagagttgatgaggatgtgtaacaagtattttcttctatcgtgcctgccagggctgaagctgcctggtatccaggaggggaatgctggtatccccatatgtctgtgtttgtttgagatttttaataataaataataaatttttgaagaatggaaaaaaaaaa

SEQ ID NO: 106 - DNA, 1635 bp, Homo sapiens
ccctcttccttttgcgcacggaagaacaaatcacaacaatcacacaccaggactgaatccatcagcagatactgccctgtgggaagggcagaggaaagagaagacagacggactgacagacaccacagaggaacaggggagttagcctgggaccaatggaggagaagtacgaaccctgggaaaaagacgtgtcagatgagaaagttccggagagtccgatgtctcatcgcaggtgttacatcatcagggtttgccattggaatactgagtggagatgggaaagagaaaagttaagggctgaaatgggaggggaatgggaagaaaaaatgagagacaagagggaaataagaaaaaacaaagagagcacaaagaccagtttaggagaaaggaccaatggggacagtggcagagtggcgaggtaggtgaaggactgaggcacagcgtcctgttgtggagggaggaaaggcaagcgttccgaggtggtgaaaaggaaggcctgctaggcacggtggggatgaacgaggatgccatgagtcacacaaaagacagtgctggtgaggcccagccacaggagcctcagataacttggtaaaggcatgtctcccatttgggaactgatgttcctaagatccgcactgacgctgctcagccggtccatcacacagcaaaggcgtgaggaagggtcactgcccagctggactccagggtggtccacgcatgacagtcacaccgaaccttcatgaggatgtgaactgttggctccaatttaccattcccagcaattccactcagatatttgtatactaatgttcacagcagcgtgaactccacagcaggtggagtaatgttccattgtgtgcatatgccacattttgtttatccattcatctgttgatgcacatttcggttgttcccacctttgggctattattaataatgctgctgtgaacattcccaagagaaataggaagacggctttgctaagaactaaaaaagggatggacaacaagggcatatacccaggggcagtgttctatcatgacagctttactgagagcagagtagttctgctcagaatcagaacacttgttccctatagcccccctgattgccccacaaccaccaccgcatactccccttttcccaaccatgggcagcagattgagctattaacagaagtgtcctttcgctggatttctcaaccctttcctcatcgtccacatagagaaacagtaacagattgctactcacccaacacccaggtcaagtccaatgcaggtaggaataacagcaaatccttcaatttcttgattctgctcttaaaaatcttaacagaggcttccaggttctgaaaatattttctgcataaacgtgtgacactccatcacgaaactccctttggttatctgcttaaacttatcgcaaatgtctggaacgctggtggcttccaaaatcaactcctggtgctgcttaattaaggtcagggccacccggaagataatcttcgagccttcgttaaacaaacagtcccagatccgaagcactgtctccacgggcaagatgtccacaaacaggcagatgaaccagcgggacaccagcagcgtccacagcacaccgagacgctccatcaggggg

SEQ ID NO: 107 - DNA, 1485 bp, Homo sapiens
  modified_base (13): a, c, g, t, unknown or other
tttttggtcccgncnaaagnccnaaaacccggnacccgggaagccnccccaanncnaaanttcccagttngaancccgaaggnaaaaccccggaaaagnanncngccccnaaanttcncgggcnaaaacccggccnttttttcccccccgggcggccgttttgggccccngantttccatttaaantnccnagncttgggcaacctaaccaggnttttcccccaanctggaaaaagccgggccaagttgagccgcacccgccccagaagttcaagggcccccggcctcctgcgctcctgccgccgggaccctcgacctcctcagagcagccggctgccgccccgggaagatggcgaggaggagccgccaccgcctcctcctgctgctgctgcgctacctggtggtcgccctgggctatcataaggcctatgggttttctgccccaaaagaccaacaagtagtcacagcagtagagtaccaagaggctattttagcctgcaaaaccccaaagaagactgtttcctccagattagagtggaagaaactgggtcggagtgtctcctttgtctactatcaacagactcttcaaggtgattttaaaaatcgagctgagatgatagatttcaatatccggatcaaaaatgtgacaagaagtgatgcggggaaatatcgttgtgaagttagtgccccatctgagcaaggccaaaacctggaagaggatacagtcactctggaagtattagtggctccagcagttccatcatgtgaagtaccctcttctgctctgagtggaactgtggtagagctacgatgtcaagacaaagaagggaatccagctcctgaatacacatggtttaaggatggcatccgtttgctagaaaatcccagacttggctcccaaagcaccaacagctcatacacaatgaatacaaaaactggaactctgcaatttaatactgtttccaaactggacactggagaatattcctgtgaagcccgcaattctgttggatatcgcaggtgtcctgggaaacgaatgcaagtagatgatctcaacataagtggcatcatagcagccgtagtagttgtggccttagtgatttccgtttgtggccttggtgtatgctatgctcagaggaaaggctacttttcaaaagaaacctccttccagaagagtaattcttcatctaaagccacgacaatgagtgaaaatgatttcaagcacacaaaatcctttataatttaaagactccactttagagatacaccaaagccaccgttgttacacaagttattaaactattataaaactctgctttgtccgacatttgcaaagaggtacacgaggaaatggaattggtatttcattttaattttcatgactactaactcacctgaacttgctattttaaacaaatagttctgtcgacacctaaaatataatctggcttcttgtgtctggactaagttaaaagaattaaaata

SEQ ID NO: 108 - DNA, 810 bp, Homo sapiens
cgagtgagcgcgcggcggcccctggtccgcccggccgcggccgatctaggggctgggggctggaggcgggggtgggggtctgagctgcgtcctgggctcgaggcgtcccccggggagtcgcctcttagcggtgcgtccgggctagcggcgaggggccgccccaagtcttcccaccgccgccaccttagcagcccgacttggggcctggaaagtggagcacgcggaggtgggagggccctgcacgcggcccccggtggggaaggggacgggccagggattcagactcgggctctcccctcaggatgcagcaccgaggcttcctcctcctcaccctcctcgccctgctggcgctcacctccgcggtcgccaaaaagcaagataaggtgaagaagggcggcccggggagcgagtgcgctgagtgggcctgggggccctgcacccccagcagcaaaggatttgcggcagtgggttttccgcgagggccaccttgggggggcccaagaacccaaccggcagtcctggttgaaagggttgcccctggaaagttggaaagaaaggagttttgggcacccggactttggaaagttggccaaattttttggaagaaaacttggcgggtctgccggtccgttaaatgggggaggggacaaaagaattgaaagccgaaaaaatgctttctccgccgccaagagaggtcgaacccgcgtctggcaagaagagaaaagggcgcgcccacactgttaacaacaatatggcgcctgaacagttggtggcaccacagggggagggagacacatacttgcgcgcggt

SEQ ID NO: 109 - DNA, 1064 bp, Homo sapiens
ttcctggggctccggggcgcggagaagctgcatcccagaggagcgcgtccaggagcggacccgggagtgtttcaagagccagtgacaaggaccaggggcccaagtcccaccagccatgcagacctgccccctggcattccctggccacgtttcccaggcccttgggaccctcctgtttttggctgcctccttgagtgctcagaatgaaggctgggacagccccatctgcacagagggggtagtctctgtgtcttggggcgagaacaccgtcatgtcctgcaacatctccaacgccttctcccatgtcaacatcaagctgcgtgcccacgggcaggagagcgccatcttcaatgaggtggctccaggctacttctcccgggacggctggcagctccaggttcagggaggcgtggcacagctggtgatcaaaggcgcccgggactcccatgctgggctgtacatgtggcacctcgtgggacaccagagaaataacagacaagtcacgctggaggtttcaggtgcagaaccccagtccgcccccgacactgggttctggcctgtgccagcggtggtcactgctgtcttcatcctcttggtcgctctggtcatgttcgcctggtacaggtgccgctgttcccagcaacgccgggagaagaagttcttcctcctagaaccccagatgaaggtcgcagccctcagagcgggagcccagcagggcctgagcagagcctccgctgaactgtggaccccagactccgagcccaccccaaggccgctggcactggtgttcaaaccctcaccacttggagccctggagctgctgtccccccaacccttgtttccatatgccgcagacccatagccgcctgcaaggaagagaggacacaggagtagccaccctgagtgccgacctttggtggcgggggcctgggtctctcgtccccacccggaagggcacaagacaccgggctttgcttggcaaggcttggggcctcttgtggtcaacccagttcccttgggtgccgttgcagaaccccttagccccttccaacgtcgaccaggtt

SEQ ID NO: 110 - DNA, 1031 bp, Homo sapiens
agttcctgcaggtgccggcggtgacgcgggcttacaccgcagcctgtgtcctcatccaccgccgcggtgcagctggagctcctcagcccctttcaactctacttcaacccgcaccttgtgttccggaagttccaggtgaggccgcctcgcgccgcgcacctggggcccgacccacccaccccgcacctgaccgcccgtcccccgtaggtctggaggctcgtcaccaacttcctcttcttcgggcccctgggattcagcttcttcttcaacatgctcttcgtgtatcctgcgcctgcggacacgggctgggtggagggcaggccggccgggctgggagagaggccgggacggggaaactgaggccccgcctggtggcacttcctataccgacgccgtaggttccgctactgccgcatgctggaagagggctccttccgcggccgcacggccgacttcgtcttcatgtttctcttcgggggcgtccttatgaccgtatccttcccgcaggctctggaacctcgggctagggcgcctcggcgtccagcctgtgttggtcctggggccaacacagccatgccagagagggacacagtcgctgtctccagcttagcaccgttcctgccttgggcgctcatgggcttctcgctgctgctgggcaactccatcctcgtggacctgctggggattgcggtgggccatatctactacttcctggaggacgtcttccccaaccagcctggaggcaagaggctcctgcagacccctggcttcctaaagctgctcctggatgcccctgcagaagaccccaattacctgcccctccctgaggaacagccaggaccccatctgccacccccgcagcagtgacccccacccagggccaggcctaagaggcttctggcagcttccatcctacccatgacccctacttggggcagaaaaaacccatcctaaaggctgggcccatgcaagggcccacctgaataaacagaatgagctgcaaaaaaaaaaaaaaagggcggccgtcgcga

SEQ ID NO: 111 - DNA, 2316 bp, Homo sapiens
gctggataagacaccaggggagtcactacatggttaccgcatctgtatccaggccatcctgcaagacaagcccaagattgccacggcaaacctaggcaagttcctggaactgctgaggtcccaccagagccgaccagcaaagtgtctcaccatcatgtgggccctgggtcaagcaggttttgccaacctcaccgagggactgaaagtgtggctggggatcatgctgcctgtgctgggcatcaagtctctgtctccctttgccatcacatacctggatcggctgctcctgatgcatcccaaccttaccaagggcttcggcatgattggccccaaggacttcttcccacttctggactttgcctatatgccgaacaactccctgacacccagcctgcaggagcagctgtgtcagctctacccccgactgaaaatgctggcatttggagcaaagccggattccaccctgcatacctacttcccttctttcctgtccagagccacccctagctgtccccctgagatgaagaaagagctcctgagcagcctgactgagtgcctgacggtggaccccctcagtgccagcgtctggaggcagctgtaccctaagcacctgtcacagtccagccttctgctggagcacttgctcagctcctgggagcagattcccaagaaggtacagaagtctttgcaagaaaccattcagtccctcaagcttaccaaccaggagctgctgaggaagggtagcagtaacaaccaggatgtcgtcacctgtgacatggcctgcaagggcctgttgcagcaggttcagggtcctcggctgccctggacgcggctcctcctgttgctgctggtcttcgctgtaggcttcctgtgccatgacctccggtcacacagctccttccaggcctcccttactggccggttgcttcgatcatctggcttcttacctgctagccaacaagcgtgtgccaagctctactcctacagtctgcaaggctacagctggctgggggagacactgccgctctggggctcccacctgctcaccgtggtgcggcccagcttgcagctggcctgggctcacaccaatgccacagtcagcttcctttctgcccactgtgcctctcaccttgcgtggtttggtgacagtctcaccagtctctctcagaggctacagatccagctccccgattccgtgaatcagctactccgctatctgagagagctgcccctgcttttccaccagaatgtgctgctgccactgtggcacctcttgcttgaggccctggcctgggcccaggagcactgccatgaggcatgcagaggtgaggtgacctgggactgcatgaagacacagctcagtgaggctgtccactggacctggctttgcctacaggacattacagtggctttcttggactgggcacttgccctgatatcccagcagtaggccctgccttcctggccactgatttctgcatgggtagaccatccaagactgcagcgggtagaaggtggcagttcttcatgggagtctttttaacttggtgcctgagttctctcctaggcaagtggccagttgcctccacctcagttcttccatctttggtggggacagggcccagcagcatctcagcctcctacccacaattccactgaacacttttctggccctactgcacatggcccccagcctccatccttgtgctggtagcctctcacaactccgcccttgccctctgccttccacttccttccatctcatttctaaaccccaaacagctcatctctaaaaagatagaactcccagcaggtggcttctgtgttcttctgacaaatgattcctgcttctccagactttagcagcctcctgttcccattcttggtcacagctctagccacagcagaaggaaaggggcttccagaagaatatagcaccgcattgggaaacagcagcctcacctccacctgaagcctgggtgtggctgtcagtggacatggggagctggatggaaatgcctctcacttcaaaatgcccagcctgccccaaatgcctctaagcccctccctgtcccctcccttgtagtcctacttcttccaactttccattccccatcatgctgggggtcttggtcacaaggctcagcttctctccactgtccatccctcctatcatctgtagagcagagcacaggcagttgtgtgccttgggcccagggaaccctccatcaacctgagacaggactcagtatatggttcttgggtatgccctaccaggtggaataaaggacacagatttgaaaaaaaaaaaaaaaa

SEQ ID NO: 112 - DNA, 1169 bp, Homo sapiens
agcaaggagccagaggccatgcagtggctcagggtccgtgagtcgcctggggaggccacaggacacagggtcaccatggggacagccgccctgggtcccgtctgggcagcgctcctgctctttctcctgatgtgtgagatccctatggtggagctcacctttgacagagctgtggccagcggctgccaacggtgctgtgactctgaggaccccctggatcctgcccatgtatcctcagcctcttcctccggccgcccccacgccctgcctgagatcagaccctacattaatatcaccatcctgaagggtgacaaaggggacccaggcccaatgggcctgccagggtacatgggcagggagggtccccaaggggagcctggccctcagggcagcaagggtgacaagggggagatgggcagccccggcgccccgtgccagaagcgcttcttcgccttctcagtgggccgcaagacggccctgcacagcggcgaggacttccagacgctgctcttcgaaagggtctttgtgaaccttgatgggtgctttgacatggcgaccggccagtttgctgctcccctgcgtggcatctacttcttcagcctcaatgtgcacagctggaattacaaggagacgtacgtgcacattatgcataaccagaaagaggctgtcatcctgtacgcgcagcccagcgagcgcagcatcatgcagagccagagtgtgatgctggacctggcctacggggaccgcgtctgggtgcggctcttcaagcgccagcgcgagaacgccatctacagcaacgacttcgacacctacatcaccttcagcggccacctcatcaaggccgaggacgactgagggcctctgggccaccctcccggctggagagctcaggtgctggtcccgtcccctgcagggctcagtttgcactgctgtgaagcaggaaggccagggaggtccccggggacctggcattctggggagaccctgcttctatcttggctgccatcatccctcccagcctatttctgctcctctcttctctcttggacctattttaagaagcttgctaacctaaatattctagaactttcccagcctcgtagcccagcacttctcaaacttggaaatgcatgcgaatcacccggggttcgtgttaaatgcagattctg

SEQ ID NO: 113 - DNA, 1530 bp, Homo sapiens
tcacagactgcggagtgggtcaggggctgcgagggctgccccaagtcctaccgggtttgcacgggcgcgcccggctccgcccgcaagtgcgccttcctgacttactgctgggtgcgcggggctgggggtgcgagtaccacccctgaagtctcttcctgggcgacctccggggcctcattctaggcctccttaaagagaaggatctaaattaggaaaaggaagtgcccttatccacgaccaagctcttccacctgcggagctcgcttagtctgcacctcaaccgtgcggaaagtgactgccctgtttactgaggaaaaactggggctcagaaagataccatgagtagtttgaaacaggaacaaaatcttctgaaagctcggagcagaagcctttttggtcaacatggaggaaaaaagacggcgagcccgagttcagggagcctgggctgcccctgttaaaagccaggccattgctcagccagctaccactgctaagagccatctccaccagaagcctggccagacctggaagaacaaagagcatcatctctctgacagagagtttgtgttcaaagaacctcagcaggtagtacgtagagctcctgagccacgagtgattgacagagagggtgtgtatgaaatcagcctgtcacccacaggtgtatctagggtctgtttgtatcctggctttgttgacgtgaaagaagctgactggatattggaacagctttgtcaagatgttccctggaaacagaggaccggcatcagagaggatataacttatcaacaaccaagacttacagcatggtatggagaacttccttacacttattcaagaatcactatggaaccaaatcctcactggcaccctgtgctgcgcacactaaagaaccgcattgaagagaacactggccacaccttcaactccttactctgcaatctttatcgcaatgagaaggacagcgtggactggcacagtgatgatgaaccctcactagggaggtgccccattattgcttcactaagttttggtgccacacgcacatttgagatgagaaagaagccaccaccagaagagaatggagactacacatatgtggaaagagtgaagatacccttggatcatggtaccttgttaatcatggaaggagcgacacaagctgactggcagcatcgagtgcccaaagaataccactctagagaaccgagagtgaacctgacctttcggacagtctatccagaccctcgaggggcaccctggtgacgtcagagctttgagagagaagcttcactgaaacggagcaaaccttccactgagaagccacttcaagaggctggtgctgctagatctcatgatgtggctgttgggaagatggtggggtttgtttgccagcttggagtcctattaaatgaaagccagcaactcatgttggtaataggtctactgtgggaacagttatccctaaccacagctcaaaatcgctatcatctttaggcaaattaaaatctatgtggcagtgaaaaaaaaaaa

SEQ ID NO: 114 - DNA, 1336 bp, Homo sapiens
agctcgtacccctcgagtgaaattctgaaatgaagatggaggaggcagtgggaaaagttgaagaactcattgagtccgaagccccaccaaaagcatctgaacaagagacagccaaggaggaagatggatctgtagaactggaatctcaagttcagaaagatggtgtagcggattctacagttatttcttcaatgccctgcttgttgatggaactgagaagggactcttctgagtctcagttagcatccacagagagtgacaagcctacaactggccgagtttatgagagtgacccctctaatcactgcatgctttccccttcctctagtggtcacctggctgattcagatacgttgtcttccgcagaagagaatgaaccctctcaggcagaaacggcggtagaaggagacccttcaggagtgtctggtgccacagttgggcgcaagtctaggcggtcccgatctgaaagtgaaacttccactatggctgccaagaaaaaccggcaatccagtgataaacagaatggccgagtcgccaaggttaaaggtcatcggagccaaaagcacaaggagaggatcaggctactgaggcagaaacgggaggctgctgcaaggaagaaatataacctgctgcaggacagtagtaccagtgatagtgacctgacttgtgactcaagcacgagctcatcagatgatgatgaagaggtttcagggagcagcaagacaatcactgcagagataccagatggacctccagttgtagctcattatgatatgtctgacaccaactctgacccagaagtggtaaatgtggacaatttattggcggctgcagtagttcaagagcacagtaattctgtaggcggccaggacacaggagctacctggaggaccagcgggcttctagaggagctgaatgcagaggcaggtcatttggatccaggattcctagcaagtgacaaaacatctgctggcaatgcgccactcaatgaagaaattaacattgcgtcttcagatagtgaagtagagattgtgggagttcaggaacatgcaaggtgtgttcatcctcgaggtggtgtgattcagagtgtttcttcatggaagcatggctcgggcacgcagtatgttagcaccaggcaaacacagtcatggactgctgtgactccccagcagacttgggcttcaccagcagaagttgttgaccttaccttggatgaggatagcaggcgtaaatacctactgtaatacaatgtcactgtgtttcctctgcactgttcccttccacttcctcatcctctttgtgacatggaagttcattgtcataggggtacggagct

SEQ ID NO: 115 - DNA, 1742 bp, Homo sapiens
gccccgccccctccccgcccgccttcccggtgaccttcaggggcccgggtggcgggcgcaggcccctgcggcggcggcgggatgttcgtgcaggaggagaagatcttcgcgggcaaggtgctgcggctgcacatctgcgcgtccgacggcgccgagtggctggaggaggccaccgaggacacctcggtggagaagctcaaggagcgctgcctcaagcactgtgctcatgggagcttagaagatcccaaaagtataacccatcataaattaatccacgctgcctcagagagggtgctgagtgatgccaggaccatcctggaagagaacatccaggaccaagatgtcctattattgaaaaaaaagcgtgctccatcaccacttcccaagatggctgatgtctcagcagaagaaaagaaaaaacaagaccagaaagctccagataaagaggccatactgcgggccaccgccaacctgccctcctacaacatggaccgggccgcggtccagaccaacatgagagacttccagacagaactccggaagatactggtgtctctcatcgaggtggcgcagaagctgttagcgctgaacccagatgcggtggaattgtttaagaaggcgaatgcaatgctggacgaggacgaggatgagcgtgtggacgaggctgccctgcggcagctcacggagatgggctttccggagaacagagccaccaaggcccttcagctgaaccacatgtcggtgcctcaggccatggagtggctaattgaacacgcagaagacccgaccatagacacgcctcttcctggccaagctcccccagaggccgagggggccacagcagctgcctccgaggctgccgcgggagccagcgccaccgatgaggaggccagagatgagctgacggaaatcttcaagaagatccggaggaaaagggagtttcgggctgatgctcgggccgtcatttccctgatggagatggggttcgacgagaaagaggtgatagatgccctcagagtgaacaacaaccagcagaatgccgcgtgcgagtggctgctgggggaccggaagccctctccggaggagctggacaagggcatcgaccccgacagtcctctctttcaggccatcctggataacccggtggtgcagctgggcctgaccaacccgaaaacattgctagcatttgaagacatgctggagaacccactgaacagcacccagtggatgaatgatccagaaacggggcctgtcatgctgcagatctctagaatcttccagacactaaatcgcacgtaggtggcgttgttccactcggctatcaggccacagcagccccctggtgcggcccgagaccgggcagagtggacctcacctggaaactcaccttcagcgcctcagccctggactgttagaggtgctgcagctgctcctgctctctgatcttattgcttataaactttggtgacggtagtgtgtaaggccgtatttttagcatctgacaggtgtttacaaaaaagtggttgtcgcactgggaagtggagtgatggcctcgtctccagtgctcctctgggctcttgagttgctgcttgaattgccgtgtagacatttgcttggagagtccacttgttatttgacggaggtaggtttcaacccagagttaatgtcaagcatgctaatttaactagtcactcacagatgacttttctttaataaagtcccttttcctattaaaaaaaaaa

SEQ ID NO: 116 - DNA, 1074 bp, Homo sapiens
  modified_base (546): a, c, g, t, unknown or other
gcggtgcagaggaagcacaacctctaccgggacagcatggtcatgcacaacagcgaccccaacctgcacctgctggccgagggcgcccccatcgactggggcgaggagtacagcaacagcggcgggggcggcagcccagccccagcaccccggagtcagccaccctctcggaaaagcgacggcgcgccaagcaggtggtctctgtggtccaggatgaggaggtggggctgccctttgaggctagccctgagtcaccaccacctgcgtccccggacggtgtcactgagatccgaggcctgctggcccaaggtctgcggcctgagagccccccaccagccggccccctgctcaacggggcccccgctggggagagtccccagcctaaggccgcccccgaggcctcctcgccgcctgcctcacccctccagcatctcctgcctggaaaggctgtggaccttgggccccccaagcccagcgaccaggagactggagagcaggtgtccagccccagcagccaccccgccctccacaccaccaccgaggacnantttcaaggggtgcaagaattgaagnttcntaagggccaanttgggggtccccttgacttggnttggnaanattggggcaaaaagggccggttttccccntttcccggganaccccaagggaaaggggnttcaaagcttcttngggggggaaagggggaancccttgggtnttttgttggccntttgtgancancagcgaggagagtgcaaaggtgcagagtnagttntaggncantgggtccctgactgctgcanatggtaaggncgttnncttgtggacccaaggcaggnaaagntgtggggagggaagctggtntgtgcnttgtgggtggaagcgggganggctgtgttgnanggcagggagagggcnaantgagttatttattggggttcangtgaaaagtttcttgnnccctgtnttgtgttnctgtgggattgattntaagatngnnaggggtnggtttttggggttttcctggttggtggccaaangggttggaaaatngntggggggggnttgganaat

SEQ ID NO: 117 - DNA, 1454 bp, Homo sapiens
cccgggggaggcctgaccccctccgcaccaccgtacggagccgcatttcccccgtttcccgaggggcatccagccgtgttgcctggggaggacccacccccctattcacccttaactagcccggacagtgggagtgcccctatgatcacctgccgagtctgccaatctctcatcaacgtggaaggcaagatgcatcagcatgtagtcaaatgtggtgtctgcaatgaagccaccccaatcaagaatgcacccccagggaaaaaatatgttcgatgcccctgtaactgtctccttatctgcaaagtgacatcccaacggattgcatgccctcggccctactgcaaaagaatcatcaacctggggcctgtgcatcccggacctctgagtccagaaccccaacccatgggtgtcagggttatctgtggacattgcaagaatacttttctgtggacagagttcacagaccgcactttggcacgttgtcctcactgcaggaaagtgtcatctattgggcgcagatacccacgtaagagatgtatctgctgcttcttgcttggcttgcttttggcagtcactgccactggccttgcctttggcacatggaagcatgcacggcgatatggaggcatctatgcagcctgggcatttgtcatcctgttggctgtgctgtgtttgggccgggctctttattgggcctgtatgaaggtcagccaccctgtccagaacttctcctgagcctgatgacccacagactgtgcctggcccctccctggtggggacagtgacactacgaagggagctggggtagttaaaggctcccggggcttctagaaggaagccaagcagctgccttccttttccctggggagaggtaggaaggaaccaggccctcacttaggtttggaggggcagataagagcactgctgaccatctgctttcctccaagggttgctgtgtctagggtgaagtaggcaaaacgttgcccttaaaactgggccctgaagacggttccagccttgtccttcctgtgtgctccctgagagccattcctgtcccttacacattccagggcagggtgggggtgggtagccctgggggttcccctccctcttgtgcaccattaggactttgctgctgctattgcacttcaccagaggttggctctggcctcagtaccctcagtctcctctccccacattgtgtcctgtgggggtggggtcagccgctgctctgtacagaaccacaggaactgatgtgtatataactatttaatgtgggatatgttcccctattcctgtatttcccttaattcctcctcccgaccttttttacccccccagttgcagtatttaactgggctgggtagggttgctcagtctttgggggaggttagggacttatcctgtgcttgtaaataaataaggtcatgactctaaaaaaaaaaaaagggcgg

SEQ ID NO: 118 - DNA, 2071 bp, Homo sapiens
agctttgaattcctgtatctgagaacggatcgttcgaggtggtggagggggttggaattggggacctacggaaggctcagctcttgccaggccaaattgagacatgtctgacacaagcgagagtggtgcaggtctaactcgcttccaggctgaagcttcagaaaaggacagtagctcgatgatgcagactctgttgacagtgacccagaatgtggaggtcccagagacaccgaaggcctcaaaggcactggaggtctcagaggatgtgaaggtctcaaaagcctctggggtctcaaaggccacagaggtctcaaagaccccagaggctcgggaggcacctgccacccaggcctcgtctactactcagctgactgatacccaggttctggcagctgaaaacaagagtctagcagctgacaccaagaaacagaatgctgacccgcaggctgtgacaatgcctgccactgagaccaaaaaggtcagccatgtggctgatacaaaggtcaatacaaaggctcaggagactgaggctgcaccctctcaggccccagcagatgaacctgagcctgagagtgcagctgcccagtctcaggagaatcaggatactcggcccaaggtcaaagccaagaaagcccgaaaggtgaagcatctggatggggaagaggatggcagcagtgatcagagtcaggcttctggaaccacaggtggccgaagggtctcaaaggctctaatggcctcaatggcccgcaggtttcaaggggtcccatagccttttgggcccgcaggattcaaggactcggttggctgcttgggcccggagagccttgctctccctgagatcacctaaagcccgtagggcaaggctcgccgtagagctgccaagctccagtcatcccaagagcctgaagcaccaccacctcgggatgtggcccttttgcaagggagggcaaatgatttggtgaagtaccttttggctaaagaccagacgaagattcccatcaagcgctcggacatgctgaaggacatcatcaaagaatacactgatgtgtaccccgaaatcattgaacgagcaggctattccttggagaaggtatttgggattcaattgaaggaaattgataagaatgaccacttgtacattcttctcagcaccttagagcccactgatgcaggcatactgggaacgactaaggactcacccaagctgggtctgctcatggtgcttcttagcatcatcttcatgaatggaaatcggtccagtgaggctgtcatctgggaggtgctgcgcaagttggggctgcgccctgggatacatcattcactctttggggacgtgaagaagctcatcactgatgagtttgtgaagcagaagtacctggactatgccagagtccccaatagcaatccccctgaatatgagttcttctggggcctgcgctcttactatgagaccagcaagatgaaagtcctcaagtttgcctgcaaggtacaaaagaaggatcccaaggaatgggcagctcagtaccgagaggcgatggaagcagatttgaaggctgcagctgaggctgcagctgaagccaaggctagggccgagattagagctcgaatgggcattgggctcggctcggagaatgctgccgggccctgcaactgggacgaagctgatatcggaccctgggccaaagcccggatccaggcgggagcagaagctaaagccaaagcccaagagagtggcagtgccagcactggtgccagtaccagtaccaataacagtgccagtgccagtgccagcaccagtggtggcttcagtgctggtgccagcctgaccgccactctcacatttgggctcttcgctggccttggtggagctggtgccagcaccagtggcagctctggtgcctgtggtttctcctacaagtgagattttagatattgttaatcctgccagtctttctcttcaagccagggtgcatcctcagaaacctactcaacacagcactctaggcagccactatcaatcaattgaagttgacactctgcattaaatctatttgccatttcaaaaaaaaaa

SEQ ID NO: 119 - DNA, 1236 bp, Homo sapiens
acctgggacccccagaacggccgcccctttttttttttttttttttttttttttttttttttttttttttttttttttttttttttttttttttttttagaaggttgaaaccaggcttatttattttcatcttctttctgccatcttttaaccaaccttctcagaataaaatgtgatttttgagacagaatgaaacacatatccaaattttaatacagtaagaataggtatcctgaataaatgagaactctagaaaatcaaggtttcaaaattctacccttcctgggagttaaagaagtttggcagaaacagaacaaattaatcagcagattcatcacctgccaattttttctgtacaattttcttgattctgggagcatctgggtccaggcagattttcctcccatccttcagtgtggctgcttcttgtttcatccatggaccctgcaagaaattgccccatgtttctgtttgtgcatcactgagaaaggaagcatgaaggtcgcacaggtcaggccattccattgccctcctggtgccgggtttgccctcccaatcctggggttgcttcaggggcttgtcattctccatagtcccctccacatttctcaggtttctgctcaaaagtcaccttttggaggggtctccacctgtcactgtgtttgtaagagctccttcagtttctttctagctcatctcactctggtaatgtctttgattaccaccaccatctgacctggtcttatgacctgttagctttcttcatcagacgtgagcaccaggatggcaggggcctcatctgtcctgttcctcctgtggcctgggtcctagcaccatgtctggtacagtgtagatgctcaagggaagtttactttgtaaaaccacttacctgggagatgttactgttagtctaacctgtaccattttgtaaacctccagccattttgcagactctgatcacagtgaaacgttccatgggaacttgggccatgagaaacatccttcctaaccacgtgactgcagaaacatccttatcgcgtcctcctgggcaaaggcccaacagcctgactgcagggacatccttgccatatcctgctgggcagcaagctctaccacccagatccctccctcccagtcccatgattaccccagcctgtgagtggcagttggtgctggcactaagctggtttcctcctccccagggttttgctggcaataaagatgttgctgttgaag

SEQ ID NO: 120 - DNA, 1391 bp, Homo sapiens
gtactgcccaccacctccctgggccacccctcactcagtgctccggctctctcctcctcctcttcgtcctcctccacttcatctcctgttttgggctccccctcttaccctgcttcttcccctggggcctccccccaccaccgccgtgtgcccctcagccccctgagtttgctcgcgggcccagccgacgccagaaggtcccaacagcagctgcccaaacagttttcgccaacaatgtcacccaccttgtcttccatcactcagggcgtccccctggataccagtaaactgtccactgaccagcggttacccccatacccatacagctccccaagtctggttctgcctacccagccccacaccccaaagtctctacagcagccagggctgccctctcagtcttgttcagtgcagtcctcaggtgggcagcccccaggcaggcagtctcattatgggacaccgtacccacctgggcccagtgggcatgggcaacagtcttaccaccggccaatgagtgacttcaacctggggaatctggagcagttcagcatggagagcccatcagccagcctggtgctggatccccctggcttttctgaagggcctggatttttagggggtgaggggccaatgggtggcccccaggatccccacaccttcaaccaccagaacttgacccactgttcccgccatggctcagggcctaacatcatcctcacaggggactcctctccaggtttctctaaggagattgcagcagccctggccggagtgcctggctttgaggtgtcagcagctggattggagctagggcttgggctagaagatgagctgcgcatggagccactgggcctggaagggctaaacatgctgagtgacccctgtgccctgctgcctgatcctgctgtggaggagtcattccgcagtgaccggctccaatgagggcacctcatcaccatccctcttcttggccccatcccccaccaccattcctttcctcccttccccctggcaggtagagactctactctctgtccccagatcctctttctagcatgaatgaaggatgccaagaatgagaaaaagcaaggggtttgtccaggtggcccctgaattctgcgcaagggatgggcctgggggaactcaagggagggcctaaagcacttgtaactttgaaccgtctgtctggaggtcagagcctgttggaaagcaggggtagaggggagccctggaagcagggcttttccggatgcctaggggtgggcagtgccagcccctcctcaccactcttccccttgcagtggaggagagagccagagtggatactattttttattaaatatattattatatgttaataaaaaaatcatatcaaaaaaaaaaaaaaaagg

SEQ ID NO: 121 - DNA, 2183 bp, Homo sapiens
  modified_base (2179): a, c, g, t, unknown or other
ctctgtgaacatatgatgagagaagccaagatcatgcagtataagtacctactgttcagtcttcacgccatagtgaagcttggaatccctcagaacactattttggtgcagactttgctgagggtgacccaggaacgtatcaatgagtgtgatgagatatgcctttcagttttgtcaactgttttagaggcaatggaaccatgcaagaatgttcatgttctacgaacgggattcagaatactagttgatcagcaagtttggaaaatagaagatgtcttcacattacaagttgtgatgaagtgtattggaaaagatgcaccgattgctcttaagaggaaactggagatgaaagccttgaggggattagacagattttctgttttgaatagccaacacatgtttgaagtactagctgccatgaatcaccgatctcttatactcctggatgaatgcagtaaggtggtcctagataatatccatgggtgtcctttaagaataatgatcaacatattgcagtcctgcaaagacctccagtaccataatttggatctcttcaagggacttgcagattatgtggctgcaactttcgacatctggaagttcagaaaagttctttttatcctcattttatttgaaaaccttggctttcgacctgttggtttaatggacctgtttatgaagagaatagtagaggatcctgaatccctaaacatgaaaaacattctatctattcttcatacttactcttctctcaatcatgtctacaaatgccagaacaaagaacagttcgtggaagttatggctagtgctctgactggttatcttcacactatttcttctgaaaacttattggatgcagtatattcattttgcttgatgaattactttcccctggctccttttaatcagcttctgcaaaaagacatcatcagtgagctgctgacatcagatgacatgaagaatgcttacaagctgcatactttggatacttgtctaaaacttgatgatactgtctatctgagggacatagccttgtcactcccacagctgccgcgggagctgccatcgtcacatacaaatgcaaaggtggcagaggtgctgagcagccttctgggaggtgaaggacacttctcaaaggatgtgcacttgccacacaattatcatattgattttgaaatcagaatggacactaacaggaatcaagtgctaccactttctgatgtggatacaacttctgctacagatattcaaagagtagctgtgctatgtgtttccagatctgcttattgtttgggttcaagccaccccagaggattccttgctatgaaaatgcggcatttgaatgcaatgggttttcatgtgatcttggtcaataactgggagatggacaaactagagatggaagatgcagtcacatttttgaagactaaaatctattcagtagaagctcttcctgttgctgctgtaaatgtgcaaagcacacaataaagtgaaaatcaaccttttcatattaggagacatgcatttgtaaaaattaataaagatgacaagtcagttgtcaatggaattgagctatctgctaagacaaaaaatgttacctcagttcactattaaaattaattttaggagtggaagaaatgttgttactgccatttaaaaatatgctgagaaaattccagaagggttatttttccaaccacacctattccctctagtgcccagatatttgatttgtgagctgtacgtttcaccttttcatctttgatctactaaaaactggtttcttagttgtgaggtgtcacaggcaggttgatgtgggtagtagtccttgtctttggaatctgaatatttatactcctgctctaagctgttctaagacttggggttatgcctttaaatcattttcaagcattggccaaataataattggacaaagttctaaagttgtcaagtgtgtaagaattagtgaggtagctgttgaaaatgagtgaggatggtatttgtatttgtaataagcactgcaggtagagatatttcatgggttataataagagaaacacagatgagatgtagatggtaaggagtcttactgttgttggggtccttcctttctctttcttttttcccccttacccctcccacaatttcatgaagtcttttaaattaaatatatagcttnaatt

SEQ ID NO: 122 - DNA, 2066 bp, Homo sapiens
agaaccactgcagtggagactccatgtgcaaaagaaaaaaaccaaatgtgaggtcataaagactttctgccagcatgtgggtgacattgtttctttgcagattttggctatggaaaggggaaatgttctaagcagagccccgtcaagagcccacgggacacattttggagatgacagatttgaagatctggaagaggcaaatccattctcttttagagagtttctgaagaccaagaacctcggcctctcgaaagaggatccggccagcagaatttatgcaaaggaagcctcgaggcattccctgggacttgaccacaactccccaccctcccaaaccggcgggtatggcctggagtatcagcagccatttttcgaggatccgacaggggctggtgacctcctggatgaggaggaggatgaggacaccggatggagtggggcctacctgccgtccgccatcgagcagactcaccccgagagggtccctgccggcacgtcgccctgcagcacatacctttcctttttctccaccccgtcggagctggcagggcctgagtctctgccctcgtgggcgttgagtgacactgattctcgcgtgtctccggcctctccggcagggagtcctagcgcagactttgcggttcatggagagtctctgggagacaggcacctgcggacgctgcagataagttacgacgcactgaaagatgaaaattctaagctgagaagaaagctgaatgaggttcagagcttctctgaagctcaaacagaaatggtgaggacgcttgagcggaagttagaagcaaaaatgatcaaggaggaaagcgactaccacgacctggagtcggtggttcagcaggtggagcagaacctggagctgatgaccaaacgggctgtaaaggcagaaaaccacgtcgtgaaactaaaacaggaaatcagtttgctccaggcgcaggtctccaacttccagcgagagaatgaagccctgcggtgcggccagggtgccagcctgaccgtggtgaagcagaacgccgacgtggccctgcagaacctccgggtggtcatgaacagtgcacaggcttccatcaagcaactggtttccggagctgagacactgaatcttgttgccgaaatccttaaatctatagacagaatttctgaagttaaagacgaggaggaagactcttgaggacccctgggtgttctcagcatgaagctccgtgtataccctgaggtcaccaccgctcgatctaaatgtgcagttgtgtccttaaatatgcagtcttcacccagagtaaagtgttgatcgcaagagtccagtgtcgtgccctcagccagttcttggccaccacaatgggagcagccctggccgagttgtctctgtggtttctatgcagcccttcttggcgaaattcctgcgatcttatagattctaatgagctcttggaagacattgtcataaaagccagtgattttaagaaaaagagtggttctggaatcaatgttttccagtcccatcccagaacatcagttgtaagataagtacaattggttgtccttgatttcataagtagaacaaacactaaatgtgcctctgagatggccaccccgggcagggacctgtgccttccgccgatgctcagggctccctctggctcccgggtcactcttgtggccccagtgggtggtccctgcagtcatggcctgagtgcgcaggggccaccgcgtggctgctgctgtcctcctccgggacccacggggaccaaggtcacacgttccgtgctgtgaagctgtccagatgtgcctctttggctgggggttctggtggacgtttcaagtggcattttgtacaatgcaggttagaattcaggaatttcaagtatgtgcccgggtctgtcaggtcccagttgcctttctgacggcccccctcagagggacggcgatgagcactaaatgcttttttgactattttcctatagattttttttaaaacttttttttcctcctgttccaattgatagctttcttatttaataaattctgtagttcaccgcaaaaaaaaaaaa

SEQ ID NO: 123 - DNA, 1867 bp, Homo sapiens
  modified_base (1420): a, c, g, t, unknown or other
tggccaggctggtctagaactcctgactgcaaatgatcagcccgcctcagccacccaaagtgttgggattacaggtgtgagccactgtgcccagcgtgattttttttttttttaaagcaaacttgtcctttggttttgcagaacaggcctgctccctctcatctagcccatcatttcttggggcctgaaccccagtggtccaaagtattgcttgtgaaatttaaaaaatgtgaatatgatgtggggatgggcctcttctacattaccttggcccagggggatcagctggctgggaggattagtgagcacctctgtattttgaggtctgagtcttctggagctgtgtagttaatcttcggtttctgataacccctgggtccatctggccatcagcctcagcagtgagcaaagcaataccatactcatttctatgttcctgttccttcctctgctcctcctttggagaagcaataattcatgggggatgatacagtagcactttacaaatggctccatgtcattcatcccaggggccataatctcttgcaccacctattcttacttcctgttcagctcctttacagcttttattttcaactgcttcccaacttggtggggcctcctttaaggatgagccaatagtaagaatgtggctgtaatcagcagagacccctctgaggggtatctgttctgcagcccctagtgaaatcatgtgatgtgagacagaaacctaaacatggtacttgattctaaacctgtgccagtctatagcctctgcctccccaagcagagctcaagccaaacgcttctgtcctctttccttctgcattaaccctttgctgatcctcaggggccactcccccaacacccctgtacttgggtgagggatgttggacagagcctgttttcatgtactgcaggtgggggtgtgctgacatgtttgctcttggttgatggagaaggtacagaggccagggagtgaaaatggttgacagaagagggaagagttaggtgtctcatagtcactcatagtggggtggtcaggggtaatggcatctccccactttaggcttctcaaacagacttttgacacctctcaagttcagagctctgatgtggaaagacaggaggtgtggggaaggagggggatttcgtgtgtttgcatgagtgtgcgcttcaggccttgggagttggcaagagggagggaaggaaggagagcaaaatcttcggaaggtgtttcttgtacctgagggatcctgccctgaatctccatagtctccactgtgaactgaggaggggaggggtgtgctggggaataaatcttgtatgagaacaatcaaaaatcaaacgaatcccaccgacagactgctgctcctagtgatctggactcacctagggggcatctgggctggggttccangcttacgtncgcgtgnatgngacgncanagctcttcgaaagtgtcccnaaantncaattcattggcggtggttttaaaagttcgggcctgggaaacccgggggnttacccattttatcccncttnganggcanattcccctttttccccaatttggggaaatttnccaaangggncccgtaacggttggccttttcccaaaatttnggncgcccttaattggggcgattgtgggacccgcgccctttataggggggggctttaaagcggcgcngggggttctttgggtgattaccggcgcggttgaccccgggtaaaatattgacaagggccctttagcgcgcggttccttgtggggttttcctcccatttgctttttccgcaaaagttttggcggggttttccccggaaaaggtcttaaaaagcggtgtgcccctctttgaggggg

SEQ ID NO: 124 - DNA, 1628 bp, Homo sapiens
ctctgggtctgtagcaaccgcccagcgttgaggcgcggctcatgcccccagtatcccggtccagctattccgaggacatcgtgggctctcggagaaggcgacgcagctcctcggggagcccaccatccccgcagagcagatgttcctcttgggatggctgttcccgctctcactcccgcggccgtgagggcctcaggcctccttggagtgagttggacgtgggcgctctttacccctttagtcgctctgggtcgcgagggcggctcccaagattccgcaactacgccttcgcgtcctcctggtcgacctcgtatagtggatatcgctaccatcgtcactgctatgcagaagaacggcagtcagcggaagactacgagaaggaagagagccatcggcagaggaggctgaaggagagagagaggattggggaattgggagcgcctgaagtgtgggggccgtctccaaagttccctcagctagattctgacgaacataccccagttgaggatgaagaagaggtaacgcatcagaaaagcagcagttcagattccaactcggaagaacataggaaaaagaagaccagtcgttcaagaaacaagaaaaaaagaaagaataagtcgtctaaaagaaagcataggaaatattctgatagtgacagtaactcagagtctgacacaaattctgactctgatgatgataaaaagagagttaaagccaagaagaaaaagaagaaaaagaaacacaaaacaaagaaaaagaagaataagaaaaccaaaaaagaatccagtgactcaagctgtaaagactcagaagaggacttgtcagaagctacctggatggagcagccaaatgtggcagatactatggatttaatagggccagaagcacctataatacatacctctcaagatgaaaaacctttgaagtatggccatgctttgcttcccggtgaaggtgcagctatggctgagtatgtaaaagctggaaagcgaatcccacgaagaggtgaaattgggttgacaagtgaagagatcggttcttttgaatgctcaggttatgtcatgagtggtagcaggcatcgcagaatggaggctgtacgactgcgtaaggagaaccagatctacagtgctgatgagaagagagctcttgcatcctttaaccaagaagagagacgaaagagagaaagtaagattttagccagtttccgagagatggtgcacaaaaagacaaaagagaaagatgacaagtaaggacttacttgttgcacagcaggaattttaacaacaaaaattttatgtgaccaaaagtgttaaaaggctttacagtgctactgtacttaccatattagtaagtccctcaggaaaaagcttcttttgagatatctttagcagcttattttttgttattttaactttaaaaagtaatatgtgcacatggttttaaaaatattcaaccattataggaggagagttagtaaaaagtgaatctttcactttagcccctgacacctttcccccaaaaatatatattttggtgtcttatatacagaatatacattctgtgcatatacaagagtatatgttgcagcataaagattaaaagctattaaagttttttttcgctcgtta

SEQ ID NO: 125 - DNA, 1200 bp, Homo sapiens
gtggcggcggcgaaggatgcacccggcaggcttggcggcggcggctgcggggacgccccggctgccctcgaagcggaggatccctgtgtcccagccgggcatggccgacccccaccagcttttcgatgacacaagttcagcccagagccggggctatggggcccagcgggcacctggtggcctgagttatcctgcagcctctcccacgccccatgcagccttcctggctgacccggtgtccaacatggccatggcctatgggagcagcctggccgcgcagggcaaggagctggtggataagaacatcgaccgcttcatccccatcaccaagctcaagtattactttgctgtggacaccatgtatgtgggcagaaagctgggcctgctgttcttcccctacctacaccaggactgggaagtgcagtaccaacaggacaccccggtggccccccgctttgacgtcaatgccccggacctctacattccagcaatggctttcatcacctacgttttggtggctggtcttgcgctggggacccaggataggttctccccagacctcctggggctgcaagcgagctcagccctggcctggctgaccctggaggtgctggccatcctgctcagcctctatctggtcactgtcaacaccgacctcaccaccatcgacctggtggccttcttgggctacaaatatgtcgggatgattggcggggtcctcatgggcctgctcttcgggaagattggctactacctggtgctgggctggtgctgcgtggccatctttgtgttcatgatccggacgctgcggctgaagatcttggcagacgcagcagctgagggggtcccggtgcgtggggcccggaaccagctgcgcatgtacctgaccatggcggtggcggcggcgcagcctatgctcatgtactggctcaccttccacctggtgcggtgagcgcgcccgctgaacctcccgctgctgctgctgctgctgggggccactgtggccgccgaactcatctcctgcctgcaggccccaaggtccaccctgtctggccacaggcaccgcctccatcccatgtcccgcccagccccgcccccaacccaaggtgctgagagatctccagctgcacaggccaccgccccagggcgtggccgctgttacagaaacaataaaccctgatgggcatggcaaaaaaaaaa

SEQ ID NO: 126 - DNA, 1093 bp, Homo sapiens
agagccccagccacgccggcccaggtggcctcaggtgagggggggcggacgcacctgtggggacgggacgacgagttcaagcctccgtgggtgcagttggtcgccagcgagggatgcggagacgcccctgaacgaccatggcatcggccgacgagctgaccttccatgaattcgaggaggccactaatcttctggctgacaccccagatgcagccaccaccagcagaagcgatcagctgaccccacaagggcacgtggctgtggccgtgggctcaggtggcagctatggagccgaggatgaggtggaggaggagagtgacaaggccgcgctcctgcaggagcagcagcagcagcagcagccgggattctggaccttcagctactatcagagcttctttgacgtggacacctcacaggtcctggaccggatcaaaggctcactgctgccccggcctggccacaactttgtgcggcaccatctgcggaatcggccggatctgtatggccccttctggatctgtgccacgttggcctttgtcctggccgtcactggcaacctgacgctggtgctggcccagaggagggacccctccatccactacagcccccagttccacaaggtgaccgtggcaggcatcagcatctactgctatgcgtggctggtgcccctggccctgtggggcttcctgcggtggcgcaagggtgtccaggagcgcatggggccctacaccttcctggagactgtgtgcatctacggctactccctctttgtcttcatccccatggtggtcctgtggctcatccctgtgccttggctgcagtggctctttggggcgctggccctgggcctgtcagccgccgggctggtattcaccctctggcccgtggtccgtgaggacaccaggctggtggccacagtgctgctgtccgtggtcgtgctgctccacgccctcctggccatgggctgtaagttgtacttcttccagtcgctgcctccggagaacgtggctcctccaccccaaatcacatctctgccctcaaacatcgcgctgtcccctaccttgccgcagtccctggccccctcctaggaagg

SEQ ID NO: 127 - DNA, 1121 bp, Homo sapiens
gcgggggatgacgccacggacatggtggccgagaccggcggggtgggggacgtgtcgcgcggccgggtggcctcggtcggtaccctgggcgcggacagctgcctcattagtattcgtacccacgaggcggcgcagcgggccctcggggacagcgagcgtcgcggctatggcttatcactcgggctacggagcccacggctccaagcacagggcccgggcagccccggatccccctcccctcttcgatgacacaagcggtggttattccagccagcccgggggatacccagccacaggagcagacgtggccttcagtgtcaaccacttgcttggggacccaatggccaatgtggctatggcctatggcagctccatcgcatcccatgggaaggacatggtgcacaaggagctgcaccgttttgtgtctgtgagcaaactcaagtatttttttgctgtggacacagcctacgtggccaagaagctagggctgctggtcttcccctacacacaccagaactgggaagtgcagtacagtcgtgatgctcctctgcccccccggcaagacctcaacgcccctgacctctatatccccacgatggccttcattacttacgtgctcctggctgggatggcactgggcattcagaaaaggttctccccggaggtgctgggcctgtgtgcaagcacagcgctggtgtgggtggtgatggaggtgctggccctgctcctgggcctctacctggccaccgtgcgcagtgacctgagcacctttcacctgctggcctacagtggctacaaatacgtgggaatgatcctcagtgtgctcacggggctgctgttcggcagcgatggctactacgtggcgctggcctggacctcatcggcgctcatgtacttcattgtgcgctctttgcggacagcagccctgggccccgacagcatggggggccccgtcccccggcagcgtctccagctctacctgactctgggagctgcagccttccagcccctcatcatatactggctgactttccacctggtccggtgaccccctggccccagatggcactgagtttttcattcattgaagatttgatttccttgaaaaaaaaaaaaaaagg

SEQ ID NO: 128 - DNA, 1861 bp, Homo sapiens
cgcggactgtgtctgttcccaggagtccttcggcggctgttgtgtcagtggcctgatcgcgatggggacaaaggcgcaagtcgagaggaaactgttgtgcctcttcatattggcgatcctgttgtgctccctggcattgggcagtgttacagtgcactcttctgaacctgaagtcagaattcctgagaataatcctgtgaagttgtcctgtgcctactcgggcttttcttctccccgtgtggagtggaagtttgaccaaggagacaccaccagactcgtttgctataataacaagatcacagcttcctatgaggaccgggtgaccttcttgccaactggtatcaccttcaagtccgtgacacgggaagacactgggacatacacttgtatggtctctgaggaaggcggcaacagctatggggaggtcaaggtcaagctcatcgtgcttgtgcctccatccaagcctacagttaacatcccctcctctgccaccattgggaaccgggcagtgctgacatgctcagaacaagatggttccccaccttctgaatacacctggttcaaagatgggatagtgatgcctacgaatcccaaaagcacccgtgccttcagcaactcttcctatgtcctgaatcccacaacaggagagctggtctttgatcccctgtcagcctctgatactggagaatacagctgtgaggcacggaatgggtatgggacacccatgacttcaaatgctgtgcgcatggaagctgtggagcggaatgtgggggtcatcgtggcagccgtccttgtaaccctgattctcctgggaatcttggtttttggcatctggtttgcctatagccgaggccactttgacagaacaaagaaagggacttcgagtaagaaggtgatttacagccagcctagtgcccgaagtgaaggagaattcaaacagacctcgtcattcctggtgtgagcctggtcggctcaccgcctatcatctgcatttgccttactcaggtgctaccggactctggcccctgatgtctgtagtttcacaggatgccttatttgtcttctacaccccacagggccccctacttcttcggatgtgtttttaataatgtcagctatgtgccccatcctccttcatgccctccctccctttcctaccactgctgagtggcctggaacttgtttaaagtgtttattccccatttctttgagggatcaggaaggaatcctgggtatgccattgacttcccttctaagtagacagcaaaaatggcgggggtcgcaggaatctgcactcaactgcccacctggctggcagggatctttgaataggtatcttgagcttggttctgggctctttccttgtgtactgacgaccagggccagctgttctagagcgggaattagaggctagagcggctgaaatggttgtttggtgatgacactggggtccttccatctctggggcccactctcttctgtcttcccatgggaagtgccactgggatccctctgccctgtcctcctgaatacaagctgactgacattgactgtgtctgtggaaaatgggagctcttgttgtggagagcatagtaaattttcagagaacttgaagccaaaaggatttaaaaccgctgctctaaagaaaagaaaactggaggctgggcgcagtggctcacgcctataatcccagaggctgaggcaggcggatcacctgaggtcgggagttcgggatcagcctgaccaacatggagaaaccctactgagaatacaaagttagccaggcatggtggtgcatgcctgtaatcccagctgctcaggagcctggcaacaagagcaaaactccagctcaaaaaaaaaa

SEQ ID NO: 129 - DNA, 1975 bp, Homo sapiens
gtttggaggagactcggatataccttctcagaagctgcacaggaggaaagcagtgacaaagaaagaagttgtcattctttgcacgaaactggatggcttctacagggagccaggcctctgatatagacgagatttttggattcttcaacgatggcgaacctcccaccaaaaagcccaggaagctgcttccaagcttaaaaactaagaagcctcgagaacttgtgctagtgattggaacaggcattagtgctgcagttgcgccccaagttccagccctcaaatcctggaaggggttaattcaggccttactggatgctgccattgattttgatcttttagaagatgaggagagcaaaaagtttcagaaatgtctccatgaagacaagaacctggtccatgttgcccatgaccttatccagaaactctctcctcgtaccagtaatgttcgatccacatttttcaaggactgtttatatgaagtatttgatgacttggagtcaaagatggaagattctggaaaacagctacttcagtcagttctccacctgatggaaaatggagccctcgtattaactacaaattttgataatctcttggaactgtatgcagcagatcaggggaaacagcttgaatcccttgaccttactgatgagaaaaaggtcctcgagtgggctcaggagaagcgtaagctgagcgtgttgcatattcacggagtctacaccaaccctagtggcattgtccttcatccggctggatatcagaacgtgctcaggaacactgaagtcatgagagaaattcagaaactctacgaaaacaagtcatttcttttcctgggctgtggctggactgtggatgacaccactttccaggcccttttcttggaggctgtcaagcataaatctgacctagaacatttcatgctggttcggagaggagacgtagatgagttcaaaaagcttcgagaaaacatgctggacaaggggattaaagtcatctcctatggagatgactatgccgatcttccagaatatttcaagcgactgacatgtgagatctccacaaggggtacatcagcagggatggtgagagaaggtcagctaaatggctcatctgcagcacacagtgaaataagaggctgtagtacatgagcgagctagagaaatcaccaccgtttagaccaagctgtaaggccctactacagacagtgtttaacaagtaaacttacaagaacccaacacaattcccagaaagtaacaatagccagaggttgaagggcggggtagaagaggggggaatgttgcagcgtaatccttcataccacctggttcttgatattctgccgcctgttcaagttcaagaataaaagcgacagcaggacccaaatgcagctcccaacccactccccaggctagacatgcttgtgtccacacagcacaccaatgtgatacttccactgaccggctgcagctctgcatgaaggactcggggtctggatgccatggaatcactgtggctcttgttgcagttttgtactctatacttggtttttcaattaagcttaatggcttttttaaaacatgacttgaagctctagttttctagatcttttacagtgtacagtattttacataactaagctgtattaaaagcttgttcatttacttgccaggaccctggctctacttttagagtcattgtaagaaactctaacttgcatcaaggtactaataagcttaattttaataacccaaagtttaaaggttccgatctttctccttggggtggagtgatctcattctcaggacaaccgtttacttacctgattcctcggagcattatcaacttctgctctgttgtcctgaccatacatatgtcctagaactacagttaagtgtgttgtggaattttagttttgaatccggaataaatgaagtcccaggactcaaagaagagagaaaaaaaaaaagggggcccc

SEQ ID NO: 130 - DNA, 2160 bp, Homo sapiens
tctactgtcccctgccctgtacccccaggcattgatctggagaacattgtgtactacaaggacgacacccactactttgtgatgacagccaagaagcagtgcctgctgcggctgggggtgctgcgccaggactggccagacaccaatcggctgctgggcagtgccaatgtggtgcccgaggctctgcagcgctttacccgggcagctgctgactttgccacccatggcaagctcgggaaactagagtttgcccaggatgcccatgggcagcctgatgtctctgcctttgacttcacgagcatgatgcgggcagagagttctgctcgtgtgcaagagaagcatggcgcccgcctgctgctgggactggtgggggactgcctggtggagcccttctggcccctgggcactggagtggcacggggcttcctggcagcctttgatgcagcctggatggtgaagcggtgggcagagggcgctgagtccctagaggtgttggctgagcgtgagagcctgtaccagcttctgtcacagacatccccagaaaacatgcatcgcaatgtggcccagtatgggctggacccagccacccgctaccccaacctgaacctccgggcagtgacccccaatcaggtacgagacctgtatgatgtgctagccaaggagcctgtgcagagggacaacgacaagacagatacagggatgccagccaccgggtcggcaggcacccaggaggagctgctacgctggtgccaggagcagacagctgggtacccgggagtccacgtctccgatttgtcttcctcctgggctgatgggctagctctgtgtgccctggtgtaccggctgcagcctggcctgctggaaccctcagagctgcaggggctgggagctctggaagcaactgcttgggcactaaaggtggcagagaatgagctgggcatcacaccggtggtgtctgcacaggccgtggtagcagggagtgacccactgggcctcattgcctacctcagccacttccacagtgccttcaagagcatggcccacagcccaggccctgtcagccaggcctccccagggacctccagtgctgtattattccttagtaaacttcagaggaccctgcagcgatcccgggccaaggaaaatgcagaggatgctggtggcaagaagctgcgcttggagatggaggccgagaccccaagtactgaggtgccacctgacccagagcctggtgtacccctgacacccccatcccaacaccaggaggccggtgctggggacctgtgtgcactttgtggggaacacctctatgtcctggaacgcctctgtgtcaacggccatttcttccaccggagctgcttccgctgccatacctgtgaggccacactgtggccaggtggctacgagcagcacccaggcagtagaacgtctcagttcttcttctcagctcttgtggccatggagaaggaggaaaaagagagtcccttctccagtgaagaggaagaagaagatgtgcctttggactcagatgtggaacaggccctgcagacctttgccaagacctcaggcaccatgaataactacccaacatggcgtcggactctgctgcgccgtgcgaaggaggaggagatgaagaggttctgcaaggcccagaccatccaacggcgactaaatgagattgaggctgccttgagggagctagaggccgagggcgtgaagctggagctggccttgaggcgccagagcagttccccagaacagcaaaagaaactatgggtaggacagctgctacagctcgttgacaagaaaaacagcctggtggctgaggaggccgagctcatgatcacggtgcaggaattgaatctggaggagaaacagtggcagctggaccaggagctacgaggctacatgaaccgggaagaaaacctaaagacagctgctgatcggcaggctgaggaccaggtcctgaggaagctggtggatttggtcaaccagagagatgccctcatccgcttccaggaggagcgcaggctcagcgagctggccttggggacaggggcccagggctagacgagggtgggccgtctgctttcgttcccacaaagaaagcacctcaccccagcacagtgccacccctgttcatctgggctgcctggcaga

SEQ ID NO: 131 - DNA, 546 bp, Homo sapiens
  modified_base (390): a, c, g, t, unknown or other
gaggaagaagaggaagagggggctccgattgggacccctagggatcctggagatggttgtccttcccccgacatccctcctgaaccccctccaacacacctgaggccctgccctgccagccagctccctggactcctgtcccatggcctcctggccggcctctcctttgcagtggggtcctcctctggcctcctgcccctcctgctgctgctgctgcttccattgctggcagcccagggtgggggtggcctgcaggcagcgctgctggcccttgaggtggggctggtgggtctgggggcctcctacctgctcctttgtacagccctgcacctgccctccagtcttttcctactcctggcccagggtaccgcactgggggccgtcctgggncatgagctggcgccgaaggctcatgggtgttcccctggggctttggaactgcctggttcttaagcttnggcaaggcctagctccaacctctggtggctaatggcanccgggggggaanatgggttcnggaaaaagggcccccgggtttcaccgggg

SEQ ID NO: 132 - DNA, 581 bp, Homo sapiens
gccatggaggccctgaggagggcccacgaggtcgcgctccgcctgctgctgtgtaggccgtgggcctcgcgcgccgccgcccgccccaagcccagcgcctcggaggtgctgacgcggcatctgctgcagcggcgcctgccgcactggacctccttctgcgtgccctacagcgccgtccgcaacgaccagttcggcctctcgcacttcaactggccggtgcagggcgccaactaccacgtcctgcgcaccggctgcttccccttcatcaagtaccactgctccaaggctccctggcaggacctggcccggcagaaccgcttcttcacggcgctcaaggtcgtcaacctcggtattccaactttattatatggacttggctcctggttatttgccagagtcacagagactgtgcataccagttatggacccataacagtttattttctcaataaagaagatgaaggtgccatgtattgaaagtgtgcgtcaaagaacataaatatcagtggattttctctgtgtatatgtgcagtatttatttttgatcctttaaaataaaacttttgcaaataaaaaaaaaa

SEQ ID NO: 133 - DNA, 1259 bp, Homo sapiens
  modified_base (1191): a, c, g, t, unknown or other
gggctgggccccgccgcagctccagctggccggcttggtcctgcggtcccttctctgggaggcccgaccccggccgcgcccagcccccaccatgccacccgcggggctccgccgggccgcgccgctcaccgcaatcgctctgttggtgctgggggctcccctggtgctggccggcgaggactgcctgtggtacctggaccggaatggctcctggcatccggggtttaactgcgagttcttcaccttctgctgcgggacctgctaccatcggtactgctgcagggacctgaccttgcttatcaccgagaggcagcagaagcactgcctggccttcagccccaagaccatagcaggcatcgcctcagctgtgatcctctttgttgctgtggttgccaccaccatctgctgcttcctctgttcctgttgctacctgtaccgccggcgccagcagctccagagcccatttgaaggccaggagattccaatgacaggcatcccagtgcagccagtatacccatacccccaggaccccaaagctggccctgcacccccacagcctggcttcatgtacccacctagtggtcctgctccccaatatccactctacccagctgggcccccagtctacaaccctgcagctcctcctccctatatgccaccacagccctcttacccgggagcctgaggaaccagccatgtctctgctgccccttcagtgatgccaaccttgggagatgccctcatcctgtacctgcatctggtcctgggggtggcaggagtcctccagccaccaggccccagaccaagccaagccctgggccctactggggacagagccccagggaagtggaacaggagctgaactagaactatgaggggttggggggagggcttggaattatgggctatttttactgggggcaagggagggagatgacagcctgggtcacagtgcctgttttcaaatagtccctctgctcccaagatcccagccaggaaggctggggccctactgtttgtcccctctgggctggggtggggggagggaggaggttccgtcagcagctggcagtagccctcctctctggctgccccactggccacatctctggcctgctagattaaagctgtaaagacataactcatatcagtcgcatcattggacccatccacaccttccaggaacaccgncttcagctgggcccagactgttgcccactccatattccaaaagtaggggagggccagcaccagcatcg

SEQ ID NO: 134 - DNA, 2033 bp, Homo sapiens
cggctcgaggccgcagccccatggacagtcttctgcacccccgggagcgccctggatccactgcctccgagagctcagcctctctgggcagtgagtgggacctctcagaatcttctctcagcaacctgagtcttcgccgttcctcagagcgcctcagtgacacccctggatccttccagtcaccttccctggaaattctgctgtccagctgctccctgtgccgtgcctgtgattcgctggtgtatgatgaggaaatcatggctggctgggcacctgatgactctaacctcaacacaacctgccccttctgcgcctgcccctttgtgcccctgctcagtgtccagacccttgattcccggcccagtgtccccagccccaaatctgctggtgccagtggcagcaaagatgctcctgtccctggtggtcctggccctgtgctcagtgaccgaaggctctgccttgctctggatgagcccagctctgcaacgggcacatggggggagcctcccggcgggttgagagtggggcatgggcatacctgagccccctggtgctgcgtaaggagctggagtcgctggtagagaacgagggcagtgaggtgctggcgttgcctgaactgccctctgcccaccccatcatcttctggaaccttttgtggtatttccaacggctacgcctgcccagtattctaccaggcctggtgctggcctcctgtgatgggccttcgcactcccaggccccatctccttggctaacccctgatccagcctctgttcaggtacggctgctgtgggatgtactgacccctgaccccaatagctgcccacctctctatgtgctctggagggtccacagccagatcccccagcgggtggtatggccaggccctgtacctgcatcccttagtttggcactgttggagtcagtgctgcgccatgttggactcaatgaagtgcacaaggctgtggggctcctgctggaaactctagggcccccacccactggcctgcacctgcagaggggaatctaccgtgagatattattcctgacaatggctgctctgggcaaggaccacgtggacatagtggccttcgataagaagtacaagtctgcctttaacaagctggccagcagcatgggcaaggaggagctgaggcaccggcgggcgcagatgcccactcccaaggccattgactgccgaaaatgttttggagcacctccagaatgctagagaccttaagcttccctctccagcctagggtggggaagtgaggaagaagggattctagagttaaactgcctccctgttgccttcatggagttgggaacaggctgggaaggatgcccagtcaaaggctccaagcgaggacaacaggaagagggatccactgttaccaaaagtcctgattcccccatcaccaacctacccagtttgttcgtgctgatgttgggggagatctggggggagttggtacagctctgttcttcccttgtcctataccgggaactcccctccagggtacccacagatctgcattgccctggtcattttagaagtttttgttttaaaaaacaactggaaagatgcagagctactgagcctttgccctgaatgggaggtagggatgtcattctccaccaataatggtccctcttccctgacgttgctgaaggagcccaaggctctccatgcctttctacctaagtgtttgtattttattttaaattatttattctggagccacagcccccttgcttatgaggttcttatggagagtgagaaagggaagggaaatagggcaccatggtccggtggtttgtagttccttcaaagtcaggcactgggagctagaggagtctcaagctccccttaggaagaactggtgccccctccagtcctaatttttcttgcctgccccgccttggggaatgcctcacccacccaggtcctgacctgtgcaataaggattgttccctgcgaagttttgttggatgtaaatatagtaaaagctgcttctgtctttttcaaaaaa

SEQ ID NO: 135 - DNA, 3007 bp, Homo sapiens
gcccactgggctctcccggctgcagtgccagggcgcaggacgcggccgatctcccgctcccgccacctccgccaccatgctgctcccccagctctgctggctgccgctgctcgctgggctgctcccgccggtgcccgctcagaagttctcggcgctcacgtttttgagagtggatcaagataaagacaaggattgtagcttggactgtgcgggttcgccccagaaacctctctgcgcatctgacggaaggaccttcctttcccgttgtgaatttcaacgtgccaagtgcaaagatccccagctagagattgcatatcgaggaaactgcaaagacgtgtccaggtgtgtggccgaaaggaagtatacccaggagcaagcccggaaggagtttcagcaagtgttcattcctgagtgcaatgacgacggcacctacagtcaggtccagtgtcacagctacacgggatactgctggtgcgtcacgcccaacgggaggcccatcagcggcactgccgtggcccacaagacgccccggtgcccgggttccgtaaatgaaaagttaccccaacgcgaaggcacaggaaaaacagatgatgccgcagctccagcgttggagactcagcctcaaggagatgaagaagatattgcatcacgttaccctaccctttggactgaacaggttaaaagtcggcagaacaaaaccaataagaattcagtgtcatcctgtgaccaagagcaccagtctgccctggaggaagccaagcagcccaagaacgacaatgtggtgatccctgagtgtgcgcacggcggcctctacaagccagtgcagtgccacccctccacggggtactgctggtgcgtcctggtggacacggggcgccccattcccggcacatccacaaggtacgagcagccgaaatgtgacaacacgggccagggcccacccagccaaagcccgggacctgtacaagggccgccagctacaaggttgtccgggtgccaaaaagcatgagtttctgaccagcgttctggacgcgctgtccacggacatggtccacgccgcctccgacccctcctcctcgtcaggcaggctctcagaacccgaccccagccataccctagaggagcgggtggtgcactggtacttcaaactactggataaaaactccagtggagacatcggcaaaaaggaaatcaaacccttcaagaggttccttcgcaaaaaatcaaagcccaaaaaatgtgtgaagaagtttgttgaatactgtgacgtgaataatgacaaatccatctccgtacaagaactgatgggctgcctgggcgtggcgaaagaggacggcaaagcggacaccaagaaacgccacacccccagaggtcatgctgaaagtacgtctaatagacagccaaggaaacaaggataaatggctcataccccgaaggcagttcctagacacatgggaaatttccctcaccaaagagcaattaagaaaacaaaaacagaaacacatagtatttgcactttgtactttaaatgtaaattcactttgtagaaatgagctatttaaacagactgttttaatctgtgaaaatggagagctggcttcagaaaattaatcacatacaatgtatgtgtcctcttttgaccttggaaatctgtatgtggtggagaagtatttgaatgcatttaggcttaatttcttcgccttccacatgttaacagtagagctctatgcactccggctgcaatcgtatggctttctctaacccctgcagtcacttccagatgcctgtgcttacagcattgtggaatcatgttggaagctccacatgtccatggaagtttgtgatgtacggccgaccctacaggcagttaacatgcatgggctggtttgtttcttgggattttctgttagtttgtcttgttttgctttccagagatcttgctcatacaatgaatcacgcaaccactaaagctatccagttaagtgcaggtagttcccctggaggaaataatattttcaaactgtcgttggtgtgatactttggctcaaaggatctttgcttttccattttaagcttctgttttgagttttgccctggggcttgaatgagtcccagagagtcgttcggatggtgggaggctgcctaggaggcagtaaatccagtcacagtgcctgggaggggcccatccttccaaaatgtaaatccagtcgcggtgtgaccgagctggctaacaggcttgtctgcctggttttcctcctacacgtggacattattctcctgatcctcctacctggtccaccccagggctaccggaaggtaaaatcttcacctgaaccaattatgagcagtctccttactgaaggtacagccggatacgtggtgcccccggggctggtgttggcagccggggggaggtgcctgagggtccccacggttcctttctgcttttctgaatgcatcaagggtacgagaacttgccaatgggaaattcatccgagtggcactggcagagaaggataggagtggaatgcccacacagtgaccaacagaactggtctgcgtgcataaccagctgccaccctcaggcctgggccccagagctcagggcacccagtgtcttaaggaaccatttggaggacagtctgagagcaggaacttcaagctgtgattctatctcggctcagacttttggttggaaaaagatcttcatggccccaaatcccctgagacatgccttgtagaatgattttgtgatgttgtgatgcttgtggagcatcgcgtaaggcttcttgcttatttaaactgtgcaaggtaaaaatcaagcctttggagccacagaaccagctcaagtacatgccaatgttgtttaagaaacagttatgatcctaaactttttggataatcttttatatttctgacctttgaatttaatcattgttcttagattaaaataaaatatgctattgaaactaaaaaaaaaaaagaggggagaagaaaaaaaaaaagg

SEQ ID NO: 136 - DNA, 1229 bp, Homo sapiens
ctctctgctccggtgcaggcccgcaggcgccctgggctgggagcaacgcgactgaccgtggtcgtgggcggacggcggctgcagcgtggaggagctggggtcgctgtgggtcgcgaacagagcccgggacgtgcgcgcttggtgcacgatcctgaaggggagctccgaggggcccgggtctccagggctgctgcggccattcccggagcccggcgcggggcccgcgagatactggtttaggccgtcccagggctccgggcgcacccggtggccgctgctgcagcggagggagcgcggcggcgcgggggctcggagacagcgtttctcccggaagtcttcctcgggcagcaggtgggaagtgggagccggagcggcagctggcagcgttctctccgcaggtcggcaccatgcgccctgcagccctgcgcggggccctgctgggctgcctctgcctggcgttgctttgcctgggcggtgcggacaagcgcctgcgtgacaaccatgagtggaaaaaactaattatggttcagcactggcctgagacagtatgcgagaaaattcaaaacgactgtagagaccctccggattactggacaatacatggactatggcccgataaaagtgaaggatgtaatagatcgtggcccttcaatttagaagagattaaggatcttttgccagaaatgagggcatactggcctgacgtaattcactcgtttcccaatcgcagccgcttctggaagcatgagtgggaaaagcatgggacctgcgccgcccaggtggatgcgctcaactcccagaagaagtactttggcagaagcctggaactctacagggagctggacctcaacagtgtgcttctaaaattggggataaaaccatccatcaattactaccaagttgcagattttaaagatgcccttgccagagtatatggagtgatacccaaaatccagtgccttccaccaagccaggatgaggaagtacagacaattggtcagatagaactgtgcctcactaagcaagaccagcagctgcaaaactgcaccgagccgggggagcagccgtcccccaagcaggaagtctggctggcaaatggggccgccgagagccggggtctgagagtctgtgaagatggcccagtcttctatcccccacctaaaaagaccaagcattgatgcccaagttttggaaatattctgttttaaaaagcatgaggtaggcatgtc

SEQ ID NO: 137 - DNA, 1972 bp, Homo sapiens
acaggggcttccccttcgccgccgccgccgccgccggccaagctccgccgcgcccgcggcccgcggccgccatgcagtttatgttgctttttagtcgtcagggaaagcttcgactgcaaaaatggtatgtcccactatcagacaaagagaagagaaagatcacaagagaacttgttcagaccgttttagcacggaaacctaaaatgtgcagcttccttgagtggcgagatctgaagattgtttacaaaagatatgctagtctgtatttttgctgtgctattgaggatcaggacaatgaactaattaccctggaaataattcatcgttatgtggaattacttgacaagtatttcggcagtgtctgtgaactagatatcatctttaattttgagaaggcttattttattttggatgagtttcttttgggaggggaagttcaggaaacatccaagaaaaatgtccttaaagcaattgagcaggctgatctactgcaggaggatgcgaaagaagctgaaaccccacgtagtgttcttgaagaaattggactgacataactctcctcccttgttgatgacttcttgtggcatttcacacactgtagatggtcactcccttcatgtccatgttagctcatggtgtaagatgatgtcttgtcagtattactgttttgctaagccgcttcattcatgcctacacaatttttttttaaaagggaactttagttaattaagtgataagggacttaaatatgaattagaatggtgcagaaagagataccttttctggatattttaaagtttaaaggtcagtttctcttaatctgattatgtgcacatatgaaaatggcacatcatatacatgtaaaatcaggcagtatacatttattaattactgtatttgacaaaggaaactcttaaattataatgtgaaacctggttttatgaaaccaaagactagtgcagcatttcagcatatgtaaaaaaaaaaaaaaagggaattgacatgtcacatatcaaatgaatggaaactttgttgaaactttaaaaagcaaatttactccaaagacttgtattggaaattacataccttttttttttttttttaaaggactacagattatttttaatgactaaattggagtgatacttcttacactaaaaattatttcttaggcattctgaatctgggatgagaaacaggattgtttcacaatagtaagcacataatttttaaggccaaggcacatttgactcctgagatgaattttttgtggtcataatcaaatacttagttgtttttgatgccccaaaataaagtgagaatggtaatttgccaggaattcttcataacagtatcttacaaaaaacgtgttgctctcttcacagtattatgtgtaaagtcattgtttaaagcacgaatgttccctctggggtacttgttaaagctaaatttattttgcttccctccacttagaagtgctgcacactttacagcagcttcctttctttccatggcactgcctagttaacagaagtcttataaaaatttaaaaagacacatttcttacaaaaaagagttgaatgaggtaaaatggcattagatggctctatattttttaaagctatgtaattgttcagcgtcacttttctaagtacttatacatatctaaacatgtcttcatggtttatattttcacttatatatgctgggctggattaagctttgttgtgattgtgaccaacattcaggccacgtgagcactgtcttatcacatcgccaattagttgtaataaacgttcaacgtacaaacactggagtgtgtttttatctctttccaaaagtttgtcaaactatgcagagctgctgaaggaagaatttctcatttttttttcagtaaaatgttgaaaattcccctccatttgaatatggtggttgttataagcacacacaagatacatggtggaagatctag

SEQ ID NO: 138 - DNA, 1741 bp, Homo sapiens
  modified_base (43): a, c, g, t, unknown or other
cgggttccgggctccgggctctgggtggcggcggctgtgagcngcggctganccnccgcgctgcgcancgacgcgggaatgaagcgggcgctgggcaggcgaaagggcgtgtggttgcgcctgaggaagatacttttctgtgttttggggttgtacattgccattccatttctcatcaaactatgtcctggaatacaggccaaactgattttcttgaatttcgtaagagttccctatttcattgatttgaaaaaaccacaggatcaaggtttgaatcacacgtgtaactactacctgcagccagaggaagacgtgaccattggagtctggcacaccgtccctgcagtctggtggaagaacgcccaaggcaaagaccagatgtggtatgaggatgccttggcttccagccaccctatcattctgtacctgcatgggaacgcaggtaccagaggaggcgaccaccgcgtggagctttacaaggtgctgagttcccttggttaccatgtggtcacctttgactacagaggttggggtgactcagtgggaacgccatctgagcggggcatgacctatgacgcactccacgtttttgactggatcaaagcaagaagtggtgacaaccccgtgtacatctggggccactctctgggcactggcgtggcgacaaatctggtgcggcgcctctgtgagcgagagacgcctccagatgcccttatattggaatctccattcactaatatccgtgaagaagctaagagccatccattttcagtgatatatcgatacttccctgggtttgactggttcttccttgatcctattacaagtagtggaattaaatttgcaaatgatgaaaacgtgaagcacatctcctgtcccctgctcatcctgcacgctgaggacgacccggtggtgcccttccagcttggcagaaagctctatagcatcgccgcaccagctcgaagcttccgagatttcaaagttcagtttgtgccctttcattcagaccttggctacaggcacaaatacatttacaagagccctgagctgccacggatactgagggaattcctggggaagtcggagcctgagcaccagcactgagcctggccgtgggaaggaagcatgaagacctctgccctcctcccgttttcctccagtcagcagcccggtatcctgaagccccggggggccggcacctgcaatgctcaggagcccagctcgcacctggagagcacctcagatcccaggtggggaggcccctgcaggcctgcagtgcccggaggcctgagcatggctgtgtggaaagcgtgggtggcaggcatgtggctctccttgccgcccctcaacctgagatcttgttgggagacttaatggcagcaggcagccatcactgcctggttgatgctgcactgagctggacagggggagtccgggcaggggactcttggggctcgggaccatgctgagctttttggcaccacccacagagaacgtggggtccaggttctttctgcaccttcccagcacatgcagaatgactccagtggttccatcgtcccctcctgccctgtgtacctgcttgcctttctcagctgccccacctcccctgggctggcccactcacccacagtggaagtgcccgggatctgcacttcctcccctttcacctacctgtacacctaacctggccttagactgagctttatttaagaataaaatcgtggtggtgaaaaaaaaa

SEQ ID NO: 139 - DNA, 2808 bp, Homo sapiens
ggcaagatggcggaaggggaggacgtgggatggtggcggagctggctgcagcagagctaccaagcagtcaaagagaagtcctctgaagccttggagtttatgaagcgggacctgacggagtttacccaggtggtgcagcatgacacggcctgtaccatcgcagccacggccagcgtggtcaaggagaagctggctacggaaggctcctcaggagcaacagagaagatgaagaaagggttatctgacttcctaggggtgatctcagacacctttgccccttcgccagacaaaaccatcgactgcgatgtcatcaccctgatgggcacaccgtctggcacagctgagccctatgatggcaccaaggctcgcctctatagcctgcagtcggacccagcaacctactgtaatgaaccagatgggcccccggaattgtttgacgcctggctttcccagttctgcttggaggagaagaagggggagatctcagagctccttgtaggcagcccctccatccgggccctctacaccaagatggttccagcagctgtttcccattcagaattctggcatcggtatttctataaagtccatcagttagagcaggagcaggcccggagggacgccctgaagcagcgggcggaacagagcatctctgaagagcccggctgggaggaggaggaagaggagctcatgggcatttcacccatatctccaaaagaggcaaaggttcctgtggccaaaatttctacattccctgaaggagaacctggcccccagagcccctgtgaagagaatctggtgacttcagttgagcccccagcagaggtgactccatcagagagcagtgagagcatctccctcgtgacacagatcgccaacccggccactgcacctgaggcacgagtgctacccaaggacctgtcccaaaagctgctagaggcatccttggaggaacagggcctggctgtggatgtgggtgagactggaccctcaccccctattcactccaagcccctaacgcctgctggccacaccggcggcccagagcccaggcctccagccagagtagagactctgagggaggaggcgcccacagacttacgggtgtttgagctgaactcggatagtgggaagtctacaccctccaacaatggaaagaaaggctcaagcacggacatcagtgaggactgggagaaagactttgacttggacatgactgaagaggaggtgcagatggcactttccaaagtggatgcctccggggagctggaagatgtagagtgggaggactgggagtgagggagccagagggagcagctcccccacccatggcatctctcgcctccctcgctcgtctcagcccagccctggaagactgagaatgttcccccaaatctcctctgccaaccagagctctgggcacagattctggtggctccctgctggccctcttgggcctctgctcacacctgggaaggggctctctaaatcccggccagaaactctgacttgtgccaacaataggatgacccaagggagaggaaacctatcctcctcaccagaagagcctgtgtttttctgctgaacacccactgttcctgaggactcctgctgggaagtcccaagggatagttctagcccttctgcctgtgtagacagaagctaaaccaccagtctctctcggaggaagctgagacaacatactctgtccatacataagcaggcagggagggccatgccacctacccttggctaaacagggacagtgaacacattttggttcctatcccagtgggtaagaggcacttatctctgggaaatttgcctctcttgggactctccccctcccaggcattttccattcctggaaaggctcctttggggttcagaatccagagaccaaaccctgacccacctccttcctttcctccagcccacgctggtctgtccccatgccttcccagggcttcttcatgtcagatgcacccaagtccttagcccagctgtgccacctgcaggagttcgctcttgcgtttcttcccctccccaagaagggagggggctacttcaggcccttctgtgtgttgcctggcaggataccttgtccaaccagctacccacctcaactcccctgtagtttaggacacaaaacagctaccagcggtacagagcggtgatcaaagccgagtacttacaactctggtaagcctagcttctccgcctcagcccttctgcttctggaagggctatcctgggggtgaacttgaaactctcatcaggcttctgcaaaagctcttcttcctgaagacagacccagcctttgtgctctcaccctccactctggtaaagctgcacctctgggggaatgaggggctgcaggaatctctggagagcctggtgcttcacgatgctgctctggtgattcttgtacctaatctggtgtgctcaccaatgagtgaaagggatcgtgggtcagggacaccgagagagtgaggtcacttccacttcaaaccttcagtgagggggtgggatggagagaatgctgaatcttttttttgacgggatggggtttttctctttgtaattatttctttagtttaattaaccttttggttgtttgtgcaatattatatattttaaattataatgcatctccccagagtattttgtagctgggaaaagaaaaaaggaaaaaaagaaaaaaagattctaacagctgttagttttataattaaaaaagaaagaaaaaagaactttgtcctgaaccttttacagacttgccgttaacagcattaaagtgattcaccc

SEQ ID NO: 140 - DNA, 717 bp, Homo sapiens
  modified_base (32): a, c, g, t, unknown or other
catgcgccgaccttcctcggctggatttacangttnncccttaacacccgggatttaagggacccacactaccttcccgaagttgaaggcaagcggtgattgtttgtagacggcgctttgtcatgggacctgtgcggttgggaatattgcttttcctttttttggccgtgcacgaggcttgggctgggatgttgaaggaggaggacgatgacacagaacgcttgcccagcaaatgcgaagtgtgtaagctgctgagcacagagctacaggcggaactgagtcgcaccggtcgatctcgagaggtgctggagctggggcaggtgctggatacaggcaagaggaagagacacgtgccttacagcgtttcagagacaaggctggaagaggccttagagaatttatgtgagcggatcctggactatagtgttcacgctgagcgcaagggctcactgagatatgccaagggtcagagtcagaccatggcaacactgaaaggcctagtgcagaagggggtgaaggtggatctggggatccctctggagcttttgggatgagcccagccgttgaggtcacatacctcaagaagcagtgtgagaccatgttngaggagttttgagacattgtgggagactggtacttgcaccatcaggagcagccgctacaagattttctctgtgaaggtcatgtgctgccagctgcttgaactgcatgtcgggt

SEQ ID NO: 141 - DNA, 2552 bp, Homo sapiens
ggcagggggcgcgccgggcccagcgccacgtcaccgcccagcagccctcccgattggcgggcggggcggctataaagggagggcgcaggcggcgcccggatctcttccgccgccattttaaatccagctccatacaacgctccgccgccgctgctgccgcgacccggactgcgcgccagcacccccctgccgacagctccgtcactatggaggatatgaacgagtacagcaatatagaggaattcgcagagggatccaagatcaacgcgagcaagaatcagcaggatgacggtaaaatgtttattggaggcttgagctgggatacaagcaaaaaagatctgacagagtacttgtctcgatttggggaagttgtagactgcacaattaaaacagatccagtcactgggagatcaagaggatttggatttgtgcttttcaaagatgctgctagtgttgataaggttttggaactgaaagaacacaaactggatggcaaattgatagatcccaaaagggccaaagctttaaaagggaaagaacctcccaaaaaggtttttgtgggtggattgagcccggatacttctgaagaacaaattaaagaatattttggagcctttggagagattgaaaatattgaacttcccatggatacaaaaacaaatgaaagaagaggattttgttttatcacatatactgatgaagagccagtaaaaaaattgttagaaagcagataccatcaaattggttctgggaagtgtgaaatcaaagttgcacaacccaaagaggtatataggcagcaacagcaacaacaaaaaggtggaagaggtgctgcagctggtggacgaggtggtacgaggggtcgtggccgaggtcagggccaaaactggaaccaaggatttaataactattatgatcaaggatatggaaattacaatagtgcctatggtggtgatcaaaactatagtggctatggcggatatgattatactgggtataactatgggaactatggatatggacagggatatgcagactacagtggccaacagagcacttatggcaaggcatctcgagggggtggcaatcaccaaaacaattaccagccatactaaaggagaacattggagaaaacaggtgtgtataagagtacaggaaaacagtagaaatgtctaatttaatttaaagatcaatagacaaatgaaacgtaaaaacaaaatactatgtagcctgtttttactaaattgttgattttttaattgctttatgagcctgttttgcctaaagtgtctatagatctttaactttaaagtcttatctcactttctttagtattgcagaaaaacttaagagtttttctgtttgcttttgtgtaccaggtggtctagaggaataattaaacattttagaactattaacaggtaaagtactgaaatgggtacaacttaaggaaaacaagaatgttgtcttctaactctgacattataccttgtttgtacccgccagcgggaacttcattgcaggccgtgtgtcaccctgaccacgtctatctctgggggtcgcacgttgcgggcagagcgcaaggcatacaccagaaaacgctgtcctgtggtatggtctcttccaacttcatgtaccagcgtaaagattaaagtggaaaacttcagactttggcttcatttttaatctttttggagattaagtgtctaaacttaacttaaatggttttttacaggagttaaagtacataaatgcctttttacagcttaatcattttggtcttctgtttagtgttgtatttcaattgtggagcctcattttaagtgttcattcttttaagatttaatgcttgctttttctttttatagctaatagtgaaatctacaaaccaaaacaagaacttttaaatctgggatataaattaaagatcatatgcacagatcaatttatgttcttgtaataaacttattagaaattggtgtttgtgatagcattttacttgggttactagagatgcttctagtagaccttaatctagcatagttgaacctctgaatatgggaaggttgtattcccagattctttcctgaatagatttgaatttaatgtcatttgggaactccagggtgagtttattgactacccaaactgtattttaccaataaatatgcatatgatctttaattattgaagaaaataaagtgaggacttaaaacaattcatgaaagtggacctttaaaagcttgtcagagttgcacaaatctaactggtattttgtttttgtttttaggaggagatgttaaagtaacccatcttgcaggacgacattgaagattggtcttctgttgatctaagatgattattttgtaaaagactttctagtgtacaagacaccattgtgtccaactgtatatagctgccaattagttttctttgtttttactttgtcctttgctatctgtgttatgactcaatgtggatttgtttatacacattttatttgtatcatttcatgttaaacctcaaataaatgcttccttatgtgaaaaaaaaaa

SEQ ID NO: 142 - DNA, 1046 bp, Homo sapiens
taccagtgtaaagccagagctgaggttcttgatagtccacaatgggtgaaccacagcaagtgagtgcacttccaccacctccaatgcaatatatcaaggaatatacggatgaaaatattcaagaaggcttagctcccaagcctccccctccaataaaagacagttacatgatgtttggcaatcagttccaatgtgatgatcttatcatccgccctttggaaagtcagggcatcgaacggcttcatcctatgcagtttgatcacaagaaagaactgagaaaacttaatatgtctatccttattaatttcttggaccttttagatattttaataaggagccctgggagtataaaacgagaagagaaactagaagatcttaagctgctttttgtacacgtgcatcatcttataaatgaataccgaccccaccaagcaagagagaccttgagagtcatgatggaggtccagaaacgtcaacggcttgaaacagctgagagatttcaaaagcacctggaacgagtaattgaaatgattcagaattgcttggcttctttgcctgatgatttgcctcattcagaagcaggaatgagagtaaaaactgaaccaatggatgctgatgatagcaacaattgtactggacagaatgaacatcaaagagaaaattcaggtcataggagagatcagattatagagaaagatgctgccttgtgtgtcctaattgatgagatgaatgaaagaccatgaaagatgtttctttttctttttttccttttgataatagcatcatatattagttcattttcttttggacagtcttaagagaagtttcactaaaaatgtaaacagctttaatcttgactccaaatttttcaattatgagatgtcataggcagtaatttcgctgtataacaagcatagacaaatgagtgtccctgcactaagaagaatcactttaaaaagcaaagtgttagctgctgttgtatgggacattcctatgttttagagttgcagtaaaactttgatgataacctcaaaaaaaaataaaaa

SEQ ID NO: 143 - DNA, 1864 bp, Homo sapiens
gccctgggctcgcggcggtgccgcggggatggcgggagccggagctggagccggagctcgcggcggagcggcggcgggggtcgaggctcgagctcgcgatccaccgcccgcgcaccgcgcacatcctcgccaccctcggcctgcggctcagccctcggcccgcaggatggatggcgggtcagggggcctggggtctggggacaacgccccgaccactgaggctcttttcgtggcactgggcgcgggcgtgacggcgctcagccatcccctgctctacgtgaagctgctcatccaggtgggtcatgagccgatgccccccacccttgggaccaatgtgctggggaggaaggtcctctatctgccgagcttcttcacctacgccaagtacatcgtgcaagtggatggtaagatagggctgttccgaggcctgagtccccggctgatgtccaacgccctctctactgtgactcggggtagcatgaagaaggttttccctccagatgagattgagcaggtttccaacaaggatgatatgaagacttccctgaagaaagttgtgaaggagacctcctacgagatgatgatgcagtgtgtgtcccgcatgttggcccaccccctgcatgtcatctcaatgcgctgcatggtccagtttgtgggacgggaggccaagtacagtggtgtgctgagctccattgggaagattttcaaagaggaagggctgctgggattcttcgttggattaatccctcacctcctgggcgatgtggttttcttgtggggctgtaacctgctggcccacttcatcaatgcctacctggtggatgacagcttcagccaggccctggccatccggagctataccaagttcgtgatggggattgcagtgagcatgctgacctaccccttcctgctagttggcgacctcatggctgtgaacaactgcgggctgcaagctgggctccccccttactccccagtgttcaaatcctggattcactgctggaagtacctgagtgtgcagggccagctcttccgaggctccagcctgcttttccgccgggtgtcatcaggatcgtgctttgccctggagtaacctgaatcatctaaaaaacacggtctcaacctggccaccgtgggtgaggcctgaccaccttgggacacctgcgagacgactccaacccaacaacaaccagatgtgctccagcccagccgggcttcagttccatatttgccatgtgtctgtccagatgtggggttgagcgggggtggggctgcacccagtggattgggtcacccggcagacctagggaaggtgaggcgaggtggggagttggcagaatccccatacctcgcagatttgctgagtctgtcttgtgcagagggccagagaatggcttatgggggcccaggttggatggggaaaggctaatggggtcagaccccaccccgtctacccctccagtcagcccagcgcccatcctgcagctcagctgggagcatcattctcctgctttgtacatagggtgtggtcccctggcacgtggccaccatcatgtctaggcctatgctaggaggcaaatggccaggctctgcctgtgtttttctcaacactacttttctgatatgagggcagcacctgcctctgaatgggaaatcatgcaactactcagaatgtgtcctcctcatctaatgctcatctgtttaatggtgatgcctcgcgtacaggatctggttacctgtgcagttgtgaatacccagaggttgggcagatcagtgtctctagtcctacccagttttaaagttcatggtaagatttgacctcatctcccgcaaataaatgtattggtgatttggaaaaaaaaaa

SEQ ID NO: 144 - DNA, 2295 bp, Homo sapiens
gtctgcagctccggccgccacttgcgcctctccagcctccgcaggcccaaccgccgccagcaccatggccagcaccatttccgcctacaaggagaagatgaaggagctgtcggtgctgtcgctcatctgctcctgcttctacacacagccgcaccccaataccgtctaccagtacggggacatggaggtgaagcagctggacaagcgggcctcaggccagagcttcgaggtcatcctcaagtccccttctgacctgtccccagagagccctatgctctcctccccacccaagaagaaggacacctccctggaggagctgcaaaagcggctggaggcagccgaggagcggaggaagacgcaggaggcgcaggtgctgaagcagctggcggacggcgcgagcacgagcgcgaggtgctgcacaaggcgctggaggagaataacaacttcagccgccaggcggaggagaagctcaactacaagatggagctcagcaaggagatccgcgaggcacacctggccgcactgcgcgagcggctgcgcgagaaggagctgcacgcggccgaggtgcgcaggaacaaggagcagcgagaagagatgtcgggctaagggcccgggacgggcggcgcccatcctgcgacggaacacgttcgggttttggttttgtttcgttcacctctgtctagatgcaacttttgttcctcctcccccaccccagcccccagcttcatgcttctcttccgcactcagccgccctgccctgtcctcgtggtgagtcgctgaccacggcttcccctgcaggagccgccgggcgtgagacgcggtccctcggtgcagacaccaggccgggcgcggctgggtcccccgggggccctgtgagagaggtggtggtgaccgtggtaaacccagggcggtggcgtgggatcgcgggtccttacgctgggctgtctggtcagcacgtgcaggtcagggcaggtcctctgagccggcgcccctggccagcaggcgaggctacagtacctgctgtctttccagggggaaggggctccccatgagggaggggcgacgggggaggggggtgatggtgcctgggagcctgcgtgtgcagccggtgcttgttgaactggcaggcgggtgggtgggggctgcagctttccttaatgtggttgcacaggggtcctctgagaccacctggcgtgaggtggacaccctgggccttcctggaagcctgcagttgggggcctgccctgagtctgctggggagtgggcattctctgccagggacccatgagcaggctgcatggtctagaggttgtgggcagcatggacagtcccccactcagaagtgcaagagttccaaagagcctctggcccaggcccctccgtgggacagccccgccgcccctccccaccagggctttgcagatgtccttgaaagacccaccctagagccctttggagtgctggcccctcctgtgccctctgccctggtggaagcggcagccacaagtcctcctcagggagccccaagggggattttgtgggaccgctgcccacagatccaggtgttggaagggcagcgggtaaggttcccaagccagccccaacacccttcccacttggcacccagagggggctgtgggtggaggcctgactccaggcctctcctgcccacaccctctgggctgagttccttctttcccttggacgcccagtgctggccttggaggacggtcagctggaggatggcggtgggggaggctgtctttgtaccactgcagcatcccccacttctccacggaagccccatcccaaagctgctgcctggccccttgctgtaaagtgtgaagggggcggctgagttctcttaggacccagagccagggccctcaacttccatcctgcgggaggccttggccgggcactgccagtgtcttccagagccacacccagggaccacgggaggatcctgacccctgcagggctcaggggtcagcagggacccactgccccatctccctctccccaccaagacagccccagaaggagcagccagctgggatgggaacccaaggctgtccacatctggcttttgtgggactcagaaagggaagcagaactgagggctgggatattcctcatggtggcagcgctcatagcgaaagcctactgtaatatgcacccatctcatccacgtagtaaagtgaacttaaaaattcaatcaaatgaacaattaaataaacacctgtgtgtttaagacaaaataaaaatggaggagaacaaaaaaaaaggggcggt

SEQ ID NO: 145 - DNA, 842 bp, Homo sapiens
cggggacggaagcagcccctgggcccgaggggctcgaggccgggccggggcgatgtggagcgcgggccgcggcggggctgcctggccggtgctgttggggctgctgctggcgctgttagtgccgggcggtggtgccgccaagaccggtgcggagctcgtgacctgcgggtcggtgctgaagctgctcaatacgcaccaccgcgtgcggctgcactcgcacgacatcaaatacggatccggcagcggccagcaatcggtgaccggcgtagaggcgtcggacgacgccaatagctactggcggatccgcggcggctcggagggcgggtgcccgcgcgggtccccggtgcgctgcgggcaggcggtgaggctcacgcatgtgcttacgggcaagaacctgcacacgcaccacttcccgtcgccgctgtccaacaaccaggaggtgagtgcctttggggaagacggcgagggcgacgacctggacctatggacagtgcgctgctctggacagcactgggagcgtgaggctgctgtgcgcttccagcatgtgggcacctctgtgttcctgtcagtcacgggtgagcagtatggaagccccatccgtgggcagcatgaggtccacggcatgcccagtgccaacacgcacaatacgtggaaggccatggaaggcatcttcatcaagcctagtgtggagccctctgcaggtcacgatgaactctgagtgtgtggatggatgggtggatggagggtggcaggtggggcgtctgcagggccactcttggcagagactttgggtttgtaggggtcctcaagtgcctttgtgattaaagaatgttggtctaaaa

SEQ ID NO: 146 - DNA, 2345 bp, Homo sapiens
gtcccgccccgcagctgcgcgcaggcgctcgacgagccgctcgcattctacgtaacggacggcggaggctacgtgaagagaggcgcggcgtgactgagctacggttctggctgcgtcctagaggcatccggggcagtaaaaccgctgcgatcgcggaggcggcggccaggccgagaggcaggccgggcaggggtgtcggacgcagggcgctgggccgggtttcggcttcggccacagctttttttctcaaggtgcaatgaaagccttccacactttctgtgttgtccttctggtgtttgggagtgtctctgaagccaagtttgatgattttgaggatgaggaggacatagtagagtatgatgataatgacttcgctgaatttgaggatgtcatggaagactctgttactgaatctcctcaacgggtcataatcactgaagatgatgaagatgagaccactgtggagttggaagggcaggatgaaaaccaagaaggagattttgaagatgcagatacccaggagggagatactgagagtgaaccatatgatgatgaagaatttgaaggttatgaagacaaaccagatacttcttctagcaaaaataaagacccaataacgattgttgatgttcctgcacacctccagaacagctgggagagttattatctagaaattttgatggtgactggtctgcttgcttatatcatgaattacatcattgggaagaataaaaacagtcgccttgcacaggcctggtttaacactcatagggagcttttggagagcaactttactttagtgggggatgatggaactaacaaagaagccacaagcacaggaaagttgaaccaggagaatgagcacatctataacctgtggtgttctggtcgagtgtgctgtgagggcatgcttatccagctgaggttcctcaagagacaagacttactgaatgtcctggcccggatgatgaggccagtgagtgatcaagtgcaaataaaagtaaccatgaatgatgaagacatggatacctacgtatttgctgttggcacacggaaagccttggtgcgactacagaaagagatgcaggatttgagtgagttttgtagtgataaacctaagtctggagcaaagtatggactgccggactctttggccatcctgtcagagatgggagaagtcacagacggaatgatggatacaaagatggttcactttcttacacactatgctgacaagattgaatctgttcatttttcagaccagttctctggtccaaaaattatgcaagaggaaggtcagcctttaaagctacctgacactaagaggacactgttgtttacatttaatgtgcctggctcaggtaacacttacccaaaggatatggaggcactgctacccctgatgaacatggtgatttattctattgataaagccaaaaagttccgactcaacagagaaggcaaacaaaaagcagataagaaccgtgcccgagtagaagagaacttcttgaaactgacacatgtgcaaagacaggaagcagcacagtctcggcgggaggagaaaaaaagagcagagaaggagcgaatcatgaatgaggaagatcctgagaaacagcgcaggctggaggaggctgcattgaggcgtgagcaaaagaagttggaaaagaagcaaatgaaaatgaaacaaatcaaagtgaaagccatgtaaagccatcccagagatttgagttctgatgccacctgtaagctctgaattcacaggaaacatgaaaaacgccagtccatttctcaaccttaaatttcagacagtcttgggcaactgagaaatccttatttcatcatctactctgtttggggtttggggttttacagagattgaagatacctggaaagggctctgtttcaagaatttttttttccagataatcaaattattttgattattttataaaaggaatgatctatgaaatctgtgtaggttttaaatattttaaaaattataatacaaatcatcagtgcttttagtacttcagtgtttaaagaaataccatgaaatttataggtagataaccagattgttgctttttgtttaaaccaagcagttgaaatggctataaagactgactctaaaccaagattctgcaaataatgattggaattgcacaataaacattgcttgatgttttcttgtatgtctacattaaacttgagaaaaagtaaaaattagaacactgtatgtagtaatgaaatttcagggacccagaacataatgtagtatatgtttttaggtgggagatgctgataacaaaattaataggaagtctgtaggcattaggatactgaca

SEQ ID NO: 147 - DNA, 2215 bp, Homo sapiens
cccacgcgtccgcccacgcgtccgttttcagtagggatttcctgtgaccagacaagttcatctgagagccagttctcaccactggaattctcaggaatggaccatgaggacatcagtgagtcagtggatgcagcatacaacctccaggacagttgccttacagactgtgatgtggaagatgggactatggatggcaatgatgaggggcactcctttgaactttgtccttctgaagcttctccttatgtaaggtcaagggagagaacctcctcttcaatagtatttgaagattctggctgtgataatgcttccagtaaagaagagccgaaaactaatcgattgcatattggcaaccattgtgctaataaactaactgctttcaagcccaccagtagcaaatcttcttctgaagctacattgtctatttctcctccaagaccaaccactttaagtttagatctcactaaaaacaccacagaaaaactccagcccagttcaccaaaggtgtatctttacattcaaatgcagctgtgcagaaaagaaaacctcaaagactggatgaatggacgatgtaccatagaggagagagagaggagcgtgtgtctgcacatcttcctgcagatcgcagaggcagtggagtttcttcacagtaaaggactgatgcacagggacctcaagccatccaacatattctttacaatggatgatgtggtcaaggttggagactttgggttagtgactgcaatggaccaggatgaggaagagcagacggttctgaccccaatgccagcttatgccagacacacaggacaagtagggaccaaactgtatatgagcccagagcagattcatggaaacagctattctcataaagtggacatcttttctttaggcctgattctatttgaattgctgtatccattcagcactcagatggagagagtcaggaccttaactgatgtaagaaatctcaaatttccaccattatttactcagaaatatccttgtgagtacgtgatggttcaagacatgctctctccatcccccatggaacgacctgaagctataaacatcattgaaaatgctgtatttgaggacttggactttccaggaaaaacagtgctcagacagaggtctcgctccttgagttcatcgggaacaaaacattcaagacagtccaacaactcccatagccctttgccaagcaattagccttaagttgtgctagcaaccctaataggtgatgcagataatagcctacttcttagaatatgcctgtccaaaattgcagacttgaaaagtttgttcttcgctcaatttttttgtggactactttttttatatcaaatttaagctggatttgggggcataacctaatttgagccaactcctgagttttgctatacttaaggaaagggctatctttgttctttgttagtctcttgaaactggctgctggccaagctttatagccctcaccatttgcctaaggaggtagcagcaatccctaatatatatatatagtgagaactaaaatggatatatttttataatgcagaagaaggaaagtccccctgtgtggtaactgtattgttctagaaatatgctttctagagatatgatgattttgaaactgatttctagaaaaagctgactccatttttgtccctggcgggtaaattaggaatctgcactattttggaggacaagtagcacaaactgtataacggtttatgtccgtagttttatagtcctatttgtagcattcaatagctttattccttagatggttctagggtgggtttacagctttttgtacttttacctccaataaagggaaaatgaagctttttatgtaaattggttgaaaggtctagttttgggaggaaaaaagccgtagtaagaaatggatcatatatattacaactaacttcttcaactatggactttttaagcctaatgaaatcttaagtgtcttatatgtaatcctgtaggttggtacttcccccaaactgattataggtaacagtttaatcatctcacttgctaacatgtttttatttttcactgtaaatatgtttatgttttatttataaaaattctgaaatcaatccatttgggttggtggtgtacagaacacacttaagtgtgttaacttgtgacttctttcaagtctaaatgatttaataaaactttttttaaattaaaaaaaaaa

SEQ ID NO: 148 - DNA, 1395 bp, Homo sapiens
ggttgacatgatgaacaatcggtttcggaaggatatgatgaaaaatgctagtgaaagtaaactttcgaaagacaaccttaaaaagagacttaaagaagaattccaacatgccatgggaggagtacctgcctgggcagagactactaagcggaaaacatcttcagatgatgaaagtgaagaggatgaagatgatttgttgcaaaggactgggaatttcatatccacatcaacttctcttccaagaggcatcttgaagatgaagaactgccagcatgcgaatgctgaacgtcctactgttgctcggatctcatctgtgcagttccatcccggtgcacagattgtgatggttgctggattagataatgctgtatcactatttcaggttgatgggaaaacaaatcctaaaattcagagcatctatttggaaaggtttccaatctttaaggcttgttttagtgctaatggggaagaagttttagccacgagtacccacagcaaggttctttatgtctatgacatgctggctggaaagttaattcctgtgcatcaagtgagaggtttgaaagagaagatagtgaggagctttgaagtctccccagatgggtccttcttgctcataaatggcattgctggatatttgcatttgctagcaatgaagaccaaagaactgattggaagcatgaaaattaatggaagggttgcagcatccacattctcttcagatagtaagaaagtatacgcctcttcgggggatggagaagtttatgtttgggatgtgaactcaaggaagtgccttaacagatttgttgatgaaggcagtttatatggattaagcattgccacatctaggaatggacagtatgttgcttgtggttctaattgtggagtggtaaatatatacaatcaagattcttgtctccaagaaacaaacccaaagccaataaaagctataatgaacttggttacaggtgttacttctctgaccttcaatcctactacagaaatcttggcaattgcttcagaaaaaatgaaagaagcagtcagattggttcatcttccttcctgtacagtattttcaaacttcccagtcattaaaaataagaatatttctcatgttcataccatggatttttctccgagaagtggatactttgccttggggaatgaaaagggcaaggccctgatgtataggttgcaccattactcagacttctaaagagactatttgaagtccagttgagtcacaagagaagcctgtcttgatatatcatctcagaaactttcctgaatatgtgataatatatggaaaatgatttatagatccagctgtgcttaagagccagtaatgtcttaataaacatgtggcagcttttgtttgaaaaaaaaaaaaaaagg

SEQ ID NO: 149 - DNA, 2609 bp, Homo sapiens
cccgccatggcactgtcgcgggggctgccccgggagctggctgaggcggtggccgggggccgggtgctggtggtgggggcgggcggcatcggctgcgagctcctcaagaatctcgtgctcaccggtttctcccacatcgacctgattgatctggatactattgatgtaagcaacctcaacagacagtttttgtttcaaaagaaacatgttggaagatcaaaggcacaggttgccaaggaaagtgtactgcagttttacccgaaagctaatatcgttgcctaccatgacagcatcatgaaccctgactataatgtggaatttttccgacagtttatactggttatgaatgctttagataacagagctgcccgaaaccatgttaatagaatgtgcctggcagctgatgttcctcttattgaaagtggaacagctgggtatcttggacaagtaactactatcaaaaagggtgtgaccgagtgttatgagtgtcatcctaagccgacccagagaacctttcctggctgtacaattcgtaacacaccttcagaacctatacattgcatcgtttgggcaaagtacttgttcaaccagttgtttggggaagaagatgctgatcaagaagtatctcctgacagagctgaccctgaagctgcctgggaaccaacggaagccgaagccagagctagagcatctaatgaagatggtgacattaaacgtatttctactaaggaatgggctaaatcaactggatatgatccagttaaactttttaccaagctttttaaagatgacatcaggtatctgttgacaatggacaaactatggcggaaaaggaaacctccagttccgttggactgggctgaagtacaaagtcaaggagaagaaacgaatgcatcagatcaacagaatgaaccccagttaggcctgaaagaccagcaggttctagatgtaaagagctatgcacgtcttttttcaaagagcatcgagactttgagagttcatttagcagaaaagggggatggagctgagctcatatgggataaggatgacccatctgcaatggattttgtcacctctgctgcaaacctcaggatgcatattttcagtatgaatatgaagagtagatttgatatcaaatcaatggcagggaacattattcctgctattgctactactaatgcagtaattgctgggttgatagtattggaaggattgaagattttatcaggaaaaatagaccagtgcagaacaatttttttgaataaacaaccaaacccaagaaagaagcttcttgtgccttgtgcactggatcctcccaaccccaattgttatgtatgtgccagcaagccagaggtgactgtgcggctgaatgtccataaagtgactgttctcaccttacaagacaagatagtgaaagaaaaatttgctatggtagcaccagatgtccaaattgaagatgggaaaggaacaatcctaatatcttccgaagagggagagacggaagctaataatcacaagaagttgtcagaatttggaattagaaatggcagccggcttcaagcagatgacttcctccaggactatactttattgatcaacatccttcatagtgaagacctaggaaaggacgttgaatttgaagttgttggtgatgccccggaaaaagtggggcccaaacaagctgaagatgctgccaaaagcataaccaatggcagtgatgatggagctcagccctccacctccacagctcaagagcaagatgacgttctcatagttgattcggatgaagaagattcttcaaataatgccgacgtcagtgaagaagagagaagccgcaagaggaaattagatgagaaagagaatctcagtgcaaagaggtcacgtatagaacagaaggaagagcttgatgatgtcatagcattagattgaacagaaatgcctctaaacagaaccctcttactatttagtttatctgggcagaaccagattgttatgtcctttgttccaaagggaaaaaattgacagcagtgacttgaaaatgattctgctccctttgaaagcattcattttgctagaactgttagacacattgcagtatgctgtattgaaagtaggaatatagttttaaaaaccctttgaacaaagtgtgtgcataaccagtcatgagataaaacaacacaatgcatgttgcctttttaatgtaaatacccttaggtatcattaatagtttcaaaatattgtggtttagtaaagttgatacctggttataaatattatgcctttatttttggctagaagaagaattatttttagcctagatctaaccattttcatactcttaactgattgaaacagattcaaagaagtatcgagtgctatgcattgaaacttgtttttaaatgttagatggcactatgtatattaatgtaaaacaatgttaatttactcaagttttcagtttgtaccgcctggtatgtctgtgtaagaagccaatttttgtgtattgttacagtttcaggttatttatattcgatgttttgtaaaactcaaataacgactatacttatggaccaaataaatggcatctgcattcttgttaaaaaaaaaaa

SEQ ID NO: 150 - DNA, 3633 bp, Homo sapiens
cctgagggatccacagagggtgcggtccttggagggaggacatgcagtgccacgtgccatggaccagccagtggaccccatggccagcaaggctgctcctggggccagtggggtggacagtcccgcccacgcaggtgactgaggtgccagtgtgggaatgaaaatgcggcctgtgctcctgggcccatgcgtctcacgctgcccttcctctccagggaagcctgtgtacctgctactttttcccgaacaattcatggtaaaaacacaaatggtatatggacaagatactgaatgtggaagaaacctacttgacagtgttggtgaaaatagggccaggatttcacacccgtgaatgctttttactgaaaagtattttgtgtttttctcccagttacagaatgtctgaaggggacagtgtgggagaatccgtccatgggaaaccttcggtggtgtacagatttttcacaagacttggacagatttatcagtcctggctagacaagtccacaccctacacggctgtgcgatgggtcgtgacactgggcctgagctttgtctacatgattcgagtttacctgctgcagggttggtacattgtgacctatgccttggggatctaccatctaaatcttttcatagcttttctttctcccaaagtggatccttccttaatggaagactcagatgacggtccttcgctacccaccaaacagaacgaggaattccgccccttcattcgaaggctcccagagtttaaattttggcatgcggctaccaagggcatccttgtggctatggtctgtactttcttcgacgctttcaacgtcccggtgttctggccgattctggtgatgtacttcatcatgctcttctgtatcacgatgaagaggcaaatcaagcacatgattaagtaccggtacatcccgttcacacatgggaagagaaggtacagaggcaaggaggatgccggcaaggccttcgccagctagaagcgggactgaggctgcctcacgtgttgcaagaacagttttgagccattgttaacaatgccttttttcttcacataaagtagttgattacgagggagtcaaattttctttttaaaaaggagcttcaatgatttgtaactgaaatatcaggttctagaagaaactggcgcttaaaccaaatcgcatggatttctttttcagtgacgttcaagtgtttctcacggatggaattctagtcagctgcaggcgggaagccaggcgggtggagcccatgggagcaagggcgagtggccggtccccgctgtgccaggtgggcaggcaggagcaaggcctgcgagggaggaacgggccgctccccgccagccgccttccccagcagccgcaggtggtgccagccactccacagagcccgagggatgatctagcctgattcctgcgtgtccgaaagaacttaacgttttaaaggtgattgtcaagtaactgtgtggggttctaatgccagtttcctaattccatctcactggagatgtttaaagttggcctctatcctaatgactcaaaacttggttcttaactaccatgattgcttttgagggcccggaattataaatatatattatattttaattgtttgagattattttgacacatttctttgatacgtagagtgttttgtttttaatttaaatctgtcctcatgcaaccctccatgaggggcagcgaagctggcagggagcagactggctttgtaggttcagcactcggccccccactgcgggagaggcggaacccacttgcatgtcagcgtttttgattcgagaaaagaaatactctcaacgttttaccaagtgattttacctccacctttactaaagtctttacctaaaacatggcagtcgctggacacaggaaagcccaccttttgtttggccttttcgaaaggtgacccatattgcacagcagaacatcacagctgtggtcccagatgagacactgacatgcgagtgaaggcctctcctcctgggccccgggctgcgcaggctcctcactctgggcggtgtttcctgtctcagaattgacacggtgaatgcttagtgtctggattttcttgtgccagtgtttacatatctgacatcgagctcctctaagaggccacgttcaagcttgtgtgtccctgacccaagatagccagtgctgctcccaggtggtacttctggtaccgtgttgagacacttgggattctcagactgtggacaggagtgtttgtcatttttcatactgttttcttaataagcgctcaggcctaaggtgtgacaggaagtcgcacgcgcttggccagagcacagtgaagcaaaggactgggtgctgatggatggagccacggcggcatctgcccacccggccgcagcccccagtgcctctcctggtggtcctcccagtctagagggtcacggcccccccgccctcctccgtctctggcaagctgaccttgactaacccaggaatacagggtcatcctcattcctaagtaagtcaaacagcaagacatggtttgcgcgggtctttgccggaagccggtcctgctggccaggtgttttacgtcagcagggaaatgtggcacacgccctcgaggcattttaacactgtgcttcaggaaatctcaagttccatcttgtgttagtaacgtacccacattttgctggagttagtttattaaagatgcctacggtgaactctctggcgcaggttaaatgcagttttgaaaacctggaaacatcaaatggaggcgggaaataggctggggccgagctgaggggctgaacacagcagtgaccgtgggtcagcaggtcgcctgcccagcaggccccccaggagagggctcgggcgcccctggcagcccccatacccccaggacctggctcgtgagtgcgtctgggtcaggaagagacctctctgtgcgtctcaggctgagatgcagatttctgttttctaaaactggaagcgaccttgacgtgtattgaaggtgtgtgtgccaaatgcttccgacggaggtgctggccttggttggtttctctctgccccgtgtggtcatcaagtcctgggggatgtgctctgcccagccgccctcggggagagcagcgccgcctcccatggggccgtggggctgctgttctcactgcactggctgaagcaacccgccagcctccgtgccccaccccacccagcacgcactcattcagtccattgccttaacacaagcctgatggggctgttttctcacaatataaacgaataaagtgtcttctggcctacttctgaattacttctcaactgtatggtttggggaagggagggaaacctaaaatcccgtccaaataagtgaaattcctgaagaagtggctgagtcctaccaggttggggttagggaaatgttctgggttcaggcgcccctcccagggctgagaaagcgcagccagggacagctttctgttctctcccagggtggctaggttagtatcttacatgacaaaaaactgagagtgttctaacttctgtgcaagcaaggttaatcctgagactaaatcttggcgttcagactcccgtagaggtcatctgtgtccaggcccacccgggcgccggctca

SEQ ID NO: 151 - DNA, 2018 bp, Homo sapiens
tggctcgctggccgctcctggaggcggcggcgggagcgcagggggcgcgcggcccggggactcgcattccccggttccccctccaccccacgcggcctggaccatggacgccagatggtgggcagtggtggtgctggctgcgttcccctccctaggggcaggtggggagactcccgaagcccctccggagtcatggacccagctatggttcttccgatttgtggtgaatgctgctggctatgccagctttatggtacctggctacctcctggtgcagtacttcaggcggaagaactacctggagaccggtaggggcctctgctttcccctggtgaaagcttgtgtgtttggcaatgagcccaaggcctctgatgaggttcccctggcgccccgaacagaggcggcagagaccaccccgatgtggcaggccctgaagctgctcttctgtgccacagggctccaggtgtcttatctgacttggggtgtgctgcaggaaagagtgatgacccgcagctatggggccacagccacatcaccgggtgagcgctttacggactcgcagttcctggtgctaatgaaccgagtgctggcactgattgtggctggcctctcctgtgttctctgcaagcagccccggcatggggcacccatgtaccggtactcctttgccagcctgtccaatgtgcttagcagctggtgccaatacgaagctcttaagttcgtcagcttccccacccaggtgctggccaaggcctctaaggtgatccctgtcatgctgatgggaaagcttgtgtctcggcgcagctacgaacactgggagtacctgacagccaccctcatctccattggggtcagcatgtttctgctatccagcggaccagagccccgcagctccccagccaccacactctcaggcctcatcttactggcaggttatattgcttttgacagcttcacctcaaactggcaggatgccctgtttgcctataagatgtcatcggtgcagatgatgtttggggtcaatttcttctcctgcctcttcacagtgggctcactgctagaacagggggccctactggagggaacccgcttcatggggcgacacagtgagtttgctgcccatgccctgctactctccatctgctccgcatgtggccagctcttcatcttttacaccattgggcagtttggggctgccgtcttcaccatcatcatgaccctccgccaggcctttgccatccttctttcctgccttctctatggccacactgtcactgtggtgggagggctgggggtggctgtggtctttgctgccctcctgctcagagtctacgcgcggggccgtctaaagcaacggggaaagaaggctgtgcctgttgagtctcctgtgcagaaggtttgagggtggaaagggcctgaggggtgaagtgaaataggaccctcccaccatccccttctgctgtaacctctgagggagctggctgaaagggcaaaatgcaggtgttttctcagtatcacagaccagctctgcagcaggggattggggagcccaggaggcagccttcccttttgccttaagtcacccatcttccagtaagcagtttattctgagccccgggggtagacagtcctcagtgaggggttttggggagtttggggtcaagagagcataggtaggttccacagttactcttcccacaagttcccttaagtcttgccctagctgtgctctgccaccttccagactcactcccctctgcaaatacctgcatttcttaccctggtgagaaaagcacaagcggtgtaggctccaatgctgctttcccaggagggtgaagatggtgctgtgctgaggaaaggggatgcagagccctgcccagcaccaccacctcctatgctcctggatccctaggctctgttccatgagcctgttgcaggttttggtactttagaaatgtaactttttgctcttataattttattttattaaattaaattactgcagtggaaaaaaaaaa

SEQ ID NO: 152 - DNA, 942 bp, Homo sapiens
cctccatcagctcgccgcgcagcggctgtatttgcggcctgtgcgagtaggcgcttgggcactcagtctccctggcgggcgacgggcagaaatctcgaaccagtggagcgcactcgtaacctggatcccagaaggtcgcgaaggcagtaccgtttcctcagcggcggactgctgcagtaagaatgtcttttccacctcatttgaatcgccctcccatgggaatcccagcactcccaccagggaccccacccccgcagtttccaggatttcctccacctgtacctccagggaccccaatgattcctgtaccaatgagcattatggctcctgctccgactgtcttagtacccactgtgtctatggttggaaagcatttgggcgcaagaaaggatcatccaggcttaaaggctaaagaaaatgatgaaaattgtggtcctactaccactgtttttgttggcaacatttccgagaaagcttcagacatgcttataagacaactcttagctaaatgtggtttggttttgagctggaagagagtacaaggtgcttccggaaagcttcaagccttcggattctgtgagtacaaggagccagaatctaccctccgtgcactcagattattacatgacctgcaaattggagagaaaaagctactcgttaaagttgatgcaaagacaaaggcacagctggatgaatggaaagcaaagaagaaagcttctaatgggaatgcaaggccagaaactgtcactaatgacgatgaagaagccttggatgaagaaacaaagaggagagatcagatgattaaaggggctattgaagttttaattcgtgaatactccagtgagctaaatgccccctcacaggaatctgattctcaccccaggaagaagaagaaggaaaagaaggaggacattttcggcagatttcagtgggcccactgat

SEQ ID NO: 153 - DNA, 2060 bp, Homo sapiens
tccccccctcagcctcccccccccccactggcatatggtcctgccccttctaccagacccatgggcccccaggcagcccctcttaccattcgagggccctcgtctgctggccagtccacccctagtccccacctggtgccttcacctgccccatctccagggcctggtccggtaccccctcgccccccagcagcagaaccacccccttgcctgcgccgaggcgccgcagctgcagacctgctctcctccagcccggagagccagcatggcggcactcagtctcctgggggtgggcagcccctgctgcagcccaccaaggtggatgcagctgagggtcgtcggccgcaggccctgcggctgattgagcgggacccctatgagcatcctgagaggctgcggcagttgcagcaggagctggaggcctttcggggtcagctgggggatgtgggagctctggacactgtctggcgagagctgcaagatgcgcaggaacatgatgcccgaggccgttccatcgccattgcccgctgctactcactgaagaaccggcaccaggatgtcatgccctatgacagtaaccgtgtggtgctgcgctcaggcaaggatgactacatcaatgccagctgcgtggaggggctctccccatactgccccccgctagtggcaacccaggccccactgcctggcacagctgctgacttctggctcatggtccatgagcagaaagtgtcagtcattgtcatgctggtttctgaggctgagatggagaagcaaaaagtggcacgctacttccccaccgagaggggccagcccatggtgcacggtgccctgagcctggcattgagcagcgtccgcagcaccgaaacccatgtggagcgcgtgctgagcctgcagttccgagaccagagcctcaagcgctctcttgtgcacctgcacttccccacttggcctgagttaggcctgcccgacagccccagcaacttgctgcgcttcatccaggaggtgcacgcacattacctgcatcagcggccgctgcacacgcccatcattgtgcactgcagctctggtgtgggccgcacgggagcctttgcactgctctatgcagctgtgcaggaggtggaggctgggaacggaatccctgagctgcctcagctggtgcggcgcatgcggcagcagagaaagcacatgctgcaggagaagctgcacctcaggttctgctatgaggcagtggtgagacacgtggagcaggtcctgcagcgccatggtgtgcctcctccatgcaaacccttggccagtgcaagcatcagccagaagaaccaccttcctcaggactcccaggacctggtcctcggtggggatgtgcccatcagctccatccaggccaccattgccaagctcagcattcggcctcctggggggttggagtccccggttgccagcttgccaggccctgcagagcccccaggcctcccgccagccagcctcccagagtctaccccaatcccatcttcctcccaaaccccctttcctccccactacctgaggctccccagcctaaggaggagccgccagtgcctgaagcccccagctcggggcccccctcctcctccctggaattgctggcctccttgaccccagaggccttctccctggacagctccctgcggggcaaacagcggatgagcaagcataactttctgcaggcccataacgggcaagggctgcgggccacccggccctctgacgaccccctcagccttctggatccactctggacactcaacaagacctgaacaggttttgcctacctggtccttacactacatcatcatcatctcatgcccacctgcccacacccagcagagcttctcagtgggcacagtctcttactcccatttctgctgcctttggccctgcctggcccagcctgcacccctgtggggtggaaatgtactgcaggctctgggtcaggttctgctcctttatgggacccgacatttttcagctctttgctattgaaataataaaccaccctgttctgtgaaaaaaaaaaaaaaaag

SEQ ID NO: 154 - DNA, 2065 bp, Homo sapiens
cgggtccccgggtctgacaggagcagcctgtgggcaccgcggcggtagttggaggcgggagagggtccgtagccgcgccgccctgccccgccatgggcctcctgtcggacccggttcgccggcgcgcgctcgcccgcctagtgctgcgcctcaacgcgccgttgtgcgtgctgagctacgtggcgggcatcgcctggttcttggcgctggttttcccgccgctgacccagcgcacttacatgtcggagaacgccatgggctccaccatggtggaggagcagtttgcgggcggagaccgtgcccgggcttttgcccgggacttcgccgcccaccgcaagaagtcgggggctctgccagtggcctggcttgaacggacgatgcggtcagtagggctggaggtctacacgcagagtttctcccggaaactgcccttcccagatgagacccacgagcgctatatggtgtcgggcaccaacgtgtacggcatcctgcgggccccgcgtgctgccagcaccgagtcgcttgtgctcaccgtgccctgtggctctgactctaccaacagccaggctgtggggctgctgctggcactggctgcccacttccgggggcagatttattgggccaaagatatcgtcttcctggtaacagaacatgaccttctgggcactgaggcttggcttgaagcctaccacgatgtcaatgtcactggcatgcagtcgtctcccctgcagggccgagctggggccattcaggcagccgtggccctggagctgagcagtgatgtggtcaccagcctcgatgtggccgtggaggggcttaacgggcagctgcccaaccttgacctgctcaatctcttccagaccttctgccagaaagggggcctgttgtgcacgcttcagggcaagctgcagcccgaggactggacatcattggatggaccgctgcagggcctgcagacactgctgctcatggttctgcggcaggcctccggccgcccccacggctcccatggcctcttcctgcgctaccgtgtggaggccctaaccctgcgtggcatcaatagcttccgccagtacaagtatgacctggtggcagtgggcaaggctttggagggcatgttccgcaagctcaaccacctcctggagcgcctgcaccagtccttcttcctctacttgctccccggcctctcccgcttcgtctccatcggcctctacatgcccgctgtcggcttcttgctcctggtccttggtctcaaggctctggaactgtggatgcagctgcatgaggctggaatgggccttgaggagcccgggggtgcccctggccccagtgtaccccttcccccatcacagggtgtggggctggcctcgctcgtggcacctctgctgatctcacaggccatgggactggccctctatgtcctgccagtgctgggccaacacgttgccacccagcacttcccagtggcagaggctgaggctgtggtgctgacactgctggcgatttatgcagctggcctggccctgccccacaatacccaccgggtggtaagcacacaggccccagacaggggctggatggcactgaagctggtagccctgatctacctagcactgcagctgggctgcatcgccctcaccaacttctcactgggcttcctgctggccaccaccatggtgcccactgctgcgcttgccaagcctcatgggccccggaccctctatgctgccctgctggtgctgaccagcccggcagccacgctccttggcagcctgttcctgtggcgggagctgcaggaggcgccactgtcactggccgagggctggcagctcttcctggcagcgctagcccagggtgtgctggagcaccacacctacggcgccctgctcttcccactgctgtccctgggcctctacccctgctggctgcttttctggaatgtgctcttctggaagtgagatctgcctgtccgggctgggacagagactccccaaggaccccattctgcctccttctggggaaataaatgagtgtctgtttcagcagctatttgatgcttgtcaca

SEQ ID NO: 155 - protein, 6 aa, Artificial Sequence
  Description of Artificial Sequence Synthetic peptide
DTAGQE

(19) United States
(12) Patent Application Publication
(10) Pub. No.: US 2009/0176707 A1
(43) Pub. Date: Jul. 9, 2009

(54) HUMAN SIGNAL PEPTIDE-CONTAINING PROTEINS

(75) Inventors: PREETI LAL, SANTA CLARA, CA (US); JENNIFER L. HILLMAN, MOUNTAIN VIEW, CA (US); NEIL C. CORLEY, MOUNTAIN VIEW, CA (US); KARL J. GUEGLER, MENLO PARK, CA (US); MARIAH R. BAUGHN, SAN JOSE, CA (US); SUSAN K. SATHER, PALO ALTO, CA (US); PURVI SHAH, SUNNYVALE, CA (US)

Correspondence Address: FOLEY AND LARDNER LLP;SUITE 500, 3000 K STREET NW, WASHINGTON, DC, 20007, US

(73) Assignee: INCYTE PHARMACEUTICALS INC.

(21) Appl. No.: 12/270,629

(22) Filed: Nov. 13, 2008

Related U.S. Application Data
(62) Division of application No. 11/386,836, filed on Mar. 23, 2006, now abandoned.
Application No. 11/386,836 is a division of application No. 09/002,485, filed on Dec. 31, 1997, now abandoned.

(51) Int. Cl. A61K 38/17; C07H 21/00; C12N 5/10; C12P 21/02; G01N 33/68; C07K 16/18

(52) U.S. Cl. 514/12; 536/23.5; 435/325; 435/69.1; 436/86; 530/387.9

(57) ABSTRACT

The invention provides a human signal peptide-containing proteins (SIGP) and polynucleotides which identify and encode SIGP. The invention also provides expression vectors, host cells, antibodies, agonists, and antagonists. The invention also provides methods for treating or preventing disorders associated with expression of SIGP.

### CROSS-REFERENCE TO RELATED APPLICATIONS

This application is a division of U.S. application Ser. No. 11/386,836 filed on Mar. 23, 2006, now abandoned, which is a division of U.S. application Ser. No. 09/002,485, filed on Dec. 31, 1997, now abandoned. The contents of these applications are hereby incorporated by reference in their entirety.

### FIELD OF THE INVENTION

This invention relates to nucleic acid and amino acid sequences of human signal peptide-containing proteins and to the use of these sequences in the diagnosis, treatment, and prevention of cancer and immunological disorders.

### BACKGROUND OF THE INVENTION

Protein transport is an essential process for all living cells. Transport of an individual protein usually occurs via an amino-terminal signal sequence which directs, or targets, the protein from its ribosomal assembly site to a particular cellular or extracellular location. Transport may involve any combination of several of the following steps: contact with a chaperone, unfolding, interaction with a receptor and/or a pore complex, addition of energy, and refolding. Moreover, an extracellular protein may be produced as an inactive precursor. Once the precursor has been exported, removal of the signal sequence by a signal peptidase and posttranslational processing (e.g., glycosylation or phosphorylation) activates the protein. Signal sequences are common to receptors, matrix molecules (e.g., adhesion, cadherin, extracellular matrix, integrin, and selectin), cytokines, hormones, growth and differentiation factors, neuropeptides, vasomediators, phosphokinases, phosphatases, phospholipases, phosphodiesterases, G and Ras-related proteins, ion channels, transporters/pumps, proteases, and transcription factors.

G-protein coupled receptors (GPCRs) are a superfamily of integral membrane proteins which transduce extracellular signals. GPCRs include receptors for biogenic amines, e.g., dopamine, epinephrine, histamine, glutamate (metabotropic effect), acetylcholine (muscarinic effect), and serotonin; for lipid mediators of inflammation such as prostaglandins, platelet activating factor, and leukotrienes; for peptide hormones such as calcitonin, C5a anaphylatoxin, follicle stimulating hormone, gonadotropin releasing hormone, neurokinin, oxytocin, and thrombin; and for sensory signal mediators, e.g., retinal photopigments and olfactory stimulatory molecules.

The structure of these highly-conserved receptors consists of seven hydrophobic transmembrane regions, cysteine disulfide bridges between the second and third extracellular loops, an extracellular N-terminus, and a cytoplasmic C-terminus. Three extracellular loops alternate with three intracellular loops to link the seven transmembrane regions. The N-terminus interacts with ligands, the disulfide bridge interacts with agonists and antagonists, and the large third intracellular loop interacts with G proteins to activate second messengers such as cyclic AMP (cAMP), phospholipase C, inositol triphosphate, or ion channel proteins. The most conserved parts of these proteins are the transmembrane regions and the first two cytoplasmic loops. A conserved, acidic-Arg-aromatic triplet present in the second cytoplasmic loop may interact with the G proteins. The consensus pattern, [GSTALIVMYWC]-[GSTANCPDE]-{EDPKRH}-x(2)-[LIVMNQGA]-x(2)-[LIVMFT]-[GTANC]-[LIVMFYWSTAC]-[DENH]-R—[FYWCSH]-x(2)-[LIVM] is characteristic of most proteins belonging to this superfamily. (Watson, S. and Arkinstall, S. (1994) The G-protein Linked Receptor Facts Book, Academic Press, San Diego, Calif., pp. 2-6; and Bolander, F. F. (1994) Molecular Endocrinology, Academic Press, San Diego, Calif., pp. 8-19.)

Tetraspanins are a superfamily of membrane proteins which facilitate the formation and stability of cell-surface signaling complexes containing lineage-specific proteins, integrins, and other tetraspanins. They are involved in cell activation, proliferation (including cancer), differentiation, adhesion, and motility. These proteins cross the membrane four times, have conserved intracellular N- and C-termini and an extracellular, non-conserved hydrophilic domain. Three highly conserved polar amino acids are located in the transmembrane domains (TM), an asparagine in TM1 and a glutamate or glutamine in TM3 and TM4. Two to three conserved charged residues, including a glutamic acid residue, are present in the cytoplasmic loop between TM2 and TM3. The extracellular loop between TM3 and TM4 contains four conserved cysteine residues: two in a conserved CCG motif located about 50 residues C-terminal to TM3; one, often preceded by glycine, 11 residues N-terminal to TM4; and one in the extracellular loop may be found in a PXSC motif. Tetraspanins include, e.g., platelet and endothelial cell membrane proteins, leukocyte surface proteins, tissue specific and tumorous antigens, and the retinitis pigmentosa-associated gene peripherin. (Maecker, H. T. et al. (1997) FASEB J. 11:428-442.) Matrix proteins (Mps) function in formation, growth, remodeling and maintenance of tissues and as important mediators and regulators of the inflammatory response. The expression and balance of MPs may be perturbed by biochemical changes that result from congenital, epigenetic, or infectious diseases. In addition, MPs affect leukocyte migration, proliferation, differentiation, and activation in immune response.

MPs encompass a variety of proteins and their functions. Extracellular matrix (ECM) proteins are multidomain proteins that play an important role in the diverse functions of the ECM. ECM proteins are frequently characterized by the presence of one or more domains which may include collagen-like domains, EGF-like domains, immunoglobulin-like domains, fibronectin-like domains, vWFA-like modules. (Ayad, S. et al. (1994) The Extracellular Matrix Facts Book, Academic Press, San Diego, Calif., pp. 2-16.) Cell adhesion molecules (CAMs) have been shown to stimulate axonal growth through homophilic and/or heterophilic interactions with other molecules. In addition, interactions between adhesion molecules and their receptors can potentiate the effects of growth factors upon cell biochemistry via shared signaling pathways. (Ruoslahti, E. (1997) Kidney Int. 51: 1413-1417.) Cadherins comprise a family of calcium-dependant glycoproteins that function in mediating cell-cell adhesion in solid tissues of multicellular organisms. Integrins are ubiquitous transmembrane adhesion molecules that link cells to the ECM by interacting with the cytoskeleton. Integrins also function as signal transduction receptors and stimulate changes in intracellular calcium levels and protein kinase activity. (Sjaastad, M. D. and Nelson, W. J. (1997) BioEssays 19:47-55.)

Lectins are proteins characterized by their ability to bind carbohydrates on cell membranes by means of discrete, modular carbohydrate recognition domains, CRDs. (Kishore, U. et al. (1997) Matrix Biol. 15:583-592.) Certain cytokines and membrane-spanning proteins have CRDs which may enhance interactions with extracellular or intracellular ligands, with proteins in secretory pathways, or with molecules in signal transduction pathways. The lipocalin superfamily constitutes a phylogenetically conserved group of more than forty proteins that function by binding to and transporting a variety of physiologically important ligands. Members of this family function as carriers of retinoids, odorants, chromophores, pheromones, and sterols, and a subset of these proteins may be multifunctional, serving as either a biosynthetic enzyme or as a specific enzyme inhibitor. (Tanaka, T. et al. (1997) J. Biol. Chem. 272:15789-15795; and van't Hof, W. et al. (1997) J. Biol. Chem. 272:1837-1841.) Selectins are a family of calcium ion-dependent lectins expressed on inflamed vascular endothelium and the surface of some leukocytes. They mediate rolling movement and adhesive contacts between blood cells and blood vessel walls. The structure of the selectins and their ligands supports the type of bond formation and dissociation that allows a cell to roll under conditions of flow. (Rossiter, H. et al. (1997) Mol. Med. Today 3:214-222.)

Protein kinases regulate many different cell proliferation, differentiation, and signaling processes by adding phosphate groups to proteins. Reversible protein phosphorylation is a key strategy for controlling protein functional activity in eukaryotic cells. The high energy phosphate which drives this activation is generally transferred from adenosine triphosphate molecules (ATP) to a particular protein by protein kinases and removed from that protein by protein phosphatases. Phosphorylation occurs in response to extracellular signals, cell cycle checkpoints, and environmental or nutritional stresses. Protein kinases may be roughly divided into two groups; protein tyrosine kinases (PTKs) which phosphorylate tyrosine residues, and serine/threonine kinases (STKs) which phosphorylate serine or threonine residues. A few protein kinases have dual specificity. A majority of kinases contain a similar 250-300 amino acid catalytic domain which can be further divided into eleven subdomains. The N-terminal domain, which contains subdomains I to IV, generally folds into a two-lobed structure which binds and orients the ATP (or GTP) donor molecule. The larger C terminal domain, which contains subdomains VIA to XI, binds the protein substrate and carries out the transfer of the gamma phosphate from ATP to the hydroxyl group of the target amino acid residue. Subdomain V links the two domains. Each of the 11 subdomains contain specific residues and motifs that are characteristic and are highly conserved. (Hardie, G. and Hanks, S. (1995) The Protein Kinase Facts Book, Vol I, pp. 7-47, Academic Press, San Diego, Calif.)

Protein phosphatases remove phosphate groups from molecules previously modified by protein kinases thus participating in cell signaling, proliferation, differentiation, contacts, and oncogenesis. Protein phosphorylation is a key strategy used to control protein functional activity in eukaryotic cells. The high energy phosphate is transferred from ATP to a protein by protein kinases and removed by protein phosphatases. There appear to be three, evolutionarily-distinct protein phosphatase gene families: protein phosphatases (PPs); protein tyrosine phosphatases (PTPs); and acid/alkaline phosphatases (APs). PPs dephosphorylate phosphoserine/threonine residues and are an important regulator of many cAMP mediated, hormone responses in cells. PTPs reverse the effects of protein tyrosine kinases and therefore play a significant role in cell cycle and cell signaling processes. Although APs dephosphorylate substrates in vitro, their role in vivo is not well known. (Carbonneau, H. and Tonks, N. K. (1992) Annu. Rev. Cell Biol. 8:463-493.)

Protein phosphatase inhibitors control the activities of specific phosphatases. A specific inhibitor of PP-I, I-1, has been identified that when phosphorylated by cAMP-dependent protein kinase (PKA) specifically binds to PP-I and inhibits its activity. Since PP-I is dephosphoryles many of the proteins phosphorylated by PKA, activation of I-1 by PKA serves to amplify the effects of PKA and the many cAMP-dependent responses mediated by PKA. In addition, since PP-I also dephosphorylates many phosphoproteins that are not phosphorylated by PKA, I-1 activation serves to exert cAMP control over other protein phosphorylations. I.sub.1 PP2A is a specific and potent inhibitor of PP-IIA. (Li, M. et al. (1996) Biochemistry 35:6998-7002.) Since PP-IIA is the main phosphatase responsible for reversing the phosphorylations of serine/threonine kinases, I.sub.1PP2A has broad effects in controlling protein phosphorylations.

Cyclic nucleotides (cAMP and cGMP) function as intracellular second messengers to transduce a variety of extracellular signals, including hormones, and light and neurotransmitters. Cyclic nucleotide phosphodiesterases (PDEs) degrade cyclic nucleotides to their corresponding monophosphates, thereby regulating the intracellular concentrations of cyclic nucleotides and their effects on signal transduction. At least seven families of mammalian PDEs have been identified based on substrate specificity and affinity, sensitivity to cofactors and sensitivity to inhibitory drugs. (Beavo, J. A. (1995) Physiological Reviews 75: 725-748.) PDEs are composed of a catalytic domain of ˜270 amino acids, an N-terminal regulatory domain responsible for binding cofactors and, in some cases, a C-terminal domain with unknown function. Within the catalytic domain, there is approximately 30% amino acid identity between PDE families and ˜85-95% identity between isozymes of the same family. Furthermore, within a family there is extensive similarity (>60%) outside the catalytic domain, while across families there is little or no sequence similarity. A variety of diseases have been attributed to increased PDE activity and inhibitors of PDEs have been used effectively as anti-inflammatory, antihypertensive, and antithrombotic agents. (Verghese, M. W. et al. (1995) Mol. Pharmacol. 47:1164-1171; and Banner, K. H. and Page, C. P. (1995) Eur. Respir. J. 8:996-1000.)

Phospholipases (PLs) are enzymes that catalyze the removal of fatty acid residues from phosphoglycerides. PLs play an important role in transmembrane signal transduction and are named according to the specific ester bond in phosphoglycerides that is hydrolyzed, i.e., A₁, A₂, C or D. PLA₂ cleaves the ester bond at position 2 of the glycerol moiety of membrane phospholipids giving rise to arachidonic acid. Arachidonic acid is the common precursor to four major classes of eicosanoids; prostaglandins, prostacyclins, thromboxanes and leukotrienes. Eicosanoids are signaling molecules involved in the contraction of smooth muscle, platelet aggregation, and pain and inflammatory responses. PLC is an important link in certain receptor-mediated, signaling transduction pathways. Extracellular signaling molecules including hormones, growth factors, neurotransmitters, and immunoglobulins bind to their respective cell surface receptors and activate PLC. Activated PLC generates second messenger molecules from the hydrolysis of inositol phospholipids that regulate cellular processes, e.g., secretion, neural activity, metabolism and proliferation. (Alberts, B. et al. (1994) Molecular Biology of The Cell, Garland Publishing, Inc., New York, N.Y., pp. 85, 211, 239-240, 642-645.)

The nucleotide cyclases, i.e., adenylate and guanylate cyclase, catalyze the synthesis of the cyclic nucleotides, cAMP and cGMP, from ATP and GTP, respectively. They act in concert with phosphodiesterases, which degrade cAMP and cGMP, to regulate the cellular levels of these molecules and their functions. cAMP and cGMP function as intracellular second messengers to transduce a variety of extracellular signals, e.g., hormones, and light and neurotransmitters. Adenylate cyclase is a plasma membrane protein that is coupled with various hormone receptors also located on the plasma membrane. Binding of a hormone to its receptor activates adenylate cyclase which, in turn, increases the levels of cAMP in the cytosol. The activation of other molecules by cAMP leads to the cellular effect of the hormone. In a similar manner, guanylate cyclase participates in the process of visual excitation and phototransduction in the eye. (Stryer, L. (1988) Biochemistry W.H. Freeman and Co., New York, pp. 975-980, 1029-1035.) Cytokines are produced in response to cell perturbation. Some cytokines are produced as precursor forms, and some form multimers in order to become active. They are produced in groups and in patterns characteristic of the particular stimulus or disease, and the members of the group interact with one another and other molecules to produce an overall biological response. Interleukins, neurotrophins, growth factors, interferons, and chemokines are all families of cytokines which work in conjunction with cellular receptors to regulate cell proliferation and differentiation and to affect such activities, e.g., leukocyte migration and function, hematopoietic cell proliferation, temperature regulation, acute response to infections, tissue remodeling, and cell survival. Studies using antibodies or other drugs that modify the activity of a particular cytokine are used to elucidate the roles of individual cytokines in pathology and physiology.

Chemokines are a small chemoattractant cytokines which are active in leukocyte trafficking. Initially, chemokines were isolated and purified from inflamed tissues, but recently several chemokines have been discovered through molecular cloning techniques. Chemokines have been shown to be active in cell activation and migration, angiogenic and angiostatic activities, suppression of hematopoiesis, HIV infectivity, and promoting Th-1 (IL-2-, interferon γ-stimulated) cytokine release.

Chemokines generally contain 70-100 amino acids and are subdivided into four subfamilies based on the presence and arrangement of conserved CXC, CC, CX3C and C motifs. The CXC (alpha), CC (beta), and CX3C chemokines contain four conserved cysteines. The CC subfamily is active on monocytes, lymphocytes, eosinophils, and mast cells; the CXC subfamily, on neutrophils; CX3C and C subfamilies, on T-cells. Many of the CC chemokines have been characterized functionally as well as structurally. (Callard, R. and Gearing, A. (1994) The Cytokine Facts Book, Academic Press, New York, N.Y., pp. 181-190, 210-213, 223-227.)

Growth and differentiation factors function in intercellular communication. Once secreted from the cell, some factors require oligomerization or association with ECM in order to function. Complex interactions among these factors and their receptors result in the stimulation or inhibition of cell division, cell differentiation, cell signaling, and cell motility. Some factors act on their cell of origin (autocrine signaling); on neighboring cells (paracrine signaling); or on distant cells (endocrine signaling).

There are three broad classes of growth and differentiation factors. The first class includes the large polypeptide growth factors, e.g., epidermal growth factor, fibroblast growth factor, transforming growth factor, insulin-like growth factor, and platelet-derived growth factor. Each of these defines a family of related molecules which stimulate cell proliferation for wound healing, bone synthesis and remodeling, and regeneration of epithelial, epidermal, and connective tissues, and induce differentiation of embryonic tissues. Nerve growth factor functions specifically as a neurotrophic factor, and all induce differentiation of embryonic tissues. The second class includes the hematopoietic growth factors which stimulate the proliferation and differentiation of blood cells such as B-lymphocytes, T-lymphocytes, erythrocytes, platelets, eosinophils, basophils, neutrophils, macrophages, and their stem cell precursors. These factors include colony-stimulating factors, erythropoietin, and cytokines, e.g., interleukins, interferons (IFNs), and tumor necrosis factor (TNF). Cytokines are secreted by cells of the immune system and function in immunomodulation. The third class includes small peptide factors e.g., bombesin, vasopressin, oxytocin, endothelin, transferrin, angiotensin II, vasoactive intestinal peptide, and bradykinin, which function as hormones to regulate cellular functions other than proliferation.

Growth and differentiation factors have been shown to play critical roles in neoplastic transformation of cells in vitro and in tumor progression in vivo. Inappropriate expression of growth factors by tumor cells may contribute to vascularization and metastasis of melanotic tumors. In hematopoiesis, growth factor misregulation can result in anemias, leukemias and lymphomas. Certain growth factors, e.g., IFN, are cytotoxic to tumor cells both in vivo and in vitro. Moreover, growth factors and/or their receptors are related both structurally and functionally related to oncoproteins. In addition, growth factors affect transcriptional regulation of both proto-oncogenes and oncosuppressor genes. (Pimentel, E. (1994) Handbook of Growth Factors, CRC Press, Ann Arbor, Mich., pp. 6-25.)

Proteolytic enzymes or proteases degrade proteins by reducing the activation energy needed for the hydrolysis of peptide bonds. The major families are the zinc, serine, cysteine, thiol, and carboxyl proteases.

Zinc proteases, e.g., carboxypeptidase A, have a zinc ion bound to the active site, recognize C-terminal residues that contain an aromatic or bulky aliphatic side chain, and hydrolyze the peptide bond adjacent to the C-terminal residues. Serine proteases have an active site serine residue and include digestive enzymes, e.g., trypsin and chymotrypsin, components of the complement and blood-clotting cascades, and enzymes that control the degradation and turnover of extracellular matrix (ECM) molecules. Subfamilies of serine proteases include tryptases (cleavage after arginine or lysine), aspases (cleavage after aspartate), chymases (cleavage after phenylalanine or leucine), metases (cleavage after methionine), and serases (cleavage after serine). Cysteine proteases (e.g. cathepsin) are produced by monocytes, macrophages and other immune cells and are involved in diverse cellular processes ranging from the processing of precursor proteins to intracellular degradation. Overproduction of these enzymes can cause the tissue destruction associated with rheumatoid arthritis and asthma. Thiol proteases, e.g., papain, contain an active site cysteine and are widely distributed within tissues. Thiol proteases effect catalysis through a thiol ester intermediate facilitated by a proximal histidine side chain. Carboxyl proteases, e.g., pepsin, are active only under acidic conditions (pH 2 to 3). The active site of pepsin contains two aspartate residues; when one aspartate is ionized and the other is not, the enzyme is active. A common feature of the carboxyl proteases is that they are inhibited by very low concentrations (10-¹⁰ M) of the inhibitor pepstatin. A substrate analog which induces structural changes at the active site of a protease functions as an antagonist or inhibitor.

Guanosine triphosphate-binding proteins (G proteins) participate in intracellular signal transduction and control regulatory pathways through cell surface receptors. These receptors respond to hormones, growth factors, neuromodulators, or other signaling molecules, by binding GTP. Binding of GTP leads to the production of cAMP which controls phosphorylation and activation of other proteins. During this process, the hydrolysis of GTP acts as an energy source as well as an on-off switch for the GTPase activity.

The G proteins are small proteins which consist of single 21-30 kDa polypeptides. They can be classified into five subfamilies: Ras, Rho, Ran, Rab, and ADP-ribosylation factor. These proteins regulate cell growth, cell cycle control, protein secretion, and intracellular vesicle interaction. In particular, the Ras proteins are essential in transducing signals from receptor tyrosine kinases to serine/threonine kinases which control cell growth and differentiation. Mutant Ras proteins, which bind but can not hydrolyze GTP, are permanently activated and cause continuous cell proliferation or cancer.

All five subfamilies share common structural features and four conserved motifs, I to IV. Motif I is the most variable and has the signature of GXXXXGK, in which lysine interacts with the β- and γ-phosphate groups of GTP. Motif II, III, and IV have DTAGQE (SEQ ID NO: 155), NKXD, and EXSAX as their respective signatures and regulate the binding of g-phosphate, GTP, and the guanine base of GTP, respectively. Most of the membrane-bound G proteins require a carboxy terminal isoprenyl group (CAAX), added posttranslationally, for membrane association and biological activity. The G proteins also have a variable effector region, located between motifs I and II, which is characterized as the interaction site for guanine nucleotide exchange factors or GTPase-activating proteins.

Eukaryotic cells are bound by a membrane and subdivided into membrane bound compartments. As membranes are impermeable to many ions and polar molecules, transport of these molecules is mediated by ion channels, ion pumps, transport proteins, or pumps. Symporters and antiporters regulate cytosolic pH by transporting ions and small molecules, e.g., amino acids, glucose, and drugs, across membranes; symporters transport small molecules and ions in the same direction, and antiporters, in the opposite direction. Transporter superfamilies include facilitative transporters and active ATP binding cassette transporters involved in multiple-drug resistance and the targeting of antigenic peptides to MHC Class 1 molecules. These transporters bind to a specific ion or other molecule and undergo conformational changes in order to transfer the ion or molecule across a membrane. Transport can occur by a passive, concentration-dependent mechanism or can be linked to an energy source such as ATP hydrolysis or an ion gradient.

Ion channels are formed by transmembrane proteins which form a lined passageway across the membrane through which water and ions, e.g., Na⁺, K⁺, Ca.²⁺, and Cl⁻, enter and exit the cell. For example, chloride channels are involved in the regulation of the membrane electric potential as well as absorption and secretion of ions across the membrane. In intracellular membranes of the Golgi apparatus and endocytic vesicles, chloride channels also regulate organelle pH. Electrophysiological and pharmacological studies suggest that a variety of chloride channels exist in different cell types and that many of these channels have one or more protein kinase phosphorylation sites.

Ion pumps are ATPases which actively maintain membrane gradients. Ion pumps can be grouped into three classes, e.g., P, V, and F, according to their structure and function. All have one or more binding sites for ATP on the cytosolic face of the membrane. The P-class ion pumps consist of two a and two β transmembrane subunits, include Ca²⁺ ATPase and Na⁺/K⁺ ATPase, and function in transporting H⁺, Na⁺, K⁺, and Ca²⁺ ions. The V- and F-class ion pumps have similar structures, a cytosolic domain formed by at least five extrinsic polypeptides and at least 2 transmembrane proteins, and only transport H⁺. F class H⁺ pumps have been identified from the membranes of mitochondria and chloroplast, and V-class H⁺ pumps regulate acidity inside lysosomes, endosomes, and plant vacuoles.

A family of structurally related intrinsic membrane proteins known as facilitative glucose transporters catalyze the movement of glucose and other selected sugars across the plasma membrane. The proteins in this family contain a highly conserved, large transmembrane domain made of 12 transmembrane α-helices, and several less conserved, asymmetric, cytoplasmic and exoplasmic domains. (Pessin, J. E., and Bell, G. I. (1992) Annu. Rev. Physiol. 54:911-930.)

Amino acid transport is mediated by Na⁺ dependent amino acid transporters. These transporters are involved in gastrointestinal and renal uptake of dietary and cellular amino acids and the re-uptake of neurotransmitters. Transport of cationic amino acids is mediated by the system y+ family members and the cationic amino acid transporter (CAT) family. Members of the CAT family share a high degree of sequence homology, and each contains 12-14 putative transmembrane domains. (Ito, K. and Groudine, M. (1997) J. Biol. Chem. 272:26780-26786.)

Proton-coupled, 12 membrane-spanning domain transporters such as PEPT 1 and PEPT 2 are responsible for gastrointestinal absorption and for renal reabsorbtion of peptides using an electrochemical H⁺ gradient as the driving force. A heterodimeric peptide transporter, consisting of TAP 1 and TAP 2, is associated with antigen processing. Peptide antigens are transported across the membrane of the endoplasmic reticulum so they can be presented to the major histocompatibility complex class 1 molecules. Each TAP protein consists of multiple hydrophobic membrane spanning segments and a highly conserved ATP-binding cassette. (Boll, M. et al. (1996) Proc. Natl. Acad. Sci. 93:284-289.)

Hormones are secreted molecules that circulate in the body fluids and bind to specific receptors on the surface of, or within, target tissue cells. Although they have diverse biochemical compositions and mechanisms of action, hormones can be grouped into two categories. One category consists of small lipophilic molecules that diffuse through the plasma membrane of target cells, bind to cytosolic or nuclear receptors, and form a complex alters gene expression. Examples of this category include retinoic acid, thyroxine, and the cholesterol derived steroid hormones, progesterone, estrogen, testosterone, cortisol, and aldosterone. These hormones have a long half-life, e.g., several hours to days, and long-term effects of their target cells. Their solubility in the blood may be increased by their association with carrier molecules. Within the target cell nucleus, hormone/receptor complexes bind to specific response elements in target gene regulatory regions.

A second category consists of hydrophilic hormones that function by binding to cell surface receptors and transducing the signal across the plasma membrane. Examples of this category include amino acid derivatives, such as catecholamines, e.g., epinephrine, norepinephrine, and histamine; peptide hormones, e.g., glucagon, insulin, gastrin, secretin, cholecystokinin, adrenocorticotropic hormone, follicle stimulating hormone, luteinizing hormone, thyroid stimulating hormone, parathormone, and vasopressin. Peptide hormones are synthesized as inactive forms and stored in secretory vesicles. These hormones are activated by protease cleavage before being released from the cell. Many hydrophilic hormones have a very short half-life and effect, e.g., seconds to hours, and are inactivated by proteases in the blood. (Lodish et al. (1995) Molecular Cell Biology, Scientific American Books Inc., New York, N.Y., pp. 856-864.)

Neuropeptides and vasomediators (NPVM) comprise a large family of endogenous signaling molecules. Included in the family are neurotransmitters such as bombesin, neuropeptide Y, neurotensin, neuromedin N, melanocortins, opioids, e.g., enkephalins, endorphins and dynorphins, galanin, somatostatin, tachykinins, vasopressin, and vasoactive intestinal peptide, and circulatory system-borne signaling molecules, e.g., angiotensin, complement, calcitonin, endothelins, formyl-methionyl peptides, glucagon, cholecystokinin and gastrin. These proteins are synthesized as “pre-pro” molecules, and are activated and inactivated by proteolytic cleavage. NP/VMs can transduce signals directly, modulate the activity or release of other neurotransmitters and hormones, and act as catalytic enzymes in cascades. The effects of NP/VMs range from extremely brief or long-lasting (melanocortin-mediated changes in skin melanin). Regulatory molecules turn individual genes or groups of genes on and off in response to various inductive mechanisms of the cell or organism; act as transcription factors by determining whether or not transcription is initiated, enhanced, or repressed; and splice transcripts as dictated in a particular cell or tissue. Although they interact with short stretches of DNA scattered throughout the entire genome, most gene expression is regulated near the site at which transcription starts or within the open reading frame of the gene being expressed. The regulated stretches of the DNA can be simple and interact with only a single protein, or they can require several proteins acting as part of a complex to regulate gene expression. The external features of the double helix which provide recognition sites are hydrogen bond donor and acceptor groups, hydrophobic patches, major and minor grooves, and regular, repeated stretches of sequences which cause distinct bends in the helix. The surface features of the regulatory molecule are complementary to those of the DNA.

Many of the transcription factors incorporate one of a set of DNA-binding structural motifs, each of which contains either α helices or β sheets and binds to the major groove of DNA. Seven of the structural motifs common to transcription factors are helix-turn-helix, homeodomains, zinc finger, steroid receptor, β sheets, leucine zipper, and helix-loop-helix. (Pabo, C. O. and R. T. Sauer (1992) Ann. Rev. Biochem. 61:1053-95.) Other domains of transcription factors may form crucial contacts with the DNA. In addition, accessory proteins provide important interactions which may convert a particular protein complex to an activator or a repressor or may prevent binding. (Alberts, B. et al. (1994) Molecular Biology of the Cell, Garland Publishing Co, New York, N.Y. pp. 401-474.)

The discovery of new human signal peptide-containing proteins and the polynucleotides encoding these molecules satisfies a need in the art by providing new compositions which are useful in the diagnosis, treatment, and prevention of cancer and immunological disorders.

### SUMMARY OF THE INVENTION

The invention features a substantially purified human signal peptide-containing protein (SIGP), having an amino acid sequence selected from the group consisting of SEQ ID NO: 1 SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, and SEQ ID NO:77.

The invention further provides isolated and substantially purified polynucleotides encoding SIGP. In a particular aspect, the polynucleotide has a nucleic acid sequence selected from the group consisting of SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO: 109, SEQ ID NO: 110, SEQ ID NO: 111, SEQ ID NO: 112, SEQ ID NO:113, SEQ ID NO:114, SEQ ID NO:115, SEQ ID NO:116, SEQ ID NO:117, SEQ ID NO: 118, SEQ ID NO: 119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, and SEQ ID NO:154.

In addition, the invention provides a polynucleotide, or fragment thereof, which hybridizes to any of the polynucleotides encoding an SIGP selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, and SEQ ID NO:77. In another aspect, the invention provides a composition comprising isolated and purified polynucleotides selected from the group consisting of SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO: 106, SEQ ID NO: 107, SEQ ID NO: 108, SEQ ID NO: 109, SEQ ID NO: 110, SEQ ID NO:111, SEQ ID NO:112, SEQ ID NO:113, SEQ ID NO:114, SEQ ID NO:115, SEQ ID NO: 116, SEQ ID NO: 117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO: 130, SEQ ID NO: 131, SEQ ID NO: 132, SEQ ID NO: 133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO: 153, and SEQ ID NO: 154, or a fragment thereof.

The invention further provides a polynucleotide comprising the complement, or fragments thereof, of any one of the polynucleotides encoding SIGP. In another aspect, the invention provides compositions comprising isolated and purified polynucleotides comprising the complement of SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO: 110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO: 128, SEQ ID NO: 129, SEQ ID NO: 130, SEQ ID NO: 131, SEQ ID NO: 132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO: 152, SEQ ID NO: 153, and SEQ ID NO: 154, or fragments thereof.

The present invention further provides an expression vector containing at least a fragment of any one of the polynucleotides selected from the group consisting of SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, and SEQ ID NO: 154. In yet another aspect, the expression vector containing the polynucleotide is contained within a host cell.

The invention also provides a method for producing a polypeptide or a fragment thereof, the method comprising the steps of: (a) culturing the host cell containing an expression vector containing at least a fragment of a polynucleotide encoding SIGP under conditions suitable for the expression of the polypeptide; and (b) recovering the polypeptide from the host cell culture.

The invention also provides a pharmaceutical composition comprising a substantially purified SIGP in conjunction with a suitable pharmaceutical carrier.

The invention further includes a purified antibody which binds to SIGP, as well as a purified agonist and a purified antagonist of SIGP.

The invention also provides a method for treating or preventing a cancer associated with the decreased expression or activity of SIGP, the method comprising the step of administering to a subject in need of such treatment an effective amount of a pharmaceutical composition containing SIGP.

The invention also provides a method for treating or preventing a cancer associated with the increased expression or activity of SIGP, the method comprising the step of administering to a subject in need of such treatment an effective amount of an antagonist of SIGP.

The invention also provides a method for treating or preventing an immune response associated with the increased expression or activity of SIGP, the method comprising the step of administering to a subject in need of such treatment an effective amount of an antagonist of SIGP.

The invention also provides a method for detecting a nucleic acid sequence which encodes a human regulatory proteins in a biological sample, the method comprising the steps of: a) hybridizing a nucleic acid sequence of the biological sample to a polynucleotide sequence complementary to the polynucleotide encoding SIGP, thereby forming a hybridization complex; and b) detecting the hybridization complex, wherein the presence of the hybridization complex correlates with the presence of the nucleic acid sequence encoding the human regulatory protein in the biological sample.

The invention also provides a microarray containing at least a fragment of at least one of the polynucleotides encoding a polypeptide having an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11; SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, and SEQ ID NO:77.

The invention also provides a method for detecting the expression level of a nucleic acid encoding a human regulatory protein in a biological sample, the method comprising the steps of hybridizing the nucleic acid sequence of the biological sample to a complementary polynucleotide, thereby forming hybridization complex; and determining expression of the nucleic acid sequence encoding a human regulatory protein in the biological sample by identifying the presence of the hybridization complex. In a preferred embodiment, prior to the hybridizing step, the nucleic acid sequences of the biological sample are amplified and labeled by the polymerase chain reaction.

### DESCRIPTION OF THE INVENTION

Before the present proteins, nucleotide sequences, and methods are described, it is understood that this invention is not limited to the particular methodology, protocols, cell lines, vectors, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are cited for the purpose of describing and disclosing the cell lines, vectors, and methodologies which are reported in the publications and which might be used in connection with the invention. Nothing herein is to be construed as an admission that the invention is not entitled to antedate such disclosure by virtue of prior invention.

### DEFINITIONS

“SIGP,” as used herein, refers to the amino acid sequences of substantially purified SIGP obtained from any species, particularly a mammalian species, including bovine, ovine, porcine, murine, equine, and preferably the human species, from any source, whether natural, synthetic, semi-synthetic, or recombinant.

The term “agonist,” as used herein, refers to a molecule which, when bound to SIGP, increases or prolongs the duration of the effect of SIGP. Agonists may include proteins, nucleic acids, carbohydrates, or any other molecules which bind to and modulate the effect of SIGP.

An “allele” or an “allelic sequence,” as these terms are used herein, is an alternative form of the gene encoding SIGP. Alleles may result from at least one mutation in the nucleic acid sequence and may result in altered mRNAs or in polypeptides whose structure or function may or may not be altered. Any given natural or recombinant gene may have none, one, or many allelic forms. Common mutational changes which give rise to alleles are generally ascribed to natural deletions, additions, or substitutions of nucleotides. Each of these types of changes may occur alone, or in combination with the others, one or more times in a given sequence.

“Altered” nucleic acid sequences encoding SIGP, as described herein, include those sequences with deletions, insertions, or substitutions of different nucleotides, resulting in a polynucleotide the same SIGP or a polypeptide with at least one functional characteristic of SIGP. Included within this definition are polymorphisms which may or may not be readily detectable using a particular oligonucleotide probe of the polynucleotide encoding SIGP, and improper or unexpected hybridization to alleles, with a locus other than the normal chromosomal locus for the polynucleotide sequence encoding SIGP. The encoded protein may also be “altered,” and may contain deletions, insertions, or substitutions of amino acid residues which produce a silent change and result in a functionally equivalent SIGP. Deliberate amino acid substitutions may be made on the basis of similarity in polarity, charge, solubility, hydrophobicity, hydrophilicity, and/or the amphipathic nature of the residues, as long as the biological or immunological activity of SIGP is retained. For example, negatively charged amino acids may include aspartic acid and glutamic acid, positively charged amino acids may include lysine and arginine, and amino acids with uncharged polar head groups having similar hydrophilicity values may include leucine, isoleucine, and valine; glycine and alanine; asparagine and glutamine; serine and threonine; and phenylalanine and tyrosine.

The terms “amino acid” or “amino acid sequence,” as used herein, refer to an oligopeptide, peptide, polypeptide, or protein sequence, or a fragment of any of these, and to naturally occurring or synthetic molecules. In this context, “fragments”, “immunogenic fragments”, or “antigenic fragments” refer to fragments of SIGP which are preferably about 5 to about 15 amino acids in length and which retain some biological activity or immunological activity of SIGP. Where “amino acid sequence” is recited herein to refer to an amino acid sequence of a naturally occurring protein molecule, “amino acid sequence” and like terms are not meant to limit the amino acid sequence to the complete native amino acid sequence associated with the recited protein molecule.

“Amplification,” as used herein, relates to the production of additional copies of a nucleic acid sequence. Amplification is generally carried out using polymerase chain reaction (PCR) technologies well known in the art. (See, e.g., Dieffenbach, C. W. and G. S. Dveksler (1995) PCR Primer, a Laboratory Manual, Cold Spring Harbor Press, Plainview, N.Y., pp. 1-5.)

The term “antagonist,” as it is used herein, refers to a molecule which, when bound to SIGP, decreases the amount or the duration of the effect of the biological or immunological activity of SIGP. Antagonists may include proteins, nucleic acids, carbohydrates, antibodies, or any other molecules which decrease the effect of SIGP.

As used herein, the term “antibody” refers to intact molecules as well as to fragments thereof, such as Fa, F(ab′)₂, and Fv fragments, which are capable of binding the epitopic determinant. Antibodies that bind SIGP polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant,” as used herein, refers to that fragment of a molecule (i.e., an epitope) that makes contact with a particular antibody. When a protein or a fragment of a protein is used to immunize a host animal, numerous regions of the protein may induce the production of antibodies which bind specifically to antigenic determinants (given regions or three-dimensional structures on the protein). An antigenic determinant may compete with the intact antigen (i.e., the immunogen used to elicit the immune response) for binding to an antibody.

The term “antisense,” as used herein, refers to any composition containing a nucleic acid sequence which is complementary to a specific nucleic acid sequence. The term “antisense strand” is used in reference to a nucleic acid strand that is complementary to the “sense” strand. Antisense molecules may be produced by any method including synthesis or transcription. Once introduced into a cell, the complementary nucleotides combine with natural sequences produced by the cell to form duplexes and to block either transcription or translation. The designation “negative” can refer to the antisense strand, and the designation “positive” can refer to the sense strand.

As used herein, the term “biologically active,” refers to a protein having structural, regulatory, or biochemical functions of a naturally occurring molecule. Likewise, “immunologically active” refers to the capability of the natural, recombinant, or synthetic SIGP, or of any oligopeptide thereof, to induce a specific immune response in appropriate animals or cells and to bind with specific antibodies.

The terms “complementary” or “complementarity,” as used herein, refer to the natural binding of polynucleotides under permissive salt and temperature conditions by base pairing. For example, the sequence “A-G-T” binds to the complementary sequence “T-C-A.” Complementarity between two single-stranded molecules may be “partial,” such that only some of the nucleic acids bind, or it may be “complete,” such that total complementarity exists between the single stranded molecules. The degree of complementarity between nucleic acid strands has significant effects on the efficiency and strength of the hybridization between the nucleic acid strands. This is of particular importance in amplification reactions, which depend upon binding between nucleic acids strands, and in the design and use of peptide nucleic acid (PNA) molecules.

A “composition comprising a given polynucleotide sequence” or a “composition comprising a given amino acid sequence,” as these terms are used herein, refer broadly to any composition containing the given polynucleotide or amino acid sequence. The composition may comprise a dry formulation, an aqueous solution, or a sterile composition. Compositions comprising polynucleotides encoding SIGP, e.g., SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, and SEQ ID NO: 154, or fragments thereof, may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., SDS) and other components (e.g., Denhardt's solution, dry milk, salmon sperm DNA, etc.).

The phrase “consensus sequence,” as used herein, refers to a nucleic acid sequence which has been resequenced to resolve uncalled bases, extended using XL-PCR™ (Perkin Elmer, Norwalk, Conn.) in the 5′ and/or the 3′ direction, and resequenced, or which has been assembled from the overlapping sequences of more than one Incyte Clone using a computer program for fragment assembly, such as the GELVIEW™ Fragment Assembly system (GCG, Madison, Wis.). Some sequences have been both extended and assembled to produce the consensus sequence.

As used herein, the term “correlates with expression of a polynucleotide” indicates that the detection of the presence of nucleic acids, the same or related to a nucleic acid sequence encoding SIGP, by northern analysis is indicative of the presence of nucleic acids encoding SIGP in a sample, and thereby correlates with expression of the transcript from the polynucleotide encoding SIGP.

The term “SIGP” refers to any or all of the human polypeptides, SIGP-1, SIGP-2, SIGP-3, SIGP-4, SIGP-5, SIGP-6, SIGP-7, SIGP-8, SIGP-9, SIGP-10, SIGP-11, SIGP-12, SIGP-13, SIGP-14, SIGP-15, SIGP-16, SIGP-17, SIGP-18, SIGP-19, SIGP-20, SIGP-21, SIGP-22, SIGP-23, SIGP-24, SIGP-25, SIGP-26, SIGP-27, SIGP-28, SIGP-29, SIGP-30, SIGP-31, SIGP-32, SIGP-33, SIGP-34, SIGP-35, SIGP-36, SIGP-37, SIGP-38, SIGP-39, SIGP-40, SIGP-41, SIGP-42, SIGP-43, SIGP-44, SIGP-45, SIGP-46, SIGP-47, SIGP-48, SIGP-49, SIGP-50, SIGP-51, SIGP-52, SIGP-53, SIGP-54, SIGP-55, SIGP-56, SIGP-57, SIGP-58, SIGP-59, SIGP-60, SIGP-61, SIGP-62, SIGP-63, SIGP-64, SIGP-65, SIGP-66, SIGP-67, SIGP-68, SIGP-69, SIGP-70, SIGP-71, SIGP-72, SIGP-73, SIGP-74, SIGP-75, SIGP-76, and SIGP-77.

A “deletion,” as the term is used herein, refers to a change in the amino acid or nucleotide sequence that results in the absence of one or more amino acid residues or nucleotides.

The term “derivative,” as used herein, refers to the chemical modification of SIGP, of a polynucleotide sequence encoding SIGP, or of a polynucleotide sequence complementary to a polynucleotide sequence encoding SIGP. Chemical modifications of a polynucleotide sequence can include, for example, replacement of hydrogen by an alkyl, acyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

The term “homology,” as used herein, refers to a degree of complementarity. There may be partial homology or complete homology. The word “identity” may substitute for the word “homology.” A partially complementary sequence that at least partially inhibits an identical sequence from hybridizing to a target nucleic acid is referred to as “substantially homologous.” The inhibition of hybridization of the completely complementary sequence to the target sequence may be examined using a hybridization assay (Southern or northern blot, solution hybridization, and the like) under conditions of reduced stringency. A substantially homologous sequence or hybridization probe will compete for and inhibit the binding of a completely homologous sequence to the target sequence under conditions of reduced stringency. This is not to say that conditions of reduced stringency are such that non-specific binding is permitted, as reduced stringency conditions require that the binding of two sequences to one another be a specific (i.e., a selective) interaction. The absence of non-specific binding may be tested by the use of a second target sequence which lacks even a partial degree of complementarity (e.g., less than about 30% homology or identity). In the absence of non-specific binding, the substantially homologous sequence or probe will not hybridize to the second non-complementary target sequence.

The phrases “percent identity” or “% identity” refer to the percentage of sequence similarity found in a comparison of two or more amino acid or nucleic acid sequences. Percent identity can be determined electronically, e.g., by using the MegAlign program (Lasergene software package, DNASTAR, Inc., Madison Wis.). The MegAlign program can create alignments between two or more sequences according to different methods, e.g., the Clustal Method. (Higgins, D. G. and Sharp, P. M. (1988) Gene 73:237-244.) The Clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage similarity. Percent identity between nucleic acid sequences can also be calculated by the Clustal Method, or by other methods known in the art, such as the Jotun Hein Method. (See, e.g., Hein, J. (1990) Methods in Enzymology 183:626-645.) Identity between sequences can also be determined by other methods known in the art, e.g., by varying hybridization conditions.

“Human artificial chromosomes” (HACs), as described herein, are linear microchromosomes which may contain DNA sequences of about 6 kb to 10 Mb in size, and which contain all of the elements required for stable mitotic chromosome segregation and maintenance. (See, e.g., Harrington, J. J. et al. (1997) Nat. Genet. 15:345-355.)

The term “humanized antibody,” as used herein, refers to antibody molecules in which the amino acid sequence in the non-antigen binding regions has been altered so that the antibody more closely resembles a human antibody, and still retains its original binding ability.

“Hybridization,” as the term is used herein, refers to any process by which a strand of nucleic acid binds with a complementary strand through base pairing.

As used herein, the term “hybridization complex” as used herein, refers to a complex formed between two nucleic acid sequences by virtue of the formation of hydrogen bonds between complementary bases. A hybridization complex may be formed in solution (e.g., C₀t or R₀t analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

The words “insertion” or “addition,” as used herein, refer to changes in an amino acid or nucleotide sequence resulting in the addition of one or more amino acid residues or nucleotides, respectively, to the sequence found in the naturally occurring molecule.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

The term “microarray,” as used herein, refers to an array of distinct polynucleotides or oligonucleotides arrayed on a substrate, such as paper, nylon or any other type of membrane, filter, chip, glass slide, or any other suitable solid support.

The term “modulate,” as it appears herein, refers to a change in the activity of SIGP. For example, modulation may cause an increase or a decrease in protein activity, binding characteristics, or any other biological, functional, or immunological properties of SIGP.

The phrases “nucleic acid” or “nucleic acid sequence,” as used herein, refer to an oligonucleotide, nucleotide, polynucleotide, or any fragment thereof, to DNA or RNA of genomic or synthetic origin which may be single-stranded or double-stranded and may represent the sense or the antisense strand, to peptide nucleic acid (PNA), or to any DNA-like or RNA-like material. In this context, “fragments” refers to those nucleic acid sequences which are greater than about 60 nucleotides in length, and most preferably are at least about 100 nucleotides, at least about 1000 nucleotides, or at least about 10,000 nucleotides in length.

The terms “operably associated” or “operably linked,” as used herein, refer to functionally related nucleic acid sequences. A promoter is operably associated or operably linked with a coding sequence if the promoter controls the transcription of the encoded polypeptide. While operably associated or operably linked nucleic acid sequences can be contiguous and in reading frame, certain genetic elements, e.g., repressor genes, are not contiguously linked to the encoded polypeptide but still bind to operator sequences that control expression of the polypeptide.

The term “oligonucleotide,” as used herein, refers to a nucleic acid sequence of at least about 6 nucleotides to 60 nucleotides, preferably about 15 to 30 nucleotides, and most preferably about 20 to 25 nucleotides, which can be used in PCR amplification or in a hybridization assay or microarray. As used herein, the term “oligonucleotide” is substantially equivalent to the terms “amplimers,” “primers,” “oligomers,” and “probes,” as these terms are commonly defined in the art.

“Peptide nucleic acid” (PNA), as used herein, refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA and RNA and stop transcript elongation, and may be pegylated to extend their lifespan in the cell. (See, e.g., Nielsen, P. E. et al. (1993) Anticancer Drug Des. 8:53-63.)

The term “sample,” as used herein, is used in its broadest sense. A biological sample suspected of containing nucleic acids encoding SIGP, or fragments thereof, or SIGP itself may comprise a bodily fluid; an extract from a cell, chromosome, organelle, or membrane isolated from a cell; a cell; genomic DNA, RNA, or cDNA, in solution or bound to a solid support; a tissue; a tissue print; etc.

As used herein, the terms “specific binding” or “specifically binding” refer to that interaction between a protein or peptide and an agonist, an antibody, or an antagonist. The interaction is dependent upon the presence of a particular structure of the protein recognized by the binding molecule (i.e., the antigenic determinant or epitope). For example, if an antibody is specific for epitope “A,” the presence of a polypeptide containing the epitope A, or the presence of free unlabeled A, in a reaction containing free labeled. A and the antibody will reduce the amount of labeled A that binds to the antibody.

As used herein, the term “stringent conditions” refers to conditions which permit hybridization between polynucleotide sequences and the claimed polynucleotide sequences. Suitably stringent conditions can be defined by, for example, the concentrations of salt or formamide in the prehybridization and hybridization solutions, or by the hybridization temperature, and are well known in the art. In particular, stringency can be increased by reducing the concentration of salt, increasing the concentration of formamide, or raising the hybridization temperature.

For example, hybridization under high stringency conditions could occur in about 50% formamide at about 37° C. to 42° C. Hybridization could occur under reduced stringency conditions in about 35% to 25% formamide at about 30° C. to 35° C. In particular, hybridization could occur under high stringency conditions at 42° C. in 50% formamide, 5×SSPE, 0.3% SDS, and 200 μg/ml sheared and denatured salmon sperm DNA. Hybridization could occur under reduced stringency conditions as described above, but in 35% formamide at a reduced temperature of 35° C. The temperature range corresponding to a particular level of stringency can be further narrowed by calculating the purine to pyrimidine ratio of the nucleic acid of interest and adjusting the temperature accordingly. Variations on the above ranges and conditions are well known in the art.

The term “substantially purified,” as used herein, refers to nucleic acid or amino acid sequences that are removed from their natural environment and are isolated or separated, and are at least about 60% free, preferably about 75% free, and most preferably about 90% free from other components with which they are naturally associated.

A “substitution,” as used herein, refers to the replacement of one or more amino acids or nucleotides by different amino acids or nucleotides, respectively.

“Transformation,” as defined herein, describes a process by which exogenous DNA enters and changes a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, viral infection, electroporation, heat shock, lipofection, and particle bombardment. The term “transformed” cells includes stably transformed cells in which the inserted DNA is capable of replication either as an autonomously replicating plasmid or as part of the host chromosome, and refers to cells which transiently express the inserted DNA or RNA for limited periods of time.

A “variant” of SIGP, as used herein, refers to an amino acid sequence that is altered by one or more amino acids. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological or immunological activity may be found using computer programs well known in the art, for example, DNASTAR software.

### The Invention

The invention is based on the discovery of new human signal peptide-containing proteins, collectively referred to as SIGP and individually as SIGP-1, SIGP-2, SIGP-3, SIGP-4, SIGP-5, SIGP-6, SIGP-7, SIGP-8, SIGP-9, SIGP-10, SIGP-11, SIGP-12, SIGP-13, SIGP-14, SIGP-15, SIGP-16, SIGP-17, SIGP-18, SIGP-19, SIGP-20, SIGP-21, SIGP-22, SIGP-23, SIGP-24, SIGP-25, SIGP-26, SIGP-27, SIGP-28, SIGP-29, SIGP-30, SIGP-31, SIGP-32, SIGP-33, SIGP-34, SIGP-35, SIGP-36, SIGP-37, SIGP-38, SIGP-39, SIGP-40, SIGP-41, SIGP-42, SIGP-43, SIGP-44, SIGP-45, SIGP-46, SIGP-47, SIGP-48, SIGP-49, SIGP-50, SIGP-51, SIGP-52, SIGP-53, SIGP-54, SIGP-55, SIGP-56, SIGP-57, SIGP-58, SIGP-59, SIGP-60, SIGP-61, SIGP-62, SIGP-63, SIGP-64, SIGP-65, SIGP-66, SIGP-67, SIGP-68, SIGP-69, SIGP-70, SIGP-71, SIGP-72, SIGP-73, SIGP-74, SIGP-75, SIGP-76, and SIGP-77; the polynucleotides encoding SIGP (SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO: 129, SEQ ID NO: 130, SEQ ID NO: 131, SEQ ID NO: 132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO: 152, SEQ ID NO: 153, and SEQ ID NO: 154); and the use of these compositions for the diagnosis, treatment, or prevention of cancer and immunological disorders. Table 1 shows the sequence identification numbers, Incyte Clone identification number, cDNA library, NCBI sequence identifier and GenBank species description for each of the human signal peptide-containing proteins disclosed herein.

Nucleic acids encoding the SIGP-1 of the present invention were first identified in

TABLE 1

| Protein | Nucleotide | Clone ID | Library | NCBI I.D. | Homolog species |
|---|---|---|---|---|---|
| SEQ ID NO: 1 | SEQ ID NO: 78 | 305841 | HEARNOT01 | GI 505652 | Homo sapiens |
| SEQ ID NO: 2 | SEQ ID NO: 79 | 322866 | EOSIHET02 | GI 180141 | Homo sapiens |
| SEQ ID NO: 3 | SEQ ID NO: 80 | 546656 | BEPINOT01 | GI 2290530 | Homo sapiens |
| SEQ ID NO: 4 | SEQ ID NO: 81 | 693453 | SYNORAT03 | GI 1419461 | Caenorhabditis elegans |
| SEQ ID NO: 5 | SEQ ID NO: 82 | 866885 | BRAITUT03 | GI 1488683 | Rattus norvegicus |
| SEQ ID NO: 6 | SEQ ID NO: 83 | 1242271 | LUNGNOT03 | GI 1523073 | Homo sapiens |
| SEQ ID NO: 7 | SEQ ID NO: 84 | 1255027 | LUNGFET03 | GI 1684845 | Canis familiaris |
| SEQ ID NO: 8 | SEQ ID NO: 85 | 1273453 | TESTTUT02 | | |
| SEQ ID NO: 9 | SEQ ID NO: 86 | 1275261 | TESTTUT02 | GI 56805 | Rattus norvegicus |
| SEQ ID NO: 10 | SEQ ID NO: 87 | 1281682 | COLNNOT16 | | |
| SEQ ID NO: 11 | SEQ ID NO: 88 | 1298305 | BRSTNOT07 | | |
| SEQ ID NO: 12 | SEQ ID NO: 89 | 1360501 | LUNGNOT12 | GI 1019433 | Trypanosoma cruzi |
| SEQ ID NO: 13 | SEQ ID NO. 90 | 1362406 | LUNGNOT12 | GI 2072705 | Mycobacterium tuberculosis |
| SEQ ID NO: 14 | SEQ ID NO: 91 | 1405329 | LATRTUT02 | | |
| SEQ ID NO: 15 | SEQ ID NO: 92 | 1415223 | BRAINOT12 | GI 205250 | Rattus norvegicus |
| SEQ ID NO: 16 | SEQ ID NO: 93 | 1416553 | BRAINOT12 | | |
| SEQ ID NO: 17 | SEQ ID NO: 94 | 1418517 | KIDNNOT09 | | |
| SEQ ID NO: 18 | SEQ ID NO: 95 | 1438165 | PANCNOT08 | GI 1515161 | Caenorhabditis elegans |
| SEQ ID NO: 19 | SEQ ID NO: 96 | 1440381 | THYRNOT03 | GI 1065459 | Caenorhabditis elegans |
| SEQ ID NO: 20 | SEQ ID NO: 97 | 1510839 | LUNGNOT14 | GI 2145052 | Plasmodium berghei |
| SEQ ID NO: 21 | SEQ ID NO: 98 | 1534876 | SPLNNOT04 | | |
| SEQ ID NO: 22 | SEQ ID NO: 99 | 1559131 | SPLNNOT04 | GI 496667 | Saccharomyces cerevisiae |
| SEQ ID NO: 23 | SEQ ID NO: 100 | 1601473 | BLADNOT03 | | |
| SEQ ID NO: 24 | SEQ ID NO: 101 | 1615809 | BRAITUT12 | | |
| SEQ ID NO: 25 | SEQ ID NO: 102 | 1634813 | COLNNOT19 | GI 2196924 | Mus musculus |
| SEQ ID NO: 26 | SEQ ID NO: 103 | 1638407 | UTRSNOT06 | GI 200547 | Mus musculus |
| SEQ ID NO: 27 | SEQ ID NO: 104 | 1653112 | PROSTUT08 | GI 49794 | Mus musculus |
| SEQ ID NO: 28 | SEQ ID NO: 105 | 1664634 | BRSTNOT09 | GI 1890375 | Caenorhabditis elegans |
| SEQ ID NO: 29 | SEQ ID NO: 106 | 1690990 | PROSTUT10 | | |
| SEQ ID NO: 30 | SEQ ID NO: 107 | 1704050 | DUODNOT02 | GI 1814277 | Homo sapiens |
| SEQ ID NO: 31 | SEQ ID NO: 108 | 1711840 | PROSNOT16 | GI 182651 | Homo sapiens |
| SEQ ID NO: 32 | SEQ ID NO: 109 | 1747327 | STOMTUT02 | GI 2062391 | Homo sapiens |
| SEQ ID NO: 33 | SEQ ID NO: 110 | 1750632 | STOMTUT02 | GI 459002 | Caenorhabditis elegans |
| SEQ ID NO: 34 | SEQ ID NO: 111 | 1812375 | PROSTUT12 | | |
| SEQ ID NO: 35 | SEQ ID NO: 112 | 1818761 | PROSNOT20 | GI 2493789 | Homo sapiens |
| SEQ ID NO: 36 | SEQ ID NO: 113 | 1824469 | GBLATUT01 | GI 2052134 | Mycobacterium tuberculosis |
| SEQ ID NO: 37 | SEQ ID NO: 114 | 1864292 | PROSNOT19 | GI 295671 | Saccharomyces cerevisiae |
| SEQ ID NO: 38 | SEQ ID NO: 115 | 1866437 | THP1NOT01 | | |
| SEQ ID NO: 39 | SEQ ID NO: 116 | 1871375 | SKINBIT01 | | |
| SEQ ID NO: 40 | SEQ ID NO: 117 | 1880830 | LEUKNOT03 | GI 1872521 | Arabidopsis thaliana |
| SEQ ID NO: 41 | SEQ ID NO: 118 | 1905325 | OVARNOT07 | GI 1754971 | Homo sapiens |
| SEQ ID NO: 42 | SEQ ID NO: 119 | 1919931 | BRSTTUT01 | GI 2104517 | Homo sapiens |
| SEQ ID NO: 43 | SEQ ID NO: 120 | 1969426 | BRSTNOT04 | | |
| SEQ ID NO: 44 | SEQ ID NO: 121 | 1969948 | UCMCL5T01 | | |
| SEQ ID NO: 45 | SEQ ID NO: 122 | 1988911 | LUNGAST01 | GI 56649 | Rattus norvegicus |
| SEQ ID NO: 46 | SEQ ID NO: 123 | 2061561 | OVARNOT03 | | |
| SEQ ID NO: 47 | SEQ ID NO: 124 | 2084489 | PANCNOT04 | GI 2262136 | Arabidopsis thaliana |
| SEQ ID NO: 48 | SEQ ID NO: 125 | 2203226 | SPLNFET02 | GI 1911776 | Homo sapiens |
| SEQ ID NO: 49 | SEQ ID NO: 126 | 2232884 | PROSNOT16 | | |
| SEQ ID NO: 50 | SEQ ID NO: 127 | 2328134 | COLNNOT11 | GI 1911776 | Homo sapiens |
| SEQ ID NO: 51 | SEQ ID NO: 128 | 2382718 | ISLTNOT01 | GI 1814277 | Homo sapiens |
| SEQ ID NO: 52 | SEQ ID NO: 129 | 2452208 | ENDANOT01 | | |
| SEQ ID NO: 53 | SEQ ID NO: 130 | 2457825 | ENDANOT01 | GI 1418625 | Caenorhabditis elegans |
| SEQ ID NO: 54 | SEQ ID NO: 131 | 2470740 | THP1NOT03 | | |
| SEQ ID NO: 55 | SEQ ID NO. 132 | 2479092 | SMCANOT01 | | |
| SEQ ID NO: 56 | SEQ ID NO: 133 | 2480544 | SMCANOT01 | GI 169345 | Phaseolus vulgaris |
| SEQ ID NO: 57 | SEQ ID NO: 134 | 2518547 | BRAITUT21 | GI 33969 | Homo sapiens |
| SEQ ID NO: 58 | SEQ ID NO: 135 | 2530650 | GBLANOT02 | GI 2204111 | Bos taurus |
| SEQ ID NO: 59 | SEQ ID NO: 136 | 2652271 | THYMNOT04 | GI 895855 | Solanum lycopersicum |
| SEQ ID NO: 60 | SEQ ID NO: 137 | 2746976 | LUNGTUT11 | GI 191983 | Mus musculus |
| SEQ ID NO: 61 | SEQ ID NO: 138 | 2753496 | THP1AZS08 | GI 987286 | Schizosaccharomyces pombe |
| SEQ ID NO: 62 | SEQ ID NO: 139 | 2781553 | OVARTUT03 | | |
| SEQ ID NO: 63 | SEQ ID NO: 140 | 2821925 | ADRETUT06 | | |
| SEQ ID NO: 64 | SEQ ID NO: 141 | 2879068 | UTRSTUT05 | GI 870749 | Homo sapiens |
| SEQ ID NO: 65 | SEQ ID NO: 142 | 2886757 | SINJNOT02 | GI 1420026 | Saccharomyces cerevisiae |
| SEQ ID NO: 66 | SEQ ID NO: 143 | 2964329 | SCORNOT04 | GI 311667 | Saccharomyces cerevisiae |
| SEQ ID NO: 67 | SEQ ID NO: 144 | 2965248 | SCORNOT04 | GI 1478503 | Homo sapiens |
| SEQ ID NO. 68 | SEQ ID NO: 145 | 3000534 | TLYMNOT06 | GI 1741868 | Homo sapiens |
| SEQ ID NO: 69 | SEQ ID NO: 146 | 3046870 | HEAANOT01 | GI 1067079 | Caenorhabditis elegans |
| SEQ ID NO: 70 | SEQ ID NO: 147 | 3057669 | PONSAZT01 | GI 260241 | |
| SEQ ID NO: 71 | SEQ ID NO: 148 | 3088178 | HEAONOT03 | GI 498997 | Saccharomyces cerevisiae |
| SEQ ID NO: 72 | SEQ ID NO: 149 | 3094321 | BRSTNOT19 | GI 793879 | Saccharomyces cerevisiae |
| SEQ ID NO: 73 | SEQ ID NO: 150 | 3115936 | LUNGTUT13 | GI 517174 | Saccharomyces cerevisiae |
| SEQ ID NO: 74 | SEQ ID NO: 151 | 3116522 | LUNGTUT13 | GI 1669560 | Homo sapiens |
| SEQ ID NO: 75 | SEQ ID NO: 152 | 3117184 | LUNGTUT13 | GI 1418628 | Caenorhabditis elegans |
| SEQ ID NO: 76 | SEQ ID NO: 153 | 3125156 | LNODNOT05 | GI 804750 | Homo sapiens |
| SEQ ID NO: 77 | SEQ ID NO: 154 | 3129120 | LUNGTUT12 | GI 1256890 | Saccharomyces cerevisiae |

Incyte Clone 305841 from the heart tissue cDNA library (HEARNOT01) using a computer search for amino acid sequence alignments. A consensus sequence, SEQ ID NO:78, was derived from Incyte Clones 305841 (HEARNOT01), 22049 (ADENINB01), 168880 (LIVRNOT01), 1321915 (BLADNOT04), and the shotgun sequences SAWA02804, SAWA02781, SAWA01969, and SAWA01937.

In one embodiment, the invention encompasses a polypeptide comprising the amino acid sequence of SEQ ID NO: 1. SIGP-1 is 348 amino acids in length and has a potential amidation site at Q120; a potential N-glycosylation site at N181; two potential casein kinase II phosphorylation sites at S19 and T279; a potential glycosaminoglycan attachment site at S35; and three potential protein kinase C phosphorylation sites at S19, S268, and S343. SIGP-1 shares 56% identity with human GP36b glycoprotein (GI 505652). The fragment of SEQ ID NO:78 including the 5′ region from about nucleotide 117 to about nucleotide 161 is useful for hybridization. Northern analysis shows the expression of this sequence in reproductive, neural, cardiovascular, hematopoietic and immune, and developmental cDNA libraries. Approximately 42% of these libraries are associated with neoplastic disorders, 28% with inflammation, and 21% with cell proliferation.

Nucleic acids encoding the SIGP-2 of the present invention were first identified in Incyte Clone 322866 from the eosinophil cDNA library (EOSIHET02) using a computer search for amino acid sequence alignments. A consensus sequence, SEQ ID NO:79, was derived from Incyte Clones 322866 (EOSIHET02), 470107 (MMLRIDT01), 873933 (LUNGAST01), and 2268817. (UTRSNOT02)

In one embodiment, the invention encompasses a polypeptide comprising the amino acid sequence of SEQ ID NO:2. SIGP-2 is 194 amino acids in length and has two potential N-glycosylation sites at N129 and N148; two potential casein kinase II phosphorylation sites at S74 and S151; four potential protein kinase C phosphorylation sites at S5, S74, S130, and S163; a potential tyrosine kinase phosphorylation site at Y171; two potential prokaryotic membrane lipoprotein lipid attachment sites at F15 and S61; and a transmembrane 4 protein family signature from G60 to L82. SIGP-2 shares 90% identity with CD53, a human cell surface antigen (GI 180141). The fragment of SEQ ID NO:79 from about nucleotide 624 to about nucleotide 686 is useful for hybridization. Northern analysis shows the expression of this sequence in hematopoietic and immune, gastrointestinal, cardiovascular, reproductive, musculoskeletal, and neural cDNA libraries. Approximately 54% of these libraries are associated with inflammation, 39% with neoplastic disorders, and 11% with cell proliferation.

Nucleic acids encoding the SIGP-3 of the present invention were first identified in Incyte Clone 546656 from the bronchial epithelium primary cell line cDNA library (BEPINOT01) using a computer search for amino acid sequence alignments. A consensus sequence, SEQ ID NO:80, was derived from Incyte Clones 546656 (BEPINOT01), 1316266 (BLADTUT02), 2095988 (BRAITUT02), 1318172 (BLADNOT04), 2809506 (TLYMNOT04), 1293412 and 1293630 (PGANNOT03), 2585048 (BRAITUT22), 2941370 (HEAONOT03), 2297230 (BRSTNOT05), 1233586 (LUNGFET03), and the shotgun sequence SAEA02986.

In one embodiment, the invention encompasses a polypeptide comprising the amino acid sequence of SEQ ID NO:3. SIGP-3 is 342 amino acids in length and has a potential amidation site at H4; a potential N-glycosylation site at N23; seven potential casein kinase II phosphorylation sites at S38, T90, T105, T124, S139, T284, and T324; three potential protein kinase C phosphorylation sites at S25, T71, and S200; two potential tyrosine kinase phosphorylation sites at Y13 and Y69; and a beta-transducin family Trp-Asp repeats signature sequence from I282 to I296. SIGP-3 shares 100% identity with human HAN11 (GI 2290530). The fragment of SEQ ID NO:80 from about nucleotide 107 to about nucleotide 139 is useful for hybridization. Northern analysis shows the expression of this sequence in reproductive, cardiovascular, hematopoietic and immune, neural, urologic, and developmental cDNA libraries. Approximately 43% of these libraries are associated with neoplastic disorders, 25% with inflammation, and 20% with cell proliferation.

Nucleic acids encoding the SIGP-4 of the present invention were first identified in Incyte Clone 693453 from the synovial membrane cDNA library (SYNORAT03) using a computer search for amino acid sequence alignments. A consensus sequence, SEQ ID NO:81, was derived from Incyte Clones 693453 (SYNORAT03), 2505458 (CONUTUT01), 1527363 (UCMCL5T01), 1275308 (TESTTUT02), 1377126 (LUNGNOT10), 538256 (LNODNOT02), 3125441 (LNODNOT05), 1955296 (CONNNOT01), 1821536 (GBLATUT01), 2055631 (BEPINOT01), and 2028161 (KERANOT02).

In one embodiment, the invention encompasses a polypeptide comprising the amino acid sequence of SEQ ID NO:4. SIGP-4 is 656 amino acids in length and has a potential N-glycosylation site at N73; nine potential casein kinase II phosphorylation sites at S140, S191, T250, T252, S330, S340, S517, S617, and T630; a potential leucine zipper pattern from L430 to L451; four potential N-myristoylation sites at G77, G246, G484, and A651; eleven potential protein kinase C phosphorylation sites at S18, T90, S93, T318, S490, S503, S532, T565, T608, S609, and T629; and a potential tyrosine kinase phosphorylation site at Y326. SIGP-4 shares 20% identity with Caenorhabditis elegans protein encoded by T10G9.4 (GI 1419461). The fragment of SEQ ID NO:81 from about nucleotide 202 to about nucleotide 255 is useful for hybridization. Northern analysis shows the expression of this sequence in reproductive, hematopoietic and immune, neural, and developmental cDNA libraries. Approximately 40% of these libraries are associated with neoplastic disorders, 30% with inflammation, and 30% with cell proliferation.

Nucleic acids encoding the SIGP-5 of the present invention were first identified in Incyte Clone 866885 from the brain tumor cDNA library (BRAITUT03) using a computer search for amino acid sequence alignments. A consensus sequence, SEQ ID NO:82, was derived from Incyte Clones 866885 (BRAITUT03), 2991983 (KIDNFET02), 067954 (HUVESTB01), and 1499109 (SINTBST01).

In one embodiment, the invention encompasses a polypeptide comprising the amino acid sequence of SEQ ID NO:5. SIGP-5 is 236 amino acids in length and has a potential N-glycosylation site at N199; two potential casein kinase II phosphorylation sites at S8 and T72; a potential N-myristoylation site at G169; and three potential protein kinase C phosphorylation sites at T43, S96, and T201. SIGP-5 shares 24% identity with rat syntaxin (GI 1488683). The fragment of SEQ ID NO:82 from about nucleotide 43 to about nucleotide 93 is useful for hybridization. Northern analysis shows the expression of this sequence in hematopoietic and immune, reproductive, gastrointestinal, neural, cardiovascular, and developmental cDNA libraries. Approximately 43% of these libraries are associated with neoplastic disorders, 26% with inflammation, and 19% with cell proliferation.

Nucleic acids encoding the SIGP-6 of the present invention were first identified in Incyte Clone 1242271 from the lung tissue cDNA library (LUNGNOT03) using a computer search for amino acid sequence alignments. A consensus sequence, SEQ ID NO:83, was derived from Incyte Clones 1242271 (LUNGNOT03), 968114 (BRSTNOT05), 1251728 (LUNGFET03), and the shotgun sequence SAZA00142.

In one embodiment, the invention encompasses a polypeptide comprising the amino acid sequence of SEQ ID NO:6. SIGP-6 is 195 amino acids in length and has a potential cAMP- and cGMP-dependent protein kinase phosphorylation site at S79; six potential casein kinase II phosphorylation sites at S79, T85, S113, T166, T171, and T188; three potential protein kinase C phosphorylation sites at S20, S150, and S185; and a potential mitochondrial energy transfer proteins signature from P25 to Y33. The fragment of SEQ ID NO:83 from about nucleotide 98 to about nucleotide 133 is useful for hybridization. Northern analysis shows the expression of this sequence in urologic, neural, reproductive, and cardiovascular cDNA libraries. Approximately 50% of these libraries are associated with neoplastic disorders, 14% with inflammation, and 21% with cell proliferation.

Nucleic acids encoding the SIGP-7 of the present invention were first identified in Incyte Clone 1255027 from the fetal lung cDNA library (LUNGFET03) using a computer search for amino acid sequence alignments. A consensus sequence, SEQ ID NO:84, was derived from Incyte Clones 1255027 (LUNGFET03), 2055704 (BEPINOT01), 1351096 (LATRTUT02), 835188 (PROSNOT07), and 1695810 (COLNNOT23).

In one embodiment, the invention encompasses a polypeptide comprising the amino acid sequence of SEQ ID NO:7. SIGP-7 is 608 amino acids in length and has a potential amidation site at T112; five potential N-glycosylation sites at N73, N110, N410, N436, and N478; two potential cAMP- and cGMP-dependent protein kinase phosphorylation sites at S123 and S185; ten potential casein kinase II phosphorylation sites at T2, S75, S166, S170, S185, S274, S463, S505, S517, and T588; and thirteen potential protein kinase C phosphorylation sites at T19, S32, S46, T112, T221, S274, S299, T337, S373, S412, S431, S438, and S555. SIGP-7 shares 16% identity with canine pinin (GI 1684845). The fragment of SEQ ID NO:84 from about nucleotide 181 to about nucleotide 219 is useful for hybridization. Northern analysis shows the expression of this sequence in reproductive, gastrointestinal, neural, cardiovascular, and developmental cDNA libraries. Approximately 43% of these libraries are associated with neoplastic disorders, 21% with inflammation, and 20% with cell proliferation.

Nucleic acids encoding the SIGP-8 of the present invention were first identified in Incyte Clone 1273453 from the testicle cDNA library (TESTTUT02) using a computer search for amino acid sequence alignments. A consensus sequence, SEQ ID NO:85, was derived from Incyte Clones 1273453 (TESTTUT02), 1970337 (UCMCL5T01), 1218926 (NEUTGMT01), 1881349 (LEUKNOT03), and 1722377 (BLADNT06).

In one embodiment, the invention encompasses a polypeptide comprising the amino acid sequence of SEQ ID NO:8. SIGP-8 is 267 amino acids in length and has a potential N glycosylation site at N230, five potential casein kinase II phosphorylation sites at S9, T45, T77, S190, and T263, and two potential protein kinase C phosphorylation sites at S232 and S236. The fragment of SEQ ID NO:85 from about nucleotide 140 to about nucleotide 175 is useful for hybridization. Northern analysis shows the expression of this sequence in reproductive, cardiovascular, and hematopoietic and immune cDNA libraries. Approximately 42% of these libraries are associated with neoplastic disorders and 40% with immune response.

Nucleic acids encoding the SIGP-9 of the present invention were first identified in Incyte Clone 1275261 from the testicle cDNA library (TESTTUT02) using a computer search for amino acid sequence alignments. A consensus sequence, SEQ ID NO:86, was derived from Incyte Clones 1275261 (TESTTUT02), 775078 (COLNNOT05), 514772 (MMLR1DT01), and 3224071 (COLNNON03).

In one embodiment, the invention encompasses a polypeptide comprising the amino acid sequence of SEQ ID NO:9. SIGP-9 is 285 amino acids in length and has a potential amidation site at S260, three potential N glycosylation sites at N85, N100 and N156, a potential cAMP- and cGMP-dependent protein kinase phosphorylation site at T168, three potential casein kinase II phosphorylation sites at T168, T215, and S230, three potential protein kinase C phosphorylation sites at S163, S230, and S260, and a potential tyrosine kinase phosphorylation site at Y72. SIGP-9 shares 24% identity with rat OX-45 antigen preprotein (GI 56805). The fragment of SEQ ID NO:86 from about nucleotide 243 to about nucleotide 293 is useful for hybridization. Northern analysis shows the expression of this sequence in reproductive, gastrointestinal, and hematopoietic and immune cDNA libraries. Approximately 50% of these libraries are associated with neoplastic disorders and 50% with immune response.

Nucleic acids encoding the SIGP-10 of the present invention were first identified in Incyte Clone 1281682 from the colon cDNA library (COLNNOT16) using a computer search for amino acid sequence alignments. A consensus sequence, SEQ ID NO:87, was derived from Incyte Clones 2681940 (SINIUCT01), 1335652 (COLNNOT13), 2079572 (UTRSNOT08), 627405 (PGANNOT01) and 1281682 and 1282887 (COLNNOT16).

In one embodiment, the invention encompasses a polypeptide comprising the amino acid sequence of SEQ ID NO: 10. SIGP-10 comprises a peptide of 76 amino acids in length, and has a potential signal peptide sequence from M1 to S18. The fragment of SEQ ID NO:87 encoding the potential signal peptide sequence from about nucleotide 908 through 970 is useful for hybridization. Northern analysis shows the expression of this sequence in gastrointestinal, neural, reproductive, and hematopoietic and immune cDNA libraries. Approximately 32% of these libraries are associated with neoplastic disorders and 53% with immune response.

Nucleic acids encoding the SIGP-11 of the present invention were first identified in Incyte Clone 1298305 from the breast cDNA library (BRSTNOT09) using a computer search for amino acid sequence alignments. A consensus sequence, SEQ ID NO:88, was derived from Incyte Clones 1298305 (BRSTNOT09), 3451203 (UTRSNON03), 2529672 (GBLAN0502), 2780863 (OVARTUT03), 927988 (BRAINOT04), 1684424 (PROSNOT15), 2243053 (PANCTUT02), and shotgun sequences SANA03310 and SANA00700.

In one embodiment, the invention encompasses a polypeptide comprising the amino acid sequence of SEQ ID NO:11. SIGP-11 is 147 amino acids in length and has a prokaryotic membrane lipoprotein lipid attachment site from L34 through C44. SIGP-11 also has a potential cAMP- and cGMP-dependent protein kinase phosphorylation site at S91, and a potential protein kinase C phosphorylation site at S13. The fragment of SEQ ID NO:88 from about nucleotide 1561 to about nucleotide 1611 is useful for hybridization. Northern analysis shows the expression of this sequence in reproductive, gastrointestinal, and neural cDNA libraries. Approximately 50% of these libraries are associated with neoplastic disorders and 22% with immune response.

Nucleic acids encoding the SIGP-12 of the present invention were first identified in Incyte Clone 1360501 from the lung cDNA library (LUNGNOT12) using a computer search for amino acid sequence alignments. A consensus sequence, SEQ ID NO:89, was derived from Incyte Clones 1360501 (LUNGNOT12), 2121661 (BRSTNOT07), 1706518 (DUODNOT02) and shotgun sequences SAJA02519, SAJA00749, SAJA01160, and SANA00513.

In one embodiment, the invention encompasses a polypeptide comprising the amino acid sequence of SEQ ID NO:12. SIGP-12 is 261 amino acids in length and has six potential N glycosylation sites at N19, N28, N98, N104, N164 and N178. SIGP-12 also has five potential casein kinase II phosphorylation sites at T82, S83, T91, T160, and S233, and nine potential protein kinase C phosphorylation sites at T35, T60, T82, S121, S131, T184, S233, S237, and T242. SIGP-12 shares 22% identity with Tryypanosoma cruzi mucin-like protein (GI 1019433). In addition, SIGP-12 shares two potential phosphorylation sites and a potential N-glycosylation site with the mucin-like protein. The fragment of SEQ ID NO:89 from about nucleotide 183 to about nucleotide 236 is useful for hybridization. Northern analysis shows the expression of this sequence in reproductive, cardiovascular, and gastrointestinal cDNA libraries. Approximately 39% of these libraries are associated with neoplastic disorders and 26% with immune response.

Nucleic acids encoding the SIGP-13 of the present invention were first identified in Incyte Clone 1362406 from the lung cDNA library (LUNGNOT12) using a computer search for amino acid sequence alignments. A consensus sequence, SEQ ID NO:90, was derived from Incyte Clones 1362406 (LUNGNOT12), 1854401 (HNT3AZT01), 1570003 (UTRSNOT05) and shotgun sequences SANA03704, SANA00366, and SANA02152.

In one embodiment, the invention encompasses a polypeptide comprising the amino acid sequence of SEQ ID NO:13. SIGP-13 is 213 amino acids in length and has three potential protein kinase C phosphorylation sites at T40, S136, and T166. In addition, SIGP-13 has a highly hydrophobic signal peptide sequence from residue M1 to E34. SIGP-13 shares 20% identity with a Mycobacterium tuberculosis membrane protein (GI 2072705). The fragment of SEQ ID NO:90 encoding the potential signal peptide sequence domain from about nucleotide 157 to about nucleotide 219 is useful for hybridization. Northern analysis shows the expression of this sequence in reproductive, developmental, neural, and cardiovascular cDNA libraries. Approximately 50% of these libraries are associated with neoplastic disorders and 18% with immune response.

Nucleic acids encoding the SIGP-14 of the present invention were first identified in Incyte Clone 1405329 from the heart cDNA library (LATRTUT02) using a computer search for amino acid sequence alignments. A consensus sequence, SEQ ID NO:91, was derived from Incyte Clones 1405329 (LATRTUT02), and 2830813 (TLYMNOT03).

In one embodiment, the invention encompasses a polypeptide comprising the amino acid sequence of SEQ ID NO:14. SIGP-14 is 67 amino acids in length and has a cell attachment sequence comprising R13 through D15. In addition, SIGP-14 has a potential casein kinase II phosphorylation site at T12, and a potential protein kinase C phosphorylation site at T42. The fragment of SEQ ID NO:91 from about nucleotide 36 to about nucleotide 95 is useful for hybridization. Northern analysis shows the expression of this sequence in cardiovascular, developmental, reproductive, and hematopoietic and immune cDNA libraries. Approximately 43% of these libraries are associated with neoplastic disorders and 21% with immune response.

Nucleic acids encoding the SIGP-15 of the present invention were first identified in Incyte Clone 1415223 from the brain cDNA library (BRAINOT12) using a computer search for amino acid sequence alignments. A consensus sequence, SEQ ID NO:92, was derived from Incyte Clones 1415223 (BRAINOT12) and 529786 (BRAINOT03).

In one embodiment, the invention encompasses a polypeptide comprising the amino acid sequence of SEQ ID NO:15. SIGP-15 is 161 amino acids in length and has a potential N-glycosylation site at N57, two potential casein kinase II phosphorylation sites at S84 and S96, and five potential protein kinase C phosphorylation sites at S11, T62, S75, S83, and S84. SIGP-15 shares 30% identity with rat Ly6C antigen (GI 205250). The fragment of SEQ ID NO:92 from about nucleotide 28 to about nucleotide 81 is useful for hybridization. Northern analysis shows the expression of this sequence in developmental, reproductive, and neural cDNA libraries. Approximately 33% of these libraries are associated with neoplastic disorders, 33% with cell proliferation, and 17% with immune response.

Nucleic acids encoding the SIGP-16 of the present invention were first identified in Incyte Clone 1416553 from the brain cDNA library (BRAINOT12) using a computer search for amino acid sequence alignments. A consensus sequence, SEQ ID NO:93, was derived from Incyte Clones 1416553 (BRAINOT12), 663124 (BRAINOT03) and shotgun sequences SANA01409, SANA03513, and SANA02713.

In one embodiment, the invention encompasses a polypeptide comprising the amino acid sequence of SEQ ID NO:16. SIGP-16 is 141 amino acids in length and has a glycosaminoglycan attachment site at S20. In addition, SIGP-16 has a potential casein kinase II phosphorylation site at S61, and a potential protein kinase C phosphorylation site at S53. The fragment of SEQ ID NO:93 from about nucleotide 784 to about nucleotide 831 is useful for hybridization. Northern analysis shows the expression of this sequence in neural cDNA libraries. Approximately 27% of these libraries are associated with neoplastic disorders, and 27% with neurological disorders.

Nucleic acids encoding the SIGP-17 of the present invention were first identified in Incyte Clone 1418517 from the kidney cDNA library (KIDNNOT09) using a computer search for amino acid sequence alignments. A consensus sequence, SEQ ID NO:94, was derived from Incyte Clones 1418517 (KIDNNOT09), 2456866 (ENDANOT01), 136927 (SYNORAB01), 1620442 (BRAITUT13), 1492394 (PROSNON01), 1534435 (SPLNNOT04), and 2505923 (CONUTUT01).

In one embodiment, the invention encompasses a polypeptide comprising the amino acid sequence of SEQ ID NO: 17. SIGP-17 is 152 amino acids in length and has a potential N glycosylation site at N76; a potential cAMP- and cGMP-dependent protein kinase phosphorylation site at T67; four potential casein kinase II phosphorylation sites at S9, T30, S107, and S124; and three potential protein kinase C phosphorylation sites at T30, S34, and T78. The fragment of SEQ ID NO:94 from about nucleotide 49 to about nucleotide 99 is useful for hybridization. Northern analysis shows the expression of this sequence in reproductive, cardiovascular, musculoskeletal, and gastrointestinal cDNA libraries. Approximately 44% of these libraries are associated with neoplastic disorders, 23% with immune response, and 20% with cell proliferation.

Nucleic acids encoding the SIGP-18 of the present invention were first identified in Incyte Clone 1438165 from the pancreas cDNA library (PANCNOT08) using a computer search for amino acid alignments. A consensus sequence, SEQ ID NO:95, was derived from Incyte Clones 360389 (SYNORAB01), 485693 (HNT2RAT01), 1233177 (LUNGFET03), 1255551 (MENITUT03), 1438165 (PANCNOT08), 1554990 (BLADTUT04), and shotgun sequences SAOA00854 and SAOA00855.

In one embodiment, the invention encompasses a polypeptide comprising the amino acid sequence of SEQ ID NO: 18. SIGP-18 is 742 amino acids in length and has a potential N-glycosylation site at N448; a microbodies C-terminal targeting signal in the triplet N740HL; twelve potential casein kinase II phosphorylation sites at S3, S53, S120, T122, T169, T178, S179, S195, T284, S290, S400, and S573; five potential protein kinase C phosphorylation sites at T178, S195, S208, S299, and S364; and two potential tyrosine kinase phosphorylation sites at Y296 and Y512. Cysteine residues, representing potential intramolecular disulfide bridging sites, are found at residues C87, C204, C312, C339, C343, C469, C497, C558, C657, C693, and C720. SIGP-18 shares 19% homology with C. elegans protein encoded by M163.4 (GI 1515161), including eight of the eleven cysteine residues found in SIGP-18. The fragment of SEQ ID NO:95 from about nucleotide 322 to about nucleotide 387 is useful for hybridization. Northern analysis shows the expression of this sequence in cardiovascular, male and female reproductive, and gastrointestinal cDNA libraries. Approximately 44% of these libraries are associated with neoplastic disorders, 23% with inflammation and the immune response, and 19% with fetal development.

Nucleic acids encoding the SIGP-19 of the present invention were first identified in Incyte Clone 1440381 from the thyroid cDNA library (THYRNOT03) using a computer search for amino acid alignments. A consensus sequence, SEQ ID NO:96, was derived from Incyte Clones 989671 (COLNNOT11), 1440381 (THYRNOT03), 3507668 (CONCNOT01), and shotgun sequences SAOA03364, SAOA02692, SAOA00489, SAOA02355, SAOA02405, SAOA01209, SAOA00809, and SAOA00274.

In one embodiment, the invention encompasses a polypeptide comprising the amino acid sequence of SEQ ID NO: 19. SIGP-19 is 805 amino acids in length and has three potential N-glycosylation sites at N211, N215, and N327; one cAMP- and cGMP-dependent protein kinase potential phosphorylation sites at T749; sixteen potential casein kinase II phosphorylation sites at S8, T54, T175, T228, S229, S250, S292, S329, T390, S401, S415, S471, S492, S671, T780, and S795; ten potential protein kinase C phosphorylation sites at S206, T396, S401, S442, T455, S600, S671, T683, S730, and S795; and two potential tyrosine kinase phosphorylation sites at Y437 and Y476. SIGP-19 shares 33% homology with a ubiquitin-conjugating, E2-like enzyme from C. elegans (GI 1065459). Both molecules share a “UBC domain” characteristic of ubiquitin-conjugating enzymes extending from approximately residue V559 to 1647 of SIGP-19, and containing an active site cysteine residue, C614, required for thiolester formation. A characteristic proline-rich region, found at the N-terminal end of the UBC domain and extending from approximately P564 to P589 in SIGP-19, is also shared by both proteins. The fragment of SEQ ID NO:96 from about nucleotide 1678 to about nucleotide 1800 is useful for hybridization. Northern analysis shows the expression of this sequence in cardiovascular and male and female reproductive cDNA libraries. Approximately 50% of these libraries are associated with neoplastic disorders, 14% with inflammation and the immune response, and 19% with fetal development.

Nucleic acids encoding the SIGP-20 of the present invention were first identified in Incyte Clone 1510839 from the lung cDNA library (LUNGNOT14) using a computer search for amino acid alignments. A consensus sequence, SEQ ID NO:97, was derived from Incyte Clones 962326 (BRSTTUT03), 1383254 (BRAITUT08), 1510839 (LUNGNOT14), 1970949 (UCMCLST01), 2214224 (SINTFET03), and shotgun sequences SAOA01059 and SAOA02595.

In one embodiment, the invention encompasses a polypeptide comprising the amino acid sequence of SEQ ID NO:20. SIGP-20 is 195 amino acids in length and has a potential signal peptide sequence between M1 and A39. SIGP-20 also has a potential N-glycosylation site at N83; and three potential casein kinase II phosphorylation sites at T161, T169, and T181; and three potential protein kinase C phosphorylation sites at T121, T143, and T153. SIGP-20 shares 21% homology with Plasmodium berghei merozoite surface protein-1 (GI 2145052). The fragment of SEQ ID NO:97 from about nucleotide 439 to about nucleotide 502 is useful for hybridization. Northern analysis shows the expression of this sequence in cardiovascular, male and female reproductive, and developmental cDNA libraries. Approximately 48% of these libraries are associated with neoplastic disorders, 13% with inflammation and the immune response, and 19% with fetal development.

Nucleic acids encoding the SIGP-21 of the present invention were first identified in Incyte Clone 1534876 from the spleen cDNA library (SPLNNOT04) using a computer search for amino acid alignments. A consensus sequence, SEQ ID NO:98, was derived from Incyte Clones 1253004 (LUNGFET03), 1382838 (BRAITUT08), 1532501 (SPLNNOT04), 1534876 (SPLNNOT04), 1705806 (DUODNOT02), 1738301 (COLNNOT22), 1926209 (BRSTNOT02), and shotgun sequences SAOA00587, SAOA02048, and SAOA03535.

In one embodiment, the invention encompasses a polypeptide comprising the amino acid sequence of SEQ ID NO:21. SIGP-21 is 161 amino acids in length and has a potential signal peptide sequence between M1 and C13. SIGP-21 also has 17 cysteine residues with the potential for forming intramolecular disulfide bridges. Six of these cysteine residues, between residues C129 and C152, are found in a signature sequence for trypsin/alpha-amylase inhibitors that form a structure with intramolecular disulfide bridges. SIGP-21 has two potential casein kinase II phosphorylation sites at T25 and S35; and two potential protein kinase C phosphorylation sites at S35 and T87. The fragment of SEQ ID NO:98 from about nucleotide 406 to about nucleotide 477, which encompasses the trypsin/alpha-amylase inhibitor signature sequence, is useful for hybridization. Northern analysis shows the expression of this sequence in gastrointestinal and male and female reproductive cDNA libraries. Approximately 45% of these libraries are associated with neoplastic disorders and 28% with inflammation and the immune response.

Nucleic acids encoding the SIGP-22 of the present invention were first identified in Incyte Clone 155913.1 from the spleen cDNA library (SPLNNOT04) using a computer search for amino acid alignments. A consensus sequence, SEQ ID NO:99, was derived from Incyte Clones 1559131 (SPLNNOT04), 1671080 (BMARNOT03), 1924001 (BRSTTUT01), and shotgun sequences SAPA01073 and SAOA02895.

In one embodiment, the invention encompasses a polypeptide comprising the amino acid sequence of SEQ ID NO:22. SIGP-22 is 160 amino acids in length and has cysteine residues capable of forming intramolecular disulfide bridges at C40, C47, C108, C114, C129, C154, and C158. SIGP-22 has one potential casein kinase II phosphorylation site at S9 and one potential protein kinase C phosphorylation site at S31. SIGP-22 shares 26% homology with C-215 protein from Saccharomyces cerevisiae (GI 496667), including four of the cysteine residues found in SIGP-22. The fragment of SEQ ID NO:99 from about nucleotide 154 to about nucleotide 193 is useful for hybridization. Northern analysis shows the expression of this sequence in hematopoietic and male and female reproductive cDNA libraries. Approximately 33% of these libraries are associated with neoplastic disorders and 67% with the immune response.

Nucleic acids encoding the SIGP-23 of the present invention were first identified in Incyte Clone 1601473 from the bladder cDNA library (BLADNOT03) using a computer search for amino acid alignments. A consensus sequence, SEQ ID NO:100, was derived from Incyte Clones 1601473 (BLADNOT03), and shotgun sequences SAOA00407, SAOA02497, SAOA02747, and SAOA02958.

In one embodiment, the invention encompasses a polypeptide comprising the amino acid sequence of SEQ ID NO:23. SIGP-23 is 76 amino acids in length and has two cysteine residues with the potential of forming an intramolecular disulfide bridge at C58 and C72. SIGP-23 has one potential casein kinase II phosphorylation site at S7 and three potential protein kinase C phosphorylation sites at S7, T29, and T46. The fragment of SEQ ID NO: 100 from about nucleotide 139 to about nucleotide 180 is useful for hybridization. Northern analysis shows the expression of this sequence in breast, brain, spleen, thyroid, and bladder cDNA libraries. Approximately 33% of these libraries are associated with neoplastic disorders, 17% with neural disorders, and 17% with immune disorders.

Nucleic acids encoding the SIGP-24 of the present invention were first identified in Incyte Clone 1615809 from the brain tumor cDNA library (BRAITUT12) using a computer search for amino acid sequence alignments. A consensus sequence, SEQ ID NO: 101, was derived from Incyte Clones 1615809 (BRAITUT12), 924499 (BRAINOT04), 1273065 (TESTTUT02), 1517058 (PANCTUT01), 1596867 (BRAINOT14), and 1361446 (LUNGNOT12), and shotgun sequence SAOA02975.

In one embodiment, the invention encompasses a polypeptide comprising the amino acid sequence of SEQ ID NO:24. SIGP-24 is 336 amino acids in length and has 13 potential phosphorylation sites at T27, T72, S74, S76, T99, S104, S109, S140, S178, S210, T281, S326, S39. SIGP-24 also has a potential signal peptide sequence between M1 and Y18. The fragment of SEQ ID NO:101 from about nucleotide 187 to about nucleotide 247 is useful for hybridization. Northern analysis shows the expression of this sequence in cardiovascular, gastrointestinal, neural, and reproductive cDNA libraries. Approximately 48% of these libraries are associated with neoplastic disorders and 21% with immune response.

Nucleic acids encoding the SIGP-25 of the present invention were first identified in Incyte Clone 1634813 from the cecal tissue cDNA library (COLNNOT19) using a computer search for amino acid sequence alignments. A consensus sequence, SEQ ID NO: 102, was derived from Incyte Clones 1634813 (COLNNOT19), 2904583 (THYMNOT05), 1634813 (COLNNOT19), and 1310492 (COLNFET02), and shotgun sequence SAPA04436.

In one embodiment, the invention encompasses a polypeptide comprising the amino acid sequence of SEQ ID NO:25. SIGP-25 is 150 amino acids in length and has one potential N-glycosylation site at N139; and five potential phosphorylation sites at T48, S118, S126, S135, and S136. SIGP-25 also has a potential signal peptide sequence encompassing residues M1-A23. SIGP-25 shares 28% identity with mouse beta chemokine, Exodus-2 (GI 2196924). The fragment of SEQ ID NO:102 from about nucleotide 175 to about nucleotide 235 is useful for hybridization. Northern analysis shows the expression of this sequence in gastrointestinal, developmental, hematopoietic, and immunological cDNA libraries. Approximately 50% of these libraries are associated with fetal development/cell proliferation and 25% with immune response.

Nucleic acids encoding the SIGP-26 of the present invention were first identified in Incyte Clone 1638407 from the myometrial tissue cDNA library (UTRSNOT06) using a computer search for amino acid sequence alignments. A consensus sequence, SEQ ID NO: 103, was derived from Incyte Clones 1638407 (UTRSNOT06), 3541410 (SEMVNOT04), 1290413 (BRAINOT11), 1467841 (PANCTUT02), 1306495 (PLACNOT02), and 1907983 (CONNTUT01).

In one embodiment, the invention encompasses a polypeptide comprising the amino acid sequence of SEQ ID NO:26. SIGP-26 is 217 amino acids in length and has seven potential phosphorylation sites at T214, S68, S148, S189, S30, S110, and Y149. SIGP-26 also has a potential signal peptide sequence between M1 and G31. SIGP-26 shares 18% identity with a mouse proline-rich protein (GI 200547). The fragment of SEQ ID NO: 103 from about nucleotide 146 to about nucleotide 206 is useful for hybridization. Northern analysis shows the expression of this sequence in gastrointestinal, hematopoietic, immunological, and reproductive cDNA libraries. Approximately 42% of these libraries are associated with neoplastic disorders and 39% with immune response.

Nucleic acids encoding the SIGP-27 of the present invention were first identified in Incyte Clone 1653112 from the prostate tumor tissue cDNA library (PROSTUT08) using a computer search for amino acid sequence alignments. A consensus sequence, SEQ ID NO: 104, was derived from Incyte Clones 1653112 (PROSTUT08), 3450102 (UTRSNON03), 1969850 (UCMCLST01), 1880259 (LEUKNOT03), 1504393 (BRAITUT07), and 394029 (TMLR2DT01).

In one embodiment, the invention encompasses a polypeptide comprising the amino acid sequence of SEQ ID NO:27. SIGP-27 is 504 amino acids in length and has eight potential phosphorylation sites at T338, T13, S38, T56, T132, T490, S33, and T472. SIGP-27 also has one potential leucine zipper pattern between L418 and L439. SIGP-27 shares 16% identity with mouse alpha-1 type-X collagen (GI 49794). The fragment of SEQ ID NO: 104 from about nucleotide 130 to about nucleotide 190 is useful for hybridization. Northern analysis shows the expression of this sequence in cardiovascular, endocrine, hematopoietic, immunological, neural, and reproductive cDNA libraries. Approximately 55% of these libraries are associated with neoplastic disorders and 22% with immune response.

Nucleic acids encoding the SIGP-28 of the present invention were first identified in Incyte Clone 1664634 from the breast tissue cDNA library (BRSTNOT09) using a computer search for amino acid sequence alignments. A consensus sequence, SEQ ID NO: 105, was derived from Incyte Clones 1664634 (BRSTNOT09) and 571656 (OVARNON01), and shotgun sequences SAPA04612, SAPA00377, and SAPA03034.

In one embodiment, the invention encompasses a polypeptide comprising the amino acid sequence of SEQ ID NO:28. SIGP-28 is 320 amino acids in length and has two potential N-glycosylation sites at N122 and N139; and eight potential phosphorylation sites at T30, S52, S109, S162, S220, S96, T258, and S280. SIGP-28 also has a potential signal peptide sequence between M1 and A21. SIGP-28 shares 28% identity with a C. elegans protein encoded by F32A7.4 (GI 1890375). The fragment of SEQ ID NO: 105 from about nucleotide 280 to about nucleotide 340 is useful for hybridization. Northern analysis shows the expression of this sequence in cardiovascular, gastrointestinal, hematopoietic, immunological, neural, and reproductive cDNA libraries. Approximately 38% of these libraries are associated with neoplastic disorders and 32% with immune response.

Nucleic acids encoding the SIGP-29 of the present invention were first identified in Incyte Clone 1690990 from the prostatic tumor tissue cDNA library (PROSTUT10) using a computer search for amino acid sequence alignments. A consensus sequence, SEQ ID NO: 106, was derived from Incyte Clone 1690990 (PROSTUT10), and shotgun sequences SAPA01051, SAPA04063, SAPA01670, SAPA02170, SAPA01946, and SAPA00282.

In one embodiment, the invention encompasses a polypeptide comprising the amino acid sequence of SEQ ID NO:29. SIGP-29 is 117 amino acids in length and has one potential N-glycosylation site at N96; four potential phosphorylation sites at S116, S34, T78, and S62; and one potential N-myristoylation site at G5. SIGP-29 also has one potential microbodies C-terminal targeting signal at S115. The fragment of SEQ ID NO: 106 from about nucleotide 1000 to about nucleotide 1062 is useful for hybridization. Northern analysis shows the expression of this sequence in gastrointestinal, reproductive, dermal, musculoskeletal, neural, and urogenital cDNA libraries. Approximately 77% of these libraries are associated with neoplastic disorders and 8% with immune response.

Nucleic acids encoding the SIGP-30 of the present invention were first identified in Incyte Clone 1704050 from the duodenal cDNA library (DUODNOT02) using a computer search for amino acid sequence alignments. A consensus sequence, SEQ ID NO:107, was derived from Incyte Clones 865233 (BRAITUT03), 1359660 (LUNGNOT12), and 1704050 (DUODNOT02) and shotgun sequence SAPA02672.

In one embodiment, the invention encompasses a polypeptide comprising the amino acid sequence of SEQ ID NO:30. SIGP-30 is 298 amino acids in length and has one potential amidation site at P226; four potential N-glycosylation sites at N98, N187, N236, and N277; seven potential casein kinase II phosphorylation sites at T39, S59, T100, T149, S205, T284, and S286; three potential protein kinase C phosphorylation sites at T52, S58, and S279; a potential signal sequence from M1 to G22; and a potential transmembrane spanning region from M230 to A261. SIGP-30 contains two potential immunoglobulin superfamily domains, from about F29 to about L131 and from about S138 to about R224. SIGP-30 shares 25% identity with the human A33 antigen precursor expressed in normal human colonic and small bowel epithelium and in human colon cancers (GI 1814277). In addition, the position of the hydrophobic transmembrane domain is conserved between these molecules. The cysteine residues at C50, C109, C139, C155, C214, and C254 are conserved between these molecules. The fragment of SEQ ID NO: 107 from about nucleotide 1150 to about nucleotide 1209 is useful for hybridization. Northern analysis shows the expression of this sequence in neural, reproductive, cardiovascular, and endocrine cDNA libraries. Approximately 68% of these libraries are associated with cancer and 9% with immune response.

Nucleic acids encoding the SIGP-31 of the present invention were first identified in Incyte Clone 1711840 from the prostate cDNA library (PROSNOT16) using a computer search for amino acid sequence alignments. A consensus sequence, SEQ ID NO:108, was derived from Incyte Clones 1711840 (PROSNOT16) and 2550483 (LUNGTUT06) and shotgun sequence SAQA03185.

In one embodiment, the invention encompasses a polypeptide comprising the amino acid sequence of SEQ ID NO:31. SIGP-31 is 118 amino acids in length and has three potential protein kinase C phosphorylation sites at S48, T103, and S109; and a potential signal peptide sequence from M1 to A20. SIGP-31 shares 61% identity with human midkine, a retinoic acid-responsive heparin binding factor involved in regulation of growth and differentiation (GI 182651). The fragment of SEQ ID NO:108 from about nucleotide 511 to about nucleotide 555 is useful for hybridization. Northern analysis shows the expression of this sequence in reproductive, gastrointestinal, developmental, neural, and cardiovascular cDNA libraries. Approximately 58% of these libraries are associated with cancer, 16% with immune response, and 23% with fetal/proliferating cells.

Nucleic acids encoding the SIGP-32 of the present invention were first identified in Incyte Clone 1747327 from the stomach tumor cDNA library (STOMTUT02) using a computer search for amino acid sequence alignments. A consensus sequence, SEQ ID NO: 109, was derived from Incyte Clones 475228 (MMLR2DT01), 1500771 (SINTBST01), 1880656 (LEUKNOT03), 1747327 (STOMTUT02), and 2720285 (LUNGTUT10).

In one embodiment, the invention encompasses a polypeptide comprising the amino acid sequence of SEQ ID NO:32. SIGP-32 is 248 amino acids in length and has one potential N-glycosylation site at N56; three potential casein kinase II phosphorylation sites at S46, S134, and S140; and one potential protein kinase C phosphorylation site at T217. SIGP-32 shares 100% identity with human K12 protein precursor which is expressed in breast cancer cells and peripheral blood leukocytes (GI 2062391). Northern analysis shows the expression of this sequence in gastrointestinal, reproductive, hematopoietic/immune, and cardiovascular cDNA libraries. Approximately 59% of these libraries are associated with cancer and 35% with immune response.

Nucleic acids encoding the SIGP-33 of the present invention were first identified in Incyte Clone 1750632 from the stomach tumor cDNA library (STOMTUT02) using a computer search for amino acid sequence alignments. A consensus sequence, SEQ ID NO: 110, was derived from Incyte Clones 1521122 (BLADTUT04) and 1750632 (STOMTUT02) and shotgun sequences SAEA02182 and SAEA10021.

In one embodiment, the invention encompasses a polypeptide comprising the amino acid sequence of SEQ ID NO:33. SIGP-33 is 150 amino acids in length and has one potential protein kinase C phosphorylation site at S6. SIGP-33 shares 49% identity with the C. elegans protein encoded by R151.6 (GI 459002). The fragment of SEQ ID NO: 110 from about nucleotide 514 to about nucleotide 573 is useful for hybridization. Northern analysis shows the expression of this sequence in cardiovascular and gastrointestinal cDNA libraries. Approximately 88% of these libraries are associated with cancer and 13% with immune response.

Nucleic acids encoding the SIGP-34 of the present invention were first identified in Incyte Clone 1812375 from the prostate tumor cDNA library (PROSTUT12) using a computer search for amino acid sequence alignments. A consensus sequence, SEQ ID NO: 111, was derived from Incyte Clones 775001 (COLNNOT05), 834305 (PROSNOT07), 1504623 (BRAITUT07), and 1812375 (PROSTUT12) and shotgun sequences SAQA02414, SATA00657, and SATA01478.

In one embodiment, the invention encompasses a polypeptide comprising the amino acid sequence of SEQ ID NO:34. SIGP-34 is 431 amino acids in length and has four potential N-glycosylation sites at N11, N49, N73, and N312; one potential cAMP- and cGMP-dependent protein kinase phosphorylation site at S197; six potential casein kinase II phosphorylation sites at T38, S79, S130, S165, S177, and T188; three potential protein kinase C phosphorylation sites at S184, T254, and S337; and a potential high affinity calcium ion-binding, vitamin K-dependent carboxylation domain between W371 and W408. The fragments of SEQ ID NO:1 II from about nucleotide 222 to about nucleotide 282 and the potential carboxylation domain encoded from about nucleotide 1267 to about nucleotide 1380 are useful for hybridization. Northern analysis shows the expression of this sequence in reproductive, neural, gastrointestinal, cardiovascular, and hematopoietic/immune DNA libraries. Approximately 52% of these libraries are associated with cancer, 24% with immune response, and 20% with fetal/proliferating cells.

Nucleic acids encoding the SIGP-35 of the present invention were first identified in Incyte Clone 1818761 from the prostate cDNA library (PROSNOT20) using a computer search for amino acid sequence alignments. A consensus sequence, SEQ ID NO:112, was derived from Incyte Clone 1818761 (PROSNOT20) and shotgun sequences SAJA00040, SAJA00601, SAJA01791, and SAJA02873.

In one embodiment, the invention encompasses a polypeptide comprising the amino acid sequence of SEQ ID NO:35. SIGP-35 is 278 amino acids in length and has one potential N-glycosylation site at N91; three potential casein kinase II phosphorylation sites at S9, S125, and S156; two potential protein kinase C phosphorylation sites at S77 and S224; one potential tyrosine kinase phosphorylation site at Y258; and a potential signal sequence from M1 to A30. SIGP-35 has fourteen consecutive collagen repeats (G-X-P or G-X-X) from G97 to P138 which could form a triple helical structure. SIGP-35 shares 28% identity with the human adipocyte complement-related protein precursor (Acrp30) (GI 2493789). The fragment of SEQ ID NO:112 from about nucleotide 157 to about nucleotide 210 is useful for hybridization. Northern analysis shows the expression of this sequence in developmental, dermal, gastrointestinal, hematopoietic/immune, neural, and reproductive cDNA libraries. Approximately 29% of these libraries are associated with cancer, 43% with immune response, and 29% with fetal development.

Nucleic acids encoding the SIGP-36 of the present invention were first identified in Incyte Clone 1824469 from the gallbladder tumor cDNA library (GBLADTUT01) using a computer search for amino acid sequence alignments. A consensus sequence, SEQ ID NO:113, was derived from Incyte Clones 1664262 (BRSTNOT09), 1733422 (BRSTTUT08), 1824469 (GBLADTUT01), 2057044 (BEPINOT01), and 2449822 (ENDANOT01).

In one embodiment, the invention encompasses a polypeptide comprising the amino acid sequence of SEQ ID NO:36. SIGP-36 is 286 amino acids in length and has one potential N-glycosylation site at N271; four potential casein kinase II phosphorylation sites at S50, S192, T230, and T251; and five potential protein kinase C phosphorylation sites at T29, T41, S50, T160, and T273. SIGP-36 shares 24% identity with the Mycobacterium tuberculosis protein encoded by MTC1237.14c (GI 2052134). The fragment of SEQ ID NO:113 from about nucleotide 415 to about nucleotide 468 is useful for hybridization. Northern analysis shows the expression of this sequence in reproductive, gastrointestinal, hematopoietic/immune, and neural cDNA libraries. Approximately 49% of these libraries are associated with cancer, 21% with immune response, and 21% with fetal/proliferating cells.

Nucleic acids encoding the SIGP-37 of the present invention were first identified in Incyte Clone 1864292 from the diseased prostate cDNA library (PROSNOT19) using a computer search for amino acid sequence alignments. A consensus sequence, SEQ ID NO: 114, was derived from Incyte Clone 1864292 (PROSNOT19) and shotgun sequences SARA02195, SARA03070, SARA03675, and SATA02454.

In one embodiment, the invention encompasses a polypeptide comprising the amino acid sequence of SEQ ID NO:37. SIGP-37 is 404 amino acids in length and has one potential amidation site at VI 36; one potential cAMP- and cGMP-dependent protein kinase phosphorylation site at S66; twenty potential casein kinase II phosphorylation sites at S23, T27, T74, S110, S111, S118, T122, S143, S145, S205, S207, S218, S219, S220, T252, S254, S328, S330, S385, and T393; and twelve potential protein kinase C phosphorylation sites at T27, S76, T81, S140, S161, S176, S229, T285, S309, S356, S367, and S398. SIGP-37 shares 18% identity with the S. cerevisiae protein encoded by SRP40, a weak suppressor of a mutant of the subunit AC40 of DNA-dependent RNA polymerases I and II (GI 295671). The fragment of SEQ ID NO: 114 from about nucleotide 193 to about nucleotide 222 is useful for hybridization. Northern analysis shows the expression of this sequence in reproductive, cardiovascular, and hematopoietic/immune cDNA libraries. Approximately 75% of these libraries are associated with cancer and 25% with immune response.

Nucleic acids encoding the SIGP-38 of the present invention were first identified in Incyte Clone 1866437 from the human promonocyte cell line cDNA library (THPINOT01) using a computer search for amino acid sequence alignments. A consensus sequence, SEQ ID NO: 115, was derived from Incyte Clones 817970 (OVARTUT01), 825684 (PROSNOT06), 1866437 (THP1NOT01), 2190170 (PROSNOT26), and 3137972 (SMCCNOT02).

In one embodiment, the invention encompasses a polypeptide comprising the amino acid sequence of SEQ ID NO:38. SIGP-38 is 405 amino acids in length and has one potential N-glycosylation site at N378; one potential cAMP- and cGMP-phosphorylation site at S332; nine potential casein kinase II phosphorylation sites at T34, S51, T77, S107, S158, S264, T266, S296, and S332; and one potential protein kinase C phosphorylation site at S68. The fragment of SEQ ID NO: 115 from about nucleotide 85 to about nucleotide 144 is useful for hybridization. Northern analysis shows the expression of this sequence in reproductive, hematopoietic/immune, neural, and developmental cDNA libraries. Approximately 37% of these libraries are associated with cancer, 33% with immune response, and 22% with fetal/proliferating cells.

Nucleic acids encoding the SIGP-39 of the present invention were first identified in Incyte Clone 1871375 from the leg skin erythema nodosum cDNA library (SKINBIT01) using a computer search for amino acid sequence alignments. A consensus sequence, SEQ ID NO: 116, was derived from Incyte Clones 1428052 (SINTBST01), 1871375 (SKINBIT01), and 3210563 (BLADNOT08).

In one embodiment, the invention encompasses a polypeptide comprising the amino acid sequence of SEQ ID NO:39. SIGP-39 is 177 amino acids in length and has one potential casein kinase II phosphorylation site at S133; one potential glycosaminoglycan attachment site at S28GGG; and four potential protein kinase C phosphorylation sites at S44, S82, S115, and T148. SIGP-39 contains a signature sequence shared by the binding domains of receptors for lymphokines, hematopoietic growth factors and growth hormone-related molecules at S52RWSLWS. The fragment of SEQ ID NO:116 encoding the sequence surrounding the receptor binding domain signature from about nucleotide 190 to about nucleotide 249 is useful for hybridization. Northern analysis shows the expression of this sequence in reproductive, cardiovascular, gastrointestinal, and developmental cDNA libraries. Approximately 44% of these libraries are associated with cancer and 19% with immune response.

Nucleic acids encoding the SIGP-40 of the present invention were first identified in Incyte Clone 1880830 from the leukocyte cDNA library (LEUKNOT03) using a computer search for amino acid sequence alignments. A consensus sequence, SEQ ID NO:117, was derived from Incyte Clones 361577 (PROSNOT01); 2113591 (BRAITUT03); 1880830 (LEUKNOT03) and shotgun sequences SATA03292 and SATA00377.

In one embodiment, the invention encompasses a polypeptide comprising the amino acid sequence of SEQ ID NO:40. SIGP-40 is 197 amino acids in length and has a potential cAMP- and cGMP-dependent protein kinase phosphorylation site at S121; and four potential protein kinase C phosphorylation sites at T3, S57, T107, and T153. SIGP-40 shares 15% identity with the Arabidopsis thaliana zinc-finger protein Lsd1 (GI 1872521). The fragment of SEQ ID NO:117 from about nucleotide 567 to about nucleotide 621 is useful for hybridization. Northern analysis shows the expression of this sequence in neural and reproductive cDNA libraries. Approximately 49% of these libraries are associated with neoplastic disorders, 24% with immune response, and 16% with fetal development.

Nucleic acids encoding the SIGP-41 of the present invention were first identified in Incyte Clone 1905325 from the ovary cDNA library (OVARNOT07) using a computer search for amino acid sequence alignments. A consensus sequence, SEQ ID NO:118, was derived from Incyte Clones 1905325 (OVARNOT07); 621454 (PGANNOT01); 621326 (PGANNOT01); 1264490 (SYNORAT05); 487357 (HNT2AGT01); 773311 (COLNCRT01); and shotgun sequence SATA03582.

In one embodiment, the invention encompasses a polypeptide comprising the amino acid sequence of SEQ ID NO:41. SIGP-41 is 302 amino acids in length and has two potential N-glycosylation sites at N80 and N252; three potential casein kinase II phosphorylation sites at S46, T58, and S143; and four potential protein kinase C phosphorylation sites at T58, S62, T147, and S300. SIGP-41 shares 27% identity with human necdin-related protein (GI 1754971). The fragment of SEQ ID NO: 118 from about nucleotide 1701 to about nucleotide 1800 is useful for hybridization. Northern analysis shows the expression of this sequence in reproductive, neural, and gastrointestinal cDNA libraries. Approximately 51% of these libraries are associated with neoplastic disorders and 20% with immune response, and 18% with fetal development.

Nucleic acids encoding the SIGP-42 of the present invention were first identified in Incyte Clone 1919931 from the breast tumor cDNA library (BRSTTUT01) using a computer search for amino acid sequence alignments. A consensus sequence, SEQ ID NO:119, was derived from Incyte Clones 1919931 (BRSTTUT01) and shotgun sequences SATA02529, SATA01526 and SATA00892.

In one embodiment, the invention encompasses a polypeptide comprising the amino acid sequence of SEQ ID NO:42. SIGP-42 is 164 amino acids in length and has one potential casein kinase II phosphorylation site at T68; and two potential protein kinase C phosphorylation sites at T81 and S85. SIGP-42 shares 12% identity with human chemokine receptor (GI 2104517). The fragment of SEQ ID NO:119 from about nucleotide 585 to about nucleotide 630 is useful for hybridization. Northern analysis shows the expression of this sequence in hematopoietic/immune, reproductive, and neural cDNA libraries. Approximately 50% of these libraries are associated with neoplastic disorders and 38% with immune response.

Nucleic acids encoding the SIGP-43 of the present invention were first identified in Incyte Clone 1969426 from the breast tissue cDNA library (BRSTNOT04) using a computer search for amino acid sequence alignments. A consensus sequence, SEQ ID NO: 120, was derived from Incyte Clones 1969426 (BRSTNOT04), 2373191 (ADRENOT07), 1225516 (COLNTUT02), 1555912 (BLADTUT04), 1449240 (PLACNOT02), and shotgun sequences SAZA01457 and SAZA00207.

In one embodiment, the invention encompasses a polypeptide comprising the amino acid sequence of SEQ ID NO:43. SIGP-43 is 235 amino acids in length and has one potential N-glycosylation site at N146; one potential glycosaminoglycan attachment site at S82; and four potential protein kinase C phosphorylation sites at T16, T43, S228, and S231. The fragment of SEQ ID NO:120 from about nucleotide 243 to about nucleotide 282 is useful for hybridization. Northern analysis shows the expression of this sequence in neural, reproductive, hematopoietic/immune, cardiovascular, gastrointestinal, and muscle cDNA libraries. Approximately 46% of these libraries are associated with neoplastic disorders and 28% with immune response.

Nucleic acids encoding the SIGP-44 of the present invention were first identified in Incyte Clone 1969948 from the umbilical cord cDNA library (UCMCL5T01) using a computer search for amino acid sequence alignments. A consensus sequence, SEQ ID NO: 121, was derived from Incyte Clones 1969948 (UCMCL5T01) and shotgun sequences SATA01513 and SATA00507.

In one embodiment, the invention encompasses a polypeptide comprising the amino acid sequence of SEQ ID NO:44. SIGP-44 is 203 amino acids in length and has three potential casein kinase II phosphorylation sites at T23, S114, and S120; one potential protein kinase C phosphorylation site at T105; and one potential tyrosine kinase phosphorylation site at Y47. The fragment of SEQ ID NO: 121 from about nucleotide 162 to about nucleotide 216 is useful for hybridization. Northern analysis shows the expression of this sequence in gastrointestinal, hematopoietic/immune, reproductive, and cardiovascular cDNA libraries. Approximately 35% of these libraries are associated with neoplastic disorders and 24% with immune response.

Nucleic acids encoding the SIGP-45 of the present invention were first identified in Incyte Clone 1988911 from the lung cDNA library (LUNGAST01) using a computer search for amino acid sequence alignments. A consensus sequence, SEQ ID NO:122, was derived from Incyte Clones 1988911 (LUNGAST01), 860576 (BRAITUT03), 3188894 (THYMNON04), 1466606 (PANCTUT02), 1920945 (BRSTTUT01), 1502970 (BRAITUT07), and shotgun sequence SAZC00040.

In one embodiment, the invention encompasses a polypeptide comprising the amino acid sequence of SEQ ID NO:45. SIGP-45 is 359 amino acids in length and has nine potential casein kinase II phosphorylation sites at S34, S47, S115, T120, T141, S157, S182, S214, and S331; three potential protein kinase C phosphorylation sites at S34, T259, and S325; and one potential tyrosine kinase phosphorylation site at Y241. SIGP-45 shares 16% identity with rat myosin heavy chain (GI 56649). The fragment of SEQ ID NO: 122 from about nucleotide 477 to about nucleotide 558 is useful for hybridization. Northern analysis shows the expression of this sequence in reproductive, hematopoietic/immune, gastrointestinal, and cardiovascular cDNA libraries. Approximately 47% of these libraries are associated with neoplastic disorders, 33% with immune response, and 20% with fetal development.

Nucleic acids encoding the SIGP-46 of the present invention were first identified in Incyte Clone 2061561 from the ovary cDNA library (OVARNOT03) using a computer search for amino acid sequence alignments. A consensus sequence, SEQ ID NO:123, was derived from Incyte Clones 2061561 (OVARNOT03), 2208104 (SINTFET03), 2058750 (OVARNOT03), and shotgun sequences SAZA00915, SAZA00150, and SAZA00799.

In one embodiment, the invention encompasses a polypeptide comprising the amino acid sequence of SEQ ID NO:46. SIGP-46 is 150 amino acids in length and has two potential amidation sites at F57 and W74; one potential cAMP- and cGMP-dependent protein kinase phosphorylation site at T62; two potential casein kinase II phosphorylation sites at T101 and T110; and two potential protein kinase C phosphorylation sites at T28 and T97. The fragment of SEQ ID NO: 123 from about nucleotide 82 to about nucleotide 168 is useful for hybridization. Northern analysis shows the expression of this sequence in reproductive, neural, gastrointestinal, and cardiovascular cDNA libraries. Approximately 54% of these libraries are associated with neoplastic disorders and 22% with immune response.

Nucleic acids encoding the SIGP-47 of the present invention were first identified in Incyte Clone 2084489 from the pancreas cDNA library (PANCNOT04) using a computer search for amino acid sequence alignments. A consensus sequence, SEQ ID NO: 124, was derived from Incyte Clones 2084489 (PANCNOT04) and shotgun sequences SAJA00837, SAJA00793, SAJA01402, SAJA01533, and SAJA01490.

In one embodiment, the invention encompasses a polypeptide comprising the amino acid sequence of SEQ ID NO:47. SIGP-47 is 402 amino acids in length and has one potential N-glycosylation site at N191; seven potential cAMP- and cGMP-dependent protein kinase phosphorylation sites at S22, S23, T80, S81, S202, S248, and S382; twenty-two potential casein kinase II phosphorylation sites at S8, S35, S56, S107, T152, S166, S170, S202, S206, S208, T212, S214, S216, T244, S252, S256, T264, T287, S288, T327, S362, S387; ten potential protein kinase C phosphorylation sites at S16, S116, S140, T180, S193, S194, T236, T244, S252, and S387; and one potential tyrosine kinase phosphorylation site at Y361. SIGP-47 shares 28% identity with an A. thaliana protein of unknown function (GI 2262136). The most conserved region, residues 296 to 386 of SIGP-47, shares 70% identity with residues 299 to 386 of the A. thaliana protein. In addition, the potential amidation site at A314 in SIGP-47 is conserved as one potential amidation site at Q317 in the A. thaliana protein; and four potential protein kinase C or cAMP- and cGMP dependent protein kinase phosphorylation sites at S193, T236, S252 and Y361 in SIGP-47 are conserved as potential phosphorylation sites at S165, S219, T247, and Y364 respectively in the A. thaliana protein. The fragment of SEQ ID NO: 124 from about nucleotide 468 to about nucleotide 531 is useful for hybridization. Northern analysis shows the expression of this sequence in neural, gastrointestinal and cardiovascular cDNA libraries. Approximately 50% of these libraries are associated with neoplastic disorders and 20% with trauma.

Nucleic acids encoding the SIGP-48 of the present invention were first identified in Incyte Clone 2203226 from the fetal spleen cDNA library (SPLNFET02) using a computer search for amino acid sequence alignments. A consensus sequence, SEQ ID NO:125, was derived from Incyte Clones 2203226 (SPLNFET02), 2215960 (SINTFET03), 1291348 (BRAINOT11), 1874915 (LEUKNOT02), and 275828 (TESTNOT03).

In one embodiment, the invention encompasses a polypeptide comprising the amino acid sequence of SEQ ID NO:48. SIGP-48 is 311 amino acids in length and has one potential amidation site at V117; one potential casein kinase II phosphorylation site at T215; and three potential protein kinase C phosphorylation sites at T13, S18, and T263. SIGP-48 shares 32% identity with a human putative Rab5 interacting protein (GI 1911776). The fragment of SEQ ID NO: 125 from about nucleotide 747 to about nucleotide 846 is useful for hybridization. Northern analysis shows the expression of this sequence in reproductive, cardiovascular, neural, and gastrointestinal cDNA libraries. Approximately 44% of these libraries are associated with neoplastic disorders, 30% with fetal/proliferative cells and tissues, and 23% with immune response.

Nucleic acids encoding the SIGP-49 of the present invention were first identified in Incyte Clone 2232884 from the prostate cDNA library (PROSNOT16) using a computer search for amino acid sequence alignments. A consensus sequence, SEQ ID NO:126, was derived from Incyte Clones 2232884 (PROSNOT16), 2728528 (OVARTUT05), 2232884 (PROSNOT16), and shotgun sequences SASA00238 and SASA00455.

In one embodiment, the invention encompasses a polypeptide comprising the amino acid sequence of SEQ ID NO:49. SIGP-49 is 316 amino acids in length and has one potential N-glycosylation site at N140; five potential casein kinase II phosphorylation sites at S3, T8, S29, S85, and T198; and two potential protein kinase C phosphorylation sites at T28 and S60. The fragment of SEQ ID NO: 126 from about nucleotide 180 to about nucleotide 279 is useful for hybridization. Northern analysis shows the expression of this sequence in reproductive, urologic, and neural cDNA libraries. Approximately 77% of these libraries are associated with neoplastic disorders.

Nucleic acids encoding the SIGP-50 of the present invention were first identified in Incyte Clone 2328134 from the colon cDNA library (COLNNOT11) using a computer search for amino acid sequence alignments. A consensus sequence, SEQ ID NO:127, was derived from Incyte Clones 2328134 (COLNNOT11), 1870180 (SKINBIT01), 081403 (SYNORAB01), and 851547 (NGANNOT01).

In one embodiment, the invention encompasses a polypeptide comprising the amino acid sequence of SEQ ID NO:50. SIGP-50 is 346 amino acids in length and has two potential cAMP- and cGMP-dependent protein kinase phosphorylation sites at residues S43 and S217; one potential casein kinase II phosphorylation site at residue T96; and five potential protein kinase C phosphorylation sites at residues T2, T15, T39, T247, and S301. SIGP-50 shares 33% identity with the human putative rab5-interacting protein (GI 1911776) and the casein kinase II phosphorylation site at residue T96. The fragment of SEQ ID NO: 127 encoding the potential extracellular ligand binding domain from about nucleotide 16 to about nucleotide 76 is useful for hybridization. Northern analysis shows the expression of this sequence in reproductive, gastrointestinal, cardiovascular, and neural cDNA libraries. Approximately 44% of these libraries are associated with cancer, 28% are associated with immune response, and 20% with fetal disorders.

Nucleic acids encoding the SIGP-51 of the present invention were first identified in Incyte Clone 2382718 from the pancreatic cDNA library (ISLTNOT01) using a computer search for amino acid sequence alignments. A consensus sequence, SEQ ID NO:128, was derived from Incyte Clones 2382718 (ISLTNOT01), 3472492 (LUNGNOT27), 014756 (THPIPLB01), 1731885 (BRSTTUT08), 1889866 (BLADTUT07), and 1447744 (PLACNOT02).

In one embodiment, the invention encompasses a polypeptide comprising the amino acid sequence of SEQ ID NO:51. SIGP-51 is 299 amino acids in length and has one potential N-glycosylation site at residue N185; one cAMP- and cGMP-dependent protein kinase phosphorylation site at T273; nine potential casein kinase II phosphorylation sites at S34, S82, T100, S118, T152, S154, T193, S203, and S287; eight potential protein kinase C phosphorylation sites at S57, T69, T95, S179, T269, S274, S275, and S284; and a potential signal peptide sequence from M1 to G27. SIGP-51 shares 26% identity with a human antigen precursor protein (GI 1814277); the protein kinase C phosphorylation sites at residues S57 and T69; and the casein kinase II phosphorylation site at residue T100. The fragment of SEQ ID NO: 128 encoding the potential extracellular ligand binding domain from about nucleotide 88 to about nucleotide 148 is useful for hybridization. Northern analysis shows the expression of this sequence in reproductive, gastrointestinal, and cardiovascular cDNA libraries. Approximately 48% of these libraries are associated with cancer, 29% are associated with immune response, and 20% with fetal disorders.

Nucleic acids encoding the SIGP-52 of the present invention were first identified in Incyte Clone 2452208 from the cardiovascular cDNA library (ENDANOT01) using a computer search for amino acid sequence alignments. A consensus sequence, SEQ ID NO: 129, was derived from Incyte Clones 2452280 (ENDANOT01), 1505094 (BRAITUT07), 1521239 (BLADTUT04), and 1309844 (COLNFET02).

In one embodiment, the invention encompasses a polypeptide comprising the amino acid sequence of SEQ ID NO:52. SIGP-52 is 351 amino acids in length and has two potential N-glycosylation sites at N241 and N337; two potential cAMP- and cGMP-dependent protein kinase phosphorylation sites at S201 and T318; six potential casein kinase II phosphorylation sites at S9, S136, T162, T252, S270, and S302; eight potential protein kinase C phosphorylation sites at T25, S34, T37, S64, S87, S112, S141, and S322; and one potential cell attachment sequence at R280GD. The fragment of SEQ ID NO: 129 encoding the potential extracellular ligand binding domain from about nucleotide 97 to about nucleotide 157 is useful for hybridization. Northern analysis shows the expression of this sequence in reproductive, gastrointestinal, cardiovascular, and neural cDNA libraries. Approximately 33% of these libraries are associated with cancer, 33% are associated with immune response, and 26% with fetal disorders.

Nucleic acids encoding the SIGP-53 of the present invention were first identified in Incyte Clone 2457825 from the aortic endothelial cell cDNA library (ENDANOT01) using a computer search for amino acid sequence alignments. A consensus sequence, SEQ ID NO: 130, was derived from Incyte Clone 2457825 (ENDANOT01) and shotgun sequences SASA00641, SASA02817, SASA01973, SASA03121, SASA01350, and SASA00693.

In one embodiment, the invention encompasses a polypeptide comprising the amino acid sequence of SEQ ID NO:53. SIGP-53 is 662 amino acids in length and has three potential cAMP- and cGMP-dependent protein kinase phosphorylation sites at S555, S578, and S652; ten potential casein kinase II phosphorylation sites at S67, T151, T215, S241, S470, S471, S482, S556, T589, and T618; one potential leucine zipper pattern from L572 to L593; four potential protein kinase C phosphorylation sites at T2, T21, S80, and T503; and one potential LIM domain signature site from C402 to L436. SIGP-53 shares 10% identity with the C. elegans protein encoded by WO4D2.1 (GI 1418625); and the casein kinase II phosphorylation site at residue S241. The fragment of SEQ ID NO: 130 encoding the potential extracellular ligand binding domain from about nucleotide 88 to about nucleotide 148 is useful for hybridization. Northern analysis shows the expression of this sequence in hematopoietic, gastrointestinal, reproductive, and cardiovascular cDNA libraries. Approximately 43% of these libraries are associated with cancer, 35% are associated with immune response, and 22% with fetal disorders.

Nucleic acids encoding the SIGP-54 of the present invention were first identified in Incyte Clone 2470740 from the hematopoietic cDNA library (THP1NOT03) using a computer search for amino acid sequence alignments. A consensus sequence, SEQ ID NO: 131, was derived from Incyte Clone 2470740 (THP1NOT03).

In one embodiment, the invention encompasses a polypeptide comprising the amino acid sequence of SEQ ID NO:54. SIGP-54 is 115 amino acids in length and has one potential protein kinase C phosphorylation site at S85; and one potential insulin family signature site from C23 to C37. The fragment of SEQ ID NO:131 encoding the potential extracellular ligand binding domain from about nucleotide 151 to about nucleotide 211 is useful for hybridization. Northern analysis shows the expression of this sequence in neural and developmental cDNA libraries. Approximately 33% of these libraries are associated with cancer and 33% are associated with fetal disorders.

Nucleic acids encoding the SIGP-55 of the present invention were first identified in Incyte Clone 2479092 from the aortic endothelial cell cDNA library (SMCANOT01) using a computer search for amino acid sequence alignments. A consensus sequence, SEQ ID NO: 132, was derived from Incyte Clone 2479092 (SMCANOT01) and 1981954 (LUNGTUT03).

In one embodiment, the invention encompasses a polypeptide comprising the amino acid sequence of SEQ ID NO:55. SIGP-55 is 157 amino acids in length and has one potential casein kinase II phosphorylation site at S31; one potential tyrosine kinase phosphorylation site at K150; and a potential signal peptide sequence from M1 to A26. The fragment of SEQ ID NO: 132 encoding the potential extracellular ligand binding domain from about nucleotide 97 to about nucleotide 157 is useful for hybridization. Northern analysis shows the expression of this sequence in reproductive, gastrointestinal, hematopoietic, and urologic cDNA libraries. Approximately 47% of these libraries are associated with cancer and 29% with immune response.

Nucleic acids encoding the SIGP-56 of the present invention were first identified in Incyte Clone 2480544 from the aortic smooth muscle cell cDNA library (SMCANOT01) using a computer search for amino acid sequence alignments. A consensus sequence, SEQ ID NO: 133, was derived from Incyte Clones 2480544 (SMCANOT01), 2472409 (THP1NOT03), 1516031 (PANCTUT01), 855817 (NGANNOT01), 1865287 (PROSNOT19), and 677835 (CRBLNOT01).

In one embodiment, the invention encompasses a polypeptide comprising the amino acid sequence of SEQ ID NO:56. SIGP-56 is 197 amino acids in length and has one potential N glycosylation site at N38; one potential casein kinase II phosphorylation site at S123; two potential protein kinase C phosphorylation sites at T71 and S82; and a potential signal peptide sequence from M1 to A27. SIGP-56 shares 15% identity with a Phaseolus vulgaris protein involved in the stress response (GI 169345) and shows conservation of proline and tyrosine residues in the C-terminal region. The fragment of SEQ ID NO: 133 from about nucleotide 125 to about nucleotide 160 is useful for hybridization. Northern analysis shows the expression of this sequence in neural, reproductive, and cardiovascular cDNA libraries. Approximately 49% of these libraries are associated with neoplastic disorders and 14% with immune response.

Nucleic acids encoding the SIGP-57 of the present invention were first identified in Incyte Clone 2518547 from the brain tumor cDNA library (BRAITUT21) using a computer search for amino acid sequence alignments. A consensus sequence, SEQ ID NO: 134, was derived from Incyte Clones 2518547 (BRAITUT21), 1509622 (LUNGNOT14), 1562945 (SPLNNOT04), 1640136 (UTRSNOT06), and 1432014 (BEPINON01).

In one embodiment, the invention encompasses a polypeptide comprising the amino acid sequence of SEQ ID NO:57. SIGP-57 is 245 amino acids in length and has one potential casein kinase II phosphorylation site at S27; and two potential protein kinase C phosphorylation sites at S5 and T229. SIGP-57 shares 36% identity with a human protein that binds a regulatory element of the c-myc gene (GI 33969). In addition, the potential protein kinase C phosphorylation site at T229 is conserved as a potential protein kinase A phosphorylation site at S176 in the human protein. The fragment of SEQ ID NO: 134 from about nucleotide 742 to about nucleotide 775 is useful for hybridization. Northern analysis shows the expression of this sequence in hematopoietic, reproductive, and neural cDNA libraries. Approximately 50% of these libraries are associated with neoplastic disorders and 28% with immune response.

Nucleic acids encoding the SIGP-58 of the present invention were first identified in Incyte Clone 2530650 from the gallbladder cDNA library (GBLANOT02) using a computer search for amino acid sequence alignments. A consensus sequence, SEQ ID NO: 135, was derived from Incyte Clones 2530650 (GBLANOT02), 2617724 (GBLANOT01), 3105644 (BRSTTUT15), 2903466 (DRGCNOT01), 1545010 (PROSTUT04), 2313837 (NGANNOT01), 1804413 (SINTNOT13), 3207379 (PENCNOT03), 2347051 (TESTTUT02), 2602493 (UTRSNOT10), 1259341 (MENITUT03), and 81943 (SYNORAB01).

In one embodiment, the invention encompasses a polypeptide comprising the amino acid sequence of SEQ ID NO:58. SIGP-58 is 310 amino acids in length and has one potential N glycosylation site at N₂O₆; one potential cAMP- and cGMP-dependent protein kinase phosphorylation site at T97; five potential casein kinase II phosphorylation sites at S62, S156, S214, S222, and T274; five potential protein kinase C phosphorylation sites at T150, T167, T208, T265, and S273; one potential tyrosine kinase phosphorylation site at Y96; one thyroglobulin type-1 repeat signature from F109 to G143; and a potential signal peptide sequence from M1 to A21. SIGP-58 shares 18% identity with bovine thyroglobulin (GI 2204111) and 46% identity between F109 and G 143, the thyroglobulin type-1 repeat signature. The fragment of SEQ ID NO: 135 from about nucleotide 92 to about nucleotide 127 is useful for hybridization. Northern analysis shows the expression of this sequence in reproductive and cardiovascular cDNA libraries. Approximately 67% of these libraries are associated with neoplastic disorders and 19% with immune response.

Nucleic acids encoding the SIGP-59 of the present invention were first identified in Incyte Clone 2652271 from the thymus cDNA library (THYMNOT04) using a computer search for amino acid sequence alignments. A consensus sequence, SEQ ID NO:136, was derived from Incyte Clones 2652271 (THYMNOT04), 2742813 (BRSTTUT14), 763431 (BRAITUT02), 1272403 (TESTTUT02), 1240531 (LUNGNOT03), and 1318448 (BLADNOT04).

In one embodiment, the invention encompasses a polypeptide comprising the amino acid sequence of SEQ ID NO:59. SIGP-59 is 256 amino acids in length and has three potential N glycosylation sites at N76, N106, and N212; three potential casein kinase II phosphorylation sites at T46, S188, and T204; two potential protein kinase C phosphorylation sites at S130 and S221; two potential ribonuclease T2 family histidine active sites from W62 to P69 and from F110 to C121; and a potential signal peptide sequence from M1 to A24. SIGP-59 shares 24% identity with Solanum lycopersicum ribonuclease LE (GI 895855); 80% identity between W62 and P75, one of the two ribonuclease T2 family histidine active sites; and 92% identity between F110 and C121, the second of the two ribonuclease T2 family histidine active sites. The fragment of SEQ ID NO: 136 from about nucleotide 462 to about nucleotide 494 is useful for hybridization. Northern analysis shows the expression of this sequence in reproductive, hematopoietic, and gastrointestinal cDNA libraries. Approximately 53% of these libraries are associated with neoplastic disorders and 28% with immune response.

Nucleic acids encoding the SIGP-60 of the present invention were first identified in Incyte Clone 2746976 from the lung tumor cDNA library (LUNGTUT11) using a computer search for amino acid sequence alignments. A consensus sequence, SEQ ID NO: 137, was derived from Incyte Clones 2746976 (LUNGTUT11), 488049 (HNT2AGT01), 1907738 (CONNTUT01), 782645 (MYOMNOT01), and 823864 (PROSNOT06).

In one embodiment, the invention encompasses a polypeptide comprising the amino acid sequence of SEQ ID NO:60. SIGP-60 is 160 amino acids in length and has one potential cAMP- and cGMP-dependent protein kinase phosphorylation site at T31; four potential casein kinase II phosphorylation sites at S23, S47, S96, and S152; four potential protein kinase C phosphorylation sites at S23, T125, S126, and T149; and a clathrin adaptor complex small chain signature from I56 to F66. SIGP-60 shares 84% identity with mouse clathrin-associated protein 19 (GI 191983) and 91% identity with the clathrin adaptor complex small chain signature between I56 and F66. In addition, all potential casein kinase II and protein kinase C phosphorylation sites are conserved between SIGP-60 and the mouse protein. The fragments of SEQ ID NO: 137 from about nucleotide 144 to about nucleotide 170 and from about nucleotide 495 to about nucleotide 521 are useful for hybridization. Northern analysis shows the expression of this sequence in hematopoietic, cardiovascular, and reproductive cDNA libraries. Approximately 39% of these libraries are associated with neoplastic disorders and 39% with immune response.

Nucleic acids encoding the SIGP-61 of the present invention were first identified in Incyte Clone 2753496 from the THP-1 promonocyte cDNA library (THP1AZS08) using a computer search for amino acid sequence alignments. A consensus sequence, SEQ ID NO: 138, was derived from Incyte Clones 2753496 (THP1AZS08), 2642512 (LUNGTUT08), 1367244 (SCORNON02), 474-458 (MMLRIDT01), 1349777 (LATRTUT02), 1380831 (BRAITUT08), and 832934 (PROSTUT04).

In one embodiment, the invention encompasses a polypeptide comprising the amino acid sequence of SEQ ID NO:61. SIGP-61 is 341 amino acids in length and has one potential N glycosylation site at N66; four potential casein kinase II phosphorylation sites at T157, T207, S296, and S335; two potential protein kinase C phosphorylation sites at S159 and S296; and one potential tyrosine kinase phosphorylation site at Y184. SIGP-61 shares 17% identity with Schizosaccharomyces pombe BEM46, a protein involved in cell polarity (GI 987286) and the potential phosphorylation sites at T157 and S296. The fragment of SEQ ID NO: 138 from about nucleotide 79 to about nucleotide 114 is useful for hybridization. Northern analysis shows the expression of this sequence in reproductive, gastrointestinal, and neural cDNA libraries. Approximately 52% of these libraries are associated with neoplastic disorders and 25% with immune response.

Nucleic acids encoding the SIGP-62 of the present invention were first identified in Incyte Clone 2781553 from the ovarian tumor cDNA library (OVARTUT03) using a computer search for amino acid sequence alignments. A consensus sequence, SEQ ID NO: 139, was derived from Incyte Clones 2781553 (OVARTUT03), 1413079 (BRAINOT12), 894971 (BRSTNOT05), 2696043 (UTRSNOT12), 1267806 (BRAINOT09), 1961608 (BRSTNOT04), 1755817 (LIVRTUT01), 1793882 (PROSTUT05), 1251515 (LUNGFET03), 1560984 (SPLNNOT04), and 1872574 (LEUKNOT02).

In one embodiment, the invention encompasses a polypeptide comprising the amino acid sequence of SEQ ID NO:62. SIGP-62 is 430 amino acids in length and has one potential cAMP- and cGMP-dependent protein kinase phosphorylation site at S387; thirteen potential casein kinase II phosphorylation sites at S182, S214, S235, T248, S258, T266, T275, T294, S313, T356, S387, T404, and S413; six potential protein kinase C phosphorylation sites at T71, S168, S235, S306, T356, and S374; and a mitochondrial energy transfer protein signature from P114 to L122. Northern analysis shows the expression of this sequence in reproductive, neural, and hematopoietic cDNA libraries. Approximately 47% of these libraries are associated with neoplastic disorders and 19% with immune response.

Nucleic acids encoding the SIGP-63 of the present invention were first identified in Incyte Clone 2821925 from the adrenal tumor cDNA library (ADRETUT06) using a computer search for amino acid sequence alignments. A consensus sequence, SEQ ID NO: 140, was derived from Incyte Clones 2821925 (ADRETUT06), 933799 (CERVNOT01), and 136467 (SYNORAB01).

In one embodiment, the invention encompasses a polypeptide comprising the amino acid sequence of SEQ ID NO:63. SIGP-63 is 143 amino acids in length and has one potential cAMP- and cGMP-dependent protein kinase phosphorylation site at S109; three potential casein kinase II phosphorylation sites at S36, S80, and T84; five potential protein kinase C phosphorylation sites at T31, T55, T70, S109, and T122; and a potential signal peptide sequence from M1 to A21. Northern analysis shows the expression of this sequence in reproductive, musculoskeletal and cardiovascular cDNA libraries. Approximately 50% of these libraries are associated with neoplastic disorders and 27% with immune response.

Nucleic acids encoding the SIGP-64 of the present invention were first identified in Incyte Clone 2879068 from the uterine tumor cDNA library (UTRSTUT05) using a computer search for amino acid sequence alignments. A consensus sequence, SEQ ID NO:141, was derived from Incyte Clones 2879068 (UTRSTUT05), 2910155 (KIDNTUT15), 488673 (HNT2AGT01), 1285407 (COLNNOT16), 1415890 (BRAINOT12), 1352662 (LATRTUT02), 41046 (TBLYNOT01), and 2686554 (LUNGNOT23).

In one embodiment, the invention encompasses a polypeptide comprising the amino acid sequence of SEQ ID NO:64. SIGP-64 is 301 amino acids in length and has two potential N glycosylation sites at N20 and N251; five potential casein kinase II phosphorylation sites at S8, S41, T125, T161, and T163; five potential protein kinase C phosphorylation sites at T40, S41, T59, T66, and S181; one potential tyrosine kinase phosphorylation site at Y176; one potential glycosaminoglycan attachment site at S253; and two putative RNP-1 RNA-binding signatures from R70 to F77 and from R155 to Y162. SIGP-64 shares 59% identity with human heterogeneous nuclear ribonucleoprotein D (GI 870749); 100% identity between R70 and F77, one of the two RNP-1 RNA-binding signatures; and 89% identity between R155 and Y162, the second of the two RNP-1 RNA-binding signatures. In addition, eight potential phosphorylation sites are conserved between SIGP-64 and the human ribonucleoprotein. The fragments of SEQ ID NO:141 from about nucleotide 207 to about nucleotide 248 and from about nucleotide 726 to about nucleotide 752 are useful for hybridization. Northern analysis shows the expression of this sequence in reproductive, neural, hematopoietic, and gastrointestinal cDNA libraries. Approximately 48% of these libraries are associated with neoplastic disorders and 24% with immune response.

Nucleic acids encoding the SIGP-65 of the present invention were first identified in Incyte Clone 2886757 from the small intestine cDNA library (SINJNOT02) using a computer search for amino acid sequence alignments. A consensus sequence, SEQ ID NO: 142, was derived from Incyte Clones 2886757 (SINJNOT02), 2230747 (PROSNOT16), and 899432 (BRSTTUT03).

In one embodiment, the invention encompasses a polypeptide comprising the amino acid sequence of SEQ ID NO:65. SIGP-65 is 233 amino acids in length and has two potential N-glycosylation sites at N82 and N196; one potential casein kinase II phosphorylation site at S170; and two potential protein kinase C phosphorylation sites at S102 and T134. SIGP-65 shares 22% identity with S. cerevisiae protein encoded by YOL135c (GI 1420026), and the potential casein kinase II phosphorylation site at S170 is conserved between the two proteins. The fragment of SEQ ID NO: 142 from about nucleotide 99 to about nucleotide 137 is useful for hybridization. Northern analysis shows the expression of this sequence in reproductive, cardiovascular, and gastrointestinal cDNA libraries. Approximately 59% of these libraries are associated with neoplastic disorders.

Nucleic acids encoding the SIGP-66 of the present invention were first identified in Incyte Clone 2964329 from the cervical spinal cord cDNA library (SCORNOT04) using a computer search for amino acid sequence alignments. A consensus sequence, SEQ ID NO: 143, was derived from Incyte Clones 2964329, (SCORNOT04), 1274814 (TESTTUT02), 746049 (BRAITUT01), 1395667 (THYRNOT03), 1362944 (LUNGNOT12), and 2589 (HMC1NOT01).

In one embodiment, the invention encompasses a polypeptide comprising the amino acid sequence of SEQ ID NO:66. SIGP-66 is 354 amino acids in length and has one potential cAMP- and cGMP-dependent protein kinase phosphorylation site at S346; two potential casein kinase II phosphorylation sites at S164 and T180; six potential protein kinase C phosphorylation sites at S43, S135, S150, S164, S172, and S201; and one potential tyrosine kinase phosphorylation site at Y182. SIGP-66 shares 12% identity with S. cerevisiae mitochondrial internal membrane carrier protein (GI 311667). In addition, one potential protein kinase C site is conserved between these molecules. The fragment of SEQ ID NO:143 from about nucleotide 416 to about nucleotide 442 is useful for hybridization. Northern analysis shows the expression of this sequence in reproductive, neural, hematopoietic/immune, gastrointestinal, and cardiovascular cDNA libraries. Approximately 46% of these libraries are associated with neoplastic disorders and 26% with immune response.

Nucleic acids encoding the SIGP-67 of the present invention were first identified in Incyte Clone 2965248 from the cervical spinal cord cDNA library (SCORNOT04) using a computer search for amino acid sequence alignments. A consensus sequence, SEQ ID NO:144, was derived from Incyte Clones 2965248 (SCORNOT04), 485746 (HNT2RAT01), 865684 (BRAITUT03), 1459157 (COLNFET02), 1597772 (BRAINOT14), 531430 (BRAINOT03), 725362 (SYNOOAT01), 1620429 (BRAITUT13), and 190305 (SYNORAB01).

In one embodiment, the invention encompasses a polypeptide comprising the amino acid sequence of SEQ ID NO:67 SIGP-67 is 235 amino acids in length and has seven potential cAMP- and cGMP-dependent protein kinase phosphorylation sites at S50, T80, T98, T126, S135, S136, and T194; three potential casein kinase II phosphorylation sites at S60, T80, and S81; six potential protein kinase C phosphorylation sites at S114, T119, T137, S142, S146, and S174; and a strathmin 1 family signature from P75 to E84. SIGP-67 shares 44% identity with human strathmin homolog SCG10/neuron-specific growth-associated protein in Alzheimer's disease (GI 1478503), and 71% identity between M1 and A107. In addition, one potential cAMP- and cGMP-dependent protein kinase phosphorylation site, one potential casein kinase II phosphorylation site, the strathmin 1 family signature, and the hydrophobic transmembrane domains are conserved between these molecules. TM1 extends from about L15 to about F25; and TM2, from about G196 to about P212. The fragments of SEQ ID NO: 144 from about nucleotide 158 to about nucleotide 196 and from about nucleotide 614 to about nucleotide 643 are useful for hybridization. Northern analysis shows the expression of this sequence in neural, reproductive, gastrointestinal, and hematopoietic/immune cDNA libraries. Approximately 50% of these libraries are associated with neoplastic disorders and 19% with immune response.

Nucleic acids encoding the SIGP-68 of the present invention were first identified in Incyte Clone 3000534 from the Th2 T lymphocyte cDNA library (TLYMNOT06) using a computer search for amino acid sequence alignments. A consensus sequence, SEQ ID NO: 145, was derived from Incyte Clones 3000534 (TLYMNOT06), 1830964 (THPIAZT01), 1329136 (PANCNOT07), and 2910083 (KIDNTUT15).

In one embodiment, the invention encompasses a polypeptide comprising the amino acid sequence of SEQ ID NO:68. SIGP-68 is 221 amino acids in length and has two potential casein kinase II phosphorylation sites at T31 and T70; one potential glycosaminoglycan attachment site at S62; three potential protein kinase C phosphorylation sites at T111, T146, and T199; and an endoplasmic reticulum targeting sequence at H218DEL. SIGP-68 shares 61% identity with the human stroma cell-derived secretory factor-2 (GI 1741868). In addition, one potential protein kinase C phosphorylation site and the hydrophobic transmembrane domains are conserved between these molecules. TM1 extends from about A10 to about G27; and TM2, from about T31 to about L45. The cysteines at C38, C92, C100, and C149 are conserved between both molecules. The fragments of SEQ ID NO: 145 from about nucleotide 89 to about nucleotide 118 and from about nucleotide 608 to about nucleotide 643 are useful for hybridization. Northern analysis shows the expression of this sequence in hematopoietic/immune, reproductive, cardiovascular, and gastrointestinal cDNA libraries. Approximately 41% of these libraries are associated with neoplastic disorders and 31% with immune response.

Nucleic acids encoding the SIGP-69 of the present invention were first identified in Incyte Clone 3046870 from the coronary artery cDNA library (HEAANOT01) using a computer search for amino acid sequence alignments. A consensus sequence, SEQ ID NO:146, was derived from Incyte-Clones 3046870 (HEAANOT01), 2719210 (THYRNOT09), 581291 (SATPFI006), 1961256 (BRSTNOT04), 2226972 (SEMVNOT01), 2023351 (CONNNOT01), 1379008 (LUNGNOT10), and 1943136 (HIPONOT01).

In one embodiment, the invention encompasses a polypeptide comprising the amino acid sequence of SEQ ID NO:69. SIGP-69 is 483 amino acids in length and has one potential N-glycosylation site at N178; ten potential casein kinase II phosphorylation sites at S16, S49, T60, T67, T92, T121, T170, T187, T250, and S431; and nine potential protein kinase C phosphorylation sites at S113, T170, T187, T194, S210, T265, S284, T355, and S431. Northern analysis shows the expression of this sequence in reproductive, gastrointestinal, cardiovascular, and neural cDNA libraries. Approximately 49% of these libraries are associated with neoplastic disorders and 24% with immune response.

Nucleic acids encoding the SIGP-70 of the present invention were first identified in Incyte Clone 3057669 from the pons cDNA library (PONSAZT01) using a computer search for amino acid sequence alignments. A consensus sequence, SEQ ID NO:147, was derived from Incyte Clones 3057669 (PONSAZT01), 548211 (BEPINOT01), 3702516 (PENCNOT07), 3581270 (293TF3T01), 495191 (HNT2NOT01), 2784427 (BRSTNOT13), 1515961 (PANCTUT01), 3552333 (SYNONOT01), 2838668 (DRGLNOT01), 14600680 (COLNFET02), and 285677 (EOSIHET02).

In one embodiment, the invention encompasses a polypeptide comprising the amino acid sequence of SEQ ID NO:70. SIGP-70 is 371 amino acids in length and has three potential N-glycosylation sites at N70, N125, and N362; eleven potential casein kinase II phosphorylation sites at T22, S66, S72, S73, S102, T160, T201, T215, T278, T285, and S316; seven potential protein kinase C phosphorylation sites at S72, T79, S99, T127, S134, S257, and T299; and one protein kinase signature and profile from L188 to F200. Northern analysis shows the expression of this sequence in gastrointestinal, reproductive, and neural cDNA libraries. Approximately 54% of these libraries are associated with neoplastic disorders and 14% with immune response.

Nucleic acids encoding the SIGP-71 of the present invention were first identified in Incyte Clone 3088178 from the aorta cDNA library (HEAONOTO3) using a computer search for amino acid sequence alignments. A consensus sequence, SEQ ID NO:148, was derived from Incyte Clones 3088178 (HEAONOTO3), 589421 (UTRSNOT01), 2059958 (OVARNOT03), 1550631 (PROSNOT06), and 1271480 (TESTTUT02).

In one embodiment, the invention encompasses a polypeptide comprising the amino acid sequence of SEQ ID NO:71. SIGP-71 is 402 amino acids in length and has two potential N glycosylation sites at N13 and N366; two potential cAMP- and cGMP-dependent protein kinase phosphorylation sites at T50 and S51; five potential casein kinase II phosphorylation sites at T50, S51, S52, S56, and S246; one potential glycosaminoglycan attachment site at S247; eight potential protein kinase C phosphorylation sites at T45, T46, S224, S240, S259, T279, S338, and S376; one potential tyrosine kinase phosphorylation site at Y273; and one beta-transducin family Trp-Asp repeat signature from V243 to V257. SIGP-71 shares 22% identity with S. cerevisiae protein encoded by HRE594 (GI 498997; truncated sequence). In addition, one potential N-glycosylation site, and two potential casein kinase II phosphorylation sites are conserved between these molecules. The fragment of SEQ ID NO:148 from about nucleotide 725 to about nucleotide 766 is useful for hybridization. Northern analysis shows the expression of this sequence in reproductive, neural, cardiovascular, and hematopoietic/immune cDNA libraries. Approximately 51% of these libraries are associated with neoplastic disorders and 23% with immune response.

Nucleic acids encoding the SIGP-72 of the present invention were first identified in Incyte Clone 3094321 from the breast cDNA library (BRSTNOT19) using a computer search for amino acid sequence alignments. A consensus sequence, SEQ ID NO: 149, was derived from Incyte Clones 3094321 (BRSTNOT19), 2517422H1 (BRAITUT21), 2101110 (BRAITUT02), 1303603 (PLACNOT02), 2675275 (KIDNNOT19), 1988065 (LUNGAST01), 34101 (THP1NOB10), 1815156 (PROSNOT20), 602724 (BRSTTUT01), and 1485067 (CORPNOT02).

In one embodiment, the invention encompasses a polypeptide comprising the amino acid sequence of SEQ ID NO:72. SIGP-72 is 640 amino acids in length and has four potential N-glycosylation sites at N295, N513, N568, and N619; two potential cAMP- and cGMP-dependent protein kinase phosphorylation sites at S239 and S507; sixteen potential casein kinase II phosphorylation sites at S42, T178, T220, S229, S239, T247, S289, S350, S372, S446, T463, S492, T580, S592, S604, and S625; nine potential protein kinase C phosphorylation sites at T150, T166, T174, S239, T328, S407, T451, S609, and S621; one potential tyrosine kinase phosphorylation site at Y265; and one cytochrome c family heme-binding site signature at C158YECHP. SIGP-72 shares 33% identity with an essential yeast ubiquitin-activating enzyme homolog (GI 793879). In addition, one potential N-glycosylation site, one potential casein kinase II phosphorylation site, and six potential protein kinase C phosphorylation sites are conserved between these molecules. The fragments of SEQ ID NO: 149 from about nucleotide 382 to about nucleotide 423 and from about nucleotide 1087 to about nucleotide 1113 are useful for hybridization. Northern analysis shows the expression of this sequence in reproductive, hematopoietic/immune, cardiovascular, and gastrointestinal cDNA libraries. Approximately 48% of these libraries are associated with neoplastic disorders and 24% with immune response.

Nucleic acids encoding the SIGP-73 of the present invention were first identified in Incyte Clone 3115936 from the lung cDNA library (LUNGTUT13) using a computer search for amino acid sequence alignments. A consensus sequence, SEQ ID NO:150, was derived from Incyte Clones 3115936 (LUNGTUT13) 2359411 (LUNGFET05), 2189762 (PROSNOT26), 1449756 (PLACNOT02), 541212 (LNODNOT02), 079364 (SYNORAB01), 864877 (BRAITUT03), 2697958 (UTRSNOT12), 1818830 (PROSNOT20), 1966765 (BRSTNOT04), 998279 (KIDNTUT01), 1961616 (BRSTNOT04), and 1431515 (BEPINON01).

In one embodiment, the invention encompasses a polypeptide comprising the amino acid sequence of SEQ ID NO:73. SIGP-73 is 237 amino acids in length and has five potential casein kinase II phosphorylation sites at S43, S47, S72, S131, and T177; and three potential protein kinase C phosphorylation sites at S39, S125, and T202. SIGP-73 shares 44% identity with t yeast Rer1p protein, which ensures correct localization of Sec12p integral membrane protein of the endoplasmic reticulum (GI 517174). In addition, the hydrophobic transmembrane domains are conserved among these molecules. TM1 extends from about A82 to about P126; and TM2, from about A166 to about M203. The fragment of SEQ ID NO:150 from about nucleotide 585 to about nucleotide 623 is useful for hybridization. Northern analysis shows the expression of this sequence in reproductive, neural, cardiovascular, gastrointestinal, and hematopoietic/immune cDNA libraries. Approximately 48% of these libraries are associated with neoplastic disorders and 24% with immune response.

Nucleic acids encoding the SIGP-74 of the present invention were first identified in Incyte Clone 3116522 from the lung cDNA library (LUNGTUT13) using a computer search for amino acid sequence alignments. A consensus sequence, SEQ ID NO:151, was derived from Incyte Clones 3116522 (LUNGTUTI3), 2523149 (BRAITUT21), 1513583 (PANCTUT01), 834017 (PROSNOT07), 1631796 (COLNNOT19), 1502736 (BRAITUT07), and 78850 (SYNORAB01).

In one embodiment, the invention encompasses a polypeptide comprising the amino acid sequence of SEQ ID NO:74. SIGP-74 is 432 amino acids in length and has three potential casein kinase II phosphorylation sites at S144, S257, and S317; three potential protein kinase C phosphorylation sites at T68, S231, and T372; and one potential tyrosine kinase phosphorylation site at Y240. SIGP-74 shares 28% identity with the human UDP-galactose transporter isoform (GI 1669560). In addition, one potential protein kinase C phosphorylation site and the hydrophobic transmembrane domains are conserved between these molecules. TM4 extends from about Q108 to about G127; TM5, from about S152 to about LI 73; TM6, from about K205 to about K228; TM7, from about T242 to about S257; TM8, from about T268 to about S283; TM9, from about A294 to about T328; and TM10, from about A338 to about V409. The fragment of SEQ ID NO: 151 from about nucleotide 710 to about nucleotide 736 is useful for hybridization. Northern analysis shows the expression of this sequence in reproductive, gastrointestinal, cardiovascular, hematopoietic/immune, and urologic cDNA libraries. Approximately 54% of these libraries are associated with neoplastic disorders and 25% with immune response.

Nucleic acids encoding the SIGP-75 of the present invention were first identified in Incyte Clone 3117184 from the lung cDNA library (LUNGTUT13) using a computer search for amino acid sequence alignments. A consensus sequence, SEQ ID NO: 152, was derived from Incyte Clones 3117184 (LUNGTUT13), 2494724 (ADRETUT05), and 1922002 (BRSTTUT01).

In one embodiment, the invention encompasses a polypeptide comprising the amino acid sequence of SEQ ID NO:75. SIGP-75 is 252 amino acids in length and has one potential N-glycosylation site at N93; one potential cAMP- and cGMP-dependent protein kinase phosphorylation site at S179; one potential casein kinase II phosphorylation site at T189; and five potential protein kinase C phosphorylation sites at S95, S115, S123, T140, and T200. SIGP-75 shares 39% identity with C. elegans protein encoded by WO4D2.6 (GI 1418628). In addition, one potential N-glycosylation site, and three potential protein kinase C phosphorylation sites are conserved between the molecules. The fragment of SEQ ID NO: 152 from about nucleotide 567 to about nucleotide 593 is useful for hybridization. Northern analysis shows the expression of this sequence in cardiovascular, gastrointestinal, hematopoietic/immune, and reproductive cDNA libraries. Approximately 50% of these libraries are associated with neoplastic disorders and 20% with immune response.

Nucleic acids encoding the SIGP-76 of the present invention were first identified in Incyte Clone 3125156 from the lymph node cDNA library (LNODNOT05) using a computer search for amino acid sequence alignments. A consensus sequence, SEQ ID NO: 153, was derived from Incyte Clones 3125156 (LNODNOT05), 1417459 (BRAINOT12), 1567861 (UTRSNOT05), 154233 (THP1PLB02), 872652 (LUNGAST01), 2525803 (BRAITUT21), and 1209172 (BRSTNOT02).

In one embodiment, the invention encompasses a polypeptide comprising the amino acid sequence of SEQ ID NO:76. SIGP-76 is 523 amino acids in length and has one potential N glycosylation sites at N186; nine potential casein kinase II phosphorylation sites at S63, T85, S179, S188, T210, S231, T269, T295, and S474; one potential glycosaminoglycan attachment site at S335; ten potential protein kinase C phosphorylation sites at T9, S159, S172, S179, T246, S263, S283, S416, S447, and S498; two potential tyrosine kinase phosphorylation sites at Y106 and Y170; and one tyrosine specific protein phosphatase active site at V331. SIGP-76 shares 21% identity with human T-cell protein tyrosine phosphatase (GI 804750), the N186 glycosylation site, the phosphorylation sites at S179, S188, T210, T246, S263, T295, S416, and Y170; and 50% identity between P324 and F344, the region of the tyrosine specific protein phosphatase active site. The fragments of SEQ ID NO: 153 from about nucleotide 64 to about nucleotide 183 and from about nucleotide 1087 to about nucleotide 1119 are useful for hybridization. Northern analysis shows the expression of this sequence in neural, reproductive, and gastrointestinal cDNA libraries. Approximately 55% of these libraries are associated with neoplastic disorders and 22% with immune response.

Nucleic acids encoding the SIGP-77 of the present invention were first identified in Incyte Clone 3129120 from the lung tumor cDNA library (LUNGTUT12) using a computer search for amino acid sequence alignments. A consensus sequence, SEQ ID NO: 154, was derived from Incyte Clones 3129120 (LUNGTUT12), 3744590 (THYMNOT08), 1512939 (PANCTUT01), 3220539 (COLNNON03), 1435889 (PANCNOT08), 1452745 (PENITUT01), 874548 (LUNGAST01), 1524326 (UCMCL5T01), and 811239 (LUNGNOT04).

In one embodiment, the invention encompasses a polypeptide comprising the amino acid sequence of SEQ ID NO:77. SIGP-77 is 621 amino acids in length and has two potential N glycosylation sites at N₂O₃ and N517; one potential protein kinase A or G phosphorylation site at S84; five potential casein kinase II phosphorylation sites at T45, T185, T233, T278, and S573; seven potential protein kinase C phosphorylation sites at T45, T95, S109, S299, T318, S324, and T482; and one potential leucine zipper motif from L332 to L353. SIGP-77 shares 27% identity and the phosphorylation site at T318 with S. cerevisiae membrane protein important for endocytosis (GI 1256890). The fragments of SEQ ID NO: 154 from about nucleotide 64 to about nucleotide 183 and from about nucleotide 1087 to about nucleotide 1119 are useful for hybridization. Northern analysis shows the expression of this sequence in reproductive, neural, gastrointestinal, and cardiovascular cDNA libraries. Approximately 53% of these libraries are associated with neoplastic disorders and 17% with immune response.

The invention also encompasses SIGP variants. A preferred SIGP variant is one which has at least about 80%, more preferably at least about 90%, and most preferably at least about 95% amino acid sequence identity to the SIGP amino acid sequence, and which contains at least one functional or structural characteristic of SIGP.

The invention also encompasses polynucleotides which encode SIGP. Accordingly, any nucleic acid sequence which encodes the amino acid sequence of SIGP can be used to produce recombinant molecules which express SIGP. In a particular embodiment, the invention encompasses a polynucleotide consisting of a nucleic acid sequence selected from the group consisting of SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO: 152, SEQ ID NO:153, and SEQ ID NO:154.

It will be appreciated by those skilled in the art that as a result of the degeneracy of the genetic code, a multitude of polynucleotide sequences encoding SIGP, some bearing minimal homology to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring SIGP, and all such variations are to be considered as being specifically disclosed.

Although nucleotide sequences which encode SIGP and its variants are preferably capable of hybridizing to the nucleotide sequence of the naturally occurring SIGP under appropriately selected conditions of stringency, it may be advantageous to produce nucleotide sequences encoding SIGP or its derivatives possessing a substantially different codon usage. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding SIGP and its derivatives without altering the encoded amino acid sequences include the production of RNA transcripts having more desirable properties, such as a greater half-life, than transcripts produced from the naturally occurring sequence.

The invention also encompasses production of DNA sequences which encode SIGP and SIGP derivatives, or fragments thereof, entirely by synthetic chemistry. After production, the synthetic sequence may be inserted into any of the many available expression vectors and cell systems using reagents that are well known in the art. Moreover, synthetic chemistry may be used to introduce mutations into a sequence encoding SIGP or any fragment thereof.

Also encompassed by the invention are polynucleotide sequences that are capable of hybridizing to the claimed polynucleotide sequences, and, in particular, to those shown in SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO: 118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO: 153, and SEQ ID NO: 154, under various conditions of stringency. (See, e.g., Wahl, G. M. and S. L. Berger (1987) Methods Enzymol. 152:399-407; and Kimmel, A. R. (1987) Methods Enzymol. 152:507-511.)

Methods for DNA sequencing are well known and generally available in the art and may be used to practice any of the embodiments of the invention. The methods may employ such enzymes as the Klenow fragment of DNA polymerase I, Sequenase® (US Biochemical Corp., Cleveland, Ohio), Taq polymerase (Perkin Elmer), thermostable T7 polymerase (Amersham, Chicago, Ill.), or combinations of polymerases and proofreading exonucleases such as those found in the ELONGASE Amplification System (GIBCO/BRL, Gaithersburg, Md.). Preferably, the process is automated with machines such as the Hamilton Micro Lab 2200 (Hamilton, Reno, Nev.), Peltier Thermal Cycler (PTC200; MJ Research, Watertown, Mass.) and the ABI Catalyst and 373 and 377 DNA Sequencers (Perkin Elmer).

The nucleic acid sequences encoding SIGP may be extended utilizing a partial nucleotide sequence and employing various methods known in the art to detect upstream sequences, such as promoters and regulatory elements. For example, one method which may be employed, restriction-site PCR, uses universal primers to retrieve unknown sequence adjacent to a known locus. (See, e.g., Sarkar, G. (1993) PCR Methods Applic. 2:318-322.) In particular, genomic DNA is first amplified in the presence of a primer complementary to a linker sequence within the vector and a primer specific to the region predicted to encode the gene. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR may also be used to amplify or extend sequences using divergent primers based on a known region. (See, e.g., Triglia, T. et al. (1988) Nucleic Acids Res. 16:8186.) The primers may be designed using commercially available software such as OLIGO 4.06 Primer Analysis software (National Biosciences Inc., Plymouth, Minn.) or another appropriate program to be about 22 to 30 nucleotides in length, to have a GC content of about 50% or more, and to anneal to the target sequence at temperatures of about 68° C. to 72° C. The method uses several restriction enzymes to generate a suitable fragment in the known region of a gene. The fragment is then circularized by intramolecular ligation and used as a PCR template.

Another method which may be used is capture PCR, which involves PCR amplification of DNA fragments adjacent to a known sequence in human and yeast artificial chromosome DNA. (See, e.g., Lagerstrom, M. et al. (1991) PCR Methods Applic. 1: 111-119.) In this method, multiple restriction enzyme digestions and ligations may be used to place an engineered double-stranded sequence into an unknown fragment of the DNA molecule before performing PCR. Other methods which may be used to retrieve unknown sequences are known in the art. (See, e.g., Parker, J. D. et al. (1991) Nucleic Acids Res. 19:3055-3060.) Additionally, one may use PCR, nested primers, and PromoterFinder™ libraries to walk genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable in that they will include more sequences which contain the 5′ regions of genes. Use of a randomly primed library may be especially preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different fluorescent dyes (one for each nucleotide) which are laser activated, and a charge coupled device camera for detection of the emitted wavelengths. Output/light intensity may be converted to electrical signal using appropriate software (e.g., Genotyper™ and Sequence Navigator™, Perkin Elmer), and the entire process from loading of samples to computer analysis and electronic data display may be computer controlled. Capillary electrophoresis is especially preferable for the sequencing of small pieces of DNA which might be present in limited amounts in a particular sample.

In another embodiment of the invention, polynucleotide sequences or fragments thereof which encode SIGP may be used in recombinant DNA molecules to direct expression of SIGP, or fragments or functional equivalents thereof, in appropriate host cells. Due to the inherent degeneracy of the genetic code, other DNA sequences which encode substantially the same or a functionally equivalent amino acid sequence may be produced, and these sequences may be used to clone and express SIGP.

As will be understood by those of skill in the art, it may be advantageous to produce SIGP-encoding nucleotide sequences possessing non-naturally occurring codons. For example, codons preferred by a particular prokaryotic or eukaryotic host can be selected to increase the rate of protein expression or to produce an RNA transcript having desirable properties, such as a half-life which is longer than that of a transcript generated from the naturally occurring sequence.

The nucleotide sequences of the present invention can be engineered using methods generally known in the art in order to alter SIGP-encoding sequences for a variety of reasons including, but not limited to, alterations which modify the cloning, processing, and/or expression of the gene product. DNA shuffling by random fragmentation and PCR reassembly of gene fragments and synthetic oligonucleotides may be used to engineer the nucleotide sequences. For example, site-directed mutagenesis may be used to insert new restriction sites, alter glycosylation patterns, change codon preference, produce splice variants, introduce mutations, and so forth.

In another embodiment of the invention, natural, modified, or recombinant nucleic acid sequences encoding SIGP may be ligated to a heterologous sequence to encode a fusion protein. For example, to screen peptide libraries for inhibitors of SIGP activity, it may be useful to encode a chimeric SIGP protein that can be recognized by a commercially available antibody. A fusion protein may also be engineered to contain a cleavage site located between the SIGP encoding sequence and the heterologous protein sequence, so that SIGP may be cleaved and purified away from the heterologous moiety.

In another embodiment, sequences encoding SIGP may be synthesized, in whole or in part, using chemical methods well known in the art. (See, e.g., Caruthers, M. H. et al. (1980) Nucl. Acids Res. Symp. Ser. 215-223, and Horn, T. et al. (1980) Nucl. Acids Res. Symp. Ser. 225-232.) Alternatively, the protein itself may be produced using chemical methods to synthesize the amino acid sequence of SIGP, or a fragment thereof. For example, peptide synthesis can be performed using various solid-phase techniques. (See, e.g., Roberge, J. Y. et al. (1995) Science 269:202-204.) Automated synthesis may be achieved using the ABI 431A Peptide Synthesizer (Perkin Elmer).

The newly synthesized peptide may be substantially purified by preparative high performance liquid chromatography. (See, e.g, Chiez, R. M. and F. Z. Regnier (1990) Methods Enzymol. 182:392-421.) The composition of the synthetic peptides may be confirmed by amino acid analysis or by sequencing. (See, e.g., Creighton, T. (1983) Proteins, Structures and Molecular Properties, WM Freeman and Co., New York, N.Y.) Additionally, the amino acid sequence of SIGP, or any part thereof, may be altered during direct synthesis and/or combined with sequences from other proteins, or any part thereof, to produce a variant polypeptide.

In order to express a biologically active SIGP, the nucleotide sequences encoding SIGP or derivatives thereof may be inserted into appropriate expression vector, i.e., a vector which contains the necessary elements for the transcription and translation of the inserted coding sequence.

Methods which are well known to those skilled in the art may be used to construct expression vectors containing sequences encoding SIGP and appropriate transcriptional and translational control elements. These methods include in vitro recombinant DNA techniques, synthetic techniques, and in vivo genetic recombination. (See, e.g., Sambrook, J. et al. (1989) Molecular Cloning, A Laboratory Manual, Cold Spring Harbor Press, Plainview, N.Y., ch. 4, 8, and 16-17; and Ausubel, F. M. et al. (1995, and periodic supplements) Current Protocols in Molecular Biology, John Wiley & Sons, New York, N.Y., ch. 9, 13, and 16.)

A variety of expression vector/host systems may be utilized to contain and express sequences encoding SIGP. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus (CaMV) or tobacco mosaic virus (TMV)) or with bacterial expression vectors (e.g., Ti or pBR322 plasmids); or animal cell systems. The invention is not limited by the host cell employed.

The “control elements” or “regulatory sequences” are those non-translated regions, e.g., enhancers, promoters, and 5′ and 3′ untranslated regions, of the vector and polynucleotide sequences encoding SIGP which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters, e.g., hybrid lacZ promoter of the Bluescript® phagemid (Stratagene, La Jolla, Calif.) or pSport1™ plasmid (GIBCO/BRL), may be used. The baculovirus polyhedrin promoter may be used in insect cells. Promoters or enhancers derived from the genomes of plant cells (e.g., heat shock, RUBISCO, and storage protein genes) or from plant viruses (e.g., viral promoters or leader sequences) may be cloned into the vector. In mammalian cell systems, promoters from mammalian genes or from mammalian viruses are preferable. If it is necessary to generate a cell line that contains multiple copies of the sequence encoding SIGP, vectors based on SV40 or EBV may be used with an appropriate selectable marker.

In bacterial systems, a number of expression vectors may be selected depending upon the use intended for SIGP. For example, when large quantities of SIGP are needed for the induction of antibodies, vectors which direct high level expression of fusion proteins that are readily purified may be used. Such vectors include, but are not limited to, multifunctional E. coli cloning and expression vectors such as Bluescript® (Stratagene), in which the sequence encoding SIGP may be ligated into the vector in frame with sequences for the amino-terminal Met and the subsequent 7 residues of β-galactosidase so that a hybrid protein is produced, and pIN vectors. (See, e.g., Van Heeke, G. and S. M. Schuster (1989) J. Biol. Chem. 264:5503-5509.) pGEX vectors (Pharmacia Biotech, Uppsala, Sweden) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast Saccharomyces cerevisiae, a number of vectors containing constitutive or inducible promoters, such as alpha factor, alcohol oxidase, and PGH, may be used. (See, e.g., Ausubel, supra; and Grant et al. (1987) Methods Enzymol. 153:516-544.)

In cases where plant expression vectors are used, the expression of sequences encoding SIGP may be driven by any of a number of promoters. For example, viral promoters such as the 35S and 19S promoters of CaMV may be used alone or in combination with the omega leader sequence from TMV. (Takamatsu, N. (1987) EMBO J. 6:307-311.) Alternatively, plant promoters such as the small subunit of RUBISCO or heat shock promoters may be used. (See, e.g., Coruzzi, G. et al. (1984) EMBO J. 3:1671-1680; Broglie, R. et al. (1984) Science 224:838-843; and Winter, J. et al. (1991) Results Probl. Cell Differ. 17:85-105.) These constructs can be introduced into plant cells by direct DNA transformation or pathogen-mediated transfection. Such techniques are described in a number of generally available reviews. (See, e.g., Hobbs, S. or Murry, L. E. in McGraw Hill Yearbook of Science and Technology (1992) McGraw Hill, New York, N.Y.; pp. 191-196.)

An insect system may also be used to express SIGP. For example, in one such system, Autographa californica nuclear polyhedrosis virus (AcNPV) is used as a vector to express foreign genes in Spodoptera frugiperda cells or in Trichoplusia larvae. The sequences encoding SIGP may be cloned into a non-essential region of the virus, such as the polyhedrin gene, and placed under control of the polyhedrin promoter. Successful insertion of sequences encoding SIGP will render the polyhedrin gene inactive and produce recombinant virus lacking coat protein. The recombinant viruses may then be used to infect, for example, S. frugiperda cells or Trichoplusia larvae in which SIGP may be expressed. (See, e.g., Engelhard, E. K. et al. (1994) Proc. Nat. Acad. Sci. 91:3224-3227.)

In mammalian host cells, a number of viral-based expression systems may be utilized. In cases where an adenovirus is used as an expression vector, sequences encoding SIGP may be ligated into an adenovirus transcription/translation complex consisting of the late promoter and tripartite leader sequence. Insertion in a non-essential E1 or E3 region of the viral genome may be used to obtain a viable virus which is capable of expressing SIGP in infected host cells. (See, e.g., Logan, J. and T. Shenk (1984) Proc. Natl. Acad. Sci. 81:3655-3659.) In addition, transcription enhancers, such as the Rous sarcoma virus (RSV) enhancer, may be used to increase expression in mammalian host cells.

Human artificial chromosomes (HACs) may also be employed to deliver larger fragments of DNA than can be contained and expressed in a plasmid. HACs of about 6 kb to 10 Mb are constructed and delivered via conventional delivery methods (liposomes, polycationic amino polymers, or vesicles) for therapeutic purposes.

Specific initiation signals may also be used to achieve more efficient translation of sequences encoding SIGP. Such signals include the ATG initiation codon and adjacent sequences. In cases where sequences encoding SIGP and its initiation codon and upstream sequences are inserted into the appropriate expression vector, no additional transcriptional or translational control signals may be needed. However, in cases where only coding sequence, or a fragment thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers appropriate for the particular cell system used. (See, e.g., Scharf, D. et al. (1994) Results Probl. Cell Differ. 20:125-162.)

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding, and/or function. Different host cells which have specific cellular machinery and characteristic mechanisms for post-translational activities (e.g., CHO, HeLa, MDCK, HEK293, and W138), are available from the American Type Culture Collection (ATCC, Bethesda, Md.) and may be chosen to ensure the correct modification and processing of the foreign protein.

For long term, high yield production of recombinant proteins, stable expression is preferred. For example, cell lines capable of stably expressing SIGP can be transformed using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase genes and adenine phosphoribosyltransferase genes, which can be employed in tk.sup.- or apr.sup.-cells, respectively. (See, e.g., Wigler, M. et al. (1977) Cell 11:223-232; and Lowy, I. et al. (1980) Cell 22:817-823) Also, antimetabolite, antibiotic, or herbicide resistance can be used as the basis for selection. For example, dhfr confers resistance to methotrexate; npt confers resistance to the aminoglycosides neomycin and G-418; and als or pat confer resistance to chlorsulfuron and phosphinotricin acetyltransferase, respectively. (See, e.g., Wigler, M. et al. (1980) Proc. Natl. Acad. Sci. 77:3567-3570; Colbere-Garapin, F. et al (1981) J. Mol. Biol. 150:1-14; and Murry, supra.) Additional selectable genes have been described, e.g., trpB, which allows cells to utilize indole in place of tryptophan, or hisD, which allows cells to utilize histinol in place of histidine. (See, e.g., Hartman, S. C. and R. C. Mulligan (1988) Proc. Natl. Acad. Sci. 85:8047-8051.) Recently, the use of visible markers has gained popularity with such markers as anthocyanins, β glucuronidase and its substrate GUS, luciferase and its substrate luciferin. Green fluorescent proteins (GFP) (Clontech, Palo Alto, Calif.) are also used (See, e.g., Chalfie, M. et al. (1994) Science 263:802-805.) These markers can be used not only to identify transformants, but also to quantify the amount of transient or stable protein expression attributable to a specific vector system. (See, e.g., Rhodes, C. A. et al. (1995) Methods Mol. Biol. 55:121-131.)

Although the presence/absence of marker gene expression suggests that the gene of interest is also present, the presence and expression of the gene may need to be confirmed. For example, if the sequence encoding SIGP is inserted within a marker gene sequence, transformed cells containing sequences encoding SIGP can be identified by the absence of marker gene function. Alternatively, a marker gene can be placed in tandem with a sequence encoding SIGP under the control of a single promoter. Expression of the marker gene in response to induction or selection usually indicates expression of the tandem gene as well.

Alternatively, host cells which contain the nucleic acid sequence encoding SIGP and express SIGP may be identified by a variety of procedures known to those of skill in the art. These procedures include, but are not limited to, DNA-DNA or DNA-RNA hybridizations and protein bioassay or immunoassay techniques which include membrane, solution, or chip based technologies for the detection and/or quantification of nucleic acid or protein sequences.

The presence of polynucleotide sequences encoding SIGP can be detected by DNA-DNA or DNA-RNA hybridization or amplification using probes or fragments or fragments of polynucleotides encoding SIGP. Nucleic acid amplification based assays involve the use of oligonucleotides or oligomers based on the sequences encoding SIGP to detect transformants containing DNA or RNA encoding SIGP.

A variety of protocols for detecting and measuring the expression of SIGP, using either polyclonal or monoclonal antibodies specific for the protein, are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on SIGP is preferred, but a competitive binding assay may be employed. These and other assays are well described in the art. (See, e.g., Hampton, R. et al. (1990) Serological Methods, a Laboratory Manual, APS Press, St Paul, Minn., Section IV; and Maddox, D. E. et al. (1983) J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides encoding SIGP include oligolabeling, nick translation, end-labeling, or PCR amplification using a labeled nucleotide. Alternatively, the sequences encoding SIGP, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Pharmacia & Upjohn (Kalamazoo, Mich.), Promega (Madison, Wis.), and U.S. Biochemical Corp. (Cleveland, Ohio). Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with nucleotide sequences encoding SIGP may be cultured under conditions suitable for the expression and recovery of the protein from cell culture. The protein produced by a transformed cell may be secreted or contained intracellularly depending on the sequence and/or the vector used. As will be understood by those of skill in the art, expression vectors containing polynucleotides which encode SIGP may be designed to contain signal sequences which direct secretion of SIGP through a prokaryotic or eukaryotic cell membrane. Other constructions may be used to join sequences encoding SIGP to nucleotide sequences encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences, such as those specific for Factor XA or enterokinase (Invitrogen, San Diego, Calif.), between the purification domain and the SIGP encoding sequence may be used to facilitate purification. One such expression vector provides for expression of a fusion protein containing SIGP and a nucleic acid encoding 6 histidine residues preceding a thioredoxin or an enterokinase cleavage site. The histidine residues facilitate purification on immobilized metal ion affinity chromatography. (IMAC) (See, e.g., Porath, J. et al. (1992) Prot. Exp. Purif. 3: 263-281.) The enterokinase cleavage site provides a means for purifying SIGP from the fusion protein. (See, e.g., Kroll, D. J. et al. (1993) DNA Cell Biol. 12:441-453.)

Fragments of SIGP may be produced not only by recombinant production, but also by direct peptide synthesis using solid-phase techniques. (See, e.g., Creighton, T. E. (1984) Protein: Structures and Molecular Properties, pp. 55-60, W.H. Freeman and Co., New York, N.Y.) Protein synthesis may be performed by manual techniques or by automation. Automated synthesis may be achieved, for example, using the Applied Biosystems 431A Peptide Synthesizer (Perkin Elmer). Various fragments of SIGP may be synthesized separately and then combined to produce the full length molecule.

### Therapeutics

The expression of the human signal peptide-containing proteins of the invention (SIGP) is closely associated with cell proliferation. Therefore, in cancers or immune response where SIGP is an activator, transcription factor, or enhancer, and is promoting cell proliferation, it is desirable to decrease the expression of SIGP. In conditions where SIGP is an inhibitor or suppressor and is controlling or decreasing cell proliferation, it is desirable to provide the protein or to increase the expression of SIGP.

In one embodiment, where SIGP is an inhibitor, SIGP or a fragment or derivative thereof may be administered to a subject to treat or prevent a cancer such as adenocarcinoma, leukemia, lymphoma, melanoma, myeloma, sarcoma, and teratocarcinoma. Such cancers include, but are not limited to, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus.

In another embodiment, a pharmaceutical composition comprising purified SIGP may be used to treat or prevent a cancer including, but not limited to, those listed above.

In another embodiment, an agonist which is specific for SIGP may be administered to a subject to treat or prevent a cancer including, but not limited to, those cancers listed above.

In another further embodiment, a vector capable of expressing SIGP, or a fragment or a derivative thereof, may be administered to a subject to treat or prevent a cancer including, but not limited to, those cancers listed above.

In a further embodiment where SIGP is promoting cell proliferation, antagonists which decrease the expression or activity of SIGP may be administered to a subject to treat or prevent a cancer such as adenocarcinoma, leukemia, lymphoma, melanoma, myeloma, sarcoma, and teratocarcinoma. Such cancers include, but are not limited to, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. In one aspect, antibodies which specifically bind SIGP may be used directly as an antagonist or indirectly as a targeting or delivery mechanism for bringing a pharmaceutical agent to cells or tissue which express SIGP.

In another embodiment, a vector expressing the complement of the polynucleotide encoding SIGP may be administered to a subject to treat or prevent a cancer including, but not limited to, those cancers listed above.

In yet another embodiment where SIGP is promoting leukocyte activity or proliferation, antagonists which decrease the activity of SIGP may be administered to a subject to treat or prevent an immune response. Such responses include, but are not limited to, disorders such as AIDS, Addison's disease, adult respiratory distress syndrome, allergies, anemia, asthma, atherosclerosis, bronchitis, cholecystitus, Crohn's disease, ulcerative colitis, atopic dermatitis, dermatomyositis, diabetes mellitus, emphysema, atrophic gastritis, glomerulonephritis, gout, Graves' disease, hypereosinophilia, irritable bowel syndrome, lupus erythematosus, multiple sclerosis, myasthenia gravis, myocardial or pericardial inflammation, osteoarthritis, osteoporosis, pancreatitis, polymyositis, rheumatoid arthritis, scleroderma, Sjogren's syndrome, and autoimmune thyroiditis; complications of cancer, hemodialysis, extracorporeal circulation; viral, bacterial, fungal, parasitic, protozoal, and helminthic infections; and trauma. In one aspect, antibodies which specifically bind SIGP may be used directly as an antagonist or indirectly as a targeting or delivery mechanism for bringing a pharmaceutical agent to cells or tissue which express SIGP.

In another embodiment, a vector expressing the complement of the polynucleotide encoding SIGP may be administered to a subject to treat or prevent an immune response including, but not limited to, those listed above.

In other embodiments, any of the proteins, antagonists, antibodies, agonists, complementary sequences, or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of SIGP may be produced using methods which are generally known in the art. In particular, purified SIGP may be used to produce antibodies or to screen libraries of pharmaceutical agents to identify those which specifically bind SIGP. Antibodies to SIGP may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are especially preferred for therapeutic use.

For the production of antibodies, various hosts including goats, rabbits, rats, mice, humans, and others may be immunized by injection with SIGP or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to SIGP have an amino acid sequence consisting of at least about 5 amino acids, and, more preferably, of at least about 10 amino acids. It is also preferable that these oligopeptides, peptides, or fragments are identical to a portion of the amino acid sequence of the natural protein and contain the entire amino acid sequence of a small, naturally occurring molecule. Short stretches of SIGP amino acids may be fused with those of another protein, such as KLH, and antibodies to the chimeric molecule may be produced.

Monoclonal antibodies to SIGP may be prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:31-42; Cote, R. J. et al. (1983) Proc. Natl. Acad. Sci. 80:2026-2030; and Cole, S. P. et al. (1984) Mol. Cell. Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. 81:6851-6855; Neuberger, M. S. et al. (1984) Nature 312:604-608; and Takeda, S. et al. (1985) Nature 314:452-454.) Alternatively, techniques described for the production of single chain antibodies may be adapted, using methods known in the art, to produce SIGP-specific single chain antibodies. Antibodies with related specificity, but of distinct idiotypic composition, may be generated by chain shuffling from random combinatorial immunoglobulin libraries. (See, e.g., Burton D. R. (1991) Proc. Natl. Acad. Sci. 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. 86: 3833-3837; and Winter, G. et al. (1991) Nature 349:293-299.)

Antibody fragments which contain specific binding sites for SIGP may also be generated. For example, such fragments include, but are not limited to, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity. (See, e.g., Huse, W. D. et al. (1989) Science 246:1275-1281.)

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between SIGP and its specific antibody. A two-site, monoclonal-based immunoassay utilizing monoclonal antibodies reactive to two non-interfering SIGP epitopes is preferred, but a competitive binding assay may also be employed. (Maddox, supra.)

In another embodiment of the invention, the polynucleotides encoding SIGP, or any fragment or complement thereof, may be used for therapeutic purposes. In one aspect, the complement of the polynucleotide encoding SIGP may be used in situations in which it would be desirable to block the transcription of the mRNA. In particular, cells may be transformed with sequences complementary to polynucleotides encoding SIGP. Thus, complementary molecules or fragments may be used to modulate SIGP activity, or to achieve regulation of gene function. Such technology is now well known in the art, and sense or antisense oligonucleotides or larger fragments can be designed from various locations along the coding or control regions of sequences encoding SIGP.

Expression vectors derived from retroviruses, adenoviruses, or herpes or vaccinia viruses, or from various bacterial plasmids, may be used for delivery of nucleotide sequences to the targeted organ, tissue, or cell population. Methods which are well known to those skilled in the art can be used to construct vectors which will express nucleic acid sequences complementary to the polynucleotides of the gene encoding SIGP. (See, e.g., Sambrook, supra; and Ausubel, supra.)

Genes encoding SIGP can be turned off by transforming a cell or tissue with expression vectors which express high levels of a polynucleotide, or fragment thereof, encoding SIGP. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector, and may last even longer if appropriate replication elements are part of the vector system.

As mentioned above, modifications of gene expression can be obtained by designing complementary sequences or antisense molecules (DNA, RNA, or PNA) to the control, 5′, or regulatory regions of the gene encoding SIGP. Oligonucleotides derived from the transcription initiation site, e.g., between about positions −10 and +10 from the start site, are preferred. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee, J. E. et al. (1994) in Huber, B. E. and B. I. Carr, Molecular and Immunologic Approaches, Futura Publishing Co., Mt. Kisco, N.Y., pp. 163-177.) A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of sequences encoding SIGP.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding SIGP. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (See, e.g., Goldman, C. K. et al. (1997) Nature Biotechnology 15:462-466.)

Any of the therapeutic methods described above may be applied to any subject in need of such therapy, including, for example, mammals such as dogs, cats, cows, horses, rabbits, monkeys, and most preferably, humans.

An additional embodiment of the invention relates to the administration of a pharmaceutical or sterile composition, in conjunction with a pharmaceutically acceptable carrier, for any of the therapeutic effects discussed above. Such pharmaceutical compositions may consist of SIGP, antibodies to SIGP, and mimetics, agonists, antagonists, or inhibitors of SIGP. The compositions may be administered alone or in combination with at least one other agent, such as a stabilizing compound, which may be administered in any sterile, biocompatible pharmaceutical carrier including, but not limited to, saline, buffered saline, dextrose, and water. The compositions may be administered to a patient alone, or in combination with other agents, drugs, or hormones.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combining active compounds with solid excipient and processing the resultant mixture of granules (optionally, after grinding) to obtain tablets or dragee cores. Suitable auxiliaries can be added, if desired. Suitable excipients include carbohydrate or protein fillers, such as sugars, including lactose, sucrose, mannitol, and sorbitol; starch from corn, wheat, rice, potato, or other plants; cellulose, such as methyl cellulose, hydroxypropylmethyl-cellulose, or sodium carboxymethylcellulose; gums, including arabic and tragacanth; and proteins, such as gelatin and collagen. If desired, disintegrating or solubilizing agents may be added, such as the cross-linked polyvinyl pyrrolidone, agar, and alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks's solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, and succinic acid. Salts tend to be more soluble in aqueous or other protonic solvents than are the corresponding free base forms. In other cases, the preferred preparation may be a lyophilized powder which may contain any or all of the following: 1 mM to 50 mM histidine, 0.1% to 2% sucrose, and 2% to 7% mannitol, at a pH range of 4.5 to 5.5, that is combined with buffer prior to use.

After pharmaceutical compositions have been prepared, they can be placed in an appropriate container and labeled for treatment of an indicated condition. For administration of SIGP, such labeling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells or in animal models such as mice, rats, rabbits, dogs, or pigs. An animal model may also be used to determine the appropriate concentration range and route of administration. Such information can then be used to determine useful doses and routes for administration in humans.

A therapeutically effective dose refers to that amount of active ingredient, for example SIGP or fragments thereof, antibodies of SIGP, and agonists, antagonists or inhibitors of SIGP, which ameliorates the symptoms or condition. Therapeutic efficacy and toxicity may be determined by standard pharmaceutical procedures in cell cultures or with experimental animals, such as by calculating the ED50 (the dose therapeutically effective in 50% of the population) or LD50 (the dose lethal to 50% of the population) statistics. The dose ratio of therapeutic to toxic effects is the therapeutic index, and it can be expressed as the ED50/LD50 ratio. Pharmaceutical compositions which exhibit large therapeutic indices are preferred. The data obtained from cell culture assays and animal studies are used to formulate a range of dosage for human use. The dosage contained in such compositions is preferably within a range of circulating concentrations that includes the ED50 with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting pharmaceutical compositions may be administered every 3 to 4 days, every week, or biweekly depending on the half-life and clearance rate of the particular formulation.

Normal dosage amounts may vary from about 0.1.mu.g to 100,000 mu.g, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

### Diagnostics

In another embodiment, antibodies which specifically bind SIGP may be used for the diagnosis of disorders characterized by expression of SIGP, or in assays to monitor patients being treated with SIGP or agonists, antagonists, or inhibitors of SIGP. Antibodies useful for diagnostic purposes may be prepared in the same manner as described above for therapeutics. Diagnostic assays for SIGP include methods which utilize the antibody and a label to detect SIGP in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring SIGP, including ELISAs, RIAs, and FACS, are known in the art and provide a basis for diagnosing altered or abnormal levels of SIGP expression. Normal or standard values for SIGP expression are established by combining body fluids or cell extracts taken from normal mammalian subjects, preferably human, with antibody to SIGP under conditions suitable for complex formation The amount of standard complex formation may be quantitated by various methods, preferably by photometric means. Quantities of SIGP expressed in subject, control, and disease samples from biopsied tissues are compared with the standard values. Deviation between standard and subject values establishes the parameters for diagnosing disease.

In another embodiment of the invention, the polynucleotides encoding SIGP may be used for diagnostic purposes. The polynucleotides which may be used include oligonucleotide sequences, complementary RNA and DNA molecules, and PNAs. The polynucleotides may be used to detect and quantitate gene expression in biopsied tissues in which expression of SIGP may be correlated with disease. The diagnostic assay may be used to determine absence, presence, and excess expression of SIGP, and to monitor regulation of SIGP levels during therapeutic intervention.

In one aspect, hybridization with PCR probes which are capable of detecting polynucleotide sequences, including genomic sequences, encoding SIGP or closely related molecules may be used to identify nucleic acid sequences which encode SIGP. The specificity of the probe, whether it is made from a highly specific region, e.g., the 5′ regulatory region, or from a less specific region, e.g., a conserved motif, and the stringency of the hybridization or amplification (maximal, high, intermediate, or low), will determine whether the probe identifies only naturally occurring sequences encoding SIGP, alleles, or related sequences.

Probes may also be used for the detection of related sequences, and should preferably contain at least 50% of the nucleotides from any of the SIGP encoding sequences. The hybridization probes of the subject invention may be DNA or RNA and may be derived from the sequence of SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO: 114, SEQ ID NO: 115, SEQ ID NO:116, SEQ ID NO:117, SEQ ID NO: 118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, and SEQ ID NO:154, or from genomic sequences including promoters, enhancers, and introns of the SIGP gene.

Means for producing specific hybridization probes for DNAs encoding SIGP include the cloning of polynucleotide sequences encoding SIGP or SIGP derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotide sequences encoding SIGP may be used for the diagnosis of a disorder associated with either increased or decreased expression of SIGP. Examples of such a disorder include, but are not limited to, cancers such as adenocarcinoma, leukemia, lymphoma, melanoma, myeloma, sarcoma, teratocarcinoma, and cancers of the adrenal gland, bladder, bone, brain, breast, cervix, gall bladder, ganglia, gastrointestinal tract, heart, kidney, liver, lung, bone marrow, muscle, ovary, pancreas, parathyroid, penis, prostate, salivary glands, skin, spleen, testis, thymus, thyroid, and uterus; neuronal disorders such as akathesia, Alzheimer's disease, amnesia, amyotrophic lateral sclerosis, bipolar disorder, catatonia, cerebral neoplasms, dementia, depression, Down's syndrome, tardive dyskinesia, dystonias, epilepsy, Huntington's disease, multiple sclerosis, neurofibromatosis, Parkinson's disease, paranoid psychoses, schizophrenia, and Tourette's disorder; and immunological disorders such as AIDS, Addison's disease, adult respiratory distress syndrome, allergies, anemia, asthma, atherosclerosis, bronchitis, cholecystitus, Crohn's disease, ulcerative colitis, atopic dermatitis, dermatomyositis, diabetes mellitus, emphysema, atrophic gastritis, glomerulonephritis, gout, Graves' disease, hypereosinophilia, irritable bowel syndrome, lupus erythematosus, multiple sclerosis, myasthenia gravis, myocardial or pericardial inflammation, osteoarthritis, osteoporosis, pancreatitis, polymyositis, rheumatoid arthritis, scleroderma, Sjogren's syndrome, and thyroiditis. The polynucleotide sequences encoding SIGP may be used in Southern or northern analysis, dot blot, or other membrane-based technologies; in PCR technologies; in dipstick, pin, and ELISA assays; and in microarrays utilizing fluids or tissues from patients to detect altered SIGP expression. Such qualitative or quantitative methods are well known in the art.

In a particular aspect, the nucleotide sequences encoding SIGP may be useful in assays that detect the presence of associated disorders, particularly those mentioned above. The nucleotide sequences encoding SIGP may be labeled by standard methods and added to a fluid or tissue sample from a patient under conditions suitable for the formation of hybridization complexes. After a suitable incubation period, the sample is washed and the signal is quantitated and compared with a standard value. If the amount of signal in the patient sample is significantly altered in comparison to a control sample then the presence of altered levels of nucleotide sequences encoding SIGP in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of SIGP, a normal or standard profile for expression is established. This may be accomplished by combining body fluids or cell extracts taken from normal subjects, either animal or human, with a sequence, or a fragment thereof, encoding SIGP, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Additional diagnostic uses for oligonucleotides designed from the sequences encoding SIGP may involve the use of PCR. These oligomers may be chemically synthesized, generated enzymatically, or produced in vitro. Oligomers will preferably contain a fragment of a polynucleotide encoding SIGP, or a fragment of a polynucleotide complementary to the polynucleotide encoding SIGP, and will be employed under optimized conditions for identification of a specific gene or condition. Oligomers may also be employed under less stringent conditions for detection or quantitation of closely related DNA or RNA sequences.

Methods which may also be used to quantitate the expression of SIGP include radiolabeling or biotinylating nucleotides, coamplification of a control nucleic acid, and interpolating results from standard curves. (See, e.g., Melby, P. C. et al. (1993) J. Immunol. Methods 159:235-244; and Duplaa, C. et al. (1993) Anal. Biochem. 229-236.) The speed of quantitation of multiple samples may be accelerated by running the assay in an ELISA format where the oligomer of interest is presented in various dilutions and a spectrophotometric or colorimetric response gives rapid quantitation.

In further embodiments, oligonucleotides or longer fragments derived from any of the polynucleotide sequences described herein may be used as targets in a microarray. The microarray can be used to monitor the expression level of large numbers of genes simultaneously and to identify genetic variants, mutations, and polymorphisms. This information may be used to determine gene function, to understand the genetic basis of a disorder, to diagnose a disorder, and to develop and monitor the activities of therapeutic agents.

In one embodiment, the microarray is prepared and used according to methods known in the art. (See, e.g., Chee et al. (1995) PCT application WO95/11995; Lockhart, D. J. et al. (1996) Nat. Biotech. 14:1675-1680; and Schena, M. et al. (1996) Proc. Natl. Acad. Sci. 93:10614-10619.)

The microarray is preferably composed of a large number of unique single-stranded nucleic acid sequences, usually either synthetic antisense oligonucleotides or fragments of cDNAs. The oligonucleotides are preferably about 6 to 60 nucleotides in length, more preferably about 15 to 30 nucleotides in length, and most preferably about 20 to 25 nucleotides in length. It may be preferable to use oligonucleotides which are about 7 to 10 nucleotides in length. The microarray may contain oligonucleotides which cover the known 5′ or 3′ sequence, sequential oligonucleotides which cover the full length sequence, or unique oligonucleotides selected from particular areas along the length of the sequence. Polynucleotides used in the microarray may be oligonucleotides specific to a gene or genes of interest. Oligonucleotides can also be specific to one or more unidentified cDNAs associated with a particular cell type or tissue type. It may be appropriate to use pairs of oligonucleotides on a microarray. The first oligonucleotide in each pair differs from the second oligonucleotide by one nucleotide. This nucleotide is preferably located in the center of the sequence. The second oligonucleotide serves as a control. The number of oligonucleotide pairs may range from about 2 to 1,000,000.

In order to produce oligonucleotides for use on a microarray, the gene of interest is examined using a computer algorithm which starts at the 5′ end, or, more preferably, at the 3′ end of the nucleotide sequence. The algorithm identifies oligomers of defined length that are unique to the gene, have a GC content within a range suitable for hybridization, and lack secondary structure that may interfere with hybridization. In one aspect, the oligomers may be synthesized on a substrate using a light-directed chemical process. (See, e.g., Chee et al., supra.) The substrate may be any suitable solid support, e.g., paper, nylon, any other type of membrane, or a filter, chip, or glass slide.

In another aspect, the oligonucleotides may be synthesized on the surface of the substrate using a chemical coupling procedure and an ink jet application apparatus. (See, e.g., Baldeschweiler et al. (1995) PCT application WO95/251116.) An array analogous to a dot or slot blot (HYBRIDOT® apparatus, GIBCO/BRL) may be used to arrange and link cDNA fragments or oligonucleotides to the surface of a substrate using a vacuum system or thermal, UV, mechanical, or chemical bonding procedures. An array may also be produced by hand or by using available devices, materials, and machines, e.g. Brinkmann® multichannel pipettors or robotic instruments. The array may contain from 2 to 1,000,000 or any other feasible number of oligonucleotides.

In order to conduct sample analysis using the microarrays, polynucleotides are extracted from a sample. The sample may be obtained from any bodily fluid, e.g., blood, urine, saliva, phlegm, gastric juices, cultured cells, biopsies, or other tissue preparations. To produce probes, the polynucleotides extracted from the sample are used to produce nucleic acid sequences complementary to the nucleic acids on the micro array. If the micro array contains cDNAs, anti sense RNAs (aRNAs) are appropriate probes. Therefore, in one aspect, mRNA is reverse-transcribed to cDNA. The cDNA, in the presence of fluorescent label, is used to produce fragment or oligonucleotide aRNA probes. The fluorescently labeled probes are incubated with the microarray so that the probes hybridize to the microarray oligonucleotides. Nucleic acid sequences used as probes can include polynucleotides, fragments, and complementary or antisense sequences produced using restriction enzymes, PCR, or other methods known in the art.

Hybridization conditions can be adjusted so that hybridization occurs with varying degrees of complementarity. A scanner can be used to determine the levels and patterns of fluorescence after removal of any nonhybridized probes. The degree of complementarity and the relative abundance of each oligonucleotide sequence on the microarray can be assessed through analysis of the scanned images. A detection system may be used to measure the absence, presence, or level of hybridization for any of the sequences. (See, e.g., Heller, R. A. et al. (1997) Proc. Natl. Acad. Sci. 94:2150-2155.)

In another embodiment of the invention, nucleic acid sequences encoding SIGP may be used to generate hybridization probes useful in mapping the naturally occurring genomic sequence. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries. (See, e.g., Price, C. M. (1993) Blood Rev. 7:127-134; and Trask, B. J. (1991) Trends Genet. 7:149-154.)

Fluorescent in situ hybridization (FISH) may be correlated with other physical chromosome mapping techniques and genetic map data. (See, e.g., Heinz-Ulrich, et al. (1995) in Meyers, R. A. (ed.) Molecular Biology and Biotechnology, VCH Publishers New York, N.Y., pp. 965-968.) Examples of genetic map data can be found in various scientific journals or at the Online Mendelian Inheritance in Man (OMIM) site. Correlation between the location of the gene encoding SIGP on a physical chromosomal map and a specific disorder, or a predisposition to a specific disorder, may help define the region of DNA associated with that disorder. The nucleotide sequences of the invention may be used to detect differences in gene sequences among normal, carrier, and affected individuals.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the number or arm of a particular human chromosome is not known. New sequences can be assigned to chromosomal arms by physical mapping. This provides valuable information to investigators searching for disease genes using positional cloning or other gene discovery techniques. Once the disease or syndrome has been crudely localized by genetic linkage to a particular genomic region, e.g., AT to 11q22-23, any sequences mapping to that area may represent associated or regulatory genes for further investigation. (See, e.g., Gatti, R. A. et al. (1988) Nature 336:577-580.) The nucleotide sequence of the subject invention may also be used to detect differences in the chromosomal location due to translocation, inversion, etc., among normal, carrier, or affected individuals.

In another embodiment of the invention, SIGP, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between SIGP and the agent being tested may be measured.

Another technique for drug screening provides for high throughput screening of compounds having suitable binding affinity to the protein of interest. (See, e.g., Geysen, et al. (1984) PCT application WO84/03564.) In this method, large numbers of different small test compounds are synthesized on a solid substrate, such as plastic pins or some other surface. The test compounds are reacted with SIGP, or fragments thereof, and washed. Bound SIGP is then detected by methods well known in the art. Purified SIGP can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding SIGP specifically compete with a test compound for binding SIGP. In this manner, antibodies can be used to detect the presence of any peptide which shares one or more antigenic determinants with SIGP.

In additional embodiments, the nucleotide sequences which encode SIGP may be used in any molecular biology techniques that have yet to be developed, provided the new techniques rely on properties of nucleotide sequences that are currently known, including, but not limited to, such properties as the triplet genetic code and specific base pair interactions.

The examples below are provided to illustrate the subject invention and are not included for the purpose of limiting the invention.

### EXAMPLES

For purposes of example, the preparation and sequencing of the SPLNNOT04 cDNA library, from which Incyte Clones 1534876 and 1559131 were isolated, is described. Preparation and sequencing of cDNAs in libraries in the LIFESEQ™ database have varied over time, and the gradual changes involved use of kits, plasmids, and machinery available at the particular time the library was made and analyzed.

I. SPLNNOT04 cDNA Library Construction

The SPLNNOT04 cDNA library was constructed from microscopically normal spleen tissue obtained from a 2-year-old Hispanic male who died of cerebral anoxia. The patient's serologies and past medical history were negative.

The frozen tissue was homogenized and lysed using a Brinkmann Homogenizer Polytron PT-3000 (Brinkmann Instruments, Westbury, N.J.) in guanidinium isothiocyanate solution. The lysate was centrifuged over a 5.7 M CsCl cushion using an Beckman SW28 rotor in a Beckman L8-70M Ultracentrifuge (Beckman Instruments) for 18 hours at 25,000 rpm at ambient temperature. The RNA was extracted with acid phenol pH 4.0, precipitated using 0.3 M sodium acetate and 2.5 volumes of ethanol, resuspended in RNAse-free water and DNase treated at 37° C. The RNA extraction and precipitation were repeated as before. The mRNA was then isolated using the Qiagen Oligotex kit (QIAGEN Inc., Chatsworth, Calif.) and used to construct the cDNA library.

The mRNA was handled according to the recommended protocols in the SuperScript plasmid system (Cat. #18248-013, GIBCO-BRL, Gaithersburg, Md.). cDNA synthesis was initiated with a NotI-oligo d(T) primer. Double-stranded cDNA was blunted, ligated to EcoRI adaptors, digested with NotI, fractionated on a Sepharose CL4B column (Cat. #275105-01, Pharmacia), and those cDNAs exceeding 400 bp were ligated into the NotI and EcoRI sites of the pINCY 1 vector (Incyte). The plasmid pINCY 1 was subsequently transformed into DH5α™ competent cells (Cat. #18258-012, GIBCO-BRL).

II Isolation and Sequencing of cDNA Clones

Plasmid cDNA was released from the cells and purified using the REAL Prep 96 plasmid kit (Catalog #26173, QIAGEN). The recommended protocol was employed except for the following changes: 1) the bacteria were cultured in 1 ml of sterile Terrific Broth (Catalog #22711, GIBCO-BRL) with carbenicillin at 25 mg/L and glycerol at 0.4%; 2) after inoculation, the cultures were incubated for 19 hours and at the end of incubation, the cells were lysed with 0.3 ml of lysis buffer; and 3) following isopropanol precipitation, the plasmid DNA pellet was resuspended in 0.1 ml of distilled water. After the last step in the protocol, samples were transferred to a 96-well block for storage at 4° C.

cDNAs were sequenced according to the method of Sanger et al. (1975, J. Mol. Biol. 94:441 f), using the Perkin Elmer Catalyst 800 or a Hamilton Micro Lab 2200 (Hamilton, Reno, Nev.) in combination with Peltier Thermal Cyclers (PTC200 from MJ Research, Watertown, Mass.) and Applied Biosystems 377 DNA Sequencing Systems or the Perkin Elmer 373 DNA Sequencing System and the reading frame was determined.

III. Homology Searching of cDNA Clones and their Deduced Proteins

The nucleotide sequences and/or amino acid sequences of the Sequence Listing were used to query sequences in the GenBank, SwissProt, BLOCKS, and Pima II databases. These databases, which contain previously identified and annotated sequences, were searched for regions of homology using BLAST (Basic Local Alignment Search Tool). (See, e.g., Altschul, S. F. (1993) J. Mol. Evol. 36:290-300; and Altschul et al. (1990) J. Mol. Biol. 215:403-410.)

BLAST produced alignments of both nucleotide and amino acid sequences to determine sequence similarity. Because of the local nature of the alignments, BLAST was especially useful in determining exact matches or in identifying homologs which may be of prokaryotic (bacterial) or eukaryotic (animal, fungal, or plant) origin. Other algorithms could have been used when dealing with primary sequence patterns and secondary structure gap penalties. (See, e.g., Smith, T. et al. (1992) Protein Engineering 5:35-51.) The sequences disclosed in this application have lengths of at least 49 nucleotides and have no more than 12% uncalled bases (where N is recorded rather than A, C, G, or T).

The BLAST approach searched for matches between a query sequence and a database sequence. BLAST evaluated the statistical significance of any matches found, and reported only those matches that satisfy the user-selected threshold of significance. In this application, threshold was set at 10⁻²⁵ for nucleotides and 10⁻⁸ for peptides.

Incyte nucleotide sequences were searched against the GenBank databases for primate (pri), rodent (rod), and other mammalian sequences (mam), and deduced amino acid sequences from the same clones were then searched against GenBank functional protein databases, mammalian (mamp), vertebrate (vrtp), and eukaryote (eukp), for homology.

### IV. Northern Analysis

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; and Ausubel, F. M. et al. supra, ch. 4 and 16.)

Analogous computer techniques applying BLAST are used to search for identical or related molecules in nucleotide databases such as GenBank or LIFESEQ™ database (Incyte Pharmaceuticals). This analysis is much faster than multiple membrane-based hybridizations. In addition, the sensitivity of the computer search can be modified to determine whether any particular match is categorized as exact or homologous.

The basis of the search is the product score, which is defined as:

% sequence identity×% maximum BLAST score/100

The product score takes into account both the degree of similarity between two sequences and the length of the sequence match. For example, with a product score of 40, the match will be exact within a 1% to 2% error, and, with a product score of 70, the match will be exact. Homologous molecules are usually identified by selecting those which show product scores between 15 and 40, although lower scores may identify related molecules.

The results of northern analysis are reported as a list of libraries in which the transcript encoding SIGP occurs. Abundance and percent abundance are also reported. Abundance directly reflects the number of times a particular transcript is represented in a cDNA library, and percent abundance is abundance divided by the total number of sequences examined in the cDNA library.

### V. Extension of SIGP Encoding Polynucleotides

The nucleic acid sequence of one of the polynucleotides of the present invention was used to design oligonucleotide primers for extending a partial nucleotide sequence to full length. One primer was synthesized to initiate extension of an antisense polynucleotide, and the other was synthesized to initiate extension of a sense polynucleotide. Primers were used to facilitate the extension of the known sequence “outward” generating amplicons containing new unknown nucleotide sequence for the region of interest. The initial primers were designed from the cDNA using OLIGO 4.06 (National Biosciences, Plymouth, Minn.), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries (GIBCO/BRL) were used to extend the sequence. If more than one extension is necessary or desired, additional sets of primers are designed to further extend the known region.

High fidelity amplification was obtained by following the instructions for the XL-PCR kit (Perkin Elmer) and thoroughly mixing the enzyme and reaction mix. PCR was performed using the Peltier Thermal Cycler (PTC200; M.J. Research, Watertown, Mass.), beginning with 40 μmol of each primer and the recommended concentrations of all other components of the kit, with the following parameters:

| | |
|---|---|
| Step 1 | 94° C. for 1 min (initial denaturation) |
| Step 2 | 65° C. for 1 min |
| Step 3 | 68° C. for 6 min |
| Step 4 | 94° C. for 15 sec |
| Step 5 | 65° C. for 1 min |
| Step 6 | 68° C. for 7 min |
| Step 7 | Repeat steps 4 through 6 for an additional 15 cycles |
| Step 8 | 94° C. for 15 sec |
| Step 9 | 65° C. for 1 min |
| Step 10 | 68° C. for 7:15 min |
| Step 11 | Repeat steps 8 through 10 for an additional 12 cycles |
| Step 12 | 72° C. for 8 min |
| Step 13 | 4° C. (and holding) |

A 5 l to 10 μl aliquot of the reaction mixture was analyzed by electrophoresis on a low concentration (about 0.6% to 0.8%) agarose mini-gel to determine which reactions were successful in extending the sequence. Bands thought to contain the largest products were excised from the gel, purified using QIAQuick™ (QIAGEN Inc., Chatsworth, Calif.), and trimmed of overhangs using Klenow enzyme to facilitate religation and cloning.

After ethanol precipitation, the products were redissolved in 13 μl of ligation buffer, 1 μl T4-DNA ligase (15 units) and 1 μl T4 polynucleotide kinase were added, and the mixture was incubated at room temperature for 2 to 3 hours, or overnight at 16° C. Competent E. coli cells (in 40 μl of appropriate media) were transformed with 3 μl of ligation mixture and cultured in 80 μl of SOC medium. (See, e.g., Sambrook, supra, Appendix A, p. 2.) After incubation for one hour at 37° C., the E. coli mixture was plated on Luria Bertani (LB) agar (See, e.g., Sambrook, supra, Appendix A, p. 1) containing 2× Carb. The following day, several colonies were randomly picked from each plate and cultured in 150 μl of liquid LB/2× Carb medium placed in an individual well of an appropriate commercially-available sterile 96-well microtiter plate. The following day, 5 μl of each overnight culture was transferred into a non-sterile 96-well plate and, after dilution 1:10 with water, 5 μl from each sample was transferred into a PCR array.

For PCR amplification, 18 μl of concentrated PCR reaction mix (3.3×) containing 4 units of rTth DNA polymerase, a vector primer, and one or both of the gene specific primers used for the extension reaction were added to each well. Amplification was performed using the following conditions:

| | |
|---|---|
| Step 1 | 94° C. for 60 sec |
| Step 2 | 94° C. for 20 sec |
| Step 3 | 55° C. for 30 sec |
| Step 4 | 72° C. for 90 sec |
| Step 5 | Repeat steps 2 through 4 for an additional 29 cycles |
| Step 6 | 72° C. for 180 sec |
| Step 7 | 4° C. (and holding) |

Aliquots of the PCR reactions were run on agarose gels together with molecular weight markers. The sizes of the PCR products were compared to the original partial cDNAs, and appropriate clones were selected, ligated into plasmid, and sequenced.

In like manner, the nucleotide sequence of one of the nucleotide sequences of the present invention were used to obtain 5′ regulatory sequences using the procedure above, oligonucleotides designed for 5′ extension, and an appropriate genomic library.

### VI. Labeling and Use of Individual Hybridization Probes

Hybridization probes derived from one of the nucleotide sequences of the present invention are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 (National Biosciences) and labeled by combining 50 pmol of each oligomer, 250 μCi of [γ-³²P] adenosine triphosphate (Amersham, Chicago, Ill.), and T4 polynucleotide kinase (DuPont NEN®, Boston, Mass.). The labeled oligonucleotides are substantially purified using a Sephadex G-25 superfine resin column (Pharmacia & Upjohn, Kalamazoo, Mich.). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN, Boston, Mass.).

The DNA from each digest is fractionated on a 0.7 percent agarose gel and transferred to nylon membranes (Nytran Plus, Schleicher & Schuell, Durham, N.H.). Hybridization is carried out for 16 hours at 40° C. To remove nonspecific signals blots are sequentially washed at room temperature under increasingly stringent conditions up to 0.1.times. saline sodium citrate and 0.5% sodium dodecyl sulfate. After XOMAT AR™ film (Kodak, Rochester, N.Y.) is exposed to the blots to film for several hours, hybridization patterns are compared visually.

### VII. Microarrays

To produce oligonucleotides for a microarray, one of the nucleotide sequences of the present invention is examined using a computer algorithm which starts at the 3′ end of the nucleotide sequence. For each, the algorithm identifies oligomers of defined length that are unique to the nucleic acid sequence, have a GC content within a range suitable for hybridization, and lack secondary structure that would interfere with hybridization. The algorithm identifies approximately 20 oligonucleotides corresponding to each nucleic acid sequence. For each sequence-specific oligonucleotide, a pair of oligonucleotides is synthesized in which the first oligonucleotides differs from the second oligonucleotide by one nucleotide in the center of the sequence. The oligonucleotide pairs can be arranged on a substrate, e.g. a silicon chip, using a light-directed chemical process. (See, e.g., Chee, supra.)

In the alternative, a chemical coupling procedure and an ink jet device can be used to synthesize oligomers on the surface of a substrate. (See, e.g., Baldeschweiler, supra.) An array analogous to a dot or slot blot may also be used to arrange and link fragments or oligonucleotides to the surface of a substrate using or thermal, UV, mechanical, or chemical bonding procedures, or a vacuum system. A typical array may be produced by hand or using available methods and machines and contain any appropriate number of elements. After hybridization, nonhybridized probes are removed and a scanner used to determine the levels and patterns of fluorescence. The degree of complementarity and the relative abundance of each oligonucleotide sequence on the microarray may be assessed through analysis of the scanned images.

### VIII. Complementary Polynucleotides

Sequences complementary to the SIGP-encoding sequences, or any parts thereof, are used to detect, decrease, or inhibit expression of naturally occurring SIGP. Although use of oligonucleotides comprising from about 15 to 30 base pairs is described, essentially the same procedure is used with smaller or with larger sequence fragments. Appropriate oligonucleotides are designed using Oligo 4.06 software and the coding sequence of SIGP. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the SIGP-encoding transcript.

### IX. Expression of SIGP

Expression of SIGP is accomplished by subcloning the cDNA into an appropriate vector and transforming the vector into host cells. This vector contains an appropriate promoter, e.g., β-galactosidase upstream of the cloning site, operably associated with the cDNA of interest. (See, e.g., Sambrook, supra, pp. 404-433; and Rosenberg, M. et al. (1983) Methods Enzymol. 101:123-138.)

Induction of an isolated, transformed bacterial strain with isopropyl beta-D-thiogalactopyranoside (IPTG) using standard methods produces a fusion protein which consists of the first 8 residues of β-galactosidase, about 5 to 15 residues of linker, and the full length protein. The signal residues direct the secretion of SIGP into bacterial growth media which can be used directly in the following assay for activity.

### X. Production of SIGP Specific Antibodies

SIGP substantially purified using PAGE electrophoresis (see, e.g., Harrington, M. G. (1990) Methods Enzymol. 182:488-495), or other purification techniques, is used to immunize rabbits and to produce antibodies using standard protocols. The SIGP amino acid sequence is analyzed using DNASTAR software (DNASTAR Inc) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art. (See, e.g., Ausubel et al. supra, ch. 11.)

Typically, the oligopeptides are 15 residues in length, and are synthesized using an Applied Biosystems Peptide Synthesizer Model 431A using fmoc-chemistry and coupled to KLH (Sigma, St. Louis, Mo.) by reaction with N-maleimidobenzoyl-N-hydroxysuccinimide ester (MBS) to increase immunogenicity. (See, e.g., Ausubel et al. supra.) Rabbits are immunized with the oligopeptide-KLH complex in complete Freund's adjuvant. Resulting antisera are tested for antipeptide activity, for example, by binding the peptide to plastic, blocking with 1% BSA, reacting with rabbit antisera, washing, and reacting with radio-iodinated goat anti-rabbit IgG.

### XI. Purification of Naturally Occurring SIGP Using Specific Antibodies

Naturally occurring or recombinant SIGP is substantially purified by immunoaffinity chromatography using antibodies specific for SIGP. An immunoaffinity column is constructed by covalently coupling anti-SIGP antibody to an activated chromatographic resin, such as CNBr-activated Sepharose (Pharmacia & Upjohn). After the coupling, the resin is blocked and washed according to the manufacturer's instructions.

Media containing SIGP are passed over the immunoaffinity column, and the column is washed under conditions that allow the preferential absorbance of SIGP (e.g., high ionic strength buffers in the presence of detergent). The column is eluted under conditions that disrupt antibody/SIGP binding (e.g., a buffer of pH 2 to pH 3, or a high concentration of a chaotrope, such as urea or thiocyanate ion), and SIGP is collected.

XII. Identification of Molecules which Interact with SIGP

SIGP, or biologically active fragments thereof, are labeled with ¹²⁵I Bolton-Hunter reagent. (See, e.g., Bolton et al. (1973) Biochem. J. 133:529.) Candidate molecules previously arrayed in the wells of a multi-well plate are incubated with the labeled SIGP, washed, and any wells with labeled SIGP complex are assayed. Data obtained using different concentrations of SIGP are used to calculate values for the number, affinity, and association of SIGP with the candidate molecules.

Various modifications and variations of the described methods and systems of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1.-23. (canceled)

24. An isolated polynucleotide encoding a polypeptide comprising an amino acid sequence having at least about 95% sequence identity to an amino acid sequence of SEQ ID NO: 51.

25. The isolated polynucleotide of claim 24, wherein the polypeptide comprises the amino acid sequence of SEQ ID NO: 51.

26. The isolated polynucleotide of claim 25, comprising a polynucleotide sequence of SEQ ID NO: 128.

27. A recombinant polynucleotide comprising a promoter sequence operably linked to the polynucleotide of claim 24.

28. An isolated cell transformed with the recombinant polynucleotide of claim 27.

29. A method of producing the polypeptide encoded by the polynucleotide of claim 24, the method comprising:
a) culturing a cell under conditions suitable for expression of the polypeptide, wherein said cell is transformed with a recombinant polynucleotide, and said recombinant polynucleotide comprise a promoter sequence operably linked to the polynucleotide of claim 24, and
b) recovering the polypeptide so expressed.

30. The method of claim 29, wherein the polypeptide comprises the amino acid sequence of SEQ ID NO: 51.

31. The method of claim 29, wherein the recombinant polynucleotide comprises the polynucleotide sequence of SEQ ID NO: 128.

32. An isolated polynucleotide comprising a polynucleotide sequence selected from the group consisting of:
a) a polynucleotide sequence having at least about 95% sequence identity to a polynucleotide sequence of SEQ ID NO: 128;
b) a polynucleotide sequence complementary to the polynucleotide sequence of a); and
c) an RNA equivalent of the polynucleotide sequence of a) or b).

33. The isolated polynucleotide of claim 32, comprising the polynucleotide sequence of SEQ ID NO: 128.

34. An isolated polypeptide comprising an amino acid sequence having at least about 95% sequence identity to an amino acid sequence of SEQ ID NO: 51.

35. The isolated polypeptide of claim 34, comprising the amino acid sequence of SEQ ID NO: 51.

36. The isolated polypeptide of claim 34, comprising a fragment of the amino acid sequence of SEQ ID NO: 51.

37. A composition comprising the polypeptide of claim 34, and a pharmaceutically acceptable excipient.

38. A method of screening a compound for effectiveness as an agonist of the polypeptide of claim 34, the method comprising:
a) exposing a sample comprising the polypeptide of claim 34 to the compound, and
b) detecting agonist activity in the sample.

39. A method of screening a compound for effectiveness as an antagonist of the polypeptide of claim 34, the method comprising:
a) exposing a sample comprising the polypeptide of claim 34 to the compound, and
b) detecting antagonist activity in the sample.

40. A method of screening for a compound that specifically binds to the polypeptide of claim 34, the method comprising:
a) combining the polypeptide of claim 34 with at least one test compound under suitable conditions, and
b) detecting binding of the polypeptide of claim 34 to the test compound, thereby identifying a compound that specifically binds to the polypeptide of claim 34.

41. An antibody or fragment thereof which specifically binds to the polypeptide of claim 34.
